(19) United States
(12) Patent Application Publication
(10) Pub. No.: US 2024/0110191 A1
(43) Pub. Date: Apr. 4, 2024

(54) ENGINEERED MICROORGANISMS

(71) Applicant: Synlogic Operating Company, Inc.

(72) Inventors: Michael James, Cambridge, MA (US); Aida Kalantari, Cambridge, MA (US); Tea Mirabella, Cambridge, MA (US); Lauren Renaud, Cambridge, MA (US); Lee Kristensen, Cambridge, MA (US); Vincent Isabella, Cambridge, MA (US)

(73) Assignee: Synlogic Operating Company, Inc., Cambridge, MA (US)

(21) Appl. No.: 18/265,002

(22) PCT Filed: Dec. 2, 2021
(86) PCT No.: PCT/US2021/061579

§ 371 Date: Jun. 2, 2023

Related U.S. Application Data
(60) Provisional application No. 63/120,674, filed on Dec. 2, 2020.

(51) Int. Cl. C12N 15/52; C07K 14/245

(52) U.S. Cl. CPC C12N 15/52; C07K 14/245; C12R 2001/19

(57) ABSTRACT

The present invention relates to engineered microorganisms, wherein the engineered microorganisms comprise a therapeutic molecule and a modified pks island.

### BACKGROUND

Cyclomodulins are microorganismal toxins that are capable of interfering with the eukaryotic cell cycle. Fais 2018. Colibactin is a cyclomodulin that is synthetized by enzymes encoded by the pks genomic island. Id. The pks genomic island is “highly conserved” in Enterobacteriaceae. Id. In Escherichia coli, a 54-kilobase pks genomic island contains 19 genes, clbA to clbS, and encodes various enzymes that have been described as an “assembly line responsible for colibactin synthesis.” Id. The pks genomic island assembly line for colibactin synthesis includes three polyketide synthases (ClbC, ClbI, ClbO), three non-ribosomal peptide synthases (ClbH, ClbJ, ClbN), two hybrid non-ribosomal peptide/polyketide synthases (ClbB, ClbK), and nine accessory, tailoring, and editing proteins. The polyketide synthases, non-ribosomal peptide synthases, and hybrid enzymes “are usually organized in mega-complexes as an assembly line, in which the synthesized compound is transferred from one enzymatic module to the following one.” Id. Colibactin undergoes a prodrug activation mechanism that incorporates an N-terminal structural motif, which is removed during the final stage of biosynthesis.

The pks genomic island is found in various microorganisms such as members of the Enterobacteriaceae family, e.g., Escherichia, Klebsiella, Enterobacter, and Citrobacter. Id. Microorganisms such as Escherichia coli containing the pks genomic island have been identified in the gut microbiota as commensal bacteria as well as in disease states. Id. Colibactin-producing microorganisms such as Escherichia coli have been reported as overrepresented in colorectal cancer. Id. Colibactin-producing microorganisms are capable of inducing chromosomal instability, cell cycle arrest, DNA damage, epithelial cell senescence, and/or immune cell apoptosis. Id. However, colibactin-producing microorganisms have also been reported to exhibit anti-inflammatory, antibiotic, and analgesic effects. Id. In some models, a functional pks genomic island is important for probiotic microorganisms such as Escherichia coli Nissle (a strain characterized by its harmlessness and GRAS (generally recognized as safe) status) to exert anti-inflammatory effects. Id., Schultz 2008, Reister 2014. In colibactin-producing microorganisms, the production of colibactin may help promote bacterial growth and maintenance.

Microorganisms, including engineered microorganisms, may have therapeutic uses. As early as in 1917, for example, Escherichia coli Nissle was packaged into medicinal capsules, called Mutaflor, for treating gut conditions. Ukena 2007. These microorganisms may be impacted by the “pleiotropic effects” of colibactin, which include potentially beneficial and potentially deleterious effects. Rais 2018. Thus, there is a significant unmet need for effective microorganisms, including engineered microorganisms, in which potentially deleterious effect(s) of colibactin are reduced while potentially beneficial effect(s) of the microorganism are maintained.

### SUMMARY

The disclosure provides an engineered microorganism, e.g., an engineered bacterium, comprising a modified pks island, and compositions and formulations thereof. In some embodiments, the disclosure provides methods for treating a subject suffering from a disease or disorder by administering an engineered microorganism, e.g., an engineered bacterium, comprising a modified pks island, and compositions and formulations thereof. In some embodiments, the engineered microorganism, e.g., an engineered bacterium, comprising a modified pks island further comprises gene(s) for producing a therapeutic molecule. In some embodiments, the therapeutic molecule is capable of reducing one or more deleterious molecules in the subject, e.g., deleterious phenylalanine, deleterious ammonia, deleterious oxalate, deleterious methionine or deleterious leucine. In some embodiments, the engineered microorganism, e.g., an engineered bacterium, comprising a modified pks island further comprises gene(s) for producing a phenylalanine metabolizing enzyme (PME); or comprises gene(s) for producing an oxalate catabolism enzyme; or comprises gene(s) encoding a modified arginine biosynthesis pathway, e.g., deleted arginine repressor, modified arginine repressor binding sites, and/or arginine feedback resistant N-acetylglutamate synthase mutation; or comprises gene(s) for producing an anti-cancer molecule, e.g., a deadenylate cyclase gene or an enzyme capable of producing a stimulator of interferon gene agonist; or comprises an oxalate metabolic enzyme, e.g., acetyl-CoA:oxalate CoA transferase. In some embodiments, the microorganism comprising a modified pks island is capable of reducing hyperphenylalaninemia in a subject and/or treating a disease or disorder associated with hyperphenylalaninemia, e.g., phenylketonuria (PKU); or is capable of reducing oxalate in a subject and/or treating a disease or disorder associated with hyperoxaluria (HOX); or is capable of reducing excess ammonia in a subject and/or treating a disease or disorder associated with hyperammonemia, e.g., a urea cycle disorder (UCD); or is capable of producing an anti-cancer molecule, e.g., a deadenylate cyclase or an enzyme capable of producing a stimulator of interferon gene (STING) agonist, and/or treating cancer.

In some embodiments, the engineered microorganism, e.g., an engineered bacterium, comprising a modified pks island further comprises gene(s) for producing a phenylalanine metabolizing enzyme (PME); or comprises gene(s) for producing a methionine catabolism enzyme, e.g., methionine gamma lyase (MGL) or methionine decarboxylase (MDC); or comprises gene(s) for producing an oxalate metabolic enzyme, e.g., scaaE3, frc, and oxdC; or comprises gene(s) for producing a uric acid catabolism enzyme; or comprises gene(s) for producing a leucine catabolism enzyme, e.g., a decarboxylase enzyme; or comprises gene(s) for producing enzymes for the production of lactate. In some embodiments, the microorganism comprising a modified pks island is capable of reducing the levels of methionine in a subject and or is capable of treating diseases associated with amino acid metabolism, such as homocystinuria, cancer, and metabolic syndromes/diseases; or is capable of reducing the levels of uric acid in a subject and/or is capable of treating diseases associated with uric acid, such as hyperuricemia or gout; or is capable of reducing the levels of leucine in a subject and/or is capable of treating a metabolic disorder associated with the abnormal catabolism of branched chain amino acids in subjects, such as isovaleric acidemia, propionic acidemia, methylmalonic acidernia, maple syrup urine disease (MSUD), and diabetes ketoacidosis and other disorders; or is capable of elevating levels in a subject and/or is capable of treating diseases associated with inflammation in the gut, including but not limited to ulcerative colitis and Crohn's disease.

In some embodiments, the engineered microorganism, e.g., an engineered bacterium, comprising a modified pks island further comprises gene(s) for producing a methionine catabolism enzyme, e.g., methionine gamma lyase (MGL) or methionine decarboxylase (MDC), and a gene for producing a methionine transporter, e.g., metP. In some embodiments, the engineered microorganism, e.g., an engineered bacterium, comprising a modified pks island further comprises gene(s) for producing an oxalate metabolic enzyme, e.g., scaaE3, frc, and oxdC, and a gene for producing an oxalate transporter, e.g., oxlT.

In some embodiments, the engineered microorganism, e.g., an engineered bacterium, comprising the modified pks island produces less colibactin as compared a control microorganism comprising the native or unmodified pks island. In some embodiments, the engineered microorganism, e.g., an engineered bacterium, is less genotoxic compared a control microorganism comprising the native or unmodified pks island. Methods of detecting colibactin precursor(s) and metabolite(s) are also provided herein. In some embodiments, a measurement of a colibactin precursor or metabolite provides a proxy for colibactin levels. In some embodiments, the method detects N-myristoyl-D-asparagine.

In some embodiments, the engineered microorganism, e.g., an engineered bacterium, comprises a modification, e.g., a partial or full deletion, in one or more of the clb genes in the pks island, e.g., as compared to the microorganism's native clb gene(s). In some embodiments, the engineered microorganism, e.g., an engineered bacterium, comprises a modification, e.g., a partial or full deletion, in one or more of clbA, clbB, clbC, clbD, clbE, clbF, clbG, clbH, clbI, clbJ, clbK, clbL, clbM, clbN, clbO, clbP, clbQ, clbR, and clbS or operably linked promoter(s) thereof, e.g., as compared to the microorganism's native clb gene(s) and operably linked promoter(s). In some embodiments, the engineered microorganism, e.g., an engineered bacterium, comprises a modification, e.g., a partial or full deletion, in one or more of clbA, clbB, clbC, clbD, clbE, clbF, clbG, clbH, clbI, clbJ, clbK, clbL, clbM, clbN, clbO, clbP, clbQ, and clbR, or operably linked promoter(s) thereof, e.g., as compared to the microorganism's native clb gene(s) and operably linked promoter(s). In some embodiments, the engineered microorganism, e.g., an engineered bacterium, comprises a modification, e.g., a partial or full deletion, in each of clbA, clbB, clbC, clbD, clbE, clbF, clbG, clbH, clbI, clbJ, clbK, clbL, clbM, clbN, clbO, clbP, clbQ, and clbR, or operably linked promoter(s) thereof, e.g., as compared to the microorganism's native clb gene(s) and operably linked promoter(s). In some embodiments, the engineered microorganism, e.g., an engineered bacterium, comprises a partial deletion of each of clbA, clbB, clbC, clbD, clbE, clbF, clbG, clbH, clbI, clbJ, clbK, clbL, clbM, clbN, clbO, clbP, clbQ, and clbR, and operably linked promoter(s) thereof, and produces less colibactin as compared to a microorganism comprising the native clb gene(s) and operably linked promoters(s). In some embodiments, the engineered microorganism, e.g., genetically engineered bacterium, comprises a full deletion of each of clbA, clbB, clbC, clbD, clbE, clbF, clbG, clbH, clbI, clbJ, clbK, clbL, clbM, clbN, clbO, clbP, clbQ, and clbR, and operably linked promoter(s) thereof, and produces less colibactin as compared a microorganism comprising the native clb gene(s) and operably linked promoters(s). In some embodiments, the microorganism retains the native or unmodified clbS gene, optionally with a partial or full deletion of the operably linked promoter.

In another aspect, the present disclosure provides a pharmaceutical composition comprising the engineered microorganism as provided herein.

In another aspect, the present disclosure provides a method of reducing a deleterious molecule comprising administering the pharmaceutical composition as provided herein to a subject in need thereof.

In another aspect, the present disclosure provides a method of reducing a deleterious molecule comprising administering the pharmaceutical composition as provided herein to a subject in need thereof.

In another aspect, the present disclosure provides a method of reducing oxalate comprising administering the pharmaceutical composition as provided herein to a subject in need thereof, wherein the therapeutic molecule is a oxalate metabolic enzyme.

In another aspect, the present disclosure provides a method of treating HOX comprising administering the pharmaceutical composition as provided herein to a subject in need thereof, wherein the therapeutic molecule is a oxalate metabolic enzyme.

In another aspect, the present disclosure provides a method of reducing phenylalanine comprising administering the pharmaceutical composition as provided herein to a subject in need thereof, wherein the therapeutic molecule is a phenylalanine metabolizing enzyme.

In another aspect, the present disclosure provides a method of treating hyperphenylalaninemia comprising administering the pharmaceutical composition as provided herein to a subject in need thereof, wherein the therapeutic molecule is a phenylalanine metabolizing enzyme.

In another aspect, the present disclosure provides a method of treating PKU comprising administering the pharmaceutical composition as provided herein to a subject in need thereof, wherein the therapeutic molecule is a phenylalanine metabolizing enzyme.

In another aspect, the present disclosure provides a method of reducing methionine levels comprising administering the pharmaceutical composition as provided herein to a subject in need thereof, wherein the therapeutic molecule is a methionine metabolizing enzyme.

In another aspect, the present disclosure provides a method of treating homocysteinuria comprising administering the pharmaceutical composition as provided herein to a subject in need thereof, wherein the therapeutic molecule is a methionine metabolizing enzyme.

In another aspect, the present disclosure provides a method of assessing pks island activity comprising detecting N-myristoyl-D-asparagine using LC-MS/MS.

### BRIEF DESCRIPTION OF THE DRAWINGS

FIG. 1A and FIG. 1B show an exemplary pharmacokinetic profile of a Δpks strain in mouse. The Δpks E. coli Nissle (EcN) strain contains deletions in the clb sequences of the clbA-clbR genes and operably linked promoters, with the clbS gene sequence unmodified (intact) but its operably linked promoter deleted.

FIG. 2 shows an exemplary pharmacokinetic profile of the Δpks strain (deleted clbA-clbR gene and promoter sequences, intact clbS gene sequence with the operably linked promoter deleted) in Non-Human Primate.

FIGS. 3A and B show schematics of an exemplary pks island. FIG. 3A depicts an intact pks island. FIG. 3B depicts an exemplary deletion. The deletion is demarcated by dashed lines.

FIG. 4 shows exemplary clb nucleotide sequences of the pks island. In one embodiment, an engineered bacterium contains deletions in all of these clb sequences except for the clbS sequence.

FIG. 5 shows exemplary clb amino acid sequences of the pks island. In one embodiment, an engineered bacterium contains deletions in all of these clb sequences except for the clbS sequence.

FIG. 6 shows in vitro growth and competition rates for an exemplary Δpks strain (deleted clbA-clbR gene and promoter sequences, intact clbS gene sequence with the operably linked promoter deleted; called Δclb here (Δclb in this figure refers to the deletion of all genes required for production of colibactin).

FIG. 7 depicts a bar graph showing the average rate of in vitro TCA production in bacterial supernatants of parental strains SYNB1618 and SYNB1934 having intact clb sequences and in SYNB1618- and SYNB1934-derivative strains having a Δpks deletion. SYN8260 and SYN8261 are clones derived from parental clone SYNB1618 and having Δpks. SYN8256 is a clone derived from parental clone SYNB1934 and having Δpks.

FIG. 8 depicts a graph showing levels of a precursor of colibactin present in supernatants from overnight cultures and shake flask cultures of parental strains SYNB1618 and SYNB1934, having intact clb sequences, and SYNB1618 and SYNB1618-derivative strains having a Δpks deletion.

FIGS. 9A-C depicts graphs showing a time course of plasma levels of phenylalanine (Phe) (9A) and trancinnamic acid (TCA) (9B), and urinary hippurate (HA) at 6 hours post dose (9C) in NHPs upon administration of SYNB1934 and a SYNB1934 derivative strain having Δpks (SYNB1934v1, also referred to herein as SYN8256).

FIG. 10 depicts exemplary genetically engineered bacterium SYNB1618 for reducing hyperphenylalaninemia and treating disorders characterized by hyperphenylalaninemia.

FIG. 11 depicts exemplary genetically engineered bacterium SYNB1934 for reducing hyperphenylalaninemia and treating disorders characterized by hyperphenylalaninemia.

### DETAILED DESCRIPTION

The present disclosure relates to, inter alia, engineered microorganisms, e.g., engineered bacteria, comprising a modified pks island as compared to a suitable control, e.g., the native pks island in an unmodified bacterium of the same strain and/or subtype, and optionally one or more gene(s) for producing a desired therapeutic molecule, and compositions, formulations, and methods of use thereof.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, “pks genomic island” and “pks island” are used to refer to a genomic region in a microorganism, e.g., bacterium, that comprises sequences encoding proteins capable of synthesizing colibactin. The pks island contains clb sequences, e.g., clb genes and associated regulatory elements, e.g., operably linked promoters. In Escherichia coli, the 54-kilobase pks genomic island contains 19 genes, clbA to clbS, and encodes three polyketide synthases (ClbC, ClbI, ClbO), three non-ribosomal peptide synthases (ClbH, ClbJ, ClbN), two hybrid non-ribosomal peptide/polyketide synthases (ClbB, ClbK), and nine accessory, tailoring, and editing proteins. Exemplary proteins encoded by an exemplary pks island is shown in Table 1. See Fais 2018, the contents of which are hereby incorporated by reference. In some embodiments, the pks island refers to a 54-kb genomic region comprising the following genes and operably linked promoters: clbA, clbB, clbC, clbD, clbE, clbF, clbG, clbH, clbI, clbJ, clbK, clbL, clbM, clbN, clbO, clbP, clbQ, clbR, or clbS. The pks island is found in various microorganisms such as members of the Enterobacteriaceae family, e.g., Escherichia, Klebsiella, Enterobacter, and Citrobacter. Id. The pks island is “highly conserved” in Enterobacteriaceae. Id.

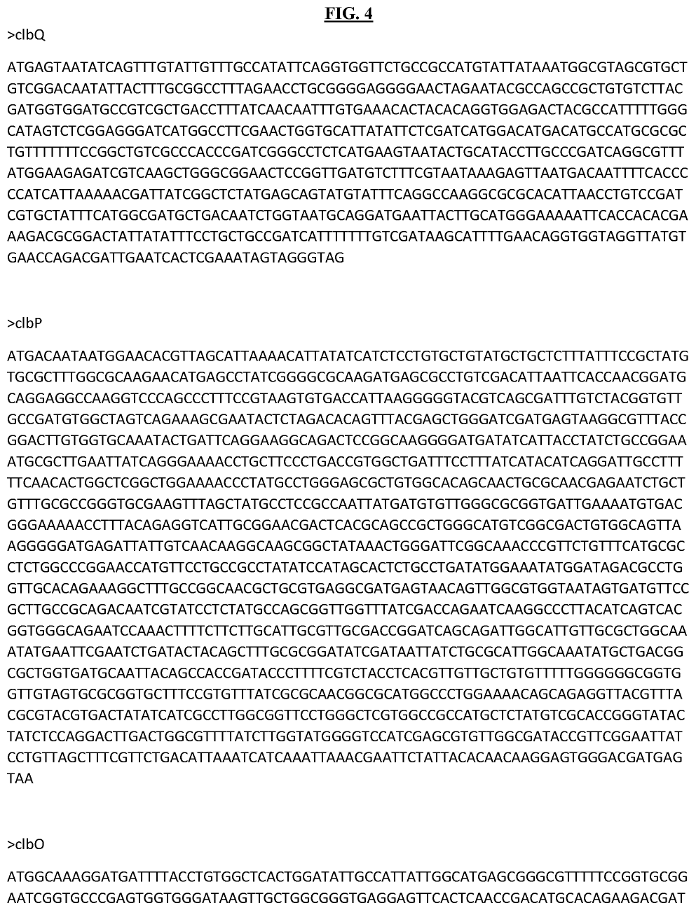

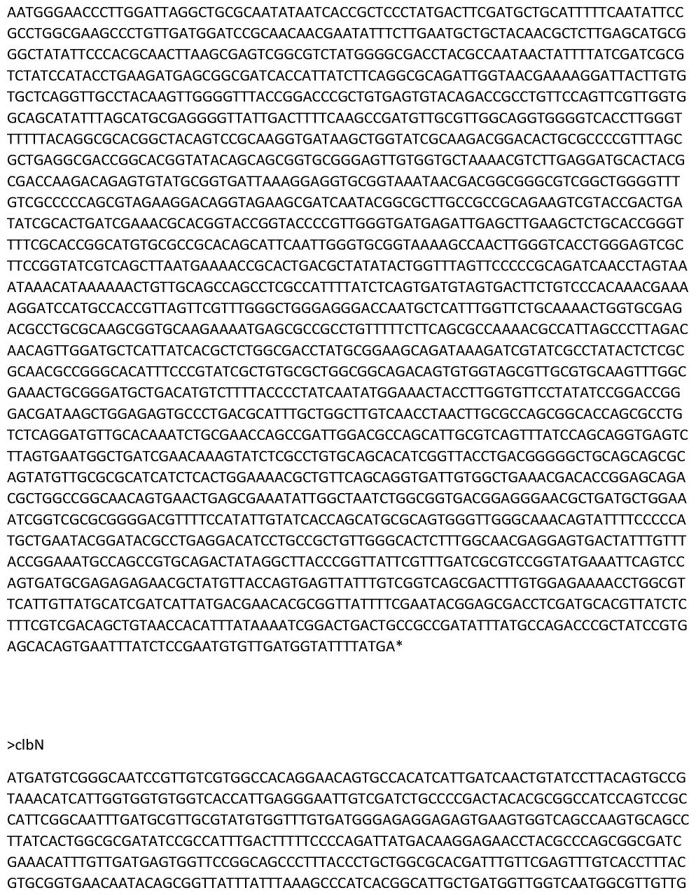

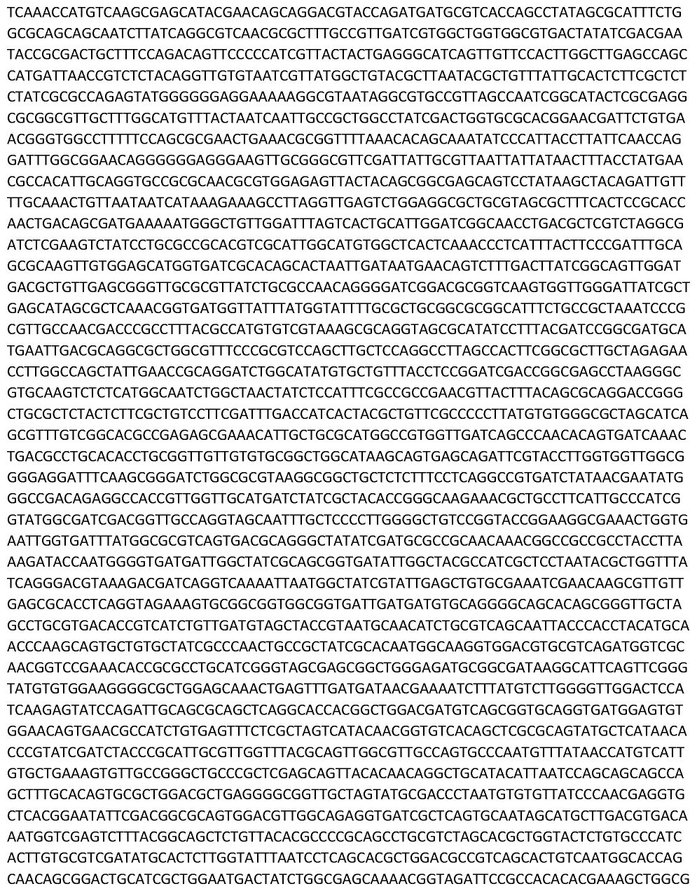

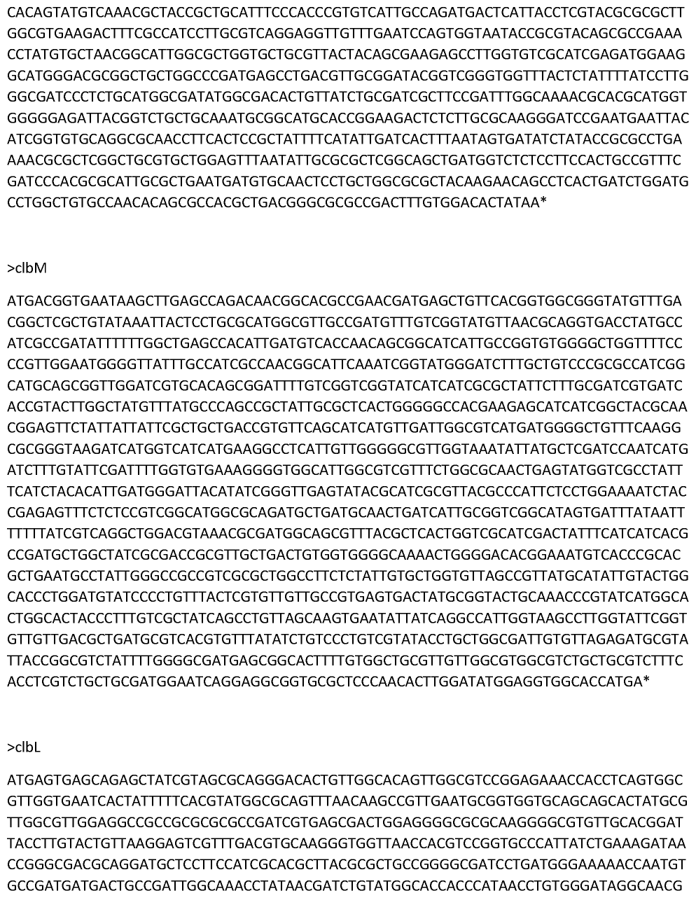

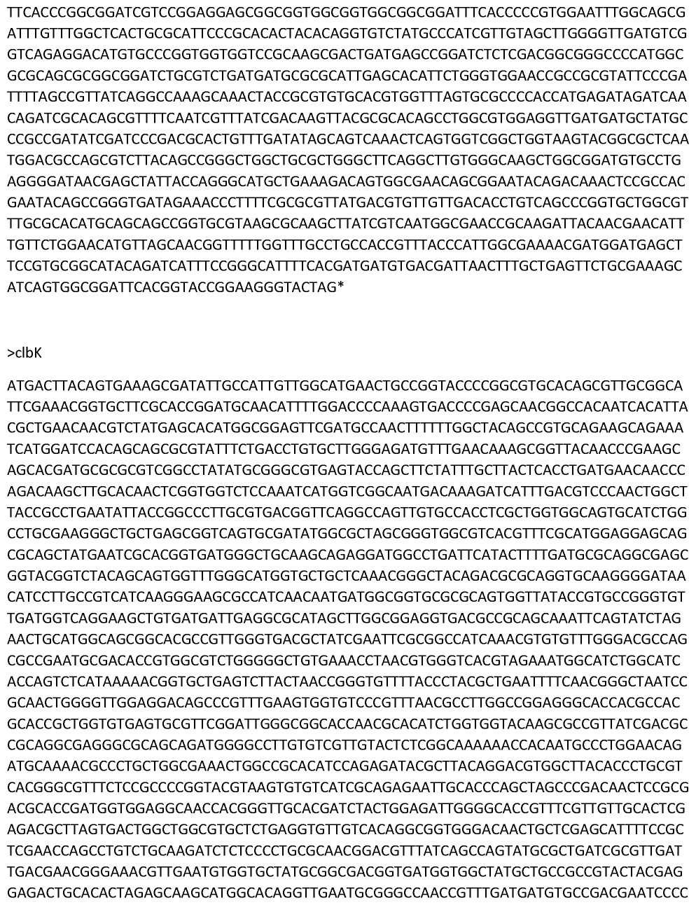

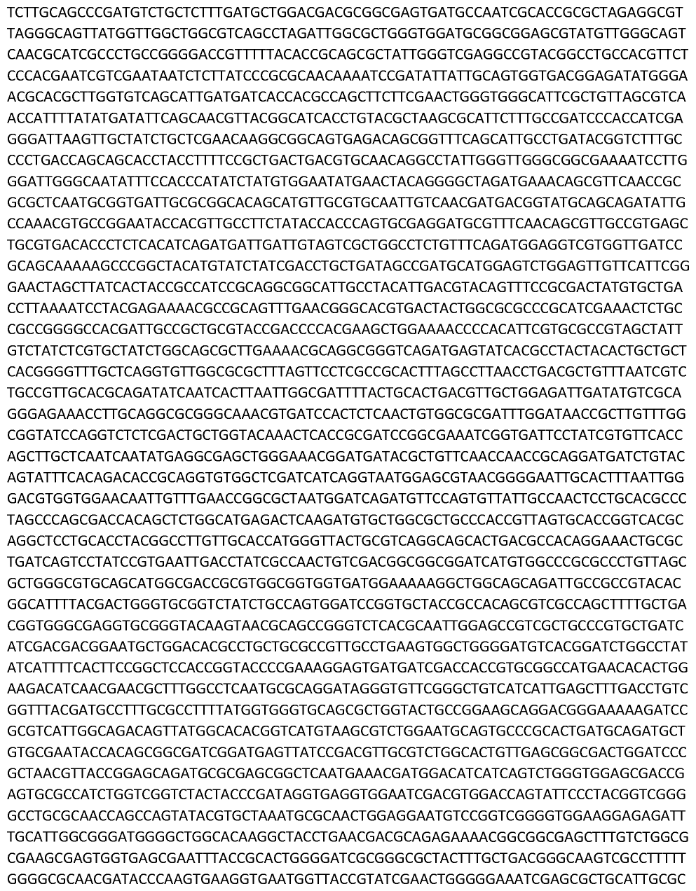

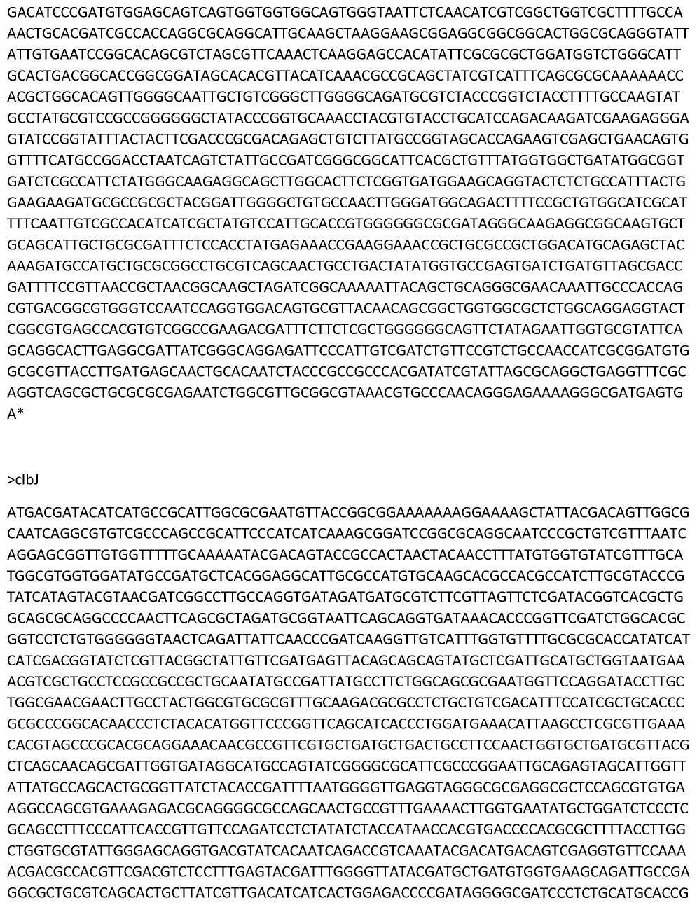

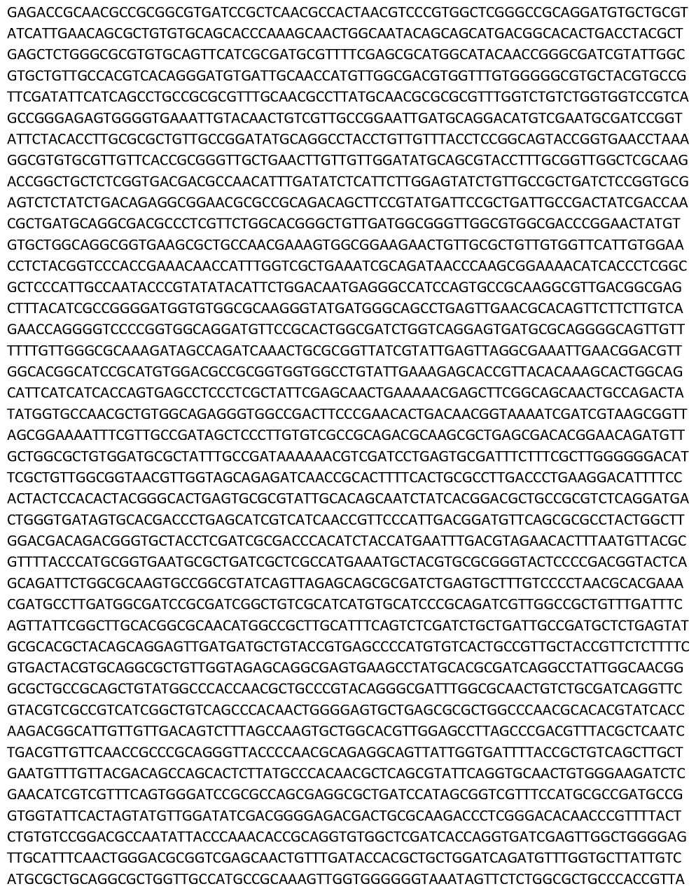

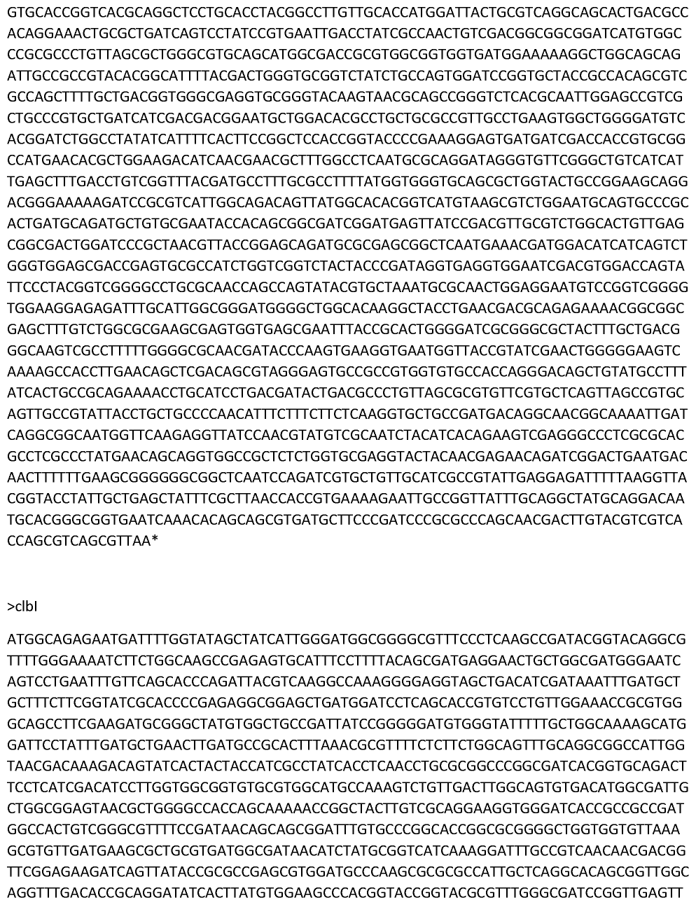

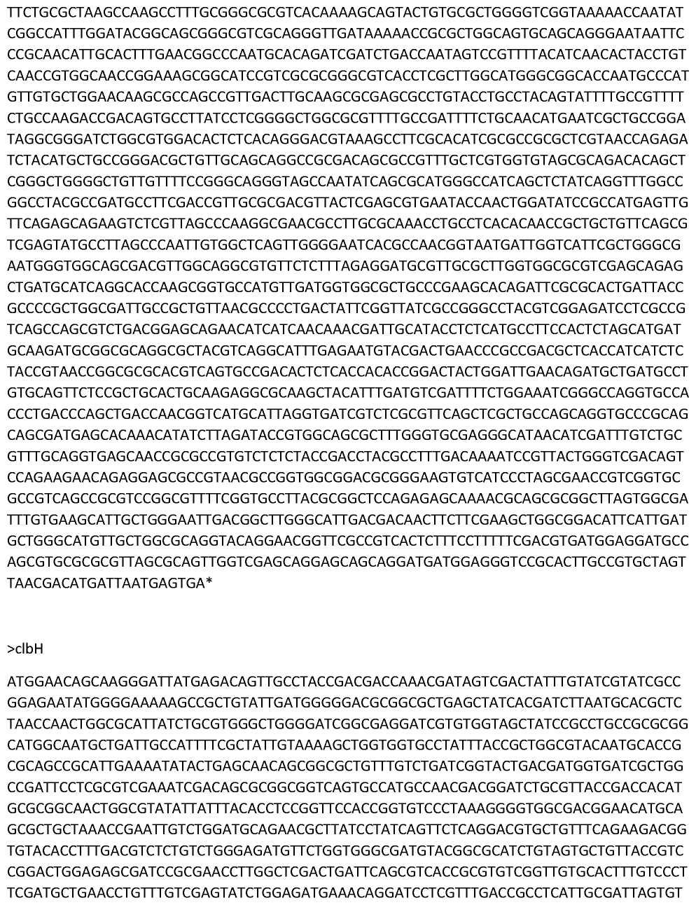

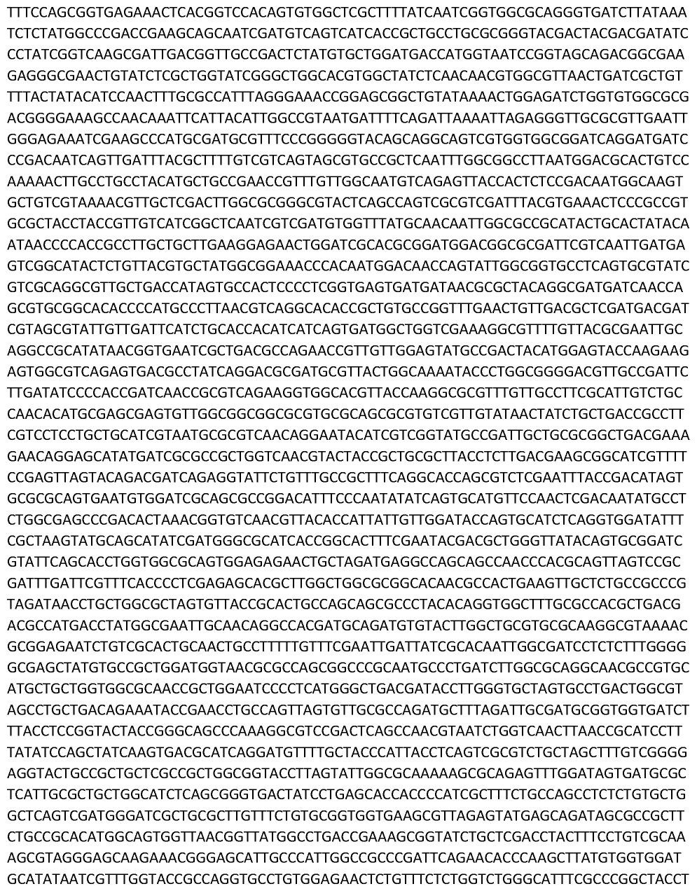

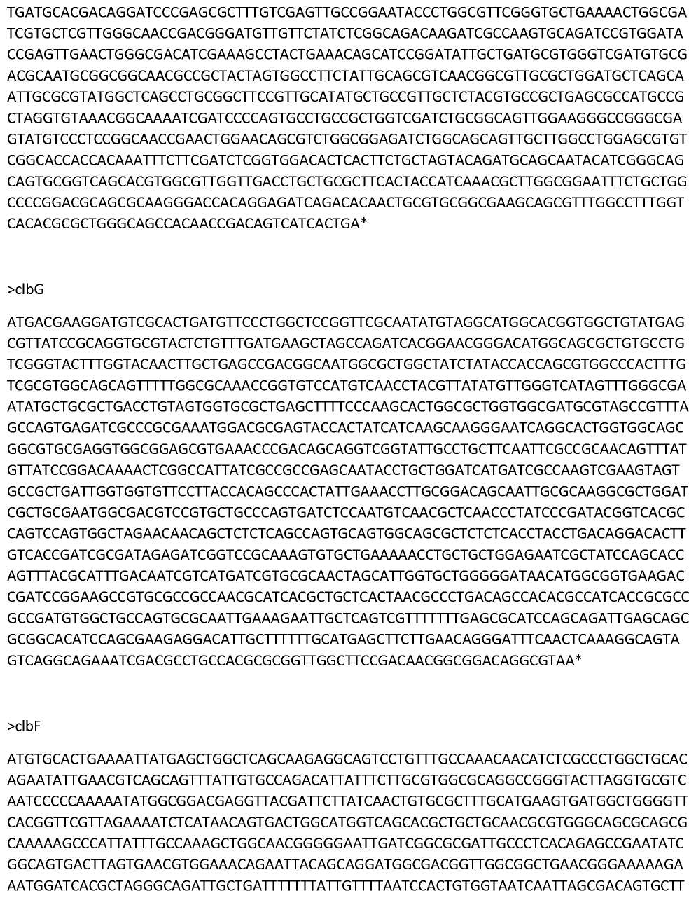

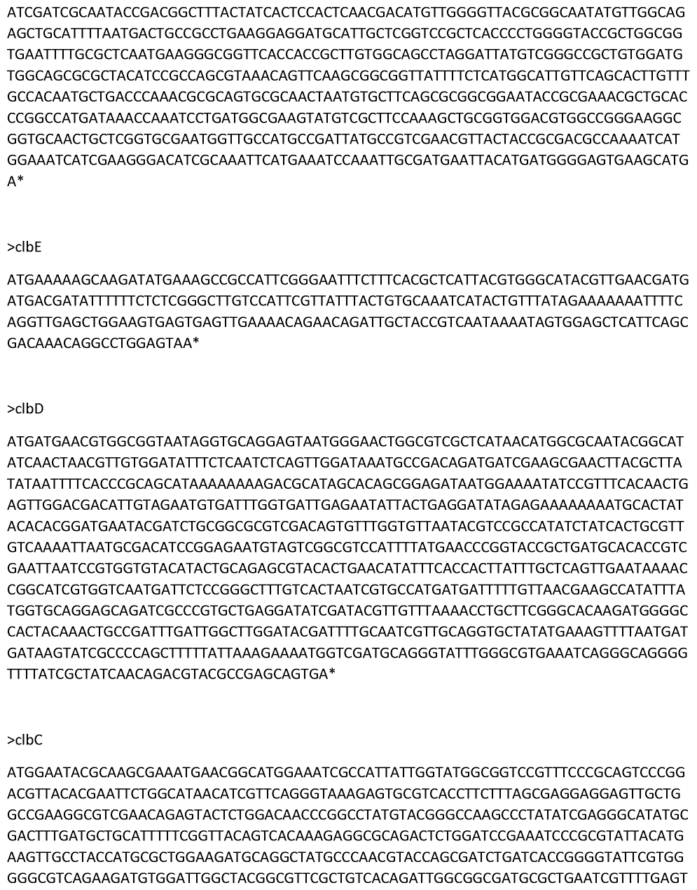

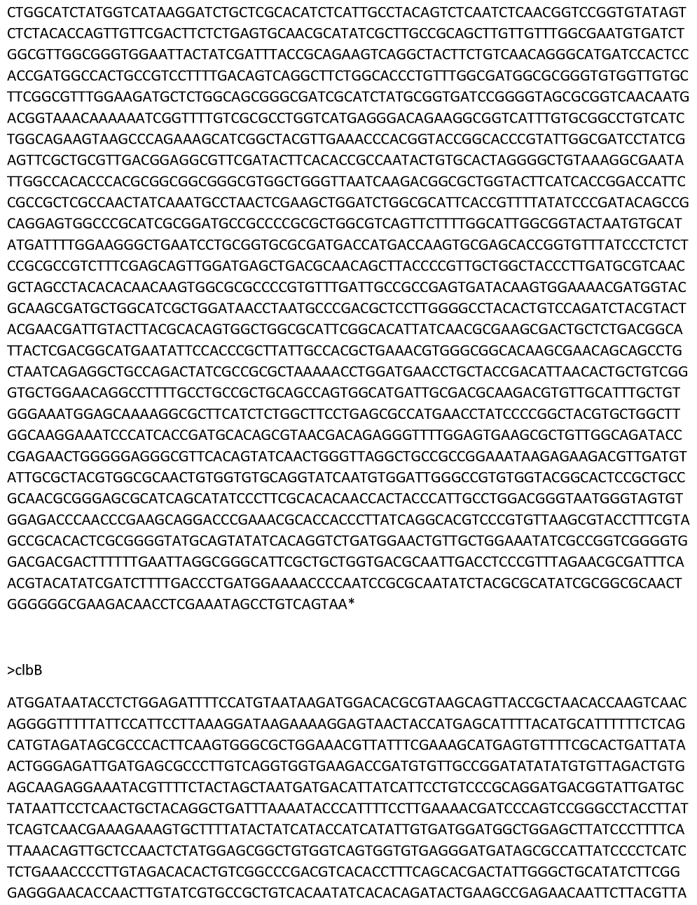

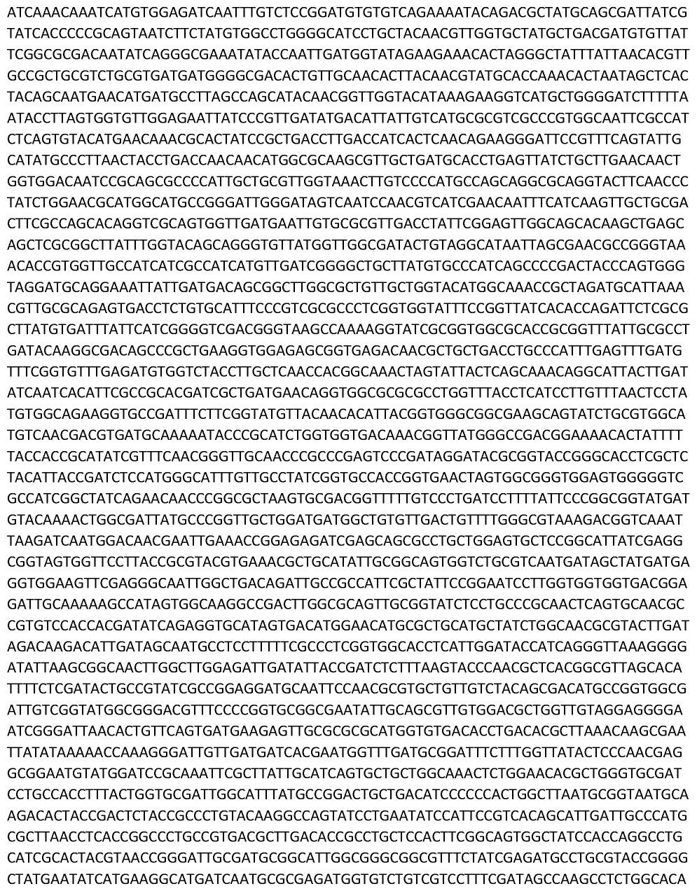

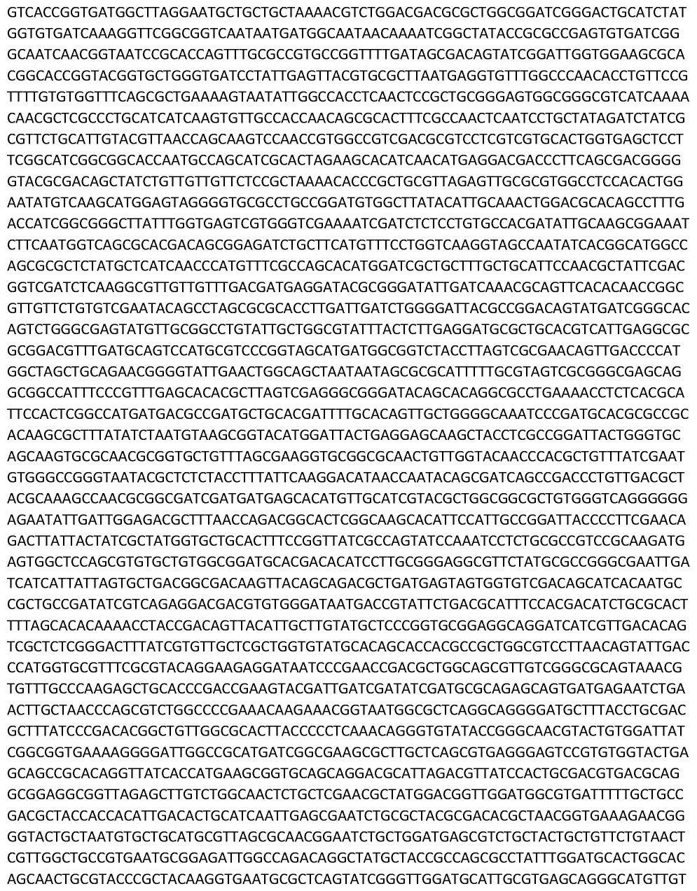

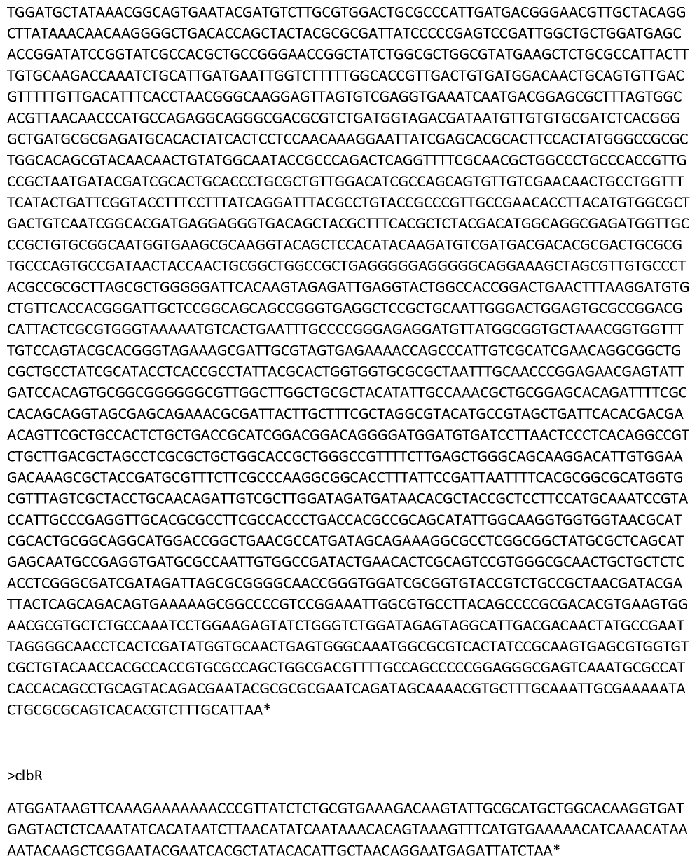

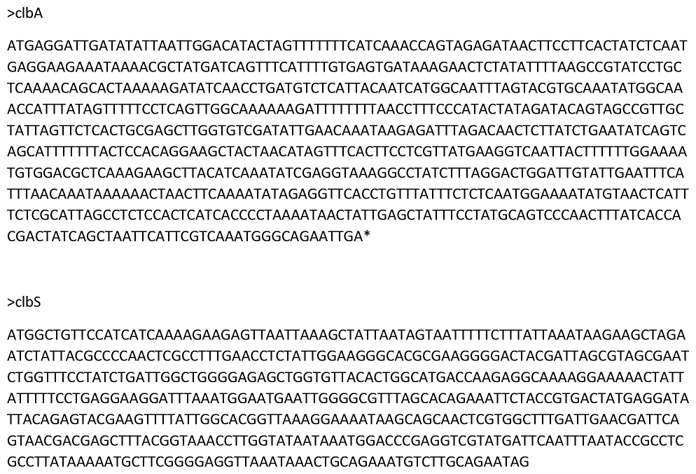

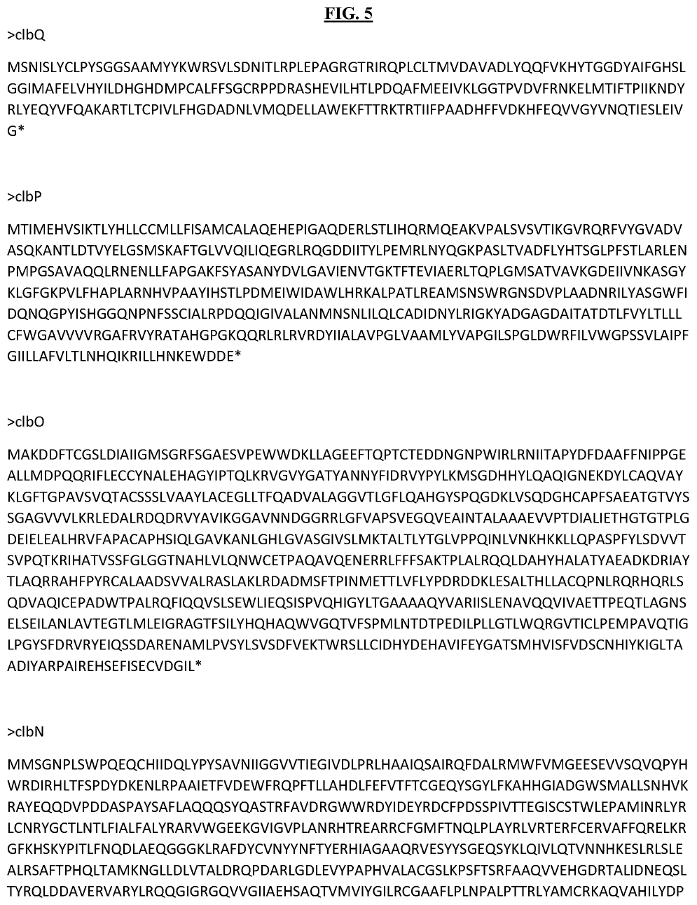

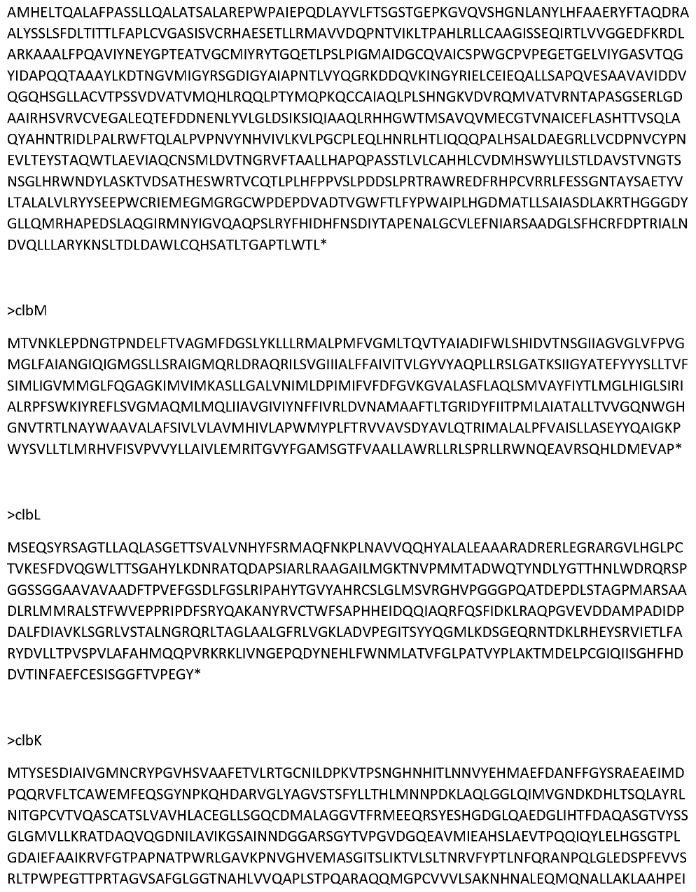

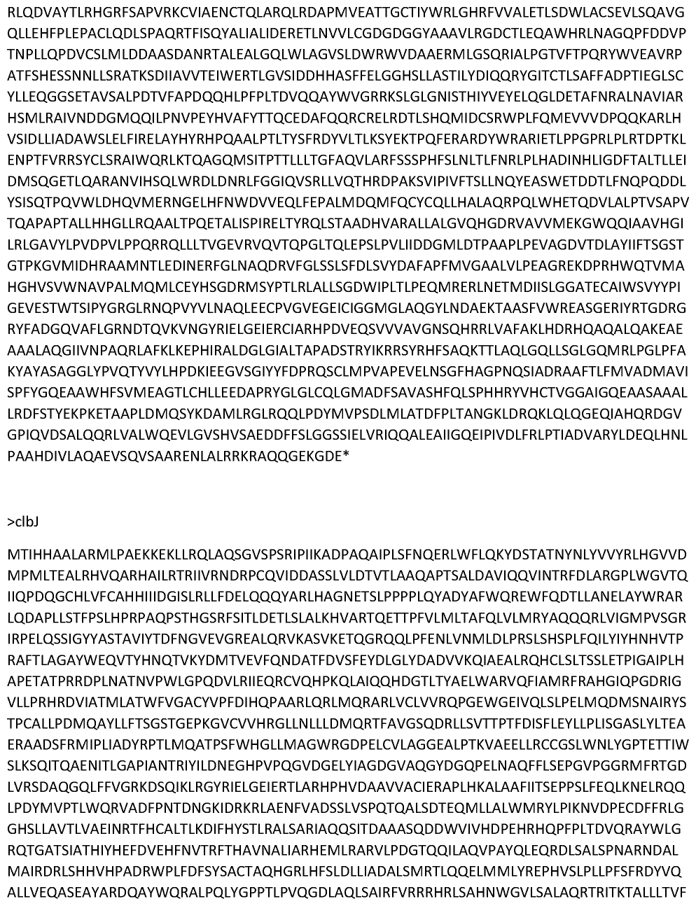

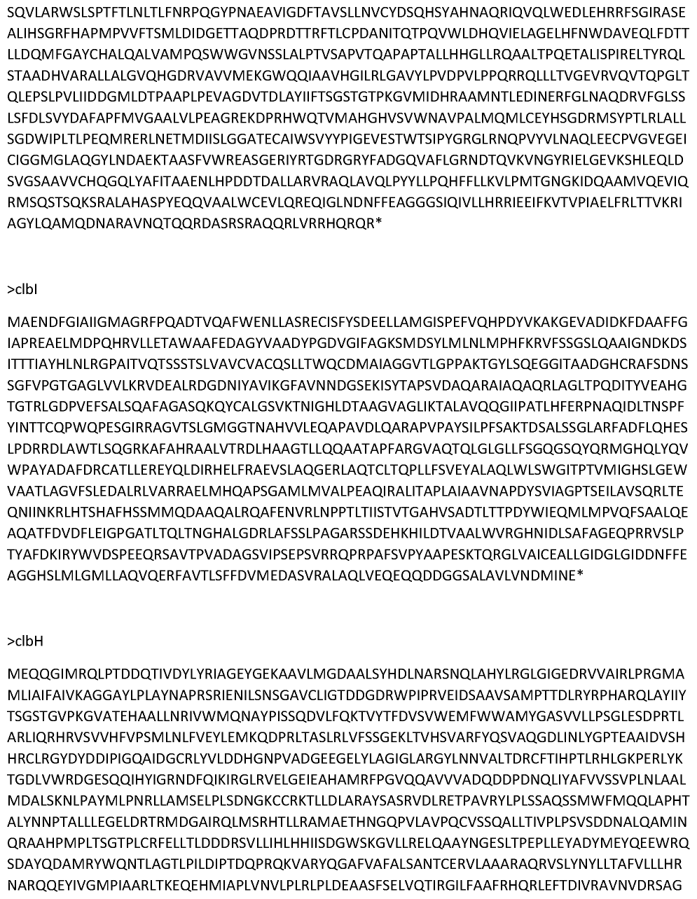

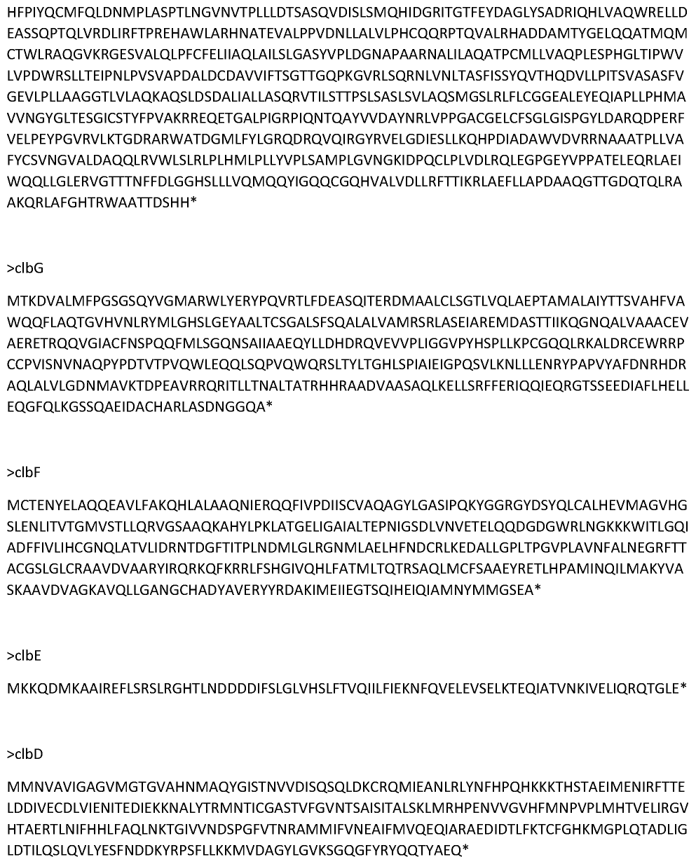

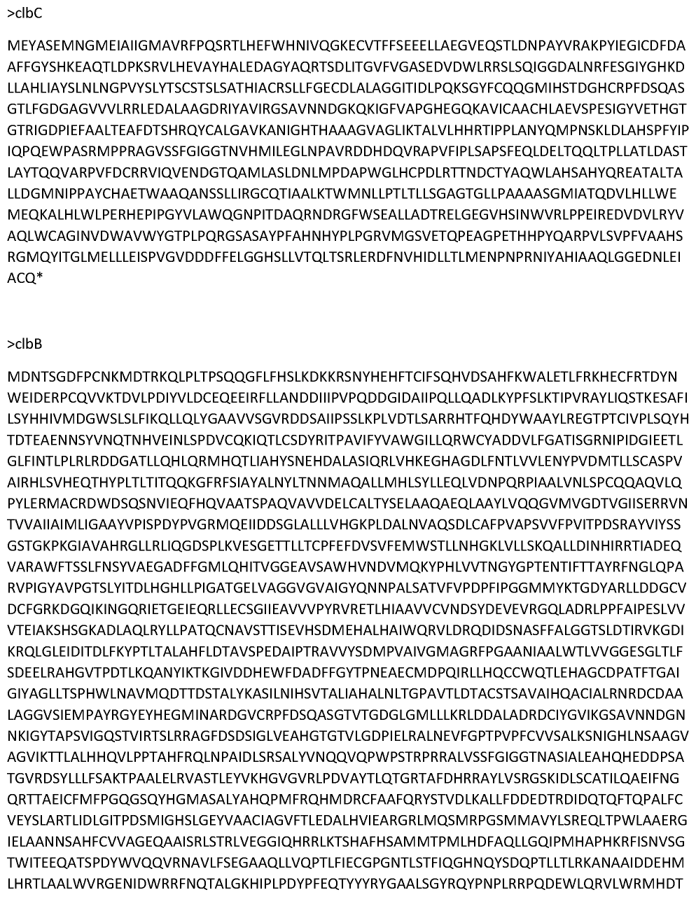

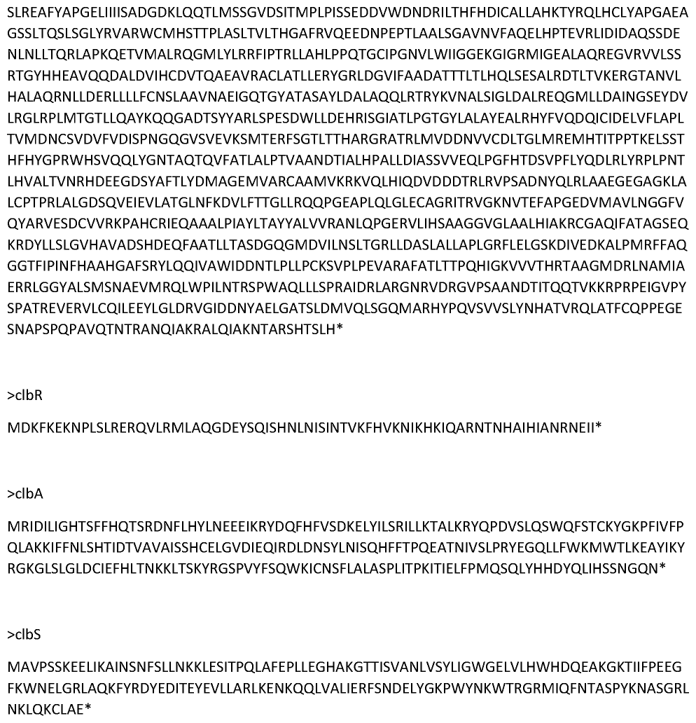

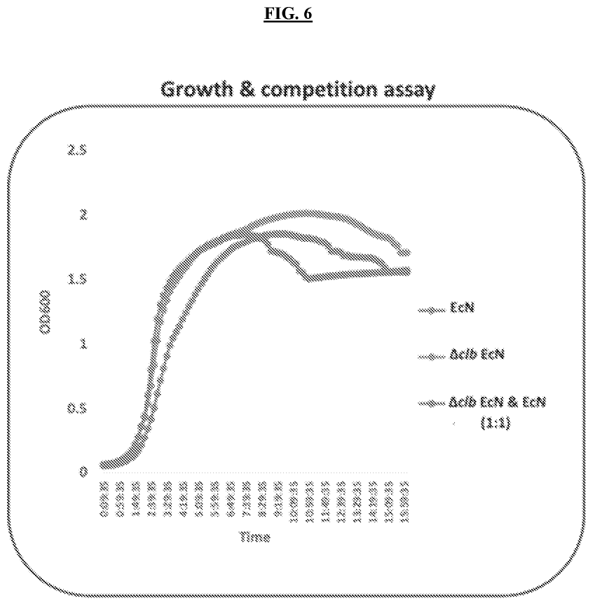

Exemplary clb nucleotide sequences are provided in FIG. 4. Exemplary clb amino acid sequences are provided in FIG. 5.

TABLE 1

| Protein | Description |
|---|---|
| ClbA | Accessory protein: phosphopantetheinyl transferase |
| | Activates other enzymes (favors placement of cofactor) |
| ClbR | Putative regulatory protein |
| ClbB | Synthesis enzyme: hybrid NRPS-PKS |
| ClbC | Synthesis enzyme: PKS |
| ClbD | Synthesis enzyme: hydroxyl acyl coA dehydrogenase |
| ClbE | Synthesis protein: acyl carrier protein |
| ClbF | Synthesis enzyme: αβ dehydrogenase |
| ClbG | Synthesis enzyme: acyl transferase |
| ClbH | Synthesis enzyme: NRPS |
| ClbI | Synthesis enzyme: PKS |
| ClbI | Synthesis enzyme: PKS |
| ClbJ | Synthesis enzyme: NRPS |
| ClbK | Synthesis enzyme: hybrid PKS-NRPS |
| ClbL | Synthesis enzyme: amidase |
| ClbM | Accessory protein: MATE transporter |
| | Transports precolibactin across the cytoplasmic membrane |
| ClbN | Synthesis enzyme: NRPS |
| ClbO | Synthesis enzyme: PKS |
| ClbP | Accessory protein: fmtA peptidase |
| | Transports precolibactin from the cytoplasm to the periplasm |
| | and clives precolibactin into genotoxic colibactin |
| ClbQ | Synthesis enzyme: thioesterase |
| ClbS | Accessory protein |
| | Inactivates colibactin in order to protect bacterial cell |

Colibactin is a cyclomodulin, which is a microorganismal toxin capable of interfering with the eukaryotic cell cycle. Fais 2018. Colibactin-producing microorganisms such as Escherichia coli have been identified in the gut microbiota as commensal bacteria as well as in disease states, e.g., colorectal cancer. Id. Colibactin-producing microorganisms are capable of inducing chromosomal instability, cell cycle arrest, DNA damage, epithelial cell senescence, and/or immune cell apoptosis. Id. However, colibactin-producing microorganisms are also capable of exhibiting anti-inflammatory, antibiotic, and analgesic effects. Id.

As used herein, a modified pks island refers to a change, e.g., in the clb sequence of a clb gene and/or regulatory element, that differs from the pks island of a suitable control, e.g., the native pks island in an unmodified bacterium of the same strain and/or subtype. For example, an engineered bacterium comprises a synthetic clbS gene or a synthetic clbS promoter in the pks island and differs from the corresponding clbS gene or promoter sequence in the pks island of a suitable control, e.g., the native pks island in an unmodified bacterium of the same strain and/or subtype.

As used herein, a partial or full deletion in the pks island refers to clb sequence, e.g., a gene and/or regulatory element, that is removed at least in part as compared to the corresponding clb sequence in the pks island of a suitable control, e.g., the native pks island in an unmodified bacterium of the same strain and/or subtype. A partial or full deletion in the pks island may be referred to as a “knockout” or “delta” (A) pks island. The clb sequence, e.g., clb gene and/or regulatory element, in the native pks island may be deleted by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%. In some embodiments, the clb sequence, e.g., clb gene and/or regulatory element, in the native pks island may be 100% deleted. For example, an engineered bacterium comprises a 100% deletion of the clbA gene in the pks island as compared to the clbA gene sequence in the pks island of a suitable control, e.g., the native pks island in an unmodified bacterium of the same strain and/or subtype. Or, for example, an engineered bacterium comprises a 50% deletion of the clbA promoter in the pks island as compared to the clbA promoter sequence in the pks island of a suitable control, e.g., the native pks island in an unmodified bacterium of the same strain and/or subtype.

As used herein, the term “N-myristoyl-D-asparagine” refers to a small molecule that is produced by the pks island during colibactin synthesis. In some embodiments, ClbN uses asparagine to generate N-myristoyl-D-asparagine, which is accepted by ClbB. In some embodiments, (s) and metabolite(s) are also provided herein. In some embodiments, activity of the pks island and/or levels of colibactin may be assessed by measuring levels of N-myristoyl-D-asparagine.

As used herein, a “therapeutic” molecule, e.g., protein, refers to a molecule that is capable of producing a therapeutic effect in a subject. For example, a therapeutic molecule such as IL-10 may be capable of reducing inflammation in a subject. In some embodiments, the therapeutic molecule is capable of reducing one or more deleterious molecules in the subject, e.g., a phenylalanine-metabolizing enzyme is capable of metabolizing excess and/or deleterious phenylalanine in a subject with PKU. In some embodiments, the therapeutic molecule is capable of reducing one or more deleterious molecules in the subject, e.g., a oxalate metabolizing enzyme is capable of catabolizing excess and/or deleterious oxalate in a subject with HOX. In some embodiments, the deleterious molecule is methionine, and the therapeutic molecule is a methionine catabolism enzyme capable of catabolizing excess and/or deleterious methionine, e.g., in a subject with homocystinuria. In some embodiments, the deleterious molecule is uric acid, and the therapeutic molecule is a uric acid degrading enzyme capable of degrading excess and/or deleterious uric acid, e.g., in a subject with as hyperuricemia or gout. In some embodiments, the deleterious molecule is leucine, and the therapeutic molecule is a leucine metabolic enzyme, e.g., leucine decarboxylase enzyme, capable of catabolizing excess and/or deleterious leucine, e.g., in a subject with isovaleric acidemia, propionic acidemia, methylmalonic acidemia, maple syrup urine disease (MSUD), and diabetes ketoacidosis and other amino acid metabolism disorders. In some embodiments, the therapeutic molecule is capable of elevating levels of a beneficial metabolite in a subject. In one embodiment, the therapeutic molecule is an enzyme for the production of lactate, e.g., for the treatment of inflammatory disorders. In some embodiments, the engineered microorganism, e.g., engineered bacterium, disclosed herein expresses one or more therapeutic molecule(s). In some embodiments, the engineered microorganisms disclosed herein, e.g., genetically engineered bacteria, express one or more therapeutic molecule(s) prior to administration to a subject. In some embodiments, the engineered microorganisms disclosed herein, e.g., genetically engineered bacteria, express one or more therapeutic molecule(s) after administration to a subject, e.g., the gene(s) for producing the therapeutic molecule are induced after administration to the subject.

As used herein, “activity” refers to a desired parameter, e.g., output of a molecule, of a cell or composition, e.g., a bacterium or a bacterial composition. In some embodiments, “therapeutic activity” refers to the production of a desired therapeutic molecule from the cell, e.g., as measured in vitro or in vivo in a cellular model, animal model, or human patient. In some embodiments, activity refers to the amount or function of a desired therapeutic molecule from the cell. In some embodiments, activity refers to the rate at which one or more desired therapeutic molecules is produced. In some embodiments, activity refers to the rate at which one or more deleterious compounds, e.g. a deleterious compound outside of the cell, is metabolized or reduced, e.g., as measured by levels of the deleterious compound or an intermediate.

“Phenylalanine” and “Phe” are used to refer to an amino acid with the formula C₆H₅CH₂CH(NH₂)COOH. Phenylalanine is a precursor for tyrosine, dopamine, norepinephrine, and epinephrine. L-phenylalanine is an essential amino acid and the form of phenylalanine primarily found in dietary protein; the stereoisomer D-phenylalanine is found is lower amounts in dietary protein; DL-phenylalanine is a combination of both forms. Phenylalanine may refer to one or more of L-phenylalanine, D-phenylalanine, and DL-phenylalanine.

Phenylalanine metabolizing enzyme” or “PME” are used to refer to an enzyme which is able to degrade phenylalanine. Any phenylalanine metabolizing enzyme known in the art may be encoded by the engineered microorganisms, genetically engineered bacteria. PMEs include, but are not limited to, phenylalanine hydroxylase (PAH), phenylalanine ammonia lyase (PAL), aminotransferase, L-amino acid deaminase (LAAD), and phenylalanine dehydrogenases, glutamic-oxaloacetic transaminase 1 (GOT1), glutamic-oxaloacetic transaminase 2 (GOT2), dopa carboxylase (DDC) or tyrosine transaminase (TAT).

Phenylalanine hydroxylase (“PAH”) converts phenylalanine into tyrosine. PAH utilizes a co-factor known as tetrahydrobiopterin (BH₄) to convert phenylalanine into tyrosine.

Phenylalanine ammonia lyase” and “PAL” are used to refer to a phenylalanine metabolizing enzyme (PME) that converts or processes phenylalanine to trans-cinnamic acid and ammonia. Trans-cinnamic acid has low toxicity and is converted by liver enzymes in mammals to hippuric acid, which is secreted in the urine. PAL may be substituted for the enzyme PAH to metabolize excess phenylalanine. PAL enzyme activity does not require THB cofactor activity. In some embodiments, PAL is encoded by a PAL gene derived from a prokaryotic species. In alternate embodiments, PAL is encoded by a PAL gene derived from a eukaryotic species. In some embodiments, PAL is encoded by a PAL gene derived from a bacterial species, including but not limited to, Achromobacter xylosoxidans, Pseudomonas aeruginosa, Photorhabdus luminescens, Anabaena variabihs, and Agrobacterium tumefaciens. In some embodiments, PAL is encoded by a PAL gene derived from Anabaena variabilis and referred to as “PAL1” herein (Moffitt et al., 2007). In some embodiments, PAL is encoded by a PAL gene derived from Photorhabdus luminescens and referred to as “PAL3” herein (Williams et al., 2005). In some embodiments, PAL is encoded by a PAL gene derived from a yeast species, e.g., Rhodosporidium toruloides (Gilbert et al., 1985). In some embodiments, PAL is encoded by a PAL gene derived from a plant species, e.g., Arabidopsis thaliana (Wanner et al., 1995). Any suitable nucleotide and amino acid sequences of PAL, or functional fragments thereof, may be used.

“L-Amino acid Deaminase” and “LAAD” are used to refer to an enzyme that catalyzes the stereospecific oxidative deamination of L-amino acids to generate their respective keto acids, ammonia, and hydrogen peroxide. For example, LAAD catalyzes the conversion of phenylalanine to phenylpyruvate. Multiple LAAD enzymes are known in the art, many of which are derived from bacteria, such as Proteus, Providencia, and Morganella, or venom. LAAD is characterized by fast reaction rate of phenylalanine degradation (Hou et al., Appl Microbiol Technol. 2015 October; 99(20): 8391-402; “Production of phenylpyruvic acid from L-phenylalanine using an L-amino acid deaminase from Proteus mirabilis: comparison of enzymatic and whole-cell biotransformation approaches”). Most eukaryotic and prokaryotic L-amino acid deaminases are extracellular; however, Proteus species LAAD are localized to the plasma membrane (inner membrane), facing outward into the periplasmic space, in which the enzymatic activity resides. As a consequence of this localization, phenylalanine transport through the inner membrane into the cytoplasm is not required for Proteus LAAD mediated phenylalanine degradation. Phenylalanine is readily taken up through the outer membrane into the periplasm without a transporter, eliminating the need for a transporter to improve substrate availability.

In some embodiments, the engineered microorganism, e.g., engineered bacterium, comprise a LAAD gene derived from a bacterial species, including but not limited to, Proteus, Providencia, and Morganella bacteria. In some embodiments, the bacterial species is Proteus mirabilis. In some embodiments, the bacterial species is Proteus vulgaris. In some embodiments, the LAAD encoded by the engineered microorganism, e.g., engineered bacterium, is localized to the plasma membrane, facing into the periplasmic space and with the catalytic activity occurring in the periplasmic space.

As used herein, “GOT1,” “GOT2,” and “TAT” convert phenylalanine into phenylpyruvate.

As used herein, “DDC” converts phenylalanine into phenylethylamine.

As used herein, the term “transporter” is meant to refer to a mechanism, e.g., protein or proteins, for importing a molecule, e.g., amino acid, toxin, metabolite, substrate, etc. into the microorganism from the extracellular milieu. For example, a phenylalanine transporter such as PheP imports phenylalanine into the microorganism.

“Phenylalanine transporter” is used to refer to a membrane transport protein that is capable of transporting phenylalanine into bacterial cells (see, e.g., Pi et al., 1991). In Escherichia coli, the pheP gene encodes a high affinity phenylalanine-specific permease responsible for phenylalanine transport (Pi et al., 1998). In some embodiments, the phenylalanine transporter is encoded by a pheP gene derived from a bacterial species, including but not limited to, Acinetobacter calcoaceticus, Salmonella enterica, and Escherichia coli. Other phenylalanine transporters include Aageneral amino acid permease, encoded by the aroP gene, transports three aromatic amino acids, including phenylalanine, with high affinity, and is thought, together with PheP, responsible for the lion share of phenylalanine import. Additionally, a low level of phenylalanine transport activity has been traced to the activity of the LIV-I/LS system, which is a branched-chain amino acid transporter consisting of two periplasmic binding proteins, the LIV-binding protein (LIV-I system) and LS-binding protein (LS system), and membrane components, LivHMGF. In some embodiments, the phenylalanine transporter is encoded by a aroP gene derived from a bacterial species. In some embodiments, the phenylalanine transporter is encoded by LIV-binding protein and LS-binding protein and LivHMGF genes derived from a bacterial species. In some embodiments, the engineered microorganism, e.g., engineered bacterium, comprise more than one type of phenylalanine transporter, selected from pheP, aroP, and the LIV-I/LS system.

“Phenylalanine metabolite” refers to a metabolite that is generated as a result of the degradation of phenylalanine. The metabolite may be generated directly from phenylalanine, by the enzyme using phenylalanine as a substrate, or indirectly by a different enzyme downstream in the metabolic pathway, which acts on a phenylalanine metabolite substrate. In some embodiments, phenylalanine metabolites are produced by the engineered microorganism, e.g., engineered bacterium, encoding a PME.

PMEs and phenylalanine transporters, as well as the nucleotide and amino acid sequences of representative examples of such enzymes and transporters, as well as exemplary promoters, are provided in WO2016183531A1 and WO2017087580A1, the contents of which are hereby incorporated by reference in their entirety. Any suitable enzymes and/or phenylalanine transporters may be used in the engineered microorganism, e.g., engineered bacterium, of the disclosure. In one embodiment, expression of one or more PME(s), e.g., PAL and/or LAAD, and/or Phe transporter(s), e.g., PheP, and/or transcriptional regulator(s), e.g., FNRS24Y, is driven by one or more thermoregulated promoter(s).

“Hyperammonemia,” “hyperammonemic,” or “excess ammonia” is used to refer to increased concentrations of ammonia in the body. Hyperammonemia is caused by decreased detoxification and/or increased production of ammonia. Decreased detoxification may result from urea cycle disorders (UCDs), such as argininosuccinic aciduria, arginase deficiency, carbamoylphosphate synthetase deficiency, citrullinemia, N-acetylglutamate synthetase deficiency, and ornithine transcarbamylase deficiency; or from bypass of the liver, e.g., open ductus hepaticus; and/or deficiencies in glutamine synthetase. See, e.g., Hoffman et al., 2013; Haberle et al., 2013. Increased production of ammonia may result from infections, drugs, neurogenic bladder, and intestinal bacterial overgrowth. See, e.g., Haberle et al., 2013. Increased production of ammonia may also be associated with a tumor microenvironment. See, e.g., Spinelli et al., 2017. Other disorders and conditions associated with hyperammonemia include, but are not limited to, liver disorders such as hepatic encephalopathy, acute liver failure, or chronic liver failure; organic acid disorders; isovaleric aciduria; 3-methylcrotonylglycinuria; methylmalonic acidemia; propionic aciduria; fatty acid oxidation defects; carnitine cycle defects; carnitine deficiency; β-oxidation deficiency; lysinuric protein intolerance; pyrroline-5-carboxylate synthetase deficiency; pyruvate carboxylase deficiency; ornithine aminotransferase deficiency; carbonic anhydrase deficiency; hyperinsulinism-hyperammonemia syndrome; mitochondrial disorders; valproate therapy; asparaginase therapy; total parenteral nutrition; cystoscopy with glycine-containing solutions; post-lung/bone marrow transplantation; portosystemic shunting; urinary tract infections; ureter dilation; multiple myeloma; and chemotherapy. See, e.g., Hoffman et al., 2013; Haberle et al., 2013; Pham et al., 2013; Lazier et al., 2014. In healthy subjects, plasma ammonia concentrations are typically less than about 50 μmol/L. See, e.g., Leonard, 2006. In some embodiments, a diagnostic signal of hyperammonemia is a plasma ammonia concentration of at least about 50 μmol/L, at least about 80 μmol/L, at least about 150 μmol/L, at least about 180 μmol/L, or at least about 200 μmol/L. See, e.g., Leonard, 2006; Hoffman et al., 2013; Haberle et al., 2013. Methods of modifying arginine biosynthesis, e.g., in engineered microorganisms, e.g., engineered bacteria, to reduce hyperammonemia, e.g., by deleting the arginine repressor, modifying the arginine repressor binding sites, and/or using arginine feedback resistant N-acetylglutamate synthase, are known in the art. See, e.g., WO2016200614, the contents of which are hereby incorporated by reference.

An “anti-cancer molecule” refers to one or more therapeutic substances or drugs of interest to be produced by an engineered microorganism, e.g., engineered bacterium, which are capable of reducing and/or inhibiting cell growth or replication. In some embodiments, the anti-cancer molecule is a therapeutic molecule that is useful for modulating or treating a cancer. In some embodiments, the anti-cancer molecule is a therapeutic molecule encoded by a gene. In alternate embodiments, the anti-cancer molecule is a therapeutic molecule produced by a biochemical or biosynthetic pathway, wherein the biosynthetic or biochemical pathway may optionally be endogenous to the microorganism. In some embodiments, the genetically engineered microorganism is capable of producing two or more anti-cancer molecules. Non-limiting examples of anti-cancer molecules include immune checkpoint inhibitors (e.g., CTLA-4 antibodies, PD-1 antibodies, PDL-1 antibodies), cytotoxic agents (e.g., Cly A, FASL, TRAIL, TNF-alpha), immunostimulatory cytokines and co-stimulatory molecules (e.g., OX40, CD28, ICOS, CCL21, IL-2, IL-18, IL-15, IL-12, IFN-gamma, IL-21, TNFs, GM-CSF), antigens and antibodies (e.g., tumor antigens, neoantigens, CtxB-PSA fusion protein, CPV-OmpA fusion protein, NY-ESO-1 tumor antigen, RAF1, antibodies against immune suppressor molecules, anti-VEGF, Anti-CXR4/CXCL12, anti-GLP1, anti-GLP2, anti-galectin1, anti-galectin3, anti-Tie2, anti-CD47, antibodies against immune checkpoints, antibodies against immunosuppressive cytokines and chemokines), DNA transfer vectors (e.g., endostatin, thrombospondin-1, TRAIL, SMAC, Stat3, Bcl2, FLT3L, GM-CSF, IL-12, AFP, VEGFR2), and enzymes (e.g., E. coli CD, HSV-TK). In some embodiments, the anti-cancer molecule includes nucleic acid molecules that mediate RNA interference, microRNA response or inhibition, TLR response, antisense gene regulation, target protein binding (aptamer or decoy oligos), gene editing, such as CRISPR interference. In some embodiments, bacteria or virus can be used as vectors to transfer DNA into mammalian cells, e.g., by bactofection. See, e.g., Bernardes et al., 2013. Engineered microorganisms, e.g., engineered bacteria, that are capable of producing an anti-cancer molecule, e.g., a deadenylate cyclase gene (e.g., dacA from Listeria monocytogenes) or an enzyme capable of producing a stimulator of interferon gene (STING) agonist, are known in the art. See, e.g., WO2018129404, the contents of which are hereby incorporated by reference.

The term “oxalate” as used herein, refers to the dianion of the formula C₂O₄²⁻. Oxalate is the conjugate base of oxalic acid. The term “oxalic acid,” as used herein, refers to a dicarboxylic acid with the chemical formula H₂C₂O₄.

In one embodiment, a “disorder in which oxalate is detrimental” is a disease or disorder involving the abnormal, e.g., increased, levels of oxalate and/or oxalic acid or molecules directly upstream, such as glyoxylate. In one embodiment, the disorder in which oxalate is detrimental is a disorder or disease in which hyperoxaluria is observed in the subject. In one embodiment the disorder in which oxalate is detrimental refers to any condition(s), disorder(s), disease(s), predisposition(s), and/or genetic mutations(s) that result in daily urinary oxalate excretion over 40 mg per 24 hours. In one embodiment the disorder in which oxalate is detrimental is a disorder or disease selected from the group consisting of: PHI, PHII, PHII, secondary hyperoxaluria, enteric hyperoxaluria, syndrome of bacterial overgrowth, Crohn's disease, inflammatory bowel disease, hyperoxaluria following renal transplantation, hyperoxaluria after a jejunoileal bypass for obesity, hyperoxaluria after gastric ulcer surgery, chronic mesenteric ischemia, gastric bypass, cystic fibrosis, short bowel syndrome, biliary/pancreatic diseases (e.g., chronic pancreatitis).

As used herein, the term “oxalate catabolism enzyme” or “oxalate metabolic enzyme” refers to any enzyme that is capable of metabolizing oxalate or capable of reducing accumulated oxalate or that can lessen, ameliorate, or prevent one or more diseases, or disease symptoms in which oxalate is detrimental. Examples of oxalate enzymes include, but are not limited to, formyl-CoA:oxalate CoA-transferase (also called formyl-CoA transferase), e.g., Frc from O. formigenes, oxalyl-CoA synthetase (also called oxalate-CoA ligase), e.g., Saccharomyces cerevisiae acyl-activating enzyme 3 (ScAAE3) from Saccharomyces cerevisiae, Oxalyl-CoA Decarboxylase, e.g., Oxc from O. formigenes (also referred to herein is oxdC or oxalate decarboxylase), acetyl-CoA:oxalate CoA-transferase (ACOCT), e.g., YfdE from E. coli and any other enzymes that catabolizes oxalate, oxalyl-CoA or any other metabolite thereof. Catabolism enzymes also include alanine glyoxalate aminotransferase (AGT, encoded by the AGXT gene, e.g. the human form), glyoxylate/hydroxypyruvate reductase (GRHPR; an enzyme having glyoxylate reductase (GR), hydroxypyruvate reductase (HPR), and D-glycerate dehydrogenase (DGDH) activities, e.g., the human form), and 4-hydroxy 2-oxoglutarate aldolase (encoded by the HOGA1 gene, e.g. in humans, and which breaks down 4-hydroxy 2-oxoglutarate into pyruvate and glyoxalate). Functional deficiencies in these proteins result in the accumulation of oxalate or its corresponding α-keto acid in cells and tissues. Oxalate metabolic enzymes of the present disclosure include both wild-type or modified oxalate metabolic enzymes and can be produced using recombinant and synthetic methods or purified from nature sources. Oxalate metabolic enzymes include full-length polypeptides and functional fragments thereof, as well as homologs and variants thereof. oxalate metabolic enzymes include polypeptides that have been modified from the wild-type sequence, including, for example, polypeptides having one or more amino acid deletions, insertions, and/or substitutions and may include, for example, fusion polypeptides and polypeptides having additional sequence, e.g., regulatory peptide sequence, linker peptide sequence, and other peptide sequence.

As used herein, the term “catabolism” refers to the cellular uptake of oxalate, and/or degradation of oxalate into its corresponding oxalyl CoA, and/or the degradation of oxalyl CoA formate and carbon dioxide. In one embodiment, the cellular uptake of oxalate occurs in the kidney. In one embodiment, the cellular uptake occurs in the liver. In one embodiment, the cellular uptake of oxalate occurs in the intestinal tract. In one embodiment, the cellular uptake of oxalate occurs in the stomach. In one embodiment, the cellular uptake is mediated by a SLC26 transporting protein (see Robijn et al. (2011). In one embodiment, the cellular uptake is mediated by the transport protein SLC26A1. In one embodiment, the cellular uptake is mediated by the transport protein SLC26A6. In one embodiment, the cellular uptake of oxalate is mediated by a paracellular transport system. In one embodiment, the cellular uptake of oxalate is mediated by a transcellular transport system.

As used herein, the term “methionine catabolism enzyme” refers to an enzyme involved in the catabolism of methionine. Specifically, when a methionine catabolism enzyme is expressed in a recombinant bacterial cell, the bacterial cell hydrolyzes more methionine into 3-methylthiopropylamine when the catabolism enzyme is expressed than unmodified bacteria of the same bacterial subtype under the same conditions. In some embodiments, methionine transporters may also be expressed or modified in the recombinant bacteria to enhance methionine import into the cell in order to increase the catabolism of methionine by the methionine catabolism enzyme. In other embodiments, methionine exporters may be knocked-out in the recombinant bacteria to decrease export of methionine and/or increase cytoplasmic concentration of methionine. In some embodiment the genetically engineered bacterial cell comprises a gene sequence encoding a methionine decarboxylase (MDC) gene sequence. In some embodiments, the bacterial cell comprises genetic modification that reduces export of methionine from the bacterial cell, e.g., a knockout of an endogenous methionine efflux pump, such as yjeH. In some embodiments, the bacterial cell further comprises a methionine importer, such as metNIQ. Non-limiting examples of such recombinant or genetically engineered bacteria are disclosed in international Patent Application PCT/US2021/017775, filed on Feb. 12, 2021, and International Patent Application PCT/US2016/032565, filed on May 13, 2016, the contents of each of which is herein incorporated by reference its entirety.

the contents of which is herein incorporated by reference its entirety.

As used herein, the term “uric acid degrading enzyme” or “uric acid degradation enzyme” refers to an enzyme capable of processing, breakdown, and/or degradation of uric acid. In one embodiment, a uric acid degradation enzyme refers to the processing, breakdown, and/or degradation of uric acid into, for example, hydroxyisourate and/or allantoin. In other embodiments, a “uric acid degrading enzyme” or “uric acid degradation enzyme” may refer to an enzyme which works upstream to degrade a precursor of uric acid, thereby decreasing downstream levels of uric acid. For example, in one embodiment, a uric acid degradation enzyme degrades guanosine. In another embodiment, a uric acid degradation enzyme degrades adenosine. Non-limiting examples of such recombinant or genetically engineered bacteria are disclosed in International Patent Application PCT/US2021/019625, filed on Feb. 25, 2021, the contents of which is herein incorporated by reference its entirety.

As used herein, the term “leucine catabolism enzyme” or “leucine catabolism enzyme” or “catabolism enzyme” or “leucine metabolic enzyme” refers to any enzyme that is capable of metabolizing leucine, and/or capable of reducing accumulated leucine, and/or that can lessen, ameliorate, or prevent one or more diseases associated with leucine catabolism or disease symptoms. Examples of leucine catabolism enzymes include, but are not limited to, leucine decarboxylase (LCD). In one embodiment, the LDC enzyme is E.C. 4.1.1.14. Non-limiting examples of such recombinant or genetically engineered bacteria are disclosed in International Patent Application PCT/US2021/022676, filed on Mar. 17, 2021, the contents of which is herein incorporated by reference its entirety.

“Operably linked” refers a nucleic acid sequence, e.g., a gene encoding a therapeutic molecule, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

An “inducible promoter” refers to a regulatory region that is operably linked to one or more genes, wherein expression of the gene(s) is increased in the presence of an inducer of said regulatory region.

In some embodiments, the engineered microorganism, e.g., engineered bacterium, comprise one or more gene(s) whose expression is controlled by a temperature sensitive mechanism. Thermoregulators are advantageous because of strong transcriptional control without the use of external chemicals or specialized media (see, e.g., Nemani et al., Magnetic nanoparticle hyperthermia induced cytosine deaminase expression in microencapsulated E. coli for enzyme-prodrug therapy; J Biotechnol. 2015 Jun. 10; 203: 32-40, and references therein). Thermoregulated protein expression using the mutant cI857 repressor and the pL and/or pR phage λ promoters may be used to engineer recombinant bacterial strains. The gene of interest is cloned downstream of the λ promoters and can be efficiently regulated by the mutant thermolabile cI857 repressor of bacteriophage λ. At temperatures below 37° C., cI857 binds to the oL or oR regions of the pR promoter and blocks transcription by RNA polymerase. At higher temperatures, the functional cI857 dimer is destabilized, binding to the oL or oR DNA sequences is abrogated, and mRNA transcription is initiated.

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., WO2017087580; Castiglione et al., 2009; Eiglmeier et al., 1989; Galimand et al., 1991; Hasegawa et al., 1998; Hoeren et al., 1993; Salmon et al., 2003). In some embodiments, the FNR-responsive promoter is PfnrS derived from the E. coli Nissle fumarate and nitrate reductase gene S (fnrS) that is known to be highly expressed under conditions of low or no environmental oxygen (Durand and Storz, 2010; Boysen et al, 2010).

As used herein, a “non-native” nucleic acid sequence refers to a nucleic acid sequence not normally present in a bacterium, e.g., an extra copy of an endogenous sequence, or a heterologous sequence such as a sequence from a different species, strain, or sub-strain of bacteria, or a sequence that is modified and/or mutated as compared to the unmodified sequence from bacteria of the same subtype. In some embodiments, the non-native nucleic acid sequence is a synthetic, non-naturally occurring sequence. See, e.g., Purcell et al., 2013, Towards a whole-cell modeling approach for synthetic biology. The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. In some embodiments, the engineered microorganism, e.g., engineered bacterium, are engineered to comprise multiple copies of the same regulatory region, promoter, gene, and/or gene cassette in order to enhance copy number or to comprise multiple different components of a gene cassette performing multiple different functions. In some embodiments, the engineered microorganism, e.g., engineered bacterium, of the invention comprise a gene encoding a phenylalanine-metabolizing enzyme that is operably linked to a inducible promoter that is not associated with said gene in nature, e.g., an FNR promoter operably linked to a gene encoding PAL or a ParaBAD promoter operably linked to LAAD.

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines. In some embodiments, the engineered microorganism, e.g., engineered bacterium, are active (e.g., express one or more heterologous genes) in the gut. In some embodiments, the engineered microorganism, e.g., engineered bacterium, are active (e.g., express one or more heterologous genes) in the large intestine. In some embodiments, the engineered microorganism, e.g., engineered bacterium, are active (e.g., express one or more heterologous genes) in the small intestine. In some embodiments, the engineered microorganism, e.g., engineered bacterium, are active in the small intestine and in the large intestine.

As used herein, the term “gene” or “gene sequence” is meant to refer to a genetic sequence, e.g., a nucleic acid sequence. The gene, gene sequence or genetic sequence is meant to include a complete gene sequence or a partial gene sequence. The gene, gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

“Microorganism” refers to an organism or microbe of microscopic, submicroscopic, or ultramicroscopic size that typically consists of a single cell. Examples of microorganisms include bacteria, yeast, viruses, parasites, fungi, certain algae, and protozoa. In some aspects, the microorganism is engineered (“engineered microorganism”) to produce one or more therapeutic molecules or proteins of interest. In certain aspects, the microorganism is engineered to take up and catabolize certain metabolites or other compounds from its environment, e.g., the gut. In certain aspects, the microorganism is engineered to synthesize certain beneficial metabolites or other compounds (synthetic or naturally occurring) and release them into its environment. In certain embodiments, the engineered microorganism is an engineered bacterium. In certain embodiments, the engineered microorganism is an engineered virus.

An “engineered” or “genetically engineered” microorganism refers to a microorganism, e.g., bacterium, that has been genetically modified from its native state. Thus, an “engineered” or “genetically engineered” bacterium refers to a bacterium that has been genetically modified from its native state, e.g., to perform a specific function, e.g., to metabolize a metabolite, e.g. phenylalanine, oxalate, methionine or leucine. In certain embodiments, the engineered microorganism, e.g., bacterium, is modified to express one or more molecules, e.g., therapeutic molecules, e.g., therapeutic proteins that have a therapeutic activity or serve a therapeutic purpose. The engineered microorganism, e.g., bacterium, may additionally have the ability to stop growing or to destroy itself once the protein(s) of interest have been expressed.

“Non-pathogenic” refers to microorganisms, for example bacteria, that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to, Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, the terms “treat” and “modulate” and their cognates refer to an amelioration of a disease, disorder, and/or condition, or at least one discernible symptom thereof. In another embodiment, “treat” and “modulate” refer to an amelioration of at least one measurable physical parameter, not necessarily discernible by the patient. In another embodiment, “treat” and “modulate” refer to inhibiting the progression of a disease, disorder, and/or condition, either physically (e.g., stabilization of a discernible symptom), physiologically (e.g., stabilization of a physical parameter), or both. In another embodiment, “treat” and “modulate” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. For example, primary hyperphenylalaninemia, e.g., PKU, is caused by inborn genetic mutations for which there are no known cures, and hyperphenylalaninemia can also be secondary to other conditions, e.g., liver diseases. Treatment may encompass reducing or eliminating one or more disease features, e.g., excess phenylalanine in primary hyperphenylalaninemia, and does not necessarily encompass the elimination of the underlying disease.

As used herein a “pharmaceutical composition” refers to a preparation of engineered microorganisms, e.g., engineered bacteria, of the invention with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., hyperphenylalaninemia. A therapeutically effective amount may, for example, be sufficient to treat, prevent, reduce the severity, delay the onset, and/or reduce the risk of occurrence of one or more symptoms of a disease or condition associated with excess phenylalanine levels. A therapeutically effective amount, as well as a therapeutically effective frequency of administration, can be determined by methods known in the art and discussed below.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “dipeptide” refers to a peptide of two linked amino acids. The term “tripeptide” refers to a peptide of three linked amino acids. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the engineered microorganisms, e.g., genetically engineered bacteria or virus, of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

The term “about”, when used before a list, modifies each member of the list. The term “about” or “approximately” means an acceptable error for a particular value as determined by one of ordinary skill in the art, which depends in part on how the value is measured or determined.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

References cited herein are incorporated by reference in their entireties.

### Engineered Microorganism Comprising Modified pks Island

In some embodiments, the disclosure provides an engineered microorganism, e.g., engineered bacterium, comprising a modified pks island, and compositions and formulations thereof. In some embodiments, the disclosure provides an engineered microorganism, e.g., engineered bacterium, comprising a modified pks island and further comprising one or more gene(s) or gene cassettes(s) encoding a therapeutic molecule, and compositions and formulations thereof.

In some embodiments, the engineered microorganism, e.g., engineered bacterium, comprises a modified pks island. In some embodiments, the engineered microorganism, e.g., engineered bacterium, comprises a modified clb sequence selected from one or more of the clbA, clbB, clbC, clbD, clbE, clbF, clbG, clbH, clbI, clbJ, clbK, clbL, clbM, clbN, clbO, clbP, clbQ, clbR, and clbS gene sequences and/or operably linked promoter sequences, as compared to a suitable control, e.g., the native pks island in an unmodified bacterium of the same strain and/or subtype. In some embodiments, the modified clb sequence is an insertion, a substitution, and/or a deletion as compared to the control.

In some embodiments, the engineered microorganism, e.g., engineered bacterium, comprises a modification in one or more of the clbA, clbB, clbC, clbD, clbE, clbF, clbG, clbH, clbI, clbJ, clbK, clbL, clbM, clbN, clbO, clbP, clbQ, and clbR gene sequences as compared to the suitable control.

In some embodiments, the engineered microorganism, e.g., engineered bacterium, comprises a modification in one or more of the promoter sequences operably linked to the clbA, clbB, clbC, clbD, clbE, clbF, clbG, clbH, clbI, clbJ, clbK, clbL, clbM, clbN, clbO, clbP, clbQ, and clbR genes as compared to the suitable control.

In some embodiments, the engineered microorganism, e.g., engineered bacterium, comprises a modification in each of the clbA, clbB, clbC, clbD, clbE, clbF, clbG, clbH, clbI, clbJ, clbK, clbL, clbM, clbN, clbO, clbP, clbQ, and clbR gene sequences as compared to the suitable control.

In some embodiments, the engineered microorganism, e.g., engineered bacterium, comprises a modification in each of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the engineered microorganism, e.g., engineered bacterium, comprises a modification in one or more of the clbA, clbB, clbC, clbD, clbE, clbF, clbG, clbH, clbI, clbJ, clbK, clbL, clbM, clbN, clbO, clbP, clbQ, clbR, and clbS gene sequences as compared to the suitable control.

In some embodiments, the engineered microorganism, e.g., engineered bacterium, comprises a modification in one or more of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the engineered microorganism, e.g., engineered bacterium, comprises a modification in each of the clbA, clbB, clbC, clbD, clbE, clbF, clbG, clbH, clbI, clbJ, clbK, clbL, clbM, clbN, clbO, clbP, clbQ, clbR, and clbS gene sequences as compared to the suitable control.

In some embodiments, the engineered microorganism, e.g., engineered bacterium, comprises a modification in each of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the engineered microorganism, e.g., engineered bacterium, comprises an unmodified clbS gene sequence, a modification in each of the clbA, clbB, clbC, clbD, clbE, clbF, clbG, clbH, clbI, clbJ, clbK, clbL, clbM, clbN, clbO, clbP, clbQ, and clbR gene sequences, and a modification in the clbS promoter sequence, as compared to the suitable control.

In some embodiments, the engineered microorganism, e.g., engineered bacterium, comprises a full or partial deletion in the pks island, e.g., a full or deletion in a clb sequence, e.g., a clb gene or promoter sequence. In some embodiments, the engineered microorganism, e.g., engineered bacterium, comprises a full or partial deletion in a clb sequence selected from one or more of the clbA, clbB, clbC, clbD, clbE, clbF, clbG, clbH, clbI, clbJ, clbK, clbL, clbM, clbN, clbO, clbP, clbQ, clbR, and clbS gene sequences and/or operably linked promoter sequences, as compared to a suitable control, e.g., the native pks island in an unmodified bacterium of the same strain and/or subtype. In some embodiments, the clb sequence, e.g., clb gene or promoter sequence, may be deleted by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% as compared to the control, e.g., the corresponding clb sequence in the native pks island in an unmodified bacterium of the same strain and/or subtype. In some embodiments, the clb sequence, e.g., clb gene or promoter sequence, may be 100% deleted as compared to the control, e.g., the corresponding clb sequence in the native pks island in an unmodified bacterium of the same strain and/or subtype

In some embodiments, the engineered microorganism, e.g., engineered bacterium, comprises a partial or full deletion in one or more of the clbA, clbB, clbC, clbD, clbE, clbF, clbG, clbH, clbI, clbJ, clbK, clbL, clbM, clbN, clbO, clbP, clbQ, and clbR gene sequences as compared to the suitable control.

In some embodiments, the engineered microorganism, e.g., engineered bacterium, comprises a partial or full deletion in one or more of the promoter sequences operably linked to the clbA, clbB, clbC, clbD, clbE, clbF, clbG, clbH, clbI, clbJ, clbK, clbL, clbM, clbN, clbO, clbP, clbQ, and clbR genes as compared to the suitable control.

In some embodiments, the engineered microorganism, e.g., engineered bacterium, comprises a partial or full deletion in each of the clbA, clbB, clbC, clbD, clbE, clbF, clbG, clbH, clbI, clbJ, clbK, clbL, clbM, clbN, clbO, clbP, clbQ, and clbR gene sequences as compared to the suitable control.

In some embodiments, the engineered microorganism, e.g., engineered bacterium, comprises a partial or full deletion in each of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the engineered microorganism, e.g., engineered bacterium, comprises a partial or full deletion in one or more of the clbA, clbB, clbC, clbD, clbE, clbF, clbG, clbH, clbI, clbJ, clbK, clbL, clbM, clbN, clbO, clbP, clbQ, clbR, and clbS gene sequences as compared to the suitable control.

In some embodiments, the engineered microorganism, e.g., engineered bacterium, comprises a partial or full deletion in one or more of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the engineered microorganism, e.g., engineered bacterium, comprises a partial or full deletion in each of the clbA, clbB, clbC, clbD, clbE, clbF, clbG, clbH, clbI, clbJ, clbK, clbL, clbM, clbN, clbO, clbP, clbQ, clbR, and clbS gene sequences as compared to the suitable control.

In some embodiments, the engineered microorganism, e.g., engineered bacterium, comprises a partial or full deletion in each of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the engineered microorganism, e.g., engineered bacterium, comprises an unmodified clbS gene sequence, a partial or full deletion in each of the clbA, clbB, clbC, clbD, clbE, clbF, clbG, clbH, clbI, clbJ, clbK, clbL, clbM, clbN, clbO, clbP, clbQ, and clbR gene sequences, and a partial or full deletion in the clbS promoter sequence, as compared to the suitable control

In some embodiments, the engineered microorganism, e.g., engineered bacterium comprising the modified pks island, expresses less colibactin compared to a suitable control, e.g., the native pks island in an unmodified bacterium of the same strain and/or subtype of the comprising the native pks island. In some embodiments, the engineered microorganism, e.g., engineered bacterium comprising the modified pks island, expresses less colibactin compared to control. In some embodiments, the engineered microorganism, e.g., engineered bacterium comprising the modified pks island, expresses less N-myristoyl-D-asparagine compared to control. In some embodiments, the engineered microorganism, e.g., engineered bacterium comprising the modified pks island, exhibits decreased accessory protein activity as compared to control. In some embodiments, the engineered microorganism, e.g., engineered bacterium comprising the modified pks island, exhibits decreased phosphopantetheinyl transferase activity as compared to control. In some embodiments, the engineered microorganism, e.g., engineered bacterium comprising the modified pks island, exhibits decreased polyketide synthase activity as compared to control. In some embodiments, the engineered microorganism, e.g., engineered bacterium comprising the modified pks island, exhibits decreased non-ribosomal peptide synthase activity as compared to control. In some embodiments, the engineered microorganism, e.g., engineered bacterium comprising the modified pks island, exhibits decreased activity of hybrid non-ribosomal peptide/polyketide synthase as compared to control. In some embodiments, the engineered microorganism, e.g., engineered bacterium comprising the modified pks island, exhibits decreased hydroxyl acyl coA dehydrogenase activity as compared to control. In some embodiments, the engineered microorganism, e.g., engineered bacterium comprising the modified pks island, exhibits decreased acyl carrier protein activity as compared to control. In some embodiments, the engineered microorganism, e.g., engineered bacterium comprising the modified pks island, exhibits decreased αβ dehydrogenase activity as compared to control. In some embodiments, the engineered microorganism, e.g., engineered bacterium comprising the modified pks island, exhibits decreased acyl transferase activity as compared to control. In some embodiments, the engineered microorganism, e.g., engineered bacterium comprising the modified pks island, exhibits decreased amidase activity as compared to control. In some embodiments, the engineered microorganism, e.g., engineered bacterium comprising the modified pks island, exhibits decreased MATE transporter activity as compared to control. In some embodiments, the engineered microorganism, e.g., engineered bacterium comprising the modified pks island, exhibits decreased fmtA peptidase activity as compared to control. In some embodiments, the engineered microorganism, e.g., engineered bacterium comprising the modified pks island, exhibits decreased thioesterase activity as compared to control. In some embodiments, the engineered microorganism, e.g., engineered bacterium comprising the modified pks island, exhibits decreased precolibactin transport across the cytoplasmic membrane as compared to control. In some embodiments, the engineered microorganism, e.g., engineered bacterium comprising the modified pks island, exhibits decreased precolibactin transport across the periplasm as compared to control. In some embodiments, the engineered microorganism, e.g., engineered bacterium comprising the modified pks island, exhibits decreased precolibactin cleavage into colibactin as compared to control.

In some embodiments, the engineered microorganism, e.g., engineered bacterium comprising the modified pks island, exhibits a similar growth rate as compared to a corresponding control comprising an unmodified pks island, e.g., in a growth and competition assay. In some embodiments, the engineered microorganism, e.g., engineered bacterium comprising the modified pks island, exhibits a similar viability as compared to a corresponding control comprising an unmodified pks island, e.g., after competitive growth. In some embodiments, the engineered microorganism, e.g., engineered bacterium comprising the modified pks island, exhibits similar ability to express therapeutic molecule as compared to a corresponding control comprising an unmodified pks island. In some embodiments, there is minimal fitness cost to an engineered bacterium comprising the modified pks island as compared to a corresponding control comprising an unmodified pks island.

In some embodiments, the engineered microorganism, e.g., engineered bacterium, further comprise one or more gene(s) for producing a desired therapeutic molecule. Engineered microorganisms, e.g., engineered bacteria, comprising one or more gene(s) for producing a desired therapeutic molecule that may further comprise a modified pks island are described in WO2016090343, WO2016200614, WO2017139697, WO2016183531, WO2017087580, WO2016141108, WO2017074566, WO2017136792, WO2017136795, WO2018129404, WO2019014391, WO2016210384, WO2017123418, WO2017123676, WO2016183531, WO2018237198, WO2016201380, US20170216370, and WO2017040719, the contents of which are hereby incorporated by reference in their entirety.

In some embodiments, the one or more gene(s) is operably linked to an inducible promoter. In some embodiments, the therapeutic molecule is capable of producing a therapeutic effect in a subject. For example, a therapeutic molecule such as IL-10 may be capable of reducing inflammation in a subject. In some embodiments, the therapeutic molecule is an anti-cancer molecule. In some embodiments, the therapeutic molecule is an enzyme capable of producing a STING agonist. In some embodiments, the therapeutic molecule is a deadenylate cyclase, e.g., dacA. In some embodiments, the therapeutic molecule is capable of reducing one or more deleterious molecules in the subject, e.g., a phenylalanine-metabolizing enzyme is capable of metabolizing excess and deleterious phenylalanine in a subject with PKU or an oxalate metabolic enzyme is capable of catabolizing excess and deleterious oxalate in a subject with HOX. In some embodiments, the engineered microorganism, e.g., engineered bacterium, comprise gene(s) encoding a modified arginine biosynthesis pathway (e.g., deleted arginine repressor, modified arginine repressor binding sites, and/or arginine feedback resistant N-acetylglutamate synthase mutation) and is capable of reducing deleterious ammonia, e.g., in a subject with UCD or in a subject with cancer. In some embodiments, the therapeutic molecule works in conjunction with another molecule to produce a therapeutic effect, e.g., a phenylalanine transporter works in conjunction with a phenylalanine-metabolizing enzyme to reduce deleterious phenylalanine in a subject with PKU. In some embodiments, the engineered microorganism, e.g., engineered bacterium, disclosed herein expresses one or more therapeutic molecule(s). In some embodiments, the engineered microorganism, e.g., engineered bacterium, disclosed herein expresses one or more therapeutic molecule(s) prior to administration to a subject. In some embodiments, the engineered microorganism, e.g., engineered bacterium, disclosed herein expresses one or more therapeutic molecule(s) after administration to a subject, e.g., the gene(s) for producing the therapeutic molecule are induced after administration to the subject.

In some embodiments, the engineered microorganism, e.g., engineered bacterium, comprises a modified pks island as described herein and one or more gene(s) and/or gene cassette(s) encoding a therapeutic molecule. In some embodiments, the engineered microorganism, e.g., engineered bacterium, comprises a modified pks island as described herein and one or more gene(s) and/or gene cassette(s) encoding at least one phenylalanine metabolizing enzyme. In some embodiments, the engineered microorganism, e.g., engineered bacterium, comprises a modified pks island as described herein and one or more gene(s) and/or gene cassette(s) encoding at least one oxalate metabolic enzyme.

In some embodiments, the engineered microorganism, e.g., engineered bacterium, comprises a modified pks island and a gene encoding PAH. In some embodiments, the engineered microorganism, e.g., engineered bacterium, comprise a gene encoding PAH, wherein the PAH gene is operably linked to an inducible promoter. In some embodiments, the microorganisms, e.g., bacteria, comprise a non-native PAH gene. In some embodiments, the microorganisms, e.g., bacteria, comprise additional copies of a native PAH gene. In some embodiments, the promoter is not associated with the PAH gene in nature. In some embodiments, the promoter is any one or more of the promoters disclosed herein. In some embodiments, the engineered microorganism, e.g., engineered bacterium that express PAH, convert phenylalanine into tyrosine.

In some embodiments, the engineered microorganism, e.g., engineered bacterium, comprises a modified pks island and a gene encoding PAL. In some embodiments, the engineered microorganism, e.g., engineered bacterium, comprise a gene encoding PAL, wherein the PAL gene is operably linked to an inducible promoter. In some embodiments, the microorganisms, e.g., bacteria, comprise a non-native PAL gene. In some embodiments, the microorganisms, e.g., bacteria, comprise additional copies of a native PAL gene. In some embodiments, the promoter is not associated with the PAL gene in nature. In some embodiments, the promoter is any one or more of the promoters disclosed herein. In some embodiments, the engineered microorganism, e.g., engineered bacterium that express PAL, convert phenylalanine into trans-cinnamate and ammonia.

In some embodiments, the engineered microorganism, e.g., engineered bacterium, comprises a modified pks island and a gene encoding LAAD. In some embodiments, the LAAD gene is operably linked to an inducible promoter. In some embodiments, the microorganisms, e.g., bacteria, comprise a non-native LAAD gene. In some embodiments, the microorganisms, e.g., bacteria, comprise additional copies of a native LAAD gene. In some embodiments, the promoter is not associated with the LAAD gene in nature. In some embodiments, the engineered microorganism, e.g., engineered bacterium that express LAAD, convert phenylalanine into phenylpyruvic acid.

In some embodiments, the engineered microorganism, e.g., engineered bacterium, comprise a modified pks island and a gene encoding a phenylalanine transporter, e.g., PheP. In some embodiments, the PheP gene is operably linked to an inducible promoter. In some embodiments, the engineered microorganism, e.g., engineered bacterium, comprise a gene encoding a non-native phenylalanine transporter gene, e.g., additional copies of a native phenylalanine transporter. In some embodiments, the microorganisms, e.g., bacteria, comprise additional copies of a native PheP gene. In some embodiments, the promoter is not associated with the PheP gene in nature. In some embodiments, the engineered microorganism, e.g., engineered bacterium that express PheP, transport phenylalanine into the bacterial cell.

In some embodiments, a single promoter controls expression of the one or more gene(s) encoding the PME and the phenylalanine transporter. In some embodiments, separate copies of the same promoter controls expression of the expression of the PME and the phenylalanine transporter. In some embodiments, different promoters control expression of the PME and the phenylalanine transporter. In some embodiments, the promoter that controls expression of PME is different from the promoter(s) that controls expression of the phenylalanine transporter. In some embodiments, the promoter(s) operably linked to the gene(s) encoding the PME and the gene(s) encoding the phenylalanine transporter are induced by exogenous environmental conditions found in a mammalian gut. In some embodiments, the promoter(s) operably linked to the gene(s) encoding the PME and the gene(s) encoding the phenylalanine transporter are induced under low-oxygen or anaerobic conditions, e.g., an FNR-responsive promoter, an ANR-responsive promoter, and a DNR-responsive promoter. In some embodiments, the promoter(s) operably linked to the gene(s) encoding the PME and the gene(s) encoding the phenylalanine transporter is a thermoregulated promoter. In some embodiments, the promoter(s) operably linked to the gene(s) encoding the PME and the gene(s) encoding the phenylalanine transporter are induced by arabinose, IPTG, tetracycline, or rhamnose. In some embodiments, the gene(s) encoding the PME, e.g., PAL and/or LAAD, is operably linked to a promoter selected from a promoter that is induced under low-oxygen or anaerobic conditions, a thermoregulated promoter, and a promoter that is induced by arabinose, IPTG, tetracycline, or rhamnose. In some embodiments, the thermoregulated promoter is capable of being induced at a temperature between 37° C. and 42° C. In some embodiments, the thermoregulated promoter is a lambda CI inducible promoter. In some embodiments, the genetically engineered bacteria further comprise one or more gene(s) encoding a temperature sensitive CI repressor mutant, which, in some embodiments, is CI857.

In some embodiments, the engineered microorganism, e.g., engineered bacterium, comprise a modified pks island and a gene encoding GOT1. In some embodiments, the engineered microorganism, e.g., engineered bacterium, comprise a gene encoding GOT1, wherein the GOT1 gene is operably linked to an inducible promoter. In some embodiments, the microorganisms, e.g., bacteria, comprise a non-native GOT1 gene. In some embodiments, the microorganisms, e.g., bacteria, comprise additional copies of a native GOT1 gene. In some embodiments, the promoter is not associated with the GOT1 gene in nature. In some embodiments, the promoter is any one or more of the promoters disclosed herein. In some embodiments, the engineered microorganism, e.g., engineered bacterium that express GOT1, convert phenylalanine into phenylpyruvate.

In some embodiments, the engineered microorganism, e.g., engineered bacterium, comprise a modified pks island and a gene encoding GOT2. In some embodiments, the engineered microorganism, e.g., engineered bacterium, comprise a gene encoding GOT2, wherein the GOT2 gene is operably linked to an inducible promoter. In some embodiments, the microorganisms, e.g., bacteria, comprise a non-native GOT2 gene. In some embodiments, the microorganisms, e.g., bacteria, comprise additional copies of a native GOT2 gene. In some embodiments, the promoter is not associated with the GOT2 gene in nature. In some embodiments, the promoter is any one or more of the promoters disclosed herein. In some embodiments, the engineered microorganism, e.g., engineered bacterium that express GOT2, convert phenylalanine into phenylpyruvate.

In some embodiments, the engineered microorganism, e.g., engineered bacterium, comprise a modified pks island and a gene encoding TAT. In some embodiments, the engineered microorganism, e.g., engineered bacterium, comprise a gene encoding TAT, wherein the TAT gene is operably linked to an inducible promoter. In some embodiments, the microorganisms, e.g., bacteria, comprise a non-native TAT gene. In some embodiments, the microorganisms, e.g., bacteria, comprise additional copies of a native TAT gene. In some embodiments, the promoter is not associated with the TAT gene in nature. In some embodiments, the promoter is any one or more of the promoters disclosed herein. In some embodiments, the engineered microorganism, e.g., engineered bacterium that express TAT, convert phenylalanine into phenylpyruvate.

In some embodiments, the engineered microorganism, e.g., engineered bacterium, comprise a modified pks island and a gene encoding DDC. In some embodiments, the engineered microorganism, e.g., engineered bacterium, comprise a gene encoding DDC, wherein the DDC gene is operably linked to an inducible promoter. In some embodiments, the microorganisms, e.g., bacteria, comprise a non-native DDC gene. In some embodiments, the microorganisms, e.g., bacteria, comprise additional copies of a native DDC gene. In some embodiments, the promoter is not associated with the DDC gene in nature. In some embodiments, the promoter is any one or more of the promoters disclosed herein. In some embodiments, the engineered microorganism, e.g., engineered bacterium that express DDC, convert phenylalanine into phenylethylamine.

In some embodiments, the engineered microorganism, e.g., engineered bacterium, comprises a modified pks island and one or more gene(s) and/or gene cassette(s) encoding at least one oxalate catabolism enzyme. In some embodiments, the engineered bacteria comprise one or more gene(s) and/or gene cassette(s) encoding at least one oxalate catabolism enzyme and are capable of converting oxalate into oxalyl-CoA. In some embodiments, the engineered bacteria comprise one or more gene(s) and/or gene cassette(s) encoding at least one oxalate catabolism enzyme and are capable of converting oxalyl-CoA into formate and carbon dioxide. In some embodiments, the engineered bacteria comprise one or more gene(s) and/or gene cassette(s) encoding at least one oxalate catabolism enzyme and are capable of converting oxalate into oxalyl-CoA, and oxalyl-CoA into formate and carbon dioxide. In some embodiments, the engineered bacteria of the disclosure comprise one or more gene(s) and or gene cassette encoding one or more oxalate catabolism enzyme(s) which convert oxalate and formyl CoA into oxalyl-CoA and formate. In some embodiments, the engineered bacteria of the disclosure comprise one or more gene(s) and/or gene cassette(s) encoding one or more oxalate catabolism enzyme(s) which convert oxalate and acetyl-coA into oxalyl-CoA and acetate. In some embodiments, the engineered bacteria of the disclosure comprise one or more gene(s) and/or gene cassette(s) encoding one or more oxalate catabolism enzyme(s) which convert oxalate and CoA into oxalyl-CoA (e.g., by converting one ATP to AMP plus diphosphate). In some embodiments, the engineered bacteria of the disclosure comprise one or more gene(s) and/or gene cassette(s) encoding one or more oxalate catabolism enzyme(s) which convert oxalyl-CoA to carbon dioxide and formyl-CoA. In some embodiments, the engineered bacteria produce formate as a result of oxalate catabolism. In some embodiments, the engineered bacteria produce formate and carbon dioxide as a result of oxalate catabolism. In some embodiments, the engineered bacteria produce acetate as a result of oxalate catabolism. In some embodiments, the engineered bacteria produce acetate and carbon dioxide as a result of oxalate catabolism. In some embodiments, the engineered bacteria produce formate, acetate, and carbon dioxide as a result of oxalate catabolism.

In some embodiments, the engineered bacteria comprise a modified pks island, as disclosed herein, and one or more gene(s) and/or gene cassette(s) encoding at least one oxalate catabolism enzyme and are capable of converting oxalate into oxalyl-CoA. In some embodiments, the engineered bacteria comprise a modified pks island, as disclosed herein, and one or more gene(s) and/or gene cassette(s) encoding at least one oxalate catabolism enzyme and are capable of converting oxalyl-CoA into formate and carbon dioxide. In some embodiments, the engineered bacteria comprise a modified pks island, as disclosed herein, and one or more gene(s) and/or gene cassette(s) encoding at least one oxalate catabolism enzyme and are capable of converting oxalate into oxalyl-CoA, and oxalyl-CoA into formate and carbon dioxide. In some embodiments, the engineered bacteria comprise a modified pks island, as disclosed herein, and one or more gene(s) and/or gene cassette(s) encoding at least one oxalate catabolism enzyme(s) which convert oxalate and formyl CoA into oxalyl-CoA and formate In some embodiments, the engineered bacteria comprise a modified pks island, as disclosed herein, and one or more gene(s) and/or gene cassette(s) encoding at least one oxalate catabolism enzyme(s) which convert oxalate and acetyl-coA into oxalyl-CoA and acetate. In some embodiments, the engineered bacteria comprise a modified pks island, as disclosed herein, and one or more gene(s) and/or gene cassette(s) encoding at least one oxalate catabolism enzyme(s) which convert oxalate and CoA into oxalyl-CoA (e.g., by converting one ATP to AMP plus diphosphate). In some embodiments, the engineered bacteria comprise a modified pks island, as disclosed herein, and one or more gene(s) and/or gene cassette(s) encoding at least one oxalate catabolism enzyme(s) which convert oxalyl-CoA to carbon dioxide and formyl-CoA. In some embodiments, the engineered bacteria, comprising a modified pks island as disclosed herein, produce formate as a result of oxalate catabolism. In some embodiments, the engineered bacteria, comprising a modified pks island as disclosed herein, produce formate and carbon dioxide as a result of oxalate catabolism. In some embodiments, the engineered bacteria, comprising a modified pks island as disclosed herein, produce acetate as a result of oxalate catabolism. In some embodiments, the engineered bacteria, comprising a modified pks island as disclosed herein, produce acetate and carbon dioxide as a result of oxalate catabolism. In some embodiments, the engineered bacteria, comprising a modified pks island as disclosed herein, produce formate, acetate, and carbon dioxide as a result of oxalate catabolism.

In some embodiments, the one or more oxalate catabolism enzyme(s) increases the rate of oxalate and/or oxalyl-CoA catabolism in the cell. In some embodiments, the one or more oxalate catabolism enzyme(s) decreases the level of oxalate in the cell. In some embodiments, the one or more oxalate catabolism enzyme(s) decreases the level of oxalyl-CoA in the cell. In some embodiments, the one or more oxalate catabolism enzyme(s) decreases the level of oxalic acid in the cell.

In some embodiments, the one or more oxalate catabolism enzyme(s) increases the rate of oxalate and/or oxalyl-CoA catabolism in the cell. In some embodiments, the one or more oxalate catabolism enzyme(s) decreases the level of oxalate in the cell. In some embodiments, the one or more oxalate catabolism enzyme(s) decreases the level of oxalyl-CoA in the cell. In some embodiments, the one or more oxalate catabolism enzyme(s) decreases the level of oxalic acid in the cell.

In some embodiments, the one or more oxalate catabolism enzyme(s) increases the level of oxalyl-CoA in the cell as compared to the level of its corresponding oxalate in the cell. In some embodiments, the one or more oxalate catabolism enzyme(s) increases the level of formate and carbon dioxide in the cell as compared to the level of its corresponding oxalyl-CoA in the cell. In some embodiments, the one or more oxalate catabolism enzyme(s) decreases the level of the oxalate and/or oxalyl CoA as compared to the level of oxalate in the cell.

Enzymes involved in the catabolism of oxalate may be expressed or modified in the bacteria of the invention in order to enhance catabolism of oxalate. Specifically, when at least one oxalate catabolism enzyme is expressed in the engineered bacterial cells of the invention, the engineered bacterial cells convert more oxalate into oxalyl-CoA, or convert more oxalyl-CoA into formate and carbon dioxide when the catabolism enzyme is expressed than unmodified bacteria of the same bacterial subtype under the same conditions. Thus, the genetically engineered bacteria comprising a heterologous gene encoding an oxalate catabolism enzyme can catabolize oxalate and/or oxalyl-CoA to treat disorders in which oxalate is detrimental, such as PHI, PHII, PHIII, and secondary hyperoxaluria, enteric hyperoxaluria, and idiopathic hyperoxaluria.

In one embodiment, the bacterial cell of the invention comprises at least one heterologous gene encoding at least one oxalate catabolism enzyme. In one embodiment, the bacterial cell of the invention comprises at least one heterologous gene encoding an importer of oxalate and at least one heterologous gene encoding at least one oxalate catabolism enzyme. In one embodiment, the bacterial cell of the invention comprises at least one heterologous gene encoding an exporter of formate and at least one heterologous gene encoding at least one oxalate catabolism enzyme. In one embodiment, the bacterial cell of the invention comprises at least one heterologous gene encoding an oxalate:formate antiporter and at least one heterologous gene encoding at least one oxalate catabolism enzyme.

Oxalate catabolism enzymes are known in the art. In some embodiments, AN oxalate catabolism enzyme is encoded by at least one gene encoding at least one oxalate catabolism enzyme derived from a bacterial species. In some embodiments, an oxalate catabolism enzyme is encoded by a gene encoding an oxalate catabolism enzyme derived from a non-bacterial species. In some embodiments, an oxalate catabolism enzyme is encoded by a gene derived from a eukaryotic species, e.g., a yeast species or a plant species. In one embodiment, an oxalate catabolism enzyme is encoded by a gene derived from a human. In one embodiment, the gene sequence(s) encoding the one or more oxalate catabolism enzyme(s) is derived from an organism of the genus or species that includes, but is not limited to, Bifidobacterium, Bordetella, Bradyrhizobium, Burkholderia, Clostridium, Enterococcus, Escherichia, Eubacterium, Lactobacillus, Magnetospirillium, Mycobacterium, Neurospora, Oxalobacter, e.g., Oxalobacter formigenes, Ralstonia, Rhodopseudomonas, Shigella, Thermoplasma, and Thauera, e.g., Bifidobacterium animalis, Bifidobacterium bifidum, Bifidobacterium infantis, Bifidobacterium lactis, Bifidobacterium longum, Bordatella bronchiseptica, Bordatella parapertussis, Burkholderia fungorum, Burkholderia xenovorans, Bradyrhizobium japonicum, Clostridium acetobutylicum, Clostridium difficile, Clostridium scindens, Clostridium sporogenes, Clostridium tentani, Enterococcus faecalis, Escherichia coli, Eubacterium lentum, Lactobacillus acidophilus, Lactobacillus bulgaricus, Lactobacillus casei, Lactobacillus gasseri, Lactobacillus plantarum, Lactobacillus rhamnosus, Lactococcus lactis, Magnetospirillium magentotaticum, Mycobacterium avium, Mycobacterium intracellulare, Mycobacterium kansasii, Mycobacterium leprae, Mycobacterium smegmatis, Mycobacterium tuberculosis, Mycobacterium ulcerans, Neurospora crassa, Oxalobacter formigenes, Providencia rettgeri, Eubacterium lentum, Ralstonia eutropha, Ralstonia metallidurans, Rhodopseudomonas palustris, Shigella flexneri, Thermoplasma volcanium, and Thauera aromatica.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.). In one embodiment, the gene or protein is at least 90%, 91%, 92%, 93%, 94$, 95%, 96%, 97%, 98%, 99% or 100% identical to a gene or protein disclosed herein.

In some embodiments, the gene encoding an oxalate catabolism enzyme is mutagenized; mutants exhibiting increased activity are selected; and the mutagenized gene encoding the oxalate catabolism enzyme is isolated and inserted into the bacterial cell. In one embodiment, spontaneous mutants that arise that allow bacteria to grow on oxalate as the sole carbon source can be screened for and selected. The gene comprising the modifications described herein may be present on a plasmid or chromosome. Non-limiting examples of oxalate catabolism enzymes of the disclosure are listed in Table 2.

TABLE 2
Oxalate Catabolism Enzyme Polynucleotide Sequences

| Description | Sequence | SEQ ID NO |
|---|---|---|
| frc (formyl-CoA | ATGACTAAACCATTAGATGGAATTAATGTGCTTG | SEQ ID NO: 1 |
| transferase from | ACTTTACCCACGTCCAGGCAGGTCCTGCCTGTAC | |
| O. formigenes | ACAGATGATGGGTTTCTTGGGCGCAAACGTCATC | |
| | AAGATTGAAAGACGTGGTTCCGGAGATATGACTC | |
| | GTGGATGGCTGCAGGACAAACCAAATGTTGATTC | |
| | CCTGTATTTCACGATGTTCAACTGTAACAAACGTT | |
| | CGATTGAACTGGACATGAAAACCCCGGAAGGCA | |
| | AAGAGCTTCTGGAACAGATGATCAAGAAAGCCG | |
| | ACGTCATGGTCGAAAACTTCGGACCAGGCGCACT | |
| | GGACCGTATGGGCTTTACTTGGGAATACATTCAG | |
| | GAACTGAATCCACGCGTCATTCTGGCTTCCGTTA | |
| | AAGGCTATGCAGAAGGCCACGCCAACGAACACC | |
| | TGAAAGTTTATGAAAACGTTGCACAGTGTTCCGG | |
| | CGGTGCTGCAGCTACCACCGGTTTCTGGGATGGT | |
| | CCTCCAACCGTTTCCGGCGCTGCTCTGGGTGACTC | |
| | CAACTCCGGTATGCACCTGATGATCGGTATTCTG | |
| | GCCGCTCTGGAAATGCGTCACAAAACCGGCCGTG | |
| | GTCAGAAAGTTGCCGTCGCTATGCAGGACGCTGT | |
| | TCTGAATCTGGTTCGTATCAAACTGCGTGACCAG | |
| | CAACGTCTGGAAAGAACCGGCATTCTGGCTGAAT | |
| | ACCCACAGGCTCAGCCTAACTTTGCCTTCGACAG | |
| | AGACGGTAACCCACTGTCCTTCGACAACATCACT | |
| | TCCGTTCCACGTGGTGGTAACGCAGGTGGCGGCG | |
| | GCCAGCCAGGCTGGATGCTGAAATGTAAAGGTTG | |
| | GGAAACCGATGCGGACTCCTACGTTTACTTCACC | |
| | ATCGCTGCAAACATGTGGCCACAGATCTGCGACA | |
| | TGATCGACAAGCCAGAATGGAAAGACGACCCAG | |
| | CCTACAACACATTCGAAGGTCGTGTTGACAAGCT | |
| | GATGGACATCTTCTCCTTCATCGAAACCAAGTTC | |
| | GCTGACAAGGACAAATTCGAAGTTACCGAATGGG | |
| | CTGCCCAGTACGGCATTCCTTGCGGTCCGGTCAT | |
| | GTCCATGAAAGAACTGGCTCACGATCCTTCCCTG | |
| | CAGAAAGTTGGTACCGTCGTTGAAGTIGTCGACG | |
| | AAATTCGTGGTAACCACCTGACCGTTGGCGCACC | |
| | GTTCAAATTCTCCGGATTCCAGCCGGAAATTACC | |
| | CGTGCTCCGCTGTTGGGCGAACATACCGACGAAG | |
| | TTCTGAAAGAACTGGGTCTTGACGATGCCAAGAT | |
| | CAAGGAACTGCATGCAAAACAGGTAGTTTGA | |
| oxc (oxalylCoA | ATGAGTAACGACGACAATGTAGAGTTGACTGATG | SEQ ID NO: 2 |
| decarboxylase from | GCTTTCATGTTTTGATCGATGCCCTGAAAATGAAT | |
| O. formigenes) also | GACATCGATACCATGTATGGTGTTGTCGGCATTC | |
| referred to as oxdc | CTATCACGAACCTGGCTCGTATGTGGCAAGATGA | |
| (oxalate | CGGTCAGCGTTTTTACAGCTTCCGTCACGAACAA | |
| decarboxylase) | CACGCAGGTTATGCAGCTTCTATCGCCGGTTACA | |
| | TCGAAGGAAAACCTGGCGTTTGCTTGACCGTTTC | |
| | CGCCCCTGGCTTCCTGAACGGCGTGACTTCCCTG | |
| | GCTCATGCAACCACCAACTGCTTCCCAATGATCC | |
| | TGTTGAGCGGTTCCAGTGAACGTGAAATCGTCGA | |
| | TTTGCAACAGGGCGATTACGAAGAAATGGATCAG | |
| | ATGAATGTTGCACGTCCACACTGCAAAGCTTCTTT | |
| | CCGTATCAACAGCATCAAAGACATTCCAATCGGT | |
| | ATCGCTCGTGCAGTTCGCACCGCTGTATCCGGAC | |
| | GTCCAGGTGGTGTTTACGTTGACTTGCCAGCAAA | |
| | ACTGTTCGGTCAGACCATTTCTGTAGAAGAAGCT | |
| | AACAAACTGCTCTTCAAACCAATCGATCCAGCTC | |
| | CGGCACAGATTCCTGCTGAAGACGCTATCGCTCG | |
| | CGCTGCTGACCTGATCAAGAACGCCAAACGTCCA | |
| | GTTATCATGCTGGGTAAAGGCGCTGCATACGCAC | |
| | AATGCGACGACGAAATCCGCGCACTGGTTGAAGA | |
| | AACCGGCATCCCATTCCTGCCAATGGGTATGGCT | |
| | AAAGGCCTGCTGCCTGACAACCATCCACAATCCG | |
| | CTGCTGCAACCCGTGCTTTCGCACTGGCACAGTG | |
| | TGACGTTTGCGTACTGATCGGCGCTCGTCTGAACT | |
| | GGCTGATGCAGCACGGTAAAGGCAAAACCTGGG | |
| | GCGACGAACTGAAGAAATACGTTCAGATCGACAT | |
| | CCAGGCTAACGAAATGGACAGCAACCAGCCTATC | |
| | GCTGCACCAGTTGTTGGTGACATCAAGTCCGCCG | |
| | TTTCCCTGCTCCGCAAAGCACTGAAAGGCGCTCC | |
| | AAAAGCTGACGCTGAATGGACCGGCGCTCTGAAA | |
| | GCCAAAGTTGACGGCAACAAAGCCAAACTGGCT | |
| | GGCAAGATGACTGCCGAAACCCCATCCGGAATGA | |
| | TGAACTACTCCAATTCCCTGGGCGTTGTTCGTGAC | |
| | TTCATGCTGGCAAATCCGGATATTTCCCTGGTTAA | |
| | CGAAGGCGCTAATGCACTCGACAACACTCGTATG | |
| | ATTGTTGACATGCTGAAACCACGCAAACGTCTTG | |
| | ACTCCGGTACCTGGGGTGTTATGGGTATTGGTAT | |
| | GGGCTACTGCGTTGCTGCAGCTGCTGTTACCGGC | |
| | AAACCGGTTATCGCTGTTGAAGGCGATAGCGCAT | |
| | TCGGTTTCTCCGGTATGGAACTGGAAACCATCTG | |
| | CCGTTACAACCTGCCAGTTACCGTTATCATCATGA | |
| | ACAATGGTGGTATCTATAAAGGTAACGAAGCAGA | |
| | TCCACAACCAGGCGTTATCTCCTGTACCCGTCTGA | |
| | CCCGTGGTCGTTACGACATGATGATGGAAGCATT | |
| | TGGCGGTAAAGGTTATGTTGCCAATACTCCAGCA | |
| | GAACTGAAAGCTGCTCTGGAAGAAGCTGTTGCTT | |
| | CCGGCAAACCATGCCTGATCAACGCGATGATCGA | |
| | TCCAGACGCTGGTGTCGAATCTGGCCGTATCAAG | |
| | AGCCTGAACGTTGTAAGTAAAGTTGGCAAGAAAT | |
| | AA | |
| ScAAE3 (oxalate-CoA | ATGACCAGTGCAGCTACGGTGACCGCGAGCTTTA | SEQ ID NO: 3 |
| ligase from | ATGACACTTTTTCTGTGAGCGATAATGTCGCGGT | |
| S. cerevisiae) | AATCGTACCGGAAACCGATACGCAGGTCACCTAC | |
| | CGTGATCTTTCCCACATGGTAGGACACTTTCAAA | |
| | CAATGTTCACGAACCCGAATAGTCCTCTGTACGG | |
| | GGCGGTCTTTCGTCAAGACACGGTAGCGATTAGC | |
| | ATGCGTAACGGCCTTGAATTTATTGTGGCTTTCCT | |
| | TGGAGCCACGATGGATGCGAAAATTGGTGCGCCA | |
| | CTGAATCCCAATTATAAAGAGAAGGAGTTTAATT | |
| | TTTACCTGAATGACTTAAAGTCCAAAGCCATCTG | |
| | CGTGCCGAAAGGCACCACCAAACTGCAAAGTTCA | |
| | GAAATTCTTAAGAGTGCGTCCACGTTCGGGTGCT | |
| | TTATTGTGGAACTGGCGTTTGACGCCACCCGTTTT | |
| | CGTGTTGAATATGACATTTACTCCCCGGAGGACA | |
| | ATTATAAACGTGTGATCTACCGCAGCCTTAACAA | |
| | TGCTAAGTTTGTCAACACAAACCCTGTCAAGTTC | |
| | CCGGGTTTCGCCCGCAGCTCGGATGTTGCACTTAT | |
| | TTTGCATACCTCAGGCACCACTAGTACCCCAAAG | |
| | ACCGTACCCCTCTTGCATCTGAATATTGTCCGTTC | |
| | AACCCTGAATATCGCCAACACTTACAAACTTACC | |
| | CCGCTGGATCGCTCCTATGTTGTAATGCCGCTGTT | |
| | TCATGTACATGGATTAATCGGCGTCTTACTGAGT | |
| | ACGTTCCGCACCCAGGGCAGTGTAGTCGTCCCGG | |
| | ACGGCTTTCATCCGAAGCTCTTCTGGGATCAGTTT | |
| | GTTAAATATAACTGCAATTGGTTTAGTTGCGTCCC | |
| | AACGATCTCTATGATTATGTTGAATATGCCCAAA | |
| | CCGAATCCGTTTCCGCACATTCGCTTTATCCGCTC | |
| | ATGTAGCAGCGCGCTGGCGCCAGCAACGTTTCAC | |
| | AAGCTGGAAAAAGAATTTAATGCCCCAGTTCTGG | |
| | AAGCGTACGCGATGACAGAAGCATCTCATCAGAT | |
| | GACCAGTAACAATCTGCCTCCCGGTAAACGTAAA | |
| | CCGGGGACCGTGGGCCAACCTCAAGGTGTAACCG | |
| | TAGTAATCCTGGATGACAACGATAACGTTCTGCC | |
| | GCCCGGCAAAGTTGGCGAGGTGTCGATCCGTGGG | |
| | GAGAACGTCACCCTGGGCTACGCTAATAACCCGA | |
| | AAGCTAACAAAGAAAACTTCACTAAACGTGAAA | |
| | ACTATTTCCGTACCGGGGATCAGGGCTACTTCGA | |
| | CCCGGAGGGCTTTCTCGTGCTGACCGGCCGCATT | |
| | AAAGAATTGATCAATCGCGGTGGTGAAAAAATTA | |
| | GTCCTATTGAACTGGACGGAATCATGCTCTCGCA | |
| | TCCTAAAATCGACGAGGCGGTGGCGTTCGGCGTT | |
| | CCAGATGATATGTATGGCCAAGTCGTTCAGGCGG | |
| | CAATCGTGTTGAAAAAGGGGGAAAAGATGACCT | |
| | ATGAAGAATTAGTGAATTTCCTGAAAAAGCATTT | |
| | AGCAAGCTTTAAAATCCCAACCAAAGTCTACTTT | |
| | GTGGATAAGCTGCCTAAAACGGCCACCGGGAAG | |
| | ATTCAACGTCGCGTAATCGCCGAAACCTTCGCGA | |
| | AATCTAGTCGCAACAAAAGCAAACTTtaa | |
| yfdE (Acetyl- | atgACAAATAATGAAAGCAAAGGGCCGTTTGAAGG | SEQ ID NO: 4 |
| CoA:oxalate CoA- | CTTATTAGTTATCGATATGACACATGTCCTTAATG | |
| transferase from | GACCTTTCGGAACTCAACTTCTTTGTAATATGGGC | |
| E coli) | GCAAGGGTAATTAAAGTTGAGCCGCCGGGTCATG | |
| | GTGATGATACCCGCACATTTGGTCCCTATGTGGA | |
| | TGGACAGTCACTCTATTACAGTTTTATTAATCATG | |
| | GCAAAGAGAGTGTGGTTCTTGATTTAAAGAATGA | |
| | TCACGATAAAAGTATATTTATAAATATGCTCAAA | |
| | CAAGCTGATGTATTAGCTGAGAATTTTCGCCCAG | |
| | GTACAATGGAAAAACTGGGGTTTTCATGGGAAAC | |
| | GCTTCAAGAAATCAACCCGCGCCTCATATATGCT | |
| | TCATCGTCAGGTTTCGGACATACCGGTCCGCTAA | |
| | AAGATGCTCCTGCCTACGATACCATCATTCAGGC | |
| | AATGAGCGGGATAATGATGGAAACAGGATATCCT | |
| | GATGCTCCGCCAGTGCGCGTTGGTACATCTCTTGC | |
| | GGATCTATGCGGCGGTGTCTATTTATTCAGCGGA | |
| | ATAGTGAGTGCACTTTATGGCCGCGAAAAGAGCC | |
| | AGAGAGGGGCGCATGTCGATATAGCGATGTTTGA | |
| | TGCCACGCTGAGTTTTCTGGAGCATGGTCTGATG | |
| | GCATATATCGCAACTGGGAAGTCACCACAACGTC | |
| | TGGGAAATCGCCATCCCTACATGGCACCTTTTGA | |
| | TGTTTTCAATACTCAGGATAAGCCGATTACGATTT | |
| | GTTGTGGTAATGACAAGCTTTTTTCTGCGTTATGC | |
| | CAGGCACTGGAGCTTACGGAACTGGTTAATGATC | |
| | CCCGATTTAGCAGCAATATTTTACGCGTACAAAA | |
| | CCAGGCTATTCTTAAACAATATATTGAGCGGACG | |
| | TTAAAAACGCAGGCAGCTGAAGTTTGGTTAGCCA | |
| | GAATACATGAAGTTGGTGTACCCGTCGCGCCGTT | |
| | ATTAAGTGTGGCTGAGGCCATTAAATTGCCACAA | |
| | ACTCAGGCGAGAAATATGTTGATTGAAGCCGGGG | |
| | GAATAATGATGCCGGGTAATCCGATAAAAATCAG | |
| | CGGCTGCGCGGACCCGCATGTTATGCCGGGAGCG | |
| | GCAACGCTCGACCAGCATGGGGAACAAATTCGCC | |
| | AGGAGTTCTCATCAtaa | |

In one embodiment, the gene sequence(s) encoding the one or more oxalate catabolism enzyme(s) comprises a formyl-CoA:oxalate CoA-transferase sequence. In one embodiment, the formyl-CoA:oxalate CoA-transferase is frc, e.g., from O. formigenes. Accordingly, in one embodiment, the frc gene has at least about 80% identity with the entire sequence of SEQ ID NO: 1. Accordingly, in one embodiment, the frc gene has at least about 90% identity with the entire sequence of SEQ ID NO: 1. Accordingly, in one embodiment, the frc gene has at least about 95% identity with the entire sequence of SEQ ID NO: 1. Accordingly, in one embodiment, the frc gene has at least about 85%, 86%, 87%, 88%, 89%, 90%, 91%, 92%, 93%, 94%, 95%, 96%, 97%, 98%, or 99% identity with the entire sequence of SEQ ID NO: 1. In another embodiment, the frc gene comprises the sequence of SEQ ID NO: 1. In yet another embodiment the frc gene consists of the sequence of SEQ ID NO:1.

In one embodiment, the gene sequence(s) encoding the one or more oxalate catabolism enzyme(s) comprises a oxalyl-CoA decarboxylase sequence. In one embodiment, the oxalyl-CoA decarboxylase is oxc, e.g., from O. formigenes. Accordingly, in one embodiment, the oxc gene has at least about 80% identity with the entire sequence of SEQ ID NO: 2. Accordingly, in one embodiment, the oxc gene has at least about 90% identity with the entire sequence of SEQ ID NO: 2. Accordingly, in one embodiment, the oxc gene has at least about 95% identity with the entire sequence of SEQ ID NO: 2. Accordingly, in one embodiment, the oxc gene has at least about 85%, 86%, 87%, 88%, 89%, 90%, 91%, 92%, 93%, 94%, 95%, 96%, 97%, 98%, or 99% identity with the entire sequence of SEQ ID NO: 2. In another embodiment, the oxc gene comprises the sequence of SEQ ID NO: 2. In yet another embodiment the oxc gene consists of the sequence of SEQ ID NO: 2. In another embodiment, the oxc gene consists of the sequence of SEQ ID NO: 2.

In one embodiment, the at least one gene encoding the at least one oxalate catabolism enzyme comprises an oxalate-CoA ligase sequence. In one embodiment, the oxalate-CoA ligase is ScAAE3 from S. cerevisiae. Accordingly, in one embodiment, the ScAAE3 gene has at least about 80% identity with the entire sequence of SEQ ID NO: 3. Accordingly, in one embodiment, the ScAAE3 gene has at least about 90% identity with the entire sequence of SEQ ID NO: 3. Accordingly, in one embodiment, the ScAAE3 gene has at least about 95% identity with the entire sequence of SEQ ID NO: 3. Accordingly, in one embodiment, the ScAAE3 gene has at least about 85%, 86%, 87%, 88%, 89%, 90%, 91%, 92%, 93%, 94%, 95%, 96%, 97%, 98%, or 99% identity with the entire sequence of SEQ ID NO: 3. In another embodiment, the ScAAE3 gene comprises the sequence of SEQ ID NO: 3. In yet another embodiment the ScAAE3 gene consists of the sequence of SEQ ID NO: 3.

In one embodiment, the at least one gene encoding the at least one oxalate catabolism enzyme comprises an acetyl-CoA:oxalate CoA-transferase sequence. In one embodiment, the acetyl-CoA:oxalate CoA-transferase is YfdE from E. coli from S. cerevisiae. Accordingly, in one embodiment, the YfdE gene has at least about 80% identity with the entire sequence of SEQ ID NO: 4. Accordingly, in one embodiment, the YfdE gene has at least about 90% identity with the entire sequence of SEQ ID NO: 4. Accordingly, in one embodiment, the YfdE gene has at least about 95% identity with the entire sequence of SEQ ID NO: 4. Accordingly, in one embodiment, the YfdE gene has at least about 85%, 86%, 87%, 88%, 89%, 90%, 91%, 92%, 93%, 94%, 95%, 96%, 97%, 98%, or 99% identity with the entire sequence of SEQ ID NO: 4. In another embodiment, the YfdE gene comprises the sequence of SEQ ID NO: 4. In yet another embodiment the YfdE gene consists of the sequence of SEQ ID NO: 4.

TABLE 3
Polypeptide Sequences of Oxalate Catabolism Enzymes

| Description | Sequence | SEQ ID NO |
|---|---|---|
| Frc (Formyl-CoA | MTKPLDGINVLDFTHVQAGPACTQMMGFLGANVIKIERR | SEQ ID NO: 5 |
| transferase | GSGDMTRGWLQDKPNVDSLYFTMFNCNKRSIELDMKTPE | |
| from | GKELLEQMIKKADVMVENFGPGALDRMGFTWEYIQELNP | |
| O. formigenes) | RVILASVKGYAEGHANEHLKVYENVAQCSGGAAATTGF | |
| | WDGPPTVSGAALGDSNSGMHLMIGILAALEMRHKTGRGQ | |
| | KVAVAMQDAVLNLVRIKLRDQQRLERTGILAEYPQAQPN | |
| | FAFDRDGNPLSFDNITSVPRGGNAGGGGQPGWMLKCKG | |
| | WETDADSYVYFTIAANMWPQICDMIDKPEWKDDPAYNTF | |
| | EGRVDKLMDIFSFIETKFADKDKFEVTEWAAQYGIPCGPV | |
| | MSMKELAHDPSLQKVGTVVEVVDEIRGNHLTVGAPFKFS | |
| | GFQPEITRAPLLGEHTDEVLKELGLDDAKIKELHAKQVV* | |
| Oxc (oxalylCoA | MSNDDNVELTDGFHVLIDALKMNDIDTMYGVVGIPITNL | SEQ ID NO: 6 |
| decarboxylase | ARMWQDDGQRFYSFRHEQHAGYAASIAGYIEGKPGVCLT | |
| from | VSAPGFLNGVTSLAHATTNCFPMILLSGSSEREIVDLQQGD | |
| O. formigenes) | YEEMDQMNVARPHCKASFRINSIKDIPIGIARAVRTAVSGR | |
| | PGGVYVDLPAKLFGQTISVEEANKLLFKPIDPAPAQIPAED | |
| | AIARAADLIKNAKRPVIMLGKGAAYAQCDDEIRALVEETG | |
| | IPFLPMGMAKGLLPDNHPQSAAATRAFALAQCDVCVLIG | |
| | ARLNWLMQHGKGKTWGDELKKYVQIDIQANEMDSNQPI | |
| | AAPVVGDIKSAVSLLRKALKGAPKADAEWTGALKAKVD | |
| | GNKAKLAGKMTAETPSGMMNYSNSLGVVRDFMLANPDI | |
| | SLVNEGANALDNTRMIVDMLKPRKRLDSGTWGVMGIGM | |
| | GYCVAAAAVTGKPVIAVEGDSAFGFSGMELETICRYNLPV | |
| | TVIIMNNGGIYKGNEADPQPGVISCTRLTRGRYDMMMEA | |
| | FGGKGYVANTPAELKAALEEAVASGKPCLINAMIDPDAG | |
| | VESGRIKSLNVVSKVGKK* | |
| ScAAE3 | MTSAATVTASFNDTFSVSDNVAVIVPETDTQVTYRDLSH | SEQ ID NO: 7 |
| (Oxalate-CoA | MVGHFQTMFTNPNSPLYGAVFRQDTVAISMRNGLEFIVAF | |
| ligase from | LGATMDAKIGAPLNPNYKEKEFNFYLNDLKSKAICVPKGTT | |
| S. cerevisiae) | KLQSSEILKSASTFGCFIVELAFDATRFRVEYDIYSPEDNY | |
| | KRVIYRSLNNAKFVNTNPVKFPGFARSSDVALILHTSGTTS | |
| | TPKTVPLLHLNIVRSTLNIANTYKLTPLDRSYVVMPLFHVH | |
| | GLIGVLLSTFRTQGSVVVPDGFHPKLFWDQFVKYNCNWF | |
| | SCVPTISMIMLNMPKPNPFPHIRFIRSCSSALAPATFHKLE | |
| | KEFNAPVLEAYAMTEASHQMTSNNLPPGKRKPGTVGQPQG | |
| | VTVVILDDNDNVLPPGKVGEVSIRGENVTLGYANNPKAN | |
| | KENFTKRENYFRTGDQGYFDPEGFLVLTGRIKELINRGGE | |
| | KISPIELDGIMLSHPKIDEAVAFGVPDDMYGQVVQAAIVL | |
| | KKGEKMTYEELVNFLKKHLASFKIPTKVYFVDKLPKTAT | |
| | GKIQRRVIAETFAKSSRNKSKL* | |
| yfdE (Acetyl- | MTNNESKGPFEGLLVIDMTHVLNGPFGTQLLCNMGARVI | SEQ ID NO: 8 |
| CoA:oxalate | KVEPPGHGDDTRTFGPYVDGQSLYYSFINHGKESVVLDLK | |
| CoA-transferase | NDHDKSIFINMLKQADVLAENFRPGTMEKLGFSWETLQEI | |
| from E. coli) | NPRLIYASSSGFGHTGPLKDAPAYDTIIQAMSGIMMETGYP | |
| | DAPPVRVGTSLADLCGGVYLFSGIVSALYGREKSQRGAH | |
| | VDIAMFDATLSFLEHGLMAYIATGKSPQRLGNRHPYMAPF | |
| | DVFNTQDKPITICCGNDKLFSALCQALELTELVNDPRFSSN | |
| | ILRVQNQAILKQYIERTLKTQAAEVWLARIHEVGVPVAPL | |
| | LSVAEAIKLPQTQARNMLIEAGGIMMPGNPIKISGCADPHV | |
| | MPGAATLDQHGEQIRQEFSS* | |
| yfdW (formyl | SYYHHHHHHLESTSLYKKAGLMSTPLQGIKVLDFTGVQS | SEQ ID NO: 9 |
| CoA transferase | GPSCTQMLAWFGADVIKIERPGVGDVTRHQLRDIPDIDAL | |
| from E. coli) | YFTMLNSNKRSIELNTKTAEGKEVMEKLIREADILVENFHP | |
| | GAIDHMGFTWEHIQEINPRLIFGSIKGFDECSPYVNVKAYE | |
| | NVAQAAGGAASTTGFWDGPPLVSAAALGDSNTGMHLLIG | |
| | LLAALLHREKTGRGQRVTMSMQDAVLNLCRVKLRDQQR | |
| | LDKLGYLEEYPQYPNGTFGDAVPRGGNAGGGGQPGWILK | |
| | CKGWETDPNAYIYFTIQEQNWENTCKAIGKPEWITDPAYS | |
| | TAHARQPHIFDIFAEIEKYTVTIDKHEAVAYLTQFDIPCAP | |
| | VLSMKEISLDPSLRQSGSVVEVEQPLRGKYLTVGCPMKFSA | |
| | FTPDIKAAPLLGEHTAAVLQELGYSDDEIAAMKQNHAI | |
| yfdU (oxalyl- | MSDQLQMTDGMHIIVEALKQNNIDTIYGVVGIPVTDMAR | SEQ ID NO: |
| CoA | HAQAEGIRYIGFRHEQSAGYAAAASGFLTQKPGICLTVSA | 10 |
| decarboxylase | PGFLNGLTALANATVNGFPMIMISGSSDRAIVDLQQGDYE | |
| E. coli) | ELDQMNAAKPYAKAAFRVNQPQDLGIALARAIRVSVSGR | |
| | PGGVYLDLPANVLAATMEKDEALTTIVKVENPSPALLPCP | |
| | KSVTSAISLLAKAERPLIILGKGAAYSQADEQLREFIESAQ | |
| | IPFLPMSMAKGILEDTHPLSAAAARSFALANADVVMLVGA | |
| | RLNWLLAHGKKGWAADTQFIQLDIEPQEIDSNRPIAVPVV | |
| | GDIASSMQGMLAELKQNTFTTPLVWRDILNIHKQQNAQK | |
| | MHEKLSTDTQPLNYFNALSAVRDVLRENQDIYLVNEGAN | |
| | TLDNARNIIDMYKPRRRLDCGTWGVMGIGMGYAIGASVT | |
| | SGSPVVAIEGDSAFGFSGMEIETICRYNLPVTIVIFNNGG | |
| | IYRGDGVDLSGAGAPSPTDLLHHARYDKLMDAFRGVGYNV | |
| | TTTDELRHALTTGIQSRKPTIINVVIDPAAGTESGHITKL | |
| | NPKQVAGN | |

In one embodiment, one or more polypeptide(s) encoded by the oxalate catabolism cassette(s) and expressed by the genetically engineered bacteria comprises a formyl-CoA transferase, e.g. frc from O. formigenes. In one embodiment the polypeptide(s) have at least about 80% identity with SEQ ID NO: 5. In another embodiment, one or more polypeptide(s) encoded by the oxalate catabolism gene(s) or gene cassette(s) and expressed by the genetically engineered bacteria have at least about 85% identity with SEQ ID NO: 5. In one embodiment, one or more polypeptide(s) encoded by the oxalate catabolism gene(s) or gene cassette(s) and expressed by the genetically engineered bacteria have at least about 90% identity with SEQ ID NO: 5. In one embodiment, one or more polypeptide(s) encoded by the oxalate catabolism gene(s) or gene cassette(s) and expressed by the genetically engineered bacteria have at least about 95% identity with SEQ ID NO: 5. In another embodiment, one or more polypeptide(s) encoded by the oxalate catabolism gene(s) or gene cassette(s) and expressed by the genetically engineered bacteria have at least about 96%, 97%, 98%, or 99% identity with SEQ ID NO: 5. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 5. In another embodiment, one or more polypeptide(s) encoded by the oxalate catabolism gene(s) or gene cassette(s) and expressed by the genetically engineered bacteria comprise the sequence of SEQ ID NO: 5. In yet another embodiment one or more polypeptide(s) encoded by the oxalate catabolism gene(s) or gene cassette(s) and expressed by the genetically engineered bacteria consist of the sequence of SEQ ID NO: 5.

In one embodiment, one or more polypeptide(s) encoded by the oxalate catabolism cassette(s) and expressed by the engineered bacteria comprises a oxalyl-CoA decarboxylase, e.g. oxc from O. formigenes. In one embodiment the polypeptide(s) have at least about 80% identity with SEQ ID NO: 6. In another embodiment, one or more polypeptide(s) encoded by the oxalate catabolism gene(s) or gene cassette(s and expressed by the engineered bacteria have at least about 85% identity with SEQ ID NO: 6. In one embodiment, one or more polypeptide(s) encoded by the oxalate catabolism gene(s) or gene cassette(s) and expressed by the engineered bacteria have at least about 90% identity with SEQ ID NO: 6. In one embodiment, one or more polypeptide(s) encoded by the oxalate catabolism gene(s) or gene cassette(s) and expressed by the engineered bacteria have at least about 95% identity with SEQ ID NO: 6. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 6. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 6. In another embodiment, one or more polypeptide(s) encoded by the oxalate catabolism gene(s) or gene cassette(s) and expressed by the engineered bacteria comprise the sequence of SEQ ID NO: 6. In yet another embodiment one or more polypeptide(s) encoded by the oxalate catabolism gene(s) or gene cassette(s) and expressed by the genetically engineered bacteria consist of the sequence of SEQ ID NO: 6.

In one embodiment, one or more polypeptide(s) encoded by the oxalate catabolism gene(s) or gene cassette(s) and expressed by the engineered bacteria comprises an oxalate-CoA ligase, e.g. ScAAE3 from S cerevisiae. In one embodiment the polypeptide(s) have at least about 80% identity with SEQ ID NO: 7. In another embodiment, one or more polypeptide(s) encoded by the oxalate catabolism gene(s) or gene cassette(s and expressed by the engineered bacteria have at least about 85% identity with SEQ ID NO: 7. In one embodiment, one or more polypeptide(s) encoded by the oxalate catabolism gene(s) or gene cassette(s) and expressed by the engineered bacteria have at least about 90% identity with SEQ ID NO: 7. In one embodiment, one or more polypeptide(s) encoded by the oxalate catabolism gene(s) or gene cassette(s) and expressed by the engineered bacteria have at least about 95% identity with SEQ ID NO: 7. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 7. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 7. In another embodiment, one or more polypeptide(s) encoded by the oxalate catabolism gene(s) or gene cassette(s) and expressed by the engineered bacteria comprise the sequence of SEQ ID NO: 7. In yet another embodiment one or more polypeptide(s) encoded by the oxalate catabolism cassette(s) and expressed by the genetically engineered bacteria consist of the sequence of SEQ ID NO: 7.

In one embodiment, one or more polypeptide(s) encoded by the oxalate catabolism cassette(s) and expressed by the engineered bacteria comprises an Acetyl-CoA:oxalate CoA-transferase from, e.g. YfdE from E. coli. In one embodiment the polypeptide(s) have at least about 80% identity with SEQ ID NO: 8. In another embodiment, one or more polypeptide(s) encoded by the oxalate catabolism gene(s) or gene cassette(s and expressed by the engineered bacteria have at least about 85% identity with SEQ ID NO: 8. In one embodiment, one or more polypeptide(s) encoded by the oxalate catabolism gene(s) or gene cassette(s) and expressed by the engineered bacteria have at least about 90% identity with SEQ ID NO: 8. In one embodiment, one or more polypeptide(s) encoded by the oxalate catabolism gene(s) or gene cassette(s) and expressed by the engineered bacteria have at least about 95% identity with SEQ ID NO: 8. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 8. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 8. In another embodiment, one or more polypeptide(s) encoded by the oxalate catabolism gene(s) or gene cassette(s) and expressed by the engineered bacteria comprise the sequence of SEQ ID NO: 8. In yet another embodiment one or more polypeptide(s) encoded by the oxalate catabolism gene(s) or gene cassette(s) and expressed by the engineered bacteria consist of the sequence of with SEQ ID NO: 8.

In one embodiment, one or more polypeptide(s) encoded by the oxalate catabolism gene(s) or gene cassette(s) and expressed by the genetically engineered bacteria comprises a formyl CoA transferase, e.g., yfdW from E. coli. In one embodiment the polypeptide(s) have at least about 80% identity with SEQ ID NO: 9. In another embodiment, one or more polypeptide(s) encoded by the oxalate catabolism gene(s) or gene cassette(s and expressed by the engineered bacteria have at least about 85% identity with SEQ ID NO: 9. In one embodiment, one or more polypeptide(s) encoded by the oxalate catabolism gene(s) or gene cassette(s) and expressed by the engineered bacteria have at least about 90% identity with SEQ ID NO: 9. In one embodiment, one or more polypeptide(s) encoded by the oxalate catabolism gene(s) or gene cassette(s) and expressed by the engineered bacteria have at least about 95% identity with SEQ ID NO: 9. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 9. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 9. In another embodiment, one or more polypeptide(s) encoded by the oxalate catabolism gene(s) or gene cassette(s) and expressed by the engineered comprise the sequence of SEQ ID NO: 9. In yet another embodiment one or more polypeptide(s) encoded by the oxalate catabolism gene(s) or gene cassette(s) and expressed by the engineered bacteria consist of the sequence of SEQ ID NO: 9.

In one embodiment, one or more polypeptide(s) encoded by the oxalate catabolism gene(s) or gene cassette(s) and expressed by the engineered bacteria comprises a oxalyl-CoA decarboxylase, e.g., yfdU from E. coli. In one embodiment the polypeptide(s) have at least about 80% identity with SEQ ID NO: 10. In another embodiment, one or more polypeptide(s) encoded by the oxalate catabolism gene(s) or gene cassette(s and expressed by the engineered bacteria have at least about 85% identity with SEQ ID NO: 10. In one embodiment, one or more polypeptide(s) encoded by the oxalate catabolism gene(s) or gene cassette(s) and expressed by the engineered bacteria have at least about 90% identity with SEQ ID NO: 10. In one embodiment, one or more polypeptide(s) encoded by the oxalate catabolism gene(s) or gene cassette(s) and expressed by the engineered bacteria have at least about 95% identity with SEQ ID NO: 10. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 10. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 10. In another embodiment, one or more polypeptide(s) encoded by the oxalate catabolism gene(s) or gene cassette(s) and expressed by the engineered bacteria comprise the sequence of SEQ ID NO: 10. In yet another embodiment one or more polypeptide(s) encoded by the oxalate catabolism gene(s) or gene cassette(s) and expressed by the engineered bacteria consist of the sequence of SEQ ID NO: 10.

In some embodiments, the engineered microorganisms, e.g., genetically engineered bacteria, comprising a modified pks island are non-pathogenic bacteria. In some embodiments, the engineered microorganisms, e.g., genetically engineered bacteria, are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. In some embodiments, non-pathogenic bacteria are Gram-negative bacteria. In some embodiments, non-pathogenic bacteria are Gram-positive bacteria. Exemplary bacteria include, but are not limited to, Bacteroides, Bifidobacterium, Clostridium, Escherichia, Lactobacillus, and Lactococcus. In some embodiments, the genetically engineered bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added).

Unmodified E. coli Nissle or genetically engineered bacteria may be destroyed, e.g., by defense factors in the gut or blood serum (Sonnenborn et al., 2009) or by activation of a kill switch, several hours or days after administration. Thus, the composition may require continued administration. In some embodiments, the residence time is calculated for a human subject.

In some embodiments, the therapeutic molecule, e.g., PAL, may be expressed on a low-copy plasmid, a high-copy plasmid, or on the chromosome, e.g., at one or more of the following insertion sites in E. coli Nissle: malE/K, insB/I, araC/BAD, lacZ, agaI/rsmI, thyA, and malP/T. The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon. In some embodiments, more than one copy, e.g., two, three, four, five, six, seven, eight, nine, ten or more copies of the therapeutic molecule, e.g., PAL, is integrated into the bacterial chromosome at one or more integration sites in the engineered microorganisms, e.g., genetically engineered bacteria. In some embodiments, the engineered microorganisms, e.g., genetically engineered bacteria, comprise one or more gene(s) encoding a phenylalanine metabolizing enzyme (PME); one or more gene(s) for producing an anti-cancer molecule, e.g., a deadenylate cyclase gene (e.g., dacA) or an enzyme capable of producing a STING agonist; and one or more gene(s) encoding a modified arginine biosynthesis pathway, e.g., deleted arginine repressor, modified arginine repressor binding sites, and/or arginine feedback resistant N-acetylglutamate synthase mutation, for producing arginine. In some embodiments, the engineered microorganisms, e.g., genetically engineered bacteria, comprise a gene encoding PME, wherein the PME gene is operably linked to an inducible promoter. In some embodiments, the microorganisms, e.g., bacteria, comprise a non-native PME gene. In some embodiments, the microorganisms, e.g., bacteria, comprise additional copies of a native PME gene. In some embodiments, the promoter is not associated with the PME gene in nature.

In some embodiments, the engineered microorganisms, e.g., genetically engineered bacteria, are auxotrophs for one or more essential genes. For example, a mutation of, modification of, or excision of an essential gene may result in the engineered microorganisms, e.g., genetically engineered bacteria, becoming an auxotroph. An auxotrophic modification is intended to cause bacteria to die in the absence of an exogenously added nutrient essential for survival or growth because they lack the gene(s) necessary to produce that essential nutrient. In some embodiments, any of the engineered microorganisms, e.g., genetically engineered bacteria, described herein also comprise a deletion or mutation in a gene required for cell survival and/or growth.

Exemplary auxotrophs are provided in WO2016090343, WO2016200614, WO2017139697, WO2016183531, WO2017087580, WO2016141108, WO2017074566, WO2017136792, WO2017136795, WO2018129404, WO2019014391, WO2016210384, WO2017123418, WO2017123676, WO2016183531, WO2018237198, WO2016201380, US20170216370, and WO2017040719, the contents of which are hereby incorporated by reference in their entirety. In one embodiment, the essential gene is a DNA synthesis gene, for example, thyA. Thymine is a nucleic acid that is required for bacterial cell growth; in its absence, bacteria undergo cell death. The thyA gene encodes thymidylate synthetase, an enzyme that catalyzes the first step in thymine synthesis by converting dUMP to dTMP (Sat et al., 2003). In some embodiments, the microorganism, e.g., bacterial cell, is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the microorganism, e.g., bacterial cell, is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

In another embodiment, the engineered microorganisms, e.g., genetically engineered bacteria, are auxotrophs in a cell wall synthesis gene, for example, dapA. Diaminopimelic acid (DAP) is an amino acid synthetized within the lysine biosynthetic pathway and is required for bacterial cell wall growth (Meadow et al., 1959; Clarkson et al., 1971). In some embodiments, any of the engineered microorganisms, e.g., genetically engineered bacteria, described herein is a dapD auxotroph in which dapD is deleted and/or replaced with an unrelated gene. A dapD auxotroph can grow only when sufficient amounts of DAP are present, e.g., by adding DAP to growth media in vitro, or in the presence of high DAP levels found naturally in the human gut in vivo. Without sufficient amounts of DAP, the dapD auxotroph dies. In some embodiments, the auxotrophic modification is used to ensure that the microorganism, e.g., bacterial cell, does not survive in the absence of the auxotrophic gene product (e.g., outside of the gut).

In some embodiments, the engineered microorganism, e.g., genetically engineered bacterium, comprises a gene encoding phenylalanine ammonia lyase (PAL), e.g., a gene encoding PAL from Photorhabdus luminescens, e.g., stlA. Williams et al., 2005. In some embodiments, the PAL gene, e.g., stlA gene, is operably linked to an inducible promoter. In some embodiments, the PAL gene, e.g., stlA gene, is operably linked to a promoter that is inducible under low oxygen conditions, e.g., PfnrS promoter. In some embodiments, the PAL gene, e.g., stlA gene, is operably linked to a promoter that is inducible by isopropyl β-d-1-thiogalactopyranoside (IPTG), e.g., Ptac promoter. In some embodiments, the engineered microorganism, e.g., genetically engineered bacterium, comprises a gene encoding a phenylalanine transporter, e.g., a gene encoding a phenylalanine transporter from Escherichia coli, e.g., pheP. In some embodiments, the phenylalanine transporter gene, e.g., pheP gene, is operably linked to an inducible promoter. In some embodiments, the phenylalanine transporter gene, e.g., pheP gene, is operably linked to a promoter that is inducible under low oxygen conditions, e.g., PfnrS promoter. In some embodiments, the engineered microorganism, e.g., genetically engineered bacterium, comprises a gene encoding L-amino acid deaminase (LAAD), e.g., pma. In some embodiments, the LAAD gene is operably linked to an inducible promoter. In some embodiments, the LAAD gene is operably linked to a promoter that is inducible by arabinose, e.g., PBAD promoter. In some embodiments, the engineered microorganism, e.g., genetically engineered bacterium, comprises multiple copies of the genes encoding PAL, phenylalanine transporter, and/or LAAD. In some embodiments, the genes encoding PAL, phenylalanine transporter, and/or LAAD are chromosomally integrated.

In some embodiments, the genetically engineered bacterium is SYNB1618 and comprises three copies of stlA, each operably linked to a PfnrS promoter; two copies of stlA, each operably linked to a Ptac promoter; two copies of pheP, each operably linked to a PfnrS promoter; and one copy of pma operably linked to a PBAD promoter. See, e.g., Isabella et al., Development of a synthetic live bacterial therapeutic for the human metabolic disease phenylketonuria, Nature Biotechnology (2018), which is incorporated by reference in its entirety herein.

In some embodiments, the engineered microorganism, e.g., genetically engineered bacterium, comprises a PAL gene that encodes a polypeptide having modifications S92G, H133M, I167K, L432I, and V470A as compared to wild-type Photorhabdus luminescens PAL, e.g., mPAL1. See, e.g., PCT/US2021/023003, which is incorporated by reference in its entirety herein.

mPAL1:
MKAKDVQPTIIINKNGLISLEDIYDIAIKQKKVEISTEITELLTHGREKL
EEKLNSGEVIYGINTGFGGNANLVVPFEKIAEHQQNLLTFLGAGTGDYMS
KPCIKASQFTMLLSVCKGWSATRPIVAQAIVDMINHDIVPLVPRYGSVGA
SGDLIPLSYIARALCGKGKVYYMGAEIDAAEAIKRAGLTPLSLKAKEGLA
LINGTRVMSGISAITVIKLEKLFKASISAIALAVEALLASHEHYDARIQQ
VKNHPGQNAVASALRNLLAGSTQVNLLSGVKEQANKACRHQEITQLNDTL
QEVYSIRCAPQVLGIVPESLATARKILEREVISANDNPLIDPENGDVLHG
GNFMGQYVARTMDALKLDIALIANHLHAIVALMMDNRFSRGLPNSLSPTP
GMYQGFKGVQLSQTALVAAIRHDCAASGIHTIATEQYNQDIVSLGLHAAQ
DVLEMEQKLRNIVSMTILVACQAIHLRGNISEIAPETAKFYHAVREISSP
LITDRALDEDIIRIADAIINDQLPLPEIMLEE

In some embodiments, the mPAL1 gene is operably linked to an inducible promoter. In some embodiments, the mPAL1 gene is operably linked to a promoter that is inducible by isopropyl β-d-1-thiogalactopyranoside (IPTG), e.g., Ptac promoter. In some embodiments, the engineered microorganism, e.g., genetically engineered bacterium, comprises a gene encoding a phenylalanine transporter, e.g., a gene encoding a phenylalanine transporter from Escherichia coli, e.g., pheP. In some embodiments, the phenylalanine transporter gene, e.g., pheP gene, is operably linked to an inducible promoter. In some embodiments, the phenylalanine transporter gene, e.g., pheP gene, is operably linked to a promoter that is inducible by isopropyl β-d-1-thiogalactopyranoside (IPTG), e.g., Ptac promoter. In some embodiments, the engineered microorganism, e.g., genetically engineered bacterium, comprises a gene encoding L-amino acid deaminase (LAAD), e.g., pma. In some embodiments, the LAAD gene is operably linked to an inducible promoter. In some embodiments, the LAAD gene is operably linked to a promoter that is inducible by arabinose, e.g., PBAD promoter. In some embodiments, the engineered microorganism, e.g., genetically engineered bacterium, comprises multiple copies of the genes encoding mPAL1, phenylalanine transporter, and/or LAAD. In some embodiments, the genes encoding mPAL1, phenylalanine transporter, and/or LAAD are chromosomally integrated.

In some embodiments, the engineered microorganism, e.g., genetically engineered bacterium, comprises a gene encoding methionine gamma lyase (MGL), MetNIQ, and/or methionine decarboxylase (MDC), e.g., a gene encoding MGL from Brevibacterium aurantiacum, Citrobacter freundii, Porphyromonas gingivalis, or Porphyromonas gingivalis; a gene encoding MetNIQ from E. coli; and/or a gene encoding MDC from Streptomyces sp590, respectively. In some embodiments, the MGL gene is operably linked to an inducible promoter. In some embodiments, the MGL gene, is operably linked to a promoter that is inducible under low oxygen conditions, e.g., a PfnrS promoter. In some embodiments, the MGL gene is operably linked to a promoter that is inducible by isopropyl β-d-1-thiogalactopyranoside (IPTG), e.g., a Ptac promoter.

In some embodiments, the metNIQ gene is operably linked to an inducible promoter. In some embodiments, the metNIQ gene is operably linked to a promoter that is inducible under low oxygen conditions, e.g., a PfnrS promoter. In some embodiments, the metNIQ gene is operably linked to a promoter that is inducible by isopropyl β-d-1-thiogalactopyranoside (IPTG), e.g., Ptac promoter.

In some embodiments, the MDC gene is operably linked to an inducible promoter. In some embodiments, the MDC gene is operably linked to a promoter that is inducible under low oxygen conditions, e.g., a PfnrS promoter. In some embodiments, the MDC gene is operably linked to a promoter that is inducible by isopropyl β-d-1-thiogalactopyranoside (IPTG), e.g., a Ptac promoter.

In some embodiments, the engineered microorganism, e.g., genetically engineered bacterium, comprises a gene encoding a methionine transporter, e.g., a gene encoding a methionine transporter from Bacillus subtilis, e.g., a metP gene. In some embodiments, the methionine transporter gene, e.g., metP gene, is operably linked to an inducible promoter. In some embodiments, the methionine transporter gene, e.g., metP gene, is operably linked to a promoter that is inducible under low oxygen conditions, e.g., a PfnrS promoter.

In some embodiments, the engineered microorganism, e.g., genetically engineered bacterium, comprises multiple copies of the gene(s) encoding MGL, MetNIQ, MDC, and/or MetP. In some embodiments, the engineered microorganism, e.g., genetically engineered bacterium, comprises genes encoding MGL, MetNIQ, MDC, and/or MetP are on a plasmid. In some embodiments, the gene encoding MGL, MetNIQ, MDC, and/or MetP are integrated into the bacterial chromosome.

In some embodiments, the genetically engineered bacterium is SYN7349 and comprises a gene encoding MDC and a gene encoding MetNIQ, each operably linked to a Ptet promoter, and a deletion of the yjeH gene. See, e.g., PCT/US2021/017775, which is incorporated by reference in its entirety herein.

In some embodiments, the engineered microorganism, e.g., genetically engineered bacterium, comprises a gene encoding oxalate catabolism enzyme(s) e.g., oxalate-CoA ligase, (e.g., ScAAE3 from S. cerevisiae), an oxalyl-CoA decarboxylase (Oxc, e.g., from O. formigenes), and a formyl-CoA transferase (e.g., Frc, e.g., from O. formigenes). In some embodiments, the scaaE3 gene is operably linked to an inducible promoter. In some embodiments, the scaaE3 gene is operably linked to a promoter that is inducible under low oxygen conditions, e.g., a PfnrS promoter. In some embodiments, the scaaE3 gene is operably linked to a promoter that is inducible by isopropyl β-d-1-thiogalactopyranoside (IPTG), e.g., a Ptac promoter.

In some embodiments, the oxc gene is operably linked to an inducible promoter. In some embodiments, the oxc gene is operably linked to a promoter that is inducible under low oxygen conditions, e.g., a PfnrS promoter. In some embodiments, the oxc gene is operably linked to a promoter that is inducible by isopropyl β-d-1-thiogalactopyranoside (IPTG), e.g., a Ptac promoter.

In some embodiments, the frc gene is operably linked to an inducible promoter. In some embodiments, the frc gene, is operably linked to a promoter that is inducible under low oxygen conditions, e.g., PfnrS promoter. In some embodiments, the frc gene is operably linked to a promoter that is inducible by isopropyl β-d-1-thiogalactopyranoside (IPTG), e.g., Ptac promoter.

In some embodiments, the engineered microorganism, e.g., genetically engineered bacterium, comprises a gene encoding a oxalate transporter, e.g., a gene encoding a methionine transporter from O. formigenes, e.g., an oxlT gene. In some embodiments, the oxalate transporter gene, e.g., ox/T gene, is operably linked to an inducible promoter. In some embodiments, the oxalate transporter gene, e.g., ox/T gene, is operably linked to a promoter that is inducible under low oxygen conditions, e.g., a PfnrS promoter.

In some embodiments, the engineered microorganism, e.g., genetically engineered bacterium, comprises multiple copies of the genes encoding scaaE3, frc, oxc, and/or oxlT. In some embodiments, the engineered microorganism, e.g., genetically engineered bacterium, comprises genes encoding scaaE3, frc, oxc, and/or oxlT are on a plasmid. In some embodiments, the gene encoding scaaE3, frc, oxc, and/or oxlT are integrated into the bacterial chromosome.

In some embodiments, the genetically engineered bacterium is SYN7169 or SYNB8802 and comprises a genes encoding scaaE3, oxc, frc and a gene encoding oxlT, each operably linked to a FNR promoter, and a deletion of phage 3. See, e.g., PCT/US2021/013401, which is incorporated by reference in its entirety herein.

### Pharmaceutical Compositions

In some embodiments, the disclosure provides pharmaceutical compositions comprising an engineered microorganism, e.g., engineered bacterium, that comprises a modified pks island. In some embodiments, the disclosure provides pharmaceutical compositions comprising an engineered microorganism, e.g., engineered bacterium, that comprises a modified pks island and one or more gene(s) or gene cassette(s) for producing a therapeutic molecule. In some embodiments, the pharmaceutical composition may be used to treat, manage, ameliorate, and/or prevent a diseases or disorder, e.g., a cancer; or a disease associated with excess phenylalanine, e.g., PKU; or a disease associated with hyperammonemia, e.g., UCD; or a disease associated with excess oxalate, e.g., HOX, or a disease associated with other deleterious molecules described herein.

Pharmaceutical compositions comprising one or more engineered microorganisms, e.g., engineered bacteria, alone or in combination with prophylactic agents, therapeutic agents, and/or and pharmaceutically acceptable carriers are provided. In certain embodiments, the pharmaceutical composition comprises one species, strain, or subtype of microorganism, e.g., bacteria, that are engineered to comprise the genetic modifications, e.g., pks island modifications, described herein. In alternate embodiments, the pharmaceutical composition comprises two or more species, strains, and/or subtypes of microorganisms, e.g., bacteria, that are each engineered to comprise the genetic modifications, e.g., pks island modifications, described herein.

In some embodiments, the pharmaceutical compositions comprise an engineered microorganism, e.g., engineered bacterium, that comprises a modified clb sequence selected from one or more of the clbA, clbB, clbC, clbD, clbE, clbF, clbG, clbH, clbI, clbJ, clbK, clbL, clbM, clbN, clbO, clbP, clbQ, clbR, and clbS gene sequences and/or operably linked promoter sequences, as compared to a suitable control, e.g., the native pks island in an unmodified bacterium of the same strain and/or subtype. In some embodiments, the modified clb sequence is an insertion, a substitution, and/or a deletion as compared to the control.

In some embodiments, the pharmaceutical compositions comprise an engineered microorganism, e.g., engineered bacterium, that comprises a modification in one or more of the clbA, clbB, clbC, clbD, clbE, clbF, clbG, clbH, clbI, clbJ, clbK, clbL, clbM, clbN, clbO, clbP, clbQ, and clbR gene sequences as compared to the suitable control.

In some embodiments, the pharmaceutical compositions comprise an engineered microorganism, e.g., engineered bacterium, that comprises a modification in one or more of the promoter sequences operably linked to the clbA, clbB, clbC, clbD, clbE, clbF, clbG, clbH, clbI, clbJ, clbK, clbL, clbM, clbN, clbO, clbP, clbQ, and clbR genes as compared to the suitable control.

In some embodiments, the pharmaceutical compositions comprise an engineered microorganism, e.g., engineered bacterium, that comprises a modification in each of the clbA, clbB, clbC, clbD, clbE, clbF, clbG, clbH, clbI, clbJ, clbK, clbL, clbM, clbN, clbO, clbP, clbQ, and clbR gene sequences as compared to the suitable control.

In some embodiments, the pharmaceutical compositions comprise an engineered microorganism, e.g., engineered bacterium, that comprises a modification in each of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the pharmaceutical compositions comprise an engineered microorganism, e.g., engineered bacterium, that comprises a modification in one or more of the clbA, clbB, clbC, clbD, clbE, clbF, clbG, clbH, clbI, clbJ, clbK, clbL, clbM, clbN, clbO, clbP, clbQ, clbR, and clbS gene sequences as compared to the suitable control.

In some embodiments, the pharmaceutical compositions comprise an engineered microorganism, e.g., engineered bacterium, that comprises a modification in one or more of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the pharmaceutical compositions comprise an engineered microorganism, e.g., engineered bacterium, that comprises a modification in each of the clbA, clbB, clbC, clbD, clbE, clbF, clbG, clbH, clbI, clbJ, clbK, clbL, clbM, clbN, clbO, clbP, clbQ, clbR, and clbS gene sequences as compared to the suitable control.

In some embodiments, the pharmaceutical compositions comprise an engineered microorganism, e.g., engineered bacterium, that comprises a modification in each of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the pharmaceutical compositions comprise an engineered microorganism, e.g., engineered bacterium, that comprises an unmodified clbS gene sequence, a modification in each of the clbA, clbB, clbC, clbD, clbE, clbF, clbG, clbH, clbI, clbJ, clbK, clbL, clbM, clbN, clbO, clbP, clbQ, and clbR gene sequences, and a modification in the clbS promoter sequence, as compared to the suitable control.

In some embodiments, the pharmaceutical compositions comprise an engineered microorganism, e.g., engineered bacterium, that comprises a full or partial deletion in the pks island, e.g., a full or deletion in a clb sequence, e.g., a clb gene or promoter sequence. In some embodiments, the pharmaceutical compositions comprise an engineered microorganism, e.g., engineered bacterium, that comprises a full or partial deletion in a clb sequence selected from one or more of the clbA, clbB, clbC, clbD, clbE, clbF, clbG, clbH, clbI, clbJ, clbK, clbL, clbM, clbN, clbO, clbP, clbQ, clbR, and clbS gene sequences and/or operably linked promoter sequences, as compared to a suitable control, e.g., the native pks island in an unmodified bacterium of the same strain and/or subtype. In some embodiments, the clb sequence, e.g., clb gene or promoter sequence, may be deleted by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% as compared to the control, e.g., the corresponding clb sequence in the native pks island in an unmodified bacterium of the same strain and/or subtype. In some embodiments, the clb sequence, e.g., clb gene or promoter sequence, may be 100% deleted as compared to the control, e.g., the corresponding clb sequence in the native pks island in an unmodified bacterium of the same strain and/or subtype

In some embodiments, the pharmaceutical compositions comprise an engineered microorganism, e.g., engineered bacterium, that comprises a partial or full deletion in one or more of the clbA, clbB, clbC, clbD, clbE, clbF, clbG, clbH, clbI, clbJ, clbK, clbL, clbM, clbN, clbO, clbP, clbQ, and clbR gene sequences as compared to the suitable control.

In some embodiments, the pharmaceutical compositions comprise an engineered microorganism, e.g., engineered bacterium, that comprises a partial or full deletion in one or more of the promoter sequences operably linked to the clbA, clbB, clbC, clbD, clbE, clbF, clbG, clbH, clbI, clbJ, clbK, clbL, clbM, clbN, clbO, clbP, clbQ, and clbR genes as compared to the suitable control.

In some embodiments, the pharmaceutical compositions comprise an engineered microorganism, e.g., engineered bacterium, that comprises a partial or full deletion in each of the clbA, clbB, clbC, clbD, clbE, clbF, clbG, clbH, clbI, clbJ, clbK, clbL, clbM, clbN, clbO, clbP, clbQ, and clbR gene sequences as compared to the suitable control.

In some embodiments, the pharmaceutical compositions comprise an engineered microorganism, e.g., engineered bacterium, that comprises a partial or full deletion in each of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the pharmaceutical compositions comprise an engineered microorganism, e.g., engineered bacterium, that comprises a partial or full deletion in one or more of the clbA, clbB, clbC, clbD, clbE, clbF, clbG, clbH, clbI, clbJ, clbK, clbL, clbM, clbN, clbO, clbP, clbQ, clbR, and clbS gene sequences as compared to the suitable control.

In some embodiments, the pharmaceutical compositions comprise an engineered microorganism, e.g., engineered bacterium, that comprises a partial or full deletion in one or more of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the pharmaceutical compositions comprise an engineered microorganism, e.g., engineered bacterium, that comprises a partial or full deletion in each of the clbA, clbB, clbC, clbD, clbE, clbF, clbG, clbH, clbI, clbJ, clbK, clbL, clbM, clbN, clbO, clbP, clbQ, clbR, and clbS gene sequences as compared to the suitable control.

In some embodiments, the pharmaceutical composition comprises an engineered microorganism, e.g., engineered bacterium, that comprises a partial or full deletion in each of the promoter sequences operably linked to the clbA, clbB, clbC, clbD, clbE, clbF, clbG, clbH, clbI, clbJ, clbK, clbL, clbM, clbN, clbO, clbP, clbQ, clbR, and clbS genes as compared to the suitable control.

In some embodiments, the pharmaceutical composition comprises an engineered microorganism, e.g., engineered bacterium, that comprises an unmodified clbS gene sequence, a partial or full deletion in each of the clbA, clbB, clbC, clbD, clbE, clbF, clbG, clbH, clbI, clbJ, clbK, clbL, clbM, clbN, clbO, clbP, clbQ, and clbR gene sequences, and a partial or full deletion in the clbS promoter sequence, as compared to the suitable control

In some embodiments, the pharmaceutical composition comprises an engineered microorganism, e.g., engineered bacterium, that comprises a modified pks island and expresses less colibactin compared to a suitable control, e.g., an unmodified bacterium of the same strain and/or subtype comprising the native pks island. In some embodiments, the pharmaceutical composition comprises an engineered microorganism, e.g., engineered bacterium, that comprises a modified pks island and expresses less N-myristoyl-D-asparagine compared to control. In some embodiments, the pharmaceutical composition comprises an engineered microorganism, e.g., engineered bacterium, that comprises a modified pks island and exhibits decreased accessory protein activity as compared to control. In some embodiments, the pharmaceutical composition comprises an engineered microorganism, e.g., engineered bacterium, that comprises a modified pks island and exhibits decreased phosphopantetheinyl transferase activity as compared to control. In some embodiments, the pharmaceutical composition comprises an engineered microorganism, e.g., engineered bacterium, that comprises a modified pks island and exhibits decreased polyketide synthase activity as compared to control. In some embodiments, the pharmaceutical composition comprises an engineered microorganism, e.g., engineered bacterium, that comprises a modified pks island and exhibits decreased non-ribosomal peptide synthase activity as compared to control. In some embodiments, the pharmaceutical composition comprises an engineered microorganism, e.g., engineered bacterium, that comprises a modified pks island and exhibits decreased activity of hybrid non-ribosomal peptide/polyketide synthase as compared to control. In some embodiments, the pharmaceutical composition comprises an engineered microorganism, e.g., engineered bacterium, that comprises a modified pks island and exhibits decreased hydroxyl acyl coA dehydrogenase activity as compared to control. In some embodiments, the pharmaceutical composition comprises an engineered microorganism, e.g., engineered bacterium, that comprises a modified pks island and decreased acyl carrier protein activity as compared to control. In some embodiments, the pharmaceutical composition comprises an engineered microorganism, e.g., engineered bacterium, that comprises a modified pks island and decreased αβ dehydrogenase activity as compared to control. In some embodiments, the pharmaceutical composition comprises an engineered microorganism, e.g., engineered bacterium, that comprises a modified pks island and exhibits decreased acyl transferase activity as compared to control. In some embodiments, the pharmaceutical composition comprises an engineered microorganism, e.g., engineered bacterium, that comprises a modified pks island and exhibits decreased amidase activity as compared to control. In some embodiments, the pharmaceutical composition comprises an engineered microorganism, e.g., engineered bacterium, that comprises a modified pks island and exhibits decreased MATE transporter activity as compared to control. In some embodiments, the pharmaceutical composition comprises an engineered microorganism, e.g., engineered bacterium, that comprises a modified pks island and exhibits decreased fmtA peptidase activity as compared to control. In some embodiments, the pharmaceutical composition comprises an engineered microorganism, e.g., engineered bacterium, that comprises a modified pks island and exhibits decreased thioesterase activity as compared to control. In some embodiments, the pharmaceutical composition comprises an engineered microorganism, e.g., engineered bacterium, that comprises a modified pks island and exhibits decreased precolibactin transport across the cytoplasmic membrane as compared to control. In some embodiments, the pharmaceutical composition comprises an engineered microorganism, e.g., engineered bacterium, that comprises a modified pks island and exhibits decreased precolibactin transport across the periplasm as compared to control. In some embodiments, the pharmaceutical composition comprises an engineered microorganism, e.g., engineered bacterium, that comprises a modified pks island and exhibits decreased precolibactin cleavage into colibactin as compared to control.

In some embodiments, the pharmaceutical composition comprises an engineered microorganism, e.g., engineered bacterium, that comprises a modified pks island and further comprises one or more gene(s) for producing a desired therapeutic molecule as described herein. In some embodiments, the one or more gene(s) for producing a desired therapeutic molecule are operably linked to a promoter, e.g., an inducible promoter, as described herein.

In some embodiments, the pharmaceutical composition comprises an engineered microorganism, e.g., engineered bacterium, that comprises a modified pks island and further comprises one or more gene(s) or gene cassette(s) for producing at least one phenylalanine metabolizing enzyme, e.g., PAL and/or LAAD, as described herein.

In some embodiments, the pharmaceutical composition comprises an engineered microorganism, e.g., engineered bacterium, that comprises a modified pks island and further comprises one or more gene(s) encoding GOT1, GOT2, TAT, and/or DDC.

In some embodiments, the pharmaceutical composition comprises an engineered microorganism, e.g., engineered bacterium, that comprises a modified pks island and further comprises one or more gene(s) or gene cassette(s) encoding a modified arginine biosynthesis pathway, e.g., deleted arginine repressor, modified arginine repressor binding sites, and/or arginine feedback resistant N-acetylglutamate synthase mutation.

In some embodiments, the pharmaceutical composition comprises an engineered microorganism, e.g., engineered bacterium, that comprises a modified pks island and further comprises one or more gene(s) or gene cassette(s) for producing an anti-cancer molecule, e.g., a deadenylate cyclase gene or an enzyme capable of producing a stimulator of interferon gene agonist.

In some embodiments (e.g., wherein the microorganism, e.g., bacterium, is genetically engineered to comprise a PME) activity may be measured by conversion of phenylalanine to TCA, e.g., in vitro or in vivo, e.g., urinary HA. In some embodiments (e.g., wherein the microorganism, e.g., bacterium, is genetically engineered to comprise a PME), activity may be measured by conversion of phenylalanine to PPA, e.g., in vitro or in vivo. In some embodiments (e.g., wherein the microorganism, e.g., bacterium, is genetically engineered to comprise a modified arginine biosynthesis pathway, e.g., deleted arginine repressor, modified arginine repressor binding sites, and/or arginine feedback resistant N-acetylglutamate synthase mutation), the activity may be measured by assaying the levels of ammonia, arginine or citrulline, e.g., in vitro or in vivo. In some embodiments (e.g., wherein the microorganism, e.g., bacterium, is genetically engineered to comprise an anti-cancer molecule, e.g., dacA), the activity may be measured by assaying the levels of cyclic dinucleotide, e.g. cyclic di-AMP, e.g., in vitro or in vivo.

In some embodiments, the pharmaceutical composition comprises an engineered microorganism, e.g., engineered bacterium, that comprises a modified pks island and further comprises one or more gene(s) or gene cassette(s) for producing at least one oxalate metabolic enzyme, as described herein.

In some embodiments, the pharmaceutical compositions comprise engineered microorganisms that comprise a modified pks island and further comprise one or more gene(s) and/or gene cassette(s) encoding at least one oxalate catabolism enzyme and are capable of converting oxalate into oxalyl-CoA. In some embodiments, the pharmaceutical compositions comprise engineered microorganisms that comprise a modified pks island and further comprise one or more gene(s) and/or gene cassette(s) encoding at least one oxalate catabolism enzyme and are capable of converting oxalyl-CoA into formate and carbon dioxide. In some embodiments, the pharmaceutical compositions comprise engineered microorganisms that comprise a modified pks island and further comprise one or more gene(s) and/or gene cassette(s) encoding at least one oxalate catabolism enzyme and are capable of converting oxalate into oxalyl-CoA, and oxalyl-CoA into formate and carbon dioxide. In some embodiments, the pharmaceutical compositions comprise engineered microorganisms that comprise a modified pks island and further comprise one or more gene(s) and or gene cassette encoding one or more oxalate catabolism enzyme(s) which convert oxalate and formyl CoA into oxalyl-CoA and formate. In some embodiments, the pharmaceutical compositions comprise engineered microorganisms that comprise a modified pks island and further comprise one or more gene(s) and/or gene cassette(s) encoding one or more oxalate catabolism enzyme(s) which convert oxalate and acetyl-coA into oxalyl-CoA and acetate. In some embodiments, the pharmaceutical compositions comprise engineered microorganisms that comprise a modified pks island and further comprise one or more gene(s) and/or gene cassette(s) encoding one or more oxalate catabolism enzyme(s) which convert oxalate and CoA into oxalyl-CoA (e.g., by converting one ATP to AMP plus diphosphate). In some embodiments, the pharmaceutical compositions comprise engineered microorganisms that comprise a modified pks island and further comprise one or more gene(s) and/or gene cassette(s) encoding one or more oxalate catabolism enzyme(s) which convert oxalyl-CoA to carbon dioxide and formyl-CoA. In some embodiments, the pharmaceutical compositions comprise engineered microorganisms that comprise a modified pks island and further produce formate as a result of oxalate catabolism. In some embodiments, the engineered bacteria comprise a modified pks island and further produce formate and carbon dioxide as a result of oxalate catabolism. In some embodiments, the pharmaceutical compositions comprise engineered microorganisms that comprise a modified pks island and further produce acetate as a result of oxalate catabolism. In some embodiments, the pharmaceutical compositions comprise engineered microorganisms that comprise a modified pks island and further produce acetate and carbon dioxide as a result of oxalate catabolism. In some embodiments, the pharmaceutical compositions comprise engineered microorganisms that comprise a modified pks island and further produce formate, acetate, and carbon dioxide as a result of oxalate catabolism.

In some embodiments, the pharmaceutical compositions comprise engineered microorganisms, e.g., engineered bacteria, comprising a modified pks island as disclosed herein, and one or more gene(s) and/or gene cassette(s) encoding at least one oxalate catabolism enzyme and are capable of converting oxalyl-CoA into formate and carbon dioxide. In some embodiments, the pharmaceutical compositions comprise engineered microorganisms, e.g., engineered bacteria, comprising a modified pks island as disclosed herein, and one or more gene(s) and/or gene cassette(s) encoding at least one oxalate catabolism enzyme and are capable of converting oxalate into oxalyl-CoA, and oxalyl-CoA into formate and carbon dioxide. In some embodiments, the pharmaceutical compositions comprise engineered microorganisms, e.g., engineered bacteria, comprising a modified pks island as disclosed herein, and one or more gene(s) and/or gene cassette(s) encoding at least one oxalate catabolism enzyme(s) which convert oxalate and formyl CoA into oxalyl-CoA and formate. In some embodiments, the pharmaceutical compositions comprise engineered microorganisms, e.g., engineered bacteria, comprising a modified pks island as disclosed herein, and one or more gene(s) and/or gene cassette(s) encoding at least one oxalate catabolism enzyme(s) which convert oxalate and acetyl-coA into oxalyl-CoA and acetate. In some embodiments, the pharmaceutical compositions comprise engineered microorganisms, e.g., engineered bacteria, comprising a modified pks island as disclosed herein, and one or more gene(s) and/or gene cassette(s) encoding at least one oxalate catabolism enzyme(s) which convert oxalate and CoA into oxalyl-CoA (e.g., by converting one ATP to AMP plus diphosphate). In some embodiments, the pharmaceutical compositions comprise engineered microorganisms, e.g., engineered bacteria, comprising a modified pks island as disclosed herein, and one or more gene(s) and/or gene cassette(s) encoding at least one oxalate catabolism enzyme(s) which convert oxalyl-CoA to carbon dioxide and formyl-CoA. In some embodiments, the engineered bacteria, comprising a modified pks island as disclosed herein, produce formate as a result of oxalate catabolism. In some embodiments, the pharmaceutical compositions comprise engineered microorganisms, e.g., engineered bacteria, comprising a modified pks island as disclosed herein, produce formate and carbon dioxide as a result of oxalate catabolism. In some embodiments, the pharmaceutical compositions comprise engineered microorganisms, e.g., engineered bacteria, comprising a modified pks island as disclosed herein, produce acetate as a result of oxalate catabolism. In some embodiments, the pharmaceutical compositions comprise engineered microorganisms, e.g., engineered bacteria, comprising a modified pks island as disclosed herein, produce acetate and carbon dioxide as a result of oxalate catabolism. In some embodiments, the pharmaceutical compositions comprise engineered microorganisms, e.g., engineered bacteria, comprising a modified pks island as disclosed herein, produce formate, acetate, and carbon dioxide as a result of oxalate catabolism.

In some embodiments, the pharmaceutical compositions comprise engineered microorganisms, e.g., engineered bacteria, comprising a modified pks island and one or more oxalate catabolism enzyme(s) that increases the rate of oxalate and/or oxalyl-CoA catabolism in the cell. In some embodiments, the pharmaceutical compositions comprise engineered microorganisms, e.g., engineered bacteria, comprising a modified pks island and one or more oxalate catabolism enzyme(s) that decreases the level of oxalate in the cell. In some embodiments, the pharmaceutical compositions comprise engineered microorganisms, e.g., engineered bacteria, comprising a modified pks island and one or more oxalate catabolism enzyme(s) that decreases the level of oxalyl-CoA in the cell. In some embodiments, the pharmaceutical compositions comprise engineered microorganisms, e.g., engineered bacteria, comprising a modified pks island and one or more oxalate catabolism enzyme(s) that decreases the level of oxalic acid in the cell.

In some embodiments, the pharmaceutical composition comprise engineered microorganisms, e.g., genetically engineered bacteria comprising a modified pks island, as disclosed herein, and one or more oxalate catabolism enzyme(s) that decreases the level of oxalate in the cell. In some embodiments, the pharmaceutical composition comprise engineered microorganisms, e.g., genetically engineered bacteria comprising a modified pks island, as disclosed herein, and one or more oxalate catabolism enzyme(s) that decreases the level of oxalyl-CoA in the cell. In some embodiments, the pharmaceutical composition comprise engineered microorganisms, e.g., genetically engineered bacteria comprising a modified pks island, as disclosed herein, and one or more oxalate catabolism enzyme(s) that decreases the level of oxalic acid in the cell.

In some embodiments, the pharmaceutical composition comprise engineered microorganisms, e.g., genetically engineered bacteria comprising a modified pks island, as disclosed herein, and one or more oxalate catabolism enzyme(s) that increases the level of oxalyl-CoA in the cell as compared to the level of its corresponding oxalate in the cell. In some embodiments, the pharmaceutical composition comprise engineered microorganisms, e.g., genetically engineered bacteria comprising a modified pks island, as disclosed herein, and one or more oxalate catabolism enzyme(s) that increases the level of formate and carbon dioxide in the cell as compared to the level of its corresponding oxalyl-CoA in the cell. In some embodiments, the pharmaceutical composition comprise engineered microorganisms, e.g., genetically engineered bacteria comprising a modified pks island, as disclosed herein and one or more oxalate catabolism enzyme(s) that decreases the level of the oxalate and/or oxalyl CoA as compared to the level of oxalate in the cell. In some embodiments, the pharmaceutical composition comprises engineered microorganisms, e.g., engineered bacteria, comprises a modified pks island and at least one heterologous gene encoding an importer of oxalate and at least one heterologous gene encoding at least one oxalate catabolism enzyme. In some embodiments, the pharmaceutical composition comprises engineered microorganisms, e.g., engineered bacteria, comprising at least one heterologous gene encoding an exporter of formate and at least one heterologous gene encoding at least one oxalate catabolism enzyme. In some embodiments, the pharmaceutical composition comprises engineered microorganisms, e.g., engineered bacteria, comprising at least one heterologous gene encoding an oxalate:formate antiporter and at least one heterologous gene encoding at least one oxalate catabolism enzyme.

In some embodiments, the pharmaceutical composition comprise engineered microorganisms, e.g., genetically engineered bacteria comprising a modified pks island as disclosed herein, comprising at least one heterologous gene encoding at least one oxalate catabolism enzyme. In some embodiments, the pharmaceutical composition comprise engineered microorganisms, e.g., genetically engineered bacteria comprising a modified pks island as disclosed herein, comprising at least one heterologous gene encoding an importer of oxalate and at least one heterologous gene encoding at least one oxalate catabolism enzyme. In some embodiments, the pharmaceutical composition comprise engineered microorganisms, e.g., genetically engineered bacteria comprising a modified pks island as disclosed herein, comprising at least one heterologous gene encoding an exporter of formate and at least one heterologous gene encoding at least one oxalate catabolism enzyme. In some embodiments, the pharmaceutical composition comprise engineered microorganisms, e.g., genetically engineered bacteria comprising a modified pks island as disclosed herein, comprising at least one heterologous gene encoding an oxalate:formate antiporter and at least one heterologous gene encoding at least one oxalate catabolism enzyme.

In some embodiments, the pharmaceutical composition comprise engineered microorganisms, e.g., engineered bacteria comprising the gene sequence(s) encoding the one or more oxalate catabolism enzyme(s) comprises a formyl-CoA:oxalate CoA-transferase sequence. In some embodiments, the pharmaceutical composition comprises engineered microorganisms, e.g., engineered bacteria, comprising the formyl-CoA:oxalate CoA-transferase frc, e.g., from O. formigenes. In some embodiments, the pharmaceutical composition comprise engineered microorganisms, e.g., engineered bacteria, comprising the frc gene that has at least about 80% identity with the entire sequence of SEQ ID NO: 1. In some embodiments, the pharmaceutical composition comprise engineered microorganisms, e.g., engineered bacteria, comprising the frc gene that has at least about 90% identity with the entire sequence of SEQ ID NO: 1. In some embodiments, the pharmaceutical composition comprise engineered microorganisms, e.g., engineered bacteria, comprising the frc gene that has at least about 95% identity with the entire sequence of SEQ ID NO: 1. In some embodiments, the pharmaceutical composition comprise engineered microorganisms, e.g., engineered bacteria, comprising the frc gene that has at least about 85%, 86%, 87%, 88%, 89%, 90%, 91%, 92%, 93%, 94%, 95%, 96%, 97%, 98%, or 99% identity with the entire sequence of SEQ ID NO: 1. In some embodiments, the pharmaceutical composition comprise engineered microorganisms, e.g., engineered bacteria, comprises the frc gene comprising the sequence of SEQ ID NO: 1. In some embodiments, the pharmaceutical composition comprise engineered microorganisms, e.g., engineered bacteria, comprising the frc gene that consists of the sequence of SEQ ID NO:1.

In some embodiments, the pharmaceutical composition comprise engineered microorganisms, e.g., genetically engineered bacteria comprising a modified pks island, as disclosed herein, and further comprising the gene sequence(s) encoding the one or more oxalate catabolism enzyme(s) comprises a formyl-CoA:oxalate CoA-transferase sequence. In some embodiments, the pharmaceutical composition comprise engineered microorganisms, e.g., genetically engineered bacteria comprising a modified pks island, as disclosed herein, and further comprising the formyl-CoA:oxalate CoA-transferase frc, e.g., from O. formigenes. In some embodiments, the pharmaceutical composition comprise engineered microorganisms, e.g., genetically engineered bacteria comprising a modified pks island, as disclosed herein, and further comprising the frc gene that has at least about 80% identity with the entire sequence of SEQ ID NO: 1. In some embodiments, the pharmaceutical composition comprise engineered microorganisms, e.g., genetically engineered bacteria comprising a modified pks island, as disclosed herein, and further comprising the frc gene that has at least about 90% identity with the entire sequence of SEQ ID NO: 1. In some embodiments, the pharmaceutical composition comprise engineered microorganisms, e.g., genetically engineered bacteria comprising a modified pks island, as disclosed herein, and further comprising the frc gene that has at least about 95% identity with the entire sequence of SEQ ID NO: 1. In some embodiments, the pharmaceutical composition comprise engineered microorganisms, e.g., genetically engineered bacteria comprising a modified pks island, as disclosed herein, and further comprising the frc gene that has at least about 85%, 86%, 87%, 88%, 89%, 90%, 91%, 92%, 93%, 94%, 95%, 96%, 97%, 98%, or 99% identity with the entire sequence of SEQ ID NO: 1. In some embodiments, the pharmaceutical composition comprise engineered microorganisms, e.g., genetically engineered bacteria comprising a modified pks island, as disclosed herein, and further comprising the frc gene comprising the sequence of SEQ ID NO: 1. In some embodiments, the pharmaceutical composition comprise engineered microorganisms, e.g., genetically engineered bacteria comprising a modified pks island, as disclosed herein, and further comprising the frc gene that consists of the sequence of SEQ ID NO:1.

In some embodiments, the pharmaceutical composition comprises engineered microorganisms, e.g., engineered bacteria, comprising the gene sequence(s) encoding the one or more oxalate catabolism enzyme(s) comprising a oxalyl-CoA decarboxylase sequence. In some embodiments, the pharmaceutical composition comprises engineered microorganisms, e.g., engineered bacteria, comprising the oxalyl-CoA decarboxylase that is oxc, e.g., from O. formigenes. In some embodiments, the pharmaceutical composition comprises engineered microorganisms, e.g., engineered bacteria, comprising the oxc gene that has at least about 80% identity with the entire sequence of SEQ ID NO: 2. In some embodiments, the pharmaceutical composition comprise engineered microorganisms, e.g., engineered bacteria, further comprising the oxc gene that has at least about 90% identity with the entire sequence of SEQ ID NO: 2. In some embodiments, the pharmaceutical composition comprise engineered microorganisms, e.g., engineered bacteria, comprising the oxc gene that has at least about 95% identity with the entire sequence of SEQ ID NO: 2. In some embodiments, the pharmaceutical composition comprises engineered microorganisms, e.g., engineered bacteria, comprising the oxc gene that has at least about 85%, 86%, 87%, 88%, 89%, 90%, 91%, 92%, 93%, 94%, 95%, 96%, 97%, 98%, or 99% identity with the entire sequence of SEQ ID NO: 2. In some embodiments, the pharmaceutical composition comprises engineered microorganisms, e.g., engineered bacteria, comprising the oxc gene that comprises the sequence of SEQ ID NO: 2. In some embodiments, the pharmaceutical composition comprise engineered microorganisms, e.g., engineered bacteria, comprising the oxc gene that consists of the sequence of SEQ ID NO: 2. In some embodiments, the pharmaceutical composition comprises engineered microorganisms, e.g., engineered bacteria, comprising the oxc gene that consists of the sequence of SEQ ID NO: 2.

In some embodiments, the pharmaceutical composition comprises engineered microorganisms, e.g., genetically engineered bacteria comprising a modified pks island and further comprising the gene sequence(s) encoding the one or more oxalate catabolism enzyme(s) comprising an oxalyl-CoA decarboxylase sequence. In some embodiments, the pharmaceutical composition comprises engineered microorganisms, e.g., genetically engineered bacteria comprising a modified pks island as disclosed herein, and further comprising the oxalyl-CoA decarboxylase that is oxc, e.g., from O. formigenes. In some embodiments, the pharmaceutical composition comprises engineered microorganisms, e.g., genetically engineered bacteria comprising a modified pks island as disclosed herein and further comprising the oxc gene that has at least about 80% identity with the entire sequence of SEQ ID NO: 2. In some embodiments, the pharmaceutical composition comprises engineered microorganisms, e.g., genetically engineered bacteria comprising a modified pks island as disclosed herein, and further comprising the oxc gene that has at least about 90% identity with the entire sequence of SEQ ID NO: 2. In some embodiments, the pharmaceutical composition comprises engineered microorganisms, e.g., genetically engineered bacteria comprising a modified pks island as disclosed herein, and further comprising the oxc gene that has at least about 95% identity with the entire sequence of SEQ ID NO: 2. In some embodiments, the pharmaceutical composition comprises engineered microorganisms, e.g., genetically engineered bacteria comprising a modified pks island as disclosed herein, and further comprising the oxc gene that has at least about 85%, 86%, 87%, 88%, 89%, 90%, 91%, 92%, 93%, 94%, 95%, 96%, 97%, 98%, or 99% identity with the entire sequence of SEQ ID NO: 2. In some embodiments, the pharmaceutical composition comprise engineered microorganisms, e.g., genetically engineered bacteria comprising a modified pks island as disclosed herein, and further comprising the oxc gene comprising the sequence of SEQ ID NO: 2. In some embodiments, the pharmaceutical composition comprises engineered microorganisms, e.g., genetically engineered bacteria comprising a modified pks island as disclosed herein, and further comprising the oxc gene that consists of the sequence of SEQ ID NO: 2. In some embodiments, the pharmaceutical composition comprises engineered microorganisms, e.g., genetically engineered bacteria comprising a modified pks island as disclosed herein, and further comprising the oxc gene that consists of the sequence of SEQ ID NO: 2.

In some embodiments, the pharmaceutical composition comprises engineered microorganisms, e.g., engineered bacteria, comprising at least one gene encoding the at least one oxalate catabolism enzyme comprising an oxalate-CoA ligase sequence. In some embodiments, the pharmaceutical composition comprises engineered microorganisms, e.g., engineered bacteria, comprising the oxalate-CoA ligase that is ScAAE3 from S. cerevisiae. In some embodiments, the pharmaceutical composition comprises engineered microorganisms, e.g., engineered bacteria, comprising the ScAAE3 gene has at least about 80% identity with the entire sequence of SEQ ID NO: 3. In some embodiments, the pharmaceutical composition comprise engineered microorganisms, e.g., engineered bacteria, c comprising the ScAAE3 gene has at least about 90% identity with the entire sequence of SEQ ID NO: 3. In some embodiments, the pharmaceutical composition comprise engineered microorganisms, e.g., engineered bacteria, comprising the ScAAE3 gene that has at least about 95% identity with the entire sequence of SEQ ID NO: 3. In some embodiments, the pharmaceutical composition comprises engineered microorganisms, e.g., engineered bacteria, comprising the ScAAE3 gene that has at least about 85%, 86%, 87%, 88%, 89%, 90%, 91%, 92%, 93%, 94%, 95%, 96%, 97%, 98%, or 99% identity with the entire sequence of SEQ ID NO: 3. In some embodiments, the pharmaceutical composition comprise engineered microorganisms, e.g., engineered bacteria, further comprising the ScAAE3 gene comprising the sequence of SEQ ID NO: 3. In some embodiments, the pharmaceutical composition comprise engineered microorganisms, e.g., engineered bacteria, comprising the ScAAE3 gene that consists of the sequence of SEQ ID NO: 3.

In some embodiments, the pharmaceutical composition comprises engineered microorganisms, e.g., genetically engineered bacteria comprising a modified pks island as disclosed herein, and further comprising at least one gene encoding the at least one oxalate catabolism enzyme comprising an oxalate-CoA ligase sequence. In some embodiments, the pharmaceutical composition comprises engineered microorganisms, e.g., genetically engineered bacteria comprising a modified pks island, as disclosed herein, and further comprising the oxalate-CoA ligase that is ScAAE3 from S. cerevisiae. In some embodiments, the pharmaceutical composition comprise engineered microorganisms, e.g., genetically engineered bacteria comprising a modified pks island, as disclosed herein, and further comprising the ScAAE3 gene that has at least about 80% identity with the entire sequence of SEQ ID NO: 3. In some embodiments, the pharmaceutical composition comprise engineered microorganisms, e.g., genetically engineered bacteria comprising a modified pks island, as disclosed herein, and further comprising the ScAAE3 gene that has at least about 90% identity with the entire sequence of SEQ ID NO: 3. In some embodiments, the pharmaceutical composition comprise engineered microorganisms, e.g., genetically engineered bacteria comprising a modified pks island, as disclosed herein, and further comprising the ScAAE3 gene that has at least about 95% identity with the entire sequence of SEQ ID NO: 3. In some embodiments, the pharmaceutical composition comprise engineered microorganisms, e.g., genetically engineered bacteria comprising a modified pks island, as disclosed herein, and further comprising the ScAAE3 gene that has at least about 85%, 86%, 87%, 88%, 89%, 90%, 91%, 92%, 93%, 94%, 95%, 96%, 97%, 98%, or 99% identity with the entire sequence of SEQ ID NO: 3. In some embodiments, the pharmaceutical composition comprise engineered microorganisms, e.g., genetically engineered bacteria comprising a modified pks island, as disclosed herein, and further comprising the ScAAE3 gene comprising the sequence of SEQ ID NO: 3. In some embodiments, the pharmaceutical composition comprise engineered microorganisms, e.g., genetically engineered bacteria comprising a modified pks island, as disclosed herein, and further comprising the ScAAE3 gene that consists of the sequence of SEQ ID NO: 3.

In some embodiments, the pharmaceutical composition comprises engineered microorganisms, e.g., engineered bacteria, comprising at least one gene encoding the at least one oxalate catabolism enzyme comprises an acetyl-CoA:oxalate CoA-transferase sequence. In some embodiments, the pharmaceutical composition comprises engineered microorganisms, e.g., engineered bacteria, comprising the acetyl-CoA:oxalate CoA-transferase YfdE from E. coli from S. cerevisiae. In some embodiments, the pharmaceutical composition comprises engineered microorganisms, e.g., engineered bacteria, comprising the YfdE gene that has at least about 80% identity with the entire sequence of SEQ ID NO: 4. In some embodiments, the pharmaceutical composition comprise engineered microorganisms, e.g., engineered bacteria, comprising the YfdE gene that has at least about 90% identity with the entire sequence of SEQ ID NO: 4. In some embodiments, the pharmaceutical composition comprise engineered microorganisms, e.g., engineered bacteria, comprising the YfdE gene that has at least about 95% identity with the entire sequence of SEQ ID NO: 4. In some embodiments, the pharmaceutical composition comprise engineered microorganisms, e.g., engineered bacteria, comprising the YfdE gene that has at least about 85%, 86%, 87%, 88%, 89%, 90%, 91%, 92%, 93%, 94%, 95%, 96%, 97%, 98%, or 99% identity with the entire sequence of SEQ ID NO: 4. In some embodiments, the pharmaceutical composition comprise engineered microorganisms, e.g., engineered bacteria, comprising the YfdE gene comprising the sequence of SEQ ID NO: 4. In some embodiments, the pharmaceutical composition comprise engineered microorganisms, e.g., engineered bacteria, comprising the YfdE gene that consists of the sequence of SEQ ID NO: 4.

In some embodiments, the pharmaceutical composition comprise engineered microorganisms, e.g., genetically engineered bacteria comprising a modified pks island as disclosed herein, and further comprising at least one gene encoding an oxalate catabolism enzyme comprising an acetyl-CoA:oxalate CoA-transferase sequence. In some embodiments, the pharmaceutical composition comprises engineered microorganisms, e.g., genetically engineered bacteria comprising a modified pks island as disclosed herein, and further comprising the acetyl-CoA:oxalate CoA-transferase is YfdE from E. coli from S. cerevisiae. In some embodiments, the pharmaceutical composition comprise engineered microorganisms, e.g., genetically engineered bacteria comprising a modified pks island as disclosed herein, and further comprising the YfdE gene that has at least about 80% identity with the entire sequence of SEQ ID NO: 4. In some embodiments, the pharmaceutical composition comprise engineered microorganisms, e.g., genetically engineered bacteria comprising a modified pks island as disclosed herein, and further comprising the YfdE gene that has at least about 90% identity with the entire sequence of SEQ ID NO: 4. In some embodiments, the pharmaceutical composition comprise engineered microorganisms, e.g., genetically engineered bacteria comprising a modified pks island as disclosed herein, and further comprising the YfdE gene that has at least about 95% identity with the entire sequence of SEQ ID NO: 4. In some embodiments, the pharmaceutical composition comprise engineered microorganisms, e.g., genetically engineered bacteria comprising a modified pks island as disclosed herein, and further comprising the YfdE gene that has at least about 85%, 86%, 87%, 88%, 89%, 90%, 91%, 92%, 93%, 94%, 95%, 96%, 97%, 98%, or 99% identity with the entire sequence of SEQ ID NO: 4. In some embodiments, the pharmaceutical composition comprise engineered microorganisms, e.g., genetically engineered bacteria comprising a modified pks island as disclosed herein, and further comprising the YfdE gene comprising the sequence of SEQ ID NO: 4. In some embodiments, the pharmaceutical composition comprise engineered microorganisms, e.g., genetically engineered bacteria comprising a modified pks island as disclosed herein, and further comprising the YfdE gene that consists of the sequence of SEQ ID NO: 4.

In some embodiments, the pharmaceutical composition comprise engineered microorganisms, e.g., engineered bacteria, comprising one or more polypeptide(s) encoded by the oxalate catabolism cassette(s) and expressed by the genetically engineered bacteria comprising a formyl-CoA transferase, e.g. frc from O. formigenes. In some embodiments, the pharmaceutical composition comprise engineered microorganisms, e.g., engineered bacteria, comprising polypeptide(s) having at least about 80% identity with SEQ ID NO: 5. In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria have at least about 85% identity with SEQ ID NO: 5. In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria that have at least about 90% identity with SEQ ID NO: 5. In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria have at least about 95% identity with SEQ ID NO: 5. In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria have at least about 96%, 97%, 98%, or 99% identity with SEQ ID NO: 5. In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria that have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 5. In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria comprising the sequence of SEQ ID NO: 5. In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria consist of the sequence of SEQ ID NO: 5.

In some embodiments, the pharmaceutical composition comprise engineered microorganisms, e.g., engineered bacteria, comprising a modified pks island, as disclosed herein, and further comprising one or more polypeptide(s) encoded by the oxalate catabolism cassette(s) and expressed by the genetically engineered bacteria comprises a formyl-CoA transferase, e.g. frc from O. formigenes. In some embodiments, the pharmaceutical composition comprise engineered microorganisms, e.g., engineered bacteria, comprising a modified pks island, as disclosed herein, and further comprising polypeptide(s) that have at least about 80% identity with SEQ ID NO: 5. In some embodiments, the pharmaceutical composition comprise engineered microorganisms, e.g., engineered bacteria, comprising the modified pks island as disclosed herein, comprising one or more polypeptide(s) encoded by the oxalate catabolism gene(s) or gene cassette(s) and expressed by the genetically engineered bacteria have at least about 85% identity with SEQ ID NO: 5. In some embodiments, the pharmaceutical composition comprise engineered microorganisms, e.g., engineered bacteria, comprising a modified pks island as disclosed herein, and further comprising one or more polypeptide(s) encoded by the oxalate catabolism gene(s) or gene cassette(s) and expressed by the genetically engineered bacteria have at least about 90% identity with SEQ ID NO: 5. In some embodiments, the pharmaceutical composition comprise engineered microorganisms, e.g., engineered bacteria, comprising a modified pks island, as disclosed herein, and further comprising one or more polypeptide(s) encoded by the oxalate catabolism gene(s) or gene cassette(s) and expressed by the genetically engineered bacteria have at least about 95% identity with SEQ ID NO: 5. In some embodiments, the pharmaceutical composition comprise engineered microorganisms, e.g., engineered bacteria, comprising a modified pks island, as disclosed herein, and further comprising one or more polypeptide(s) encoded by the oxalate catabolism gene(s) or gene cassette(s) and expressed by the genetically engineered bacteria have at least about 96%, 97%, 98%, or 99% identity with SEQ ID NO: 5. In some embodiments, the pharmaceutical composition comprise engineered microorganisms, e.g., engineered bacteria, comprising a modified pks island, as disclosed herein, and further comprising one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 5. In some embodiments, the pharmaceutical composition comprise engineered microorganisms, e.g., engineered bacteria, comprising a modified pks island, as disclosed herein, and further comprising one or more polypeptide(s) encoded by the oxalate catabolism gene(s) or gene cassette(s) and expressed by the genetically engineered bacteria comprise the sequence of SEQ ID NO: 5. In some embodiments, the pharmaceutical composition comprise engineered microorganisms, e.g., engineered bacteria, comprising a modified pks island, as disclosed herein, and further comprising one or more polypeptide(s) encoded by the oxalate catabolism gene(s) or gene cassette(s) and expressed by the genetically engineered bacteria consist of the sequence of SEQ ID NO: 5.

In some embodiments, the pharmaceutical composition comprise engineered microorganisms, e.g., engineered bacteria, comprising one or more polypeptide(s) encoded by the oxalate catabolism cassette(s) and expressed by the engineered bacteria comprises a oxalyl-CoA decarboxylase, e.g. oxc from O. formigenes. In some embodiments, the pharmaceutical composition comprise engineered microorganisms, e.g., engineered bacteria, comprise polypeptide(s) having at least about 80% identity with SEQ ID NO: 6. In some embodiments, the pharmaceutical composition comprise engineered microorganisms, e.g., engineered bacteria, comprise polypeptide(s) having one or more polypeptide(s) encoded by the oxalate catabolism gene(s) or gene cassette(s and expressed by the engineered bacteria have at least about 85% identity with SEQ ID NO: 6. In some embodiments, the pharmaceutical composition comprise engineered microorganisms, e.g., engineered bacteria, comprise polypeptide(s) having one or more polypeptide(s) encoded by the oxalate catabolism gene(s) or gene cassette(s) and expressed by the engineered bacteria have at least about 90% identity with SEQ ID NO: 6. In some embodiments, the pharmaceutical composition comprise engineered microorganisms, e.g., engineered bacteria, comprise polypeptide(s) having one or more polypeptide(s) encoded by the oxalate catabolism gene(s) or gene cassette(s) and expressed by the engineered bacteria have at least about 95% identity with SEQ ID NO: 6. In some embodiments, the pharmaceutical composition comprise engineered microorganisms, e.g., engineered bacteria, comprise polypeptide(s) having one or more polypeptide(s) encoded by the oxalate catabolism gene(s) or gene cassette(s) and expressed by the engineered bacteria have at least about 96%, 97%, 98%, or 99% identity with SEQ ID NO: 6. In some embodiments, the pharmaceutical composition comprise engineered microorganisms, e.g., engineered bacteria, comprise polypeptide(s) having one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 6. In some embodiments, the pharmaceutical composition comprise engineered microorganisms, e.g., engineered bacteria, comprise polypeptide(s) having one or more polypeptide(s) encoded by the oxalate catabolism gene(s) or gene cassette(s) and expressed by the engineered bacteria comprise the sequence of SEQ ID NO: 6. In some embodiments, the pharmaceutical composition comprise engineered microorganisms, e.g., genetically engineered bacteria comprise polypeptide(s) having one or more polypeptide(s) encoded by the oxalate catabolism gene(s) or gene cassette(s) and expressed by the genetically engineered bacteria consist of the sequence of SEQ ID NO: 6.

In some embodiments, the pharmaceutical composition comprise engineered microorganisms, e.g., genetically engineered bacteria comprising a modified pks island as disclosed herein, and further comprising one or more polypeptide(s) encoded by the oxalate catabolism cassette(s) and expressed by the engineered bacteria comprises a oxalyl-CoA decarboxylase, e.g. oxc from O. formigenes. In some embodiments, the pharmaceutical composition comprise engineered microorganisms, e.g., genetically engineered bacteria comprising a modified pks island as disclosed herein, and further comprising polypeptide(s) having least about 80% identity with SEQ ID NO: 6. In some embodiments, the pharmaceutical composition comprise engineered microorganisms, e.g., genetically engineered bacteria comprising a modified pks island as disclosed herein, and further comprising polypeptide(s) having one or more polypeptide(s) encoded by the oxalate catabolism gene(s) or gene cassette(s and expressed by the engineered bacteria have at least about 85% identity with SEQ ID NO: 6. In some embodiments, the pharmaceutical composition comprise engineered microorganisms, e.g., genetically engineered bacteria comprising a modified pks island as disclosed herein, and further comprising polypeptide(s) having one or more polypeptide(s) encoded by the oxalate catabolism gene(s) or gene cassette(s) and expressed by the engineered bacteria have at least about 90% identity with SEQ ID NO: 6. In some embodiments, the pharmaceutical composition comprise engineered microorganisms, e.g., genetically engineered bacteria comprising a modified pks island as disclosed herein, and further comprising polypeptide(s) having one or more polypeptide(s) encoded by the oxalate catabolism gene(s) or gene cassette(s) and expressed by the engineered bacteria have at least about 95% identity with SEQ ID NO: 6. In some embodiments, the pharmaceutical composition comprise engineered microorganisms, e.g., genetically engineered bacteria comprising a modified pks island as disclosed herein, and further comprising polypeptide(s) having one or more polypeptide(s) encoded by the oxalate catabolism gene(s) or gene cassette(s) and expressed by the engineered bacteria have at least about 96%, 97%, 98%, or 99% identity with SEQ ID NO: 6. In some embodiments, the pharmaceutical composition comprise engineered microorganisms, e.g., genetically engineered bacteria comprising a modified pks island as disclosed herein, and further comprising polypeptide(s) having one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 6. In some embodiments, the pharmaceutical composition comprise engineered microorganisms, e.g., genetically engineered bacteria comprising a modified pks island as disclosed herein, and further comprising polypeptide(s) having one or more polypeptide(s) encoded by the oxalate catabolism gene(s) or gene cassette(s) and expressed by the engineered bacteria comprise the sequence of SEQ ID NO: 6. In some embodiments, the pharmaceutical composition comprise engineered microorganisms, e.g., genetically engineered bacteria comprising a modified pks island as disclosed herein, and further comprising polypeptide(s) having one or more polypeptide(s) encoded by the oxalate catabolism gene(s) or gene cassette(s) and expressed by the genetically engineered bacteria consist of the sequence of SEQ ID NO: 6.

In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria comprises an oxalate-CoA ligase, e.g. ScAAE3 from S cerevisiae. In some embodiments, the pharmaceutical composition comprise engineered microorganisms, e.g., engineered bacteria, comprising polypeptide(s) having at least about 80% identity with SEQ ID NO: 7. In some embodiments, the pharmaceutical composition comprise engineered microorganisms, e.g., engineered bacteria, comprising polypeptide(s) having one or more polypeptide(s) encoded by the oxalate catabolism gene(s) or gene cassette(s and expressed by the engineered bacteria have at least about 85% identity with SEQ ID NO: 7. In some embodiments, the pharmaceutical composition comprise engineered microorganisms, e.g., engineered bacteria, comprising polypeptide(s) having one or more polypeptide(s) encoded by the oxalate catabolism gene(s) or gene cassette(s) and expressed by the engineered bacteria have at least about 90% identity with SEQ ID NO: 7. In some embodiments, the pharmaceutical composition comprise engineered microorganisms, e.g., engineered bacteria, comprising polypeptide(s) having one or more polypeptide(s) encoded by the oxalate catabolism gene(s) or gene cassette(s) and expressed by the engineered bacteria have at least about 95% identity with SEQ ID NO: 7. In some embodiments, the pharmaceutical composition comprise engineered microorganisms, e.g., engineered bacteria, comprising polypeptide(s) having one or more polypeptide(s) encoded by the oxalate catabolism gene(s) or gene cassette(s) and expressed by the engineered bacteria have at least about 96%, 97%, 98%, or 99% identity with SEQ ID NO: 7. In some embodiments, the pharmaceutical composition comprise engineered microorganisms, e.g., engineered bacteria, comprising polypeptide(s) having one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 7. In some embodiments, the pharmaceutical composition comprise engineered microorganisms, e.g., engineered bacteria, comprising polypeptide(s) having one or more polypeptide(s) encoded by the oxalate catabolism gene(s) or gene cassette(s) and expressed by the engineered bacteria comprise the sequence of SEQ ID NO: 7. In some embodiments, the pharmaceutical composition comprise engineered microorganisms, e.g., engineered bacteria, comprising polypeptide(s) having one or more polypeptide(s) encoded by the oxalate catabolism cassette(s) and expressed by the genetically engineered bacteria consist of the sequence of SEQ ID NO: 7.

In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria comprises an oxalate-CoA ligase, e.g. ScAAE3 from S cerevisiae. In some embodiments, the pharmaceutical composition comprise engineered microorganisms, e.g., genetically engineered bacteria comprising a modified pks island as disclosed herein, and further comprising polypeptide(s) having at least about 80% identity with SEQ ID NO: 7. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s and expressed by the engineered bacteria have at least about 85% identity with SEQ ID NO: 7. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 90% identity with SEQ ID NO: 7. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 95% identity with SEQ ID NO: 7 In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 96%, 97%, 98%, or 99% identity with SEQ ID NO: 7. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 7. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria comprise the sequence of SEQ ID NO: 7. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism cassette(s) and expressed by the genetically engineered bacteria consist of the sequence of SEQ ID NO: 7.

In some embodiments, the pharmaceutical composition comprise engineered microorganisms, e.g., engineered bacteria, comprising one or more polypeptide(s) encoded by the oxalate catabolism cassette(s) and expressed by the engineered bacteria comprises an Acetyl-CoA:oxalate CoA-transferase from, e.g. YfdE from E. coli. In some embodiments, the pharmaceutical composition comprise engineered microorganisms, e.g., engineered bacteria, comprising the polypeptide(s) having at least about 80% identity with SEQ ID NO: 8. In some embodiments, the pharmaceutical composition comprise engineered microorganisms, e.g., engineered bacteria, comprising one or more polypeptide(s) encoded by the oxalate catabolism gene(s) or gene cassette(s and expressed by the engineered bacteria have at least about 85% identity with SEQ ID NO: 8. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 90% identity with SEQ ID NO: 8. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 95% identity with SEQ ID NO: 8. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 96%, 97%, 98%, or 99% identity with SEQ ID NO: 8. In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 8. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria comprise the sequence of SEQ ID NO: 8. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria consist of the sequence of with SEQ ID NO: 8.

In some embodiments, the pharmaceutical composition comprise engineered microorganisms, e.g., genetically engineered bacteria comprising a modified pks island as disclosed herein, and further comprising one or more polypeptide(s) encoded by the oxalate catabolism cassette(s) and expressed by the engineered bacteria comprises an Acetyl-CoA:oxalate CoA-transferase from, e.g. YfdE from E. coli. In some embodiments, the pharmaceutical composition comprise engineered microorganisms, e.g., genetically engineered bacteria comprising a modified pks island as disclosed herein, and further comprising polypeptide(s) having at least about 80% identity with SEQ ID NO: 8. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s and expressed by the engineered bacteria have at least about 85% identity with SEQ ID NO: 8. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 90% identity with SEQ ID NO: 8. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 95% identity with SEQ ID NO: 8. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 96%, 97%, 98%, or 99% identity with SEQ ID NO: 8. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 8. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria comprise the sequence of SEQ ID NO: 8. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria consist of the sequence of with SEQ ID NO: 8.

In some embodiments, the pharmaceutical composition comprise engineered microorganisms, e.g., engineered bacteria, comprising one or more polypeptide(s) encoded by the oxalate catabolism gene(s) or gene cassette(s) and expressed by the genetically engineered bacteria comprises a formyl CoA transferase, e.g., yfdW from E. coli. In some embodiments, the pharmaceutical composition comprise engineered microorganisms, e.g., engineered bacteria, comprising polypeptide(s) having at least about 80% identity with SEQ ID NO: 9. In some embodiments, the pharmaceutical composition comprise engineered microorganisms, e.g., engineered bacteria, comprising one or more polypeptide(s) encoded by the oxalate catabolism gene(s) or gene cassette(s and expressed by the engineered bacteria have at least about 85% identity with SEQ ID NO: 9. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 90% identity with SEQ ID NO: 9. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 95% identity with SEQ ID NO: 9. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 96%, 97%, 98%, or 99% identity with SEQ ID NO: 9. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 9. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered comprise the sequence of SEQ ID NO: 9. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria consist of the sequence of SEQ ID NO: 9.

In some embodiments, the pharmaceutical composition comprise engineered microorganisms, e.g., genetically engineered bacteria comprising a modified pks island as disclosed herein, comprising one or more polypeptide(s) encoded by the oxalate catabolism gene(s) or gene cassette(s) and expressed by the genetically engineered bacteria comprises a formyl CoA transferase, e.g., yfdW from E. coli. In some embodiments, the pharmaceutical composition comprise engineered microorganisms, e.g., genetically engineered bacteria comprising a modified pks island as disclosed herein, and further comprising polypeptide(s) having at least about 80% identity with SEQ ID NO: 9. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s and expressed by the engineered bacteria have at least about 85% identity with SEQ ID NO: 9. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 90% identity with SEQ ID NO: 9. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 95% identity with SEQ ID NO: 9. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 96%, 97%, 98%, or 99% identity with SEQ ID NO: 9. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 9. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered comprise the sequence of SEQ ID NO: 9. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria consist of the sequence of SEQ ID NO: 9.

In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria comprises a oxalyl-CoA decarboxylase, e.g., yfdU from E. coli. In some embodiments, the pharmaceutical composition comprise engineered microorganisms, e.g., engineered bacteria, comprise the polypeptide(s) having at least about 80% identity with SEQ ID NO: 10. In another embodiment, one or more polypeptide(s) encoded by the oxalate catabolism gene(s) or gene cassette(s and expressed by the engineered bacteria have at least about 85% identity with SEQ ID NO: 10. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 90% identity with SEQ ID NO: 10. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 95% identity with SEQ ID NO: 10. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 96%, 97%, 98%, or 99% identity with SEQ ID NO: 10. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 10. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria comprise the sequence of SEQ ID NO: 10. In some embodiments, the pharmaceutical composition comprise engineered microorganisms, e.g., engineered bacteria, comprising one or more polypeptide(s) encoded by the oxalate catabolism gene(s) or gene cassette(s) and expressed by the engineered bacteria consist of the sequence of SEQ ID NO: 10.

In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria comprises a oxalyl-CoA decarboxylase, e.g., yfdU from E. coli. In some embodiments, the pharmaceutical composition comprise engineered microorganisms, e.g., genetically engineered bacteria comprising a modified pks island as disclosed herein, and further comprising the polypeptide(s) having at least about 80% identity with SEQ ID NO: 10. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s and expressed by the engineered bacteria have at least about 85% identity with SEQ ID NO: 10. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 90% identity with SEQ ID NO: 10. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 95% identity with SEQ ID NO: 10. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 96%, 97%, 98%, or 99% identity with SEQ ID NO: 10. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 10. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria comprise the sequence of SEQ ID NO: 10. In some embodiments, the pharmaceutical composition comprise engineered microorganisms, e.g., genetically engineered bacteria comprising a modified pks island as disclosed herein, and further comprising one or more polypeptide(s) encoded by the oxalate catabolism gene(s) or gene cassette(s) and expressed by the engineered bacteria consist of the sequence of SEQ ID NO: 10.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The engineered microorganism, e.g., engineered bacterium, described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, immediate-release, pulsatile-release, delayed-release, or sustained release).

The engineered microorganism, e.g., engineered bacterium, may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the engineered microorganism, e.g., engineered bacterium, of the invention may be formulated in a solution of sodium bicarbonate, e.g., 1 molar solution of sodium bicarbonate (to buffer an acidic cellular environment, such as the stomach, for example). In some embodiments, the engineered microorganism comprises a phenylalanine metabolizing enzyme such as phenylalanine ammonia lyase and is formulated in a solution of sodium bicarbonate or calcium bicarbonate optionally with PPI to buffer an acidic environment (e.g., less than a pH of 1, less than a pH of 2, less than a pH of 3, less than a pH of 4, less than a pH of 5, less than a pH of 6, or less than a pH of 7) and/or to reduce the acidity of the environment (e.g., resulting in a pH of greater than 5, a pH of greater than 6, a pH of greater than 7, a pH of greater than 8, a pH of greater than 9, or a pH of greater than 10), e.g., to modulate the acidity or acidic environment of the gut in a subject. In some embodiments, the engineered microorganism comprises a phenylalanine metabolizing enzyme such as phenylalanine ammonia lyase, is formulated in a solution of sodium bicarbonate or calcium bicarbonate, and further administered with (e.g., before, concurrently with, after) an antiemetic. Examples of antiemetics include but are not limited to promethazine, meclizine, hydroxyzine, droperidol, metoclopramide, ondansetron, dolasetron, maropitant, phenotyhiazines, famotidine, ranitidine, omeprazole, pantoprazole, misoprostol proton pump inhibitors, histamine-2 receptor antagonists, serotonin (5-HT3) antagonists, antihistamines, butyrophenones, or gastrokinetic agents. The engineered microorganism, e.g., engineered bacterium, may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The pharmaceutical compositions disclosed herein may be administered topically and formulated in the form of an ointment, cream, transdermal patch, lotion, gel, shampoo, spray, aerosol, solution, emulsion, or other form well-known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The pharmaceutical compositions disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the engineered microorganism, e.g., engineered bacterium, are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the engineered microorganism, e.g., engineered bacterium, described herein.

In one embodiment, the engineered microorganism, e.g., engineered bacterium, may be formulated in a composition suitable for administration to pediatric subjects. As is well known in the art, children differ from adults in many aspects, including different rates of gastric emptying, pH, gastrointestinal permeability, etc. (Ivanovska et al., 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the engineered microorganism, e.g., engineered bacterium, may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. See, e.g., US 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The pharmaceutical composition may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The engineered microorganism, e.g., engineered bacterium, may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see, e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally trehalose. Other suitable cryoprotectants include other disaccharides (e.g., sucrose or lactose), amino acids, and polymers.

### Uses and Methods of Treatment

In some embodiments, the disclosure provides methods for treating a subject suffering from a disease or disorder, where the methods comprise administering engineered microorganisms, e.g., engineered bacteria, comprising a modified pks island. In some embodiments, the disclosure provides methods for treating a subject suffering from a disease or disorder, where the methods comprise administering engineered microorganisms, e.g., engineered bacteria, comprising a modified pks island and one or more gene(s) or gene cassette(s) encoding a therapeutic molecule.

In some embodiments, the engineered microorganisms, e.g., engineered bacteria, comprise a modified pks island as disclosed herein and further comprises one or more gene(s) or gene cassette(s) for producing an anti-cancer molecule, e.g., a deadenylate cyclase gene or an enzyme capable of producing a stimulator of interferon gene agonist; or comprising gene(s) or gene cassette(s) encoding a modified arginine biosynthesis pathway, e.g., deleted arginine repressor, modified arginine repressor binding sites, and/or arginine feedback resistant N-acetylglutamate synthase mutation; or comprising gene(s) or gene cassette(s) for producing a phenylalanine metabolizing enzyme; or comprising gene(s) or gene cassette(s) for producing a oxalate metabolic enzyme.

In some embodiments, the disclosure provides methods for reducing hyperphenylalaninemia or treating a disease associated with hyperphenylalaninemia by administering to the subject a pharmaceutical composition comprising an engineered microorganism, e.g., engineered bacterium, comprising a modified pks island and a phenylalanine metabolizing enzyme. In some embodiments, the disclosure provides methods for reducing hyperphenylalaninemia or treating a disease associated with hyperphenylalaninemia by administering a pharmaceutical composition comprising an engineered microorganism, e.g., engineered bacterium comprising a modified pks island and a phenylalanine metabolizing enzyme. In some embodiments, the disclosure provides methods for reducing hyperphenylalaninemia or treating a disease associated with hyperphenylalaninemia by administering a pharmaceutical composition comprising an engineered microorganism, e.g., engineered bacterium comprising a modified pks island and a phenylalanine transporter. In some embodiments, the disease associated with hyperphenylalaninemia is selected from phenylketonuria, classical or typical phenylketonuria, atypical phenylketonuria, permanent mild hyperphenylalaninemia, nonphenylketonuric hyperphenylalaninemia, phenylalanine hydroxylase deficiency, cofactor deficiency, dihydropteridine reductase deficiency, tetrahydropterin synthase deficiency, Segawa's disease, and liver disease.

In some embodiments, the method of treatment comprises administering engineered bacteria comprising a gene encoding at least one PME, e.g., PAL and/or LAAD, wherein the PME gene is operably linked to an inducible promoter. In some embodiments, the method of treatment comprises administering engineered bacteria that comprise a non-native PME gene, e.g., additional copies of a native PME gene. In some embodiments, the promoter is not associated with the PME gene in nature. In some embodiments, the method of treatment comprises administering engineered bacteria that further comprise a phenylalanine transporter, e.g., PheP. In some embodiments, the method of treatment comprises administering engineered bacteria that comprise a non-native phenylalanine transporter gene, e.g., additional copies of a native phenylalanine transporter gene. In some embodiments, the promoter is not associated with the phenylalanine transporter gene in nature. In some embodiments, the promoter is a thermoregulated promoter or a promoter induced under low-oxygen or anaerobic conditions. In some embodiments, the inducible promoters are induced prior to administration to the subject. In some embodiments, the inducible promoters are induced after administration to the subject. In some embodiments, the bacteria manufactured by the methods disclosed herein are auxotrophs for one or more essential genes, e.g., thyA or dapA

In some embodiments, the disclosure provides a methods of treating inflammatory bowel disease (IBD), autoimmune disorders, diarrheal diseases, related diseases, and other diseases that benefit from reduced gut inflammation and/or enhanced gut barrier function by administering to the subject a pharmaceutical composition comprising an engineered microorganism, e.g., engineered bacterium, comprising a modified pks island and gene(s) or gene cassette(s) encoding an anti-inflammatory and/or gut barrier function enhancer molecule. In some embodiments, the diarrheal disease is selected from the group consisting of acute watery diarrhea, e.g., cholera, acute bloody diarrhea, e.g., dysentery, and persistent diarrhea. In some embodiments, the IBD or related disease is selected from the group consisting of Crohn's disease, ulcerative colitis, collagenous colitis, lymphocytic colitis, diversion colitis, Behcet's disease, intermediate colitis, short bowel syndrome, ulcerative proctitis, proctosigmoiditis, left-sided colitis, pancolitis, and fulminant colitis. In some embodiments, the disease or condition is an autoimmune disorder selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal osteomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis. In some embodiments, the invention provides methods for reducing, ameliorating, or eliminating one or more symptom(s) associated with these diseases, including but not limited to diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts. In some embodiments, the invention provides methods for reducing gut inflammation and/or enhancing gut barrier function, thereby ameliorating or preventing a systemic autoimmune disorder, e.g., asthma (Arrieta et al., 2015).

In some embodiments, the disclosure provides methods for treating a disease or disorder associated with hyperammonemia by administering engineered microorganisms, e.g., engineered bacteria, comprising a modified pks island and further comprising a modified arginine biosynthesis pathway, e.g., deleted arginine repressor, modified arginine repressor binding sites, and/or arginine feedback resistant N-acetylglutamate synthase mutation. In some embodiments, the disorder is a urea cycle disorder such as argininosuccinic aciduria, arginase deficiency, carbamoylphosphate synthetase deficiency, citrullinemia, N-acetylglutamate synthetase deficiency, and ornithine, transcarbamylase deficiency. In alternate embodiments, the disorder is a liver disorder such as hepatic encephalopathy, acute liver failure, or chronic liver failure; organic acid disorders; isovaleric aciduria; 3-methylcrotonylglycinuria; methylmalonic acidemia; propionic aciduria; fatty acid oxidation defects; carnitine cycle defects; carnitine deficiency; (3-oxidation deficiency; lysinuric protein intolerance; pyrroline-5-carboxylate synthetase deficiency; pyruvate carboxylase deficiency; ornithine aminotransferase deficiency; carbonic anhydrase deficiency; hyperinsulinism-hyperammonemia syndrome; mitochondrial disorders; valproate therapy; asparaginase therapy; total parenteral nutrition; cystoscopy with glycine-containing solutions; post-lung/bone marrow transplantation; portosystemic shunting; urinary tract infections; ureter dilation; multiple myeloma; chemotherapy; infection; neurogenic bladder; or intestinal bacterial overgrowth. In some embodiments, the hyperammonemia is associated with Huntington's disease. In some embodiments, the symptom(s) associated thereof include, but are not limited to, seizures, ataxia, stroke-like lesions, coma, psychosis, vision loss, acute encephalopathy, cerebral edema, as well as vomiting, respiratory alkalosis, and hypothermia. In some embodiments, the disorder is a cancer, e.g., wherein the cancer's tumor microenvironment is associated with increased ammonia.

In some embodiments, the disclosure provides methods for treating cancer by administering engineered microorganisms, e.g., engineered bacteria, comprising a modified pks island and at least one gene for producing an anti-cancer molecule, e.g., dacA or an enzyme capable of producing a STING agonist. In some embodiments, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor.

In some embodiments, the disclosure provides methods for reducing oxalate and/or oxalic acid levels associated with HOX by administering engineered microorganisms, e.g., engineered bacteria, comprising a modified pks island and gene(s) or gene cassette(s) encoding an oxalate metabolic or oxalate catabolism enzyme. The method may comprise preparing a pharmaceutical composition with at least one genetically engineered species, strain, or subtype of bacteria described herein, and administering the pharmaceutical composition to a subject in a therapeutically effective amount. In some embodiments, the method of treating a disease or disorder associated with elevated oxalate comprises administering to a subject in need thereof an engineered bacterium comprising gene sequence(s) encoding one or more oxalate catabolism enzyme(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate transporter(s) or a pharmaceutical composition thereof. In some embodiments, the method of treating a disease or disorder associated with elevated oxalate comprises administering to a subject in need thereof an engineered bacterium comprising gene sequence(s) encoding one or more formate importers(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate:formate antiporter(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate catabolism enzyme(s) and gene sequence(s) encoding one or more of the following: (i) one or more oxalate transporter(s); (ii) one or more formate exporter(s); (iii) one or more oxalate:formate antiporter(s); and (iv) combinations thereof or a pharmaceutical composition thereof. In some embodiments, the bacterial cells disclosed herein are administered orally, e.g., in a liquid suspension. In some embodiments, the bacterial cells disclosed herein are lyophilized in a gel cap and administered orally. In some embodiments, the bacterial cells disclosed herein are administered via a feeding tube or gastric shunt. In some embodiments, the bacterial cells disclosed herein are administered rectally, e.g., by enema. In some embodiments, the genetically engineered bacteria are administered topically, intraintestinally, intrajejunally, intraduodenally, intraileally, and/or intracolically.

In some embodiments, the disclosure provides methods for reducing oxalate and/or oxalic acid levels in a subject or treating a disease associated with hyperoxaluria by administering the pharmaceutical compositions disclosed herein, e.g., genetically engineered bacteria comprising a gene encoding an oxalate catabolism enzyme. In some embodiments, the disclosure provides methods for reducing oxalate and/or oxalic acid levels in a subject or treating a disease associated with hyperoxaluria by administering the pharmaceutical compositions disclosed herein, e.g., genetically engineered bacteria comprising a oxalate catabolism enzyme and a modified pks island. The method may comprise reducing the levels of oxalate in a subject, the method comprising administering to the subject a pharmaceutical composition comprising a engineered microorganism comprising one or more gene sequences encoding one or more oxalate catabolism enzymes operably linked to a directly or indirectly first inducible promoter that is not associated with the oxalate catabolism enzyme gene in nature, thereby reducing the levels of oxalate in the subject. The method may comprise reducing the levels of oxalate in a subject, the method comprising administering to the subject a pharmaceutical composition comprising a engineered microorganism comprising a modified pks island, as disclosed herein, and one or more gene sequences encoding one or more oxalate catabolism enzymes operably linked to a directly or indirectly first inducible promoter that is not associated with the oxalate catabolism enzyme gene in nature, thereby reducing the levels of oxalate in the subject.

In some embodiments, the method involves the administration of the pharmaceutical compositions, as described herein, to reduce oxalate and/or oxalic acid levels in a subject to normal levels. In another embodiment, the pharmaceutical composition described herein is administered to reduce oxalate and/or oxalic acid levels in a subject to below a normal level. In another embodiment, the pharmaceutical composition described herein is administered to reduce the daily urinary oxalate excretion of a subject to less than 40 mg per 24 hours.

In some embodiments, the method involves the administration of the pharmaceutical compositions, as described herein, to reduce acute levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day. In one embodiment, the method reduces chronic levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day.

In certain embodiments, the method involves the administration of the pharmaceutical composition, as described herein, to reduce oxalate and/or oxalic acid levels in a subject. In some embodiments, the methods of the present disclosure reduce the oxalate and/or oxalic acid levels in a subject by at least about 10%, 20%, 25%, 30%, 40%, 50%, 60%, 70%, 75%, 80%, 85%, 90%, 95%, or more. In another embodiment, the methods of the present invention reduce the oxalate and/or oxalic acid levels in a subject by at least two-fold, three-fold, four-fold, five-fold, six-fold, seven-fold, eight-fold, nine-fold, or ten-fold. In another embodiment, the methods of the present invention reduce the daily urinary oxalate excretion of a subject to less than 40 mg per 24 hours. In some embodiments, reduction is measured by comparing the oxalate and/or oxalic acid level in a subject before and after administration of the pharmaceutical composition. In one embodiment, the oxalate and/or oxalic acid level is reduced in the gut of the subject. In one embodiment, the oxalate and/or oxalic acid level is reduced in the urine of the subject. In another embodiment, the oxalate and/or oxalic acid level is reduced in the blood of the subject. In another embodiment, the oxalate and/or oxalic acid level is reduced in the plasma of the subject. In another embodiment, the oxalate and/or oxalic acid level is reduced in the fecal matter of the subject. In another embodiment, the oxalate and/or oxalic acid level is reduced in the brain of the subject.

In one embodiment, the pharmaceutical composition described herein is administered to reduce oxalate in a subject to a normal level.

In some embodiments, the method of treating the disorder in which oxalate is detrimental, e.g., hyperoxaluria, allows one or more symptoms of the condition or disorder to improve by at least about 10%, 20%, 30%, 40%, 50%, 60%, 70%, 80%, 90%, 95%, or more. In some embodiments, the method of treating the disorder in which oxalate is detrimental, e.g., hyperoxaluria, allows one or more symptoms of the condition or disorder to improve by at least about two-fold, three-fold, four-fold, five-fold, six-fold, seven-fold, eight-fold, nine-fold, or ten-fold.

Before, during, and after the administration of the pharmaceutical composition, oxalate and/or oxalic acid levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, kidney, liver, duodenum, jejunum, ileum, cecum, colon, rectum, and anal canal. In some embodiments, the methods may include administration of the compositions disclosed herein to reduce levels of the oxalate and/or oxalic acid. In some embodiments, the methods may include administration of the compositions of the invention to reduce the oxalate and/or oxalic acid to undetectable levels in a subject. In some embodiments, the methods may include administration of the compositions of the invention to reduce the oxalate and/or oxalic acid concentrations to undetectable levels, or to less than about 1%, 2%, 5%, 10%, 20%, 25%, 30%, 40%, 50%, 60%, 70%, 75%, 80%, 85%, 90%, or 95% of the subject's oxalate and/or oxalic acid levels prior to treatment.

In some embodiments, the method involves the administration of a pharmaceutical composition comprising engineered microorganisms, e.g., engineered bacterium, as disclosed herein. The engineered microorganisms produce an oxalate catabolism enzyme under exogenous environmental conditions, such as the low-oxygen environment of the mammalian gut, to reduce levels of oxalate and/or oxalic acid in the urine, blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

In some embodiments, the method involves the administration of pharmaceutical compositions disclosed herein reduce plasma levels of oxalate will be reduced to less than 4 mg/dL. In one embodiment, the pharmaceutical compositions disclosed herein reduce plasma levels of oxalate to less than 3.9 mg/dL. In one embodiment, the pharmaceutical compositions disclosed herein reduce plasma levels of oxalate, to less than 3.8 mg/dL, 3.7 mg/dL, 3.6 mg/dL, 3.5 mg/dL, 3.4 mg/dL, 3.3 mg/dL, 3.2 mg/dL, 3.1 mg/dL, 3.0 mg/dL, 2.9 mg/dL, 2.8 mg/dL, 2.7 mg/dL, 2.6 mg/dL, 2.5 mg/dL, 2.0 mg/dL, 1.75 mg/dL, 1.5 mg/dL, 1.0 mg/dL, or 0.5 mg/dL.

In one embodiment, the subject has plasma levels of at least 4 mg/dL oxalate prior to administration of the pharmaceutical composition disclosed herein. In another embodiment, the subject has plasma levels of at least 4.1 mg/dL, 4.2 mg/dL, 4.3 mg/dL, 4.4 mg/dL, 4.5 mg/dL, 4.75 mg/dL, 5.0 mg/dL, 5.5 mg/dL, 6 mg/dL, 7 mg/dL, 8 mg/dL, 9 mg/dL, or 10 mg/dL prior to administration of the pharmaceutical composition disclosed herein.

In some embodiments, the methods disclosed herein further comprise comparing the level of the oxalate and/or oxalic acid in the plasma sample from the subject after administration of a pharmaceutical composition disclosed herein to the plasma sample from the subject before administration of a pharmaceutical composition disclosed herein. In one embodiment, a reduced level of the oxalate and/or oxalic acid in the plasma sample from the subject after administration of a pharmaceutical composition disclosed herein indicates that the plasma levels of the oxalate and/or oxalic acid are decreased, thereby treating the disorder in which oxalate and/or oxalic acid is detrimental in the subject. In one embodiment, the plasma level of oxalate and/or oxalic acid is decreased at least 10%, 20%, 30%, 40%, 50%, 60%, 70%, 80%, 90%, or 100% in the sample after administration of the pharmaceutical composition as compared to the oxalate and/or oxalic acid level in the plasma sample before administration of the pharmaceutical composition. In another embodiment, the plasma level of the oxalate and/or oxalic acid is decreased at least two-fold, three-fold, four-fold, or five-fold in the plasma sample after administration of the pharmaceutical composition as compared to the oxalate and/or oxalic acid level in the plasma sample before administration of the pharmaceutical composition.

In some embodiments, the methods disclosed herein further comprise comparing the level of the oxalate and/or oxalic acid in the urine sample from the subject after administration of a pharmaceutical composition disclosed herein to the urine sample from the subject before administration of a pharmaceutical composition disclosed herein. In one embodiment, a reduced level of the oxalate and/or oxalic acid in the urine sample from the subject after administration of a pharmaceutical composition disclosed herein indicates that the urine levels of the oxalate and/or oxalic acid are decreased, thereby treating the disorder in which oxalate and/or oxalic acid is detrimental in the subject. In one embodiment, the urine level of oxalate and/or oxalic acid is decreased at least 10%, 20%, 30%, 40%, 50%, 60%, 70%, 80%, 90%, or 100% in the sample after administration of the pharmaceutical composition as compared to the oxalate and/or oxalic acid level in the urine sample before administration of the pharmaceutical composition. In another embodiment, the urine level of the oxalate and/or oxalic acid is decreased at least two-fold, three-fold, four-fold, or five-fold in the urine sample after administration of the pharmaceutical composition as compared to the oxalate and/or oxalic acid level in the urine sample before administration of the pharmaceutical composition.

In some embodiments, the method of treatment comprises administering engineered microorganisms, e.g., engineered bacteria comprising a modified pks island and optionally a therapeutic molecule as disclosed herein, compositions or formulations thereof, wherein the composition or formulation comprises 1-20% trehalose, 1-10% trehalose, 5-15% trehalose, 7-13% trehalose, 9-11% trehalose, or about 10% trehalose in a biological buffer covering a pH range of 6-8, where the biological buffer may be PIPES, MOPS, HEPES, and/or Tris buffer. In some embodiments, the composition or formulation comprises 1-400 mM Tris buffer. In some embodiments, the composition or formulation comprises 1-300 mM Tris buffer. In some embodiments, the composition or formulation comprises 1-200 mM Tris buffer. In some embodiments, the composition or formulation comprises 1-100 mM Tris buffer. In some embodiments, the composition or formulation comprises 1-50 mM Tris buffer. In some embodiments, the composition or formulation comprises 1-10 mM Tris buffer. In some embodiments, the pharmaceutical composition comprises lyophilized bacteria. In some embodiments, the percent water content of the lyophilized bacteria is approximately 1-10%. In some embodiments, the percent water content is approximately 3-8%. In some embodiments, the percent water content is approximately 3-6%. In some embodiments, the percent water content is approximately 3-5%. In some embodiments, the percent water content is approximately 3%, approximately 4%, or approximately 5%.

Exemplary diseases, disorders, and methods of treatment are provided in WO2016090343, WO2016200614, WO2017139697, WO2016183531, WO2017087580, WO2016141108, WO2017074566, WO2017136792, WO2017136795, WO2018129404, WO2019014391, WO2016210384, WO2017123418, WO2017123676, WO2016183531, WO2018237198, WO2016201380, US20170216370, and WO2017040719, the contents of which are hereby incorporated by reference in their entirety

The disclosure also provides methods of assessing colibactin production and pks island activity, e.g., by measuring colibactin precursor(s) or metabolite(s). In some embodiments, the method of assessing colibactin levels comprises detecting N-myristoyl-D-asparagine. In some embodiments, N-myristoyl-D-asparagine is detected using mass spectrometry, e.g., quantitative LC-MS/MS.

### EXAMPLES

### Example 1

### Pharmacokinetic Profile of Δpks Strain in Mouse

A Δpks E. coli Nissle (EcN) strain was generated by deleting the clb sequences of the clbA-R genes and operably linked promoters; the clbS gene sequence was unmodified (intact), but its operably linked promoter was deleted (FIG. 3B). The constructs were generated using lambda red methods as previously described. See, e.g., PCT/US2016/032562, the contents of which is herein incorporated by reference in its entirety. The pharmacokinetic profile of a Δpks strain was investigated in naïve healthy mice and in mice pre-treated with Streptomycin, an aminoglycoside antibiotic that binds to the bacterial 30S ribosomal subunit and impairs protein synthesis, hence abolishing mouse GI-resident flora survival and growth.

The day prior to strain dosing, female wild-type C57BL/6j mice were labelled and identified with tail markings, and separated into experimental groups (Table A) based on body weight. Strep-water was made by dissolving 5 g Streptomycin in 1 L of Reverse-Osmosis (RO) water, and provided ad-libitum in drinking bottles to mice in Group 2. The following day, subgroups of mice received an oral gavage (PO dose) of 1e+10 EcN cells (either WT, Δpks or combination of both) or vehicle (Table A).

TABLE A
Mouse Study Design

| Experimental | | No. Animals | PO dose | CFU |
|---|---|---|---|---|
| groups | Treatment | per group | volume | dose |
| GROUP 1 | Vehicle | 5 | 150 uL | N/A |
| (Normal water) | WT EcN | 5 | 150 uL | 1e+10 |
| | Δpks EcN | 5 | 150 uL | 1e+10 |
| GROUP 2 | Vehicle | 5 | 150 uL | N/A |
| (Strep-water) | WT EcN + Δpks | 5 | 300 uL total | 1e+10 |
| | EcN (COMBO) | | (150 uL per | |
| | | | strain) | |

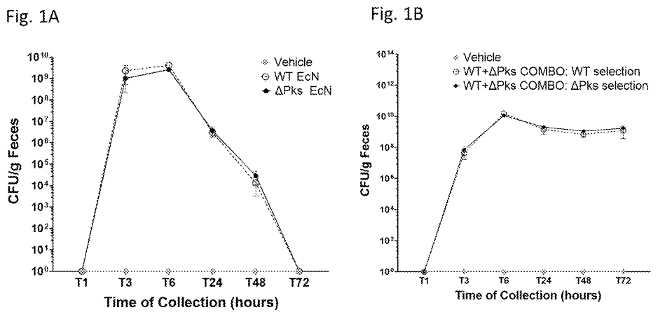

The mice were then placed into clean cages to avoid excremental cross-contamination. Fecal pellets were collected fresh by free-catch at different time points (1, 3, 6, 24, 48, 72 hours post-dose) by holding the animals over a pre-weighed Bead Beater collection tube. Pellets were then processed for CFU plating within one hour of collection, via homogenization in 500 μl of PBS with FastPrep 24 at the speed of 4 m/s for 10 seconds. Ten microliters of eight serial 1-to-10 dilutions of the homogenate were row-plated in triplicate on petri dishes of LB agar containing antibiotics selective for each strain. Specifically, fecal samples were plated on Strep-300, Kan-100 or Strep-300 & Kan-100 plates, according to whether they were obtained from mice in the WT EcN, Δpks EcN, or vehicle and combo WT+Δpks EcN group, respectively. Plates were incubated overnight at 37° C. to allow clonogenic growth of antibiotic-resistant cells. CFUs were counted the following day and normalized to the weight (g) of the fecal pellets previously collected. In naïve healthy mice (GROUP 1), both non-engineered (WT) and Δpks EcN cells completed fecal transition and excretion by 72 hrs post-dose, as indicated by the absence of CFUs at the 72 hrs collection time point (FIG. 1A). Also, Δpks and WT EcN cells showed comparable viability, as measured by the number of CFUs retrieved in fecal samples from early time points (FIG. 1A). In mice with eradicated resident enteric bacteria (GROUP 2), where WT and Δpks strains were co-dosed, the CFU recovery of non-engineered versus Δpks cells was indistinguishable across all time points, pointing at a non-observable competitive disadvantage in viability for Δpks EcN (FIG. 1B).

### Example 2

### Pharmacokinetic Profile of Δpks Strain in Non-Human Primate (NHP).

The Δpks EcN strain described in Example 1 (deleted clbA-clbR gene and promoter sequences, intact clbS gene sequence with the operably linked promoter deleted) was administered to non-human primate. The pharmacokinetic profile of the Δpks strain described above was investigated in our colony of non-naïve male Cynomolgus Macaque, hosted at the Charles River Laboratories. Six monkeys with body weight not exceeding 5 Kg, received subsequent oral gavages of Sodium Bicarbonate (5 mL), a combination of equally numbered WT EcN and Δpks EcN cells in the total amount of 1e+12 (10 mL), and water to flush residues from the syringe.

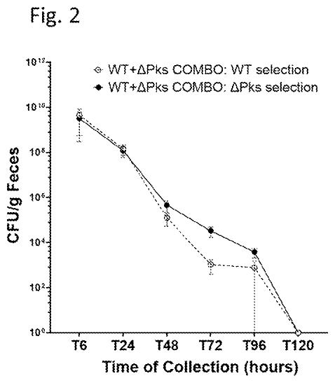

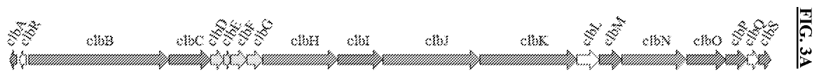

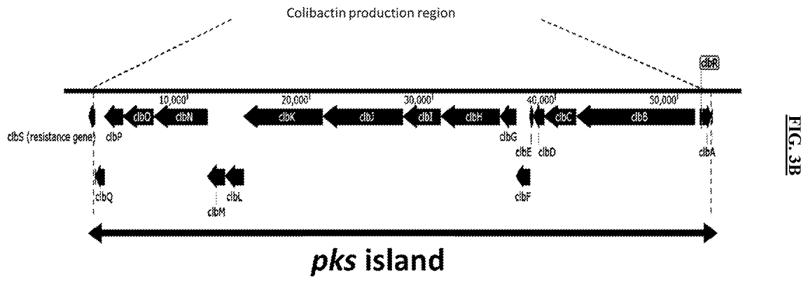

The urine and fecal pans were cleaned and changed 3-hours prior to each fecal collection. Fecal collections occurred over a 3-hour period per time-point to allow time for the animals to defecate. Collections time points were: 6-hours (T3-6), 24-hours (T21-24), 48-hours (T45-48), 72-hours (T69-72), 96-hours (T93-96), and 120-hours (T117-120) post dose. Fecal samples were weighted, packaged in ice and shipped immediately to Synlogic. Samples were homogenized in Bead Beater containing 1 ml of PBS with FastPrep 24 at the speed of 4 m/s for 40 seconds and plated according to the methodology described above (Pharmacokinetic in mouse). CFUs were counted after the overnight incubation at 37° C. No significant difference in CFU recovery was observed across time points, and by day 5 no CFUs were retrieved in the fecal homogenates plated to selectively support the growth of each single strain (FIG. 2). Thus, we concluded that both non-engineered (WT) and Δpks EcN have a similar excretion and viability profile.

### Example 3

### In Vitro Activity Profile of Δpks Strains Expressing Phenylalanine Ammonia Lyase

The Δpks EcN strain described in Example 1 (deleted clbA-clbR gene and promoter sequences, intact clbS gene sequence with the operably linked promoter deleted) and further comprising gene sequences encoding phenylalanine ammonia lyase (PAL), e.g., wild-type PAL from Photorhabdus luminescens (stlA) or mutant PAL (mPAL1) described herein. Exemplary PAL enzymes are also described in PCT/US2016/032562 filed May 13, 2016, the contents of which is herein incorporated by reference in its entirety. Degradation of phenylalanine by PAL results in the production of hippuric acid. Levels of hippurate production were measured to assess effects of Δpks on strain activity. SYNB1618 and SYNB1618 are parental strains having intact clb sequences. SYN8260 and SYN8261 are clones derived from parental clone SYNB1618 and having Δpks. SYN8256 is a clone derived from parental clone SYNB1934 and having Δpkspks.

To measure PAL activity from activated cells, frozen cell aliquots were thawed and diluted 1000× to measure cell density (OD) at absorbance of 600 nm. Using the OD values generated, cells were diluted to 10 OD in M9 buffer. For the assay, cells were further diluted to 1 OD in the assay buffer (M9 with 40 mM Phenylalanine) to a final volume of 1 mL and incubated without shaking at 37° C. for 2 hours in a 96-deep well plate. At 0, 30, 60, 90, and 120 minutes, 150 μL of the samples were collected in a v-bottom 96-well plate and centrifuged at 4000 rpm for 2 minutes. Aliquots of the supernatants (100 μL) were transferred to a clear flat-bottom 96-well plate for TCA measurement at 290 nm absorbance. A synergy neo microplate reader was used to measure absorbance at 600 and 290 nm.

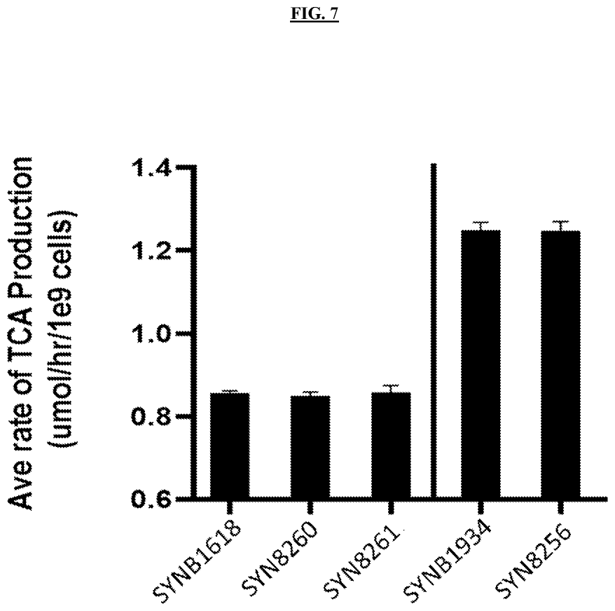

TCA standards were prepared in the assay buffer with the following concentrations: 0.625, 0.313, 0.156, 0.078, 0.039, 0.02, and 0.01 mM. A standard curve was generated with absorbance values at 290 nm for each of the concentrations and was used to convert absorbance values to mM TCA produced during the assay. TCA production rates were calculated using the quantity of TCA calculated for each of the 0-, 30-, 60-, 90-, and 120-minutes time points, and normalized by 1E9 cells/mL (assuming 1 OD=1E9 cells) per hour. Final TCA production values were reported as μmol/hr/1E9 cells. Results are shown in FIG. 7 and indicate that colibactin knock-out strains exhibit no loss in TCA production as compared to their parental strains.

### Example 4

### In Vitro Production of Colibactin

To confirm that Δpks deletions abrogated colibactin production, ability of strains having the Δpks deletions to produce colibactin was assessed. Colibactin is volatile and cannot be directly detected. A surrogate assay to assess colibactin levels was conducted, in which levels of colibactin precursor are measured. Colibactin precursor is metabolized by ClbP to colibactin and N-myristoyl-D-asparagine in a 1:2 ratio and N-myristoyl-D-asparagine is measured in bacterial supernatant by LC-MS/MS.

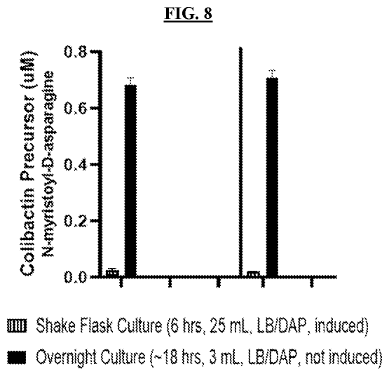

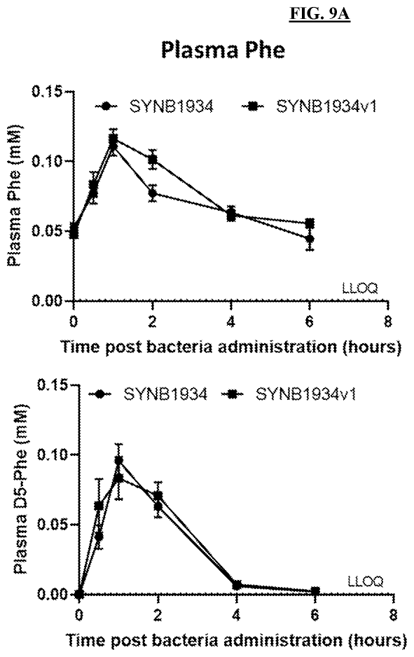

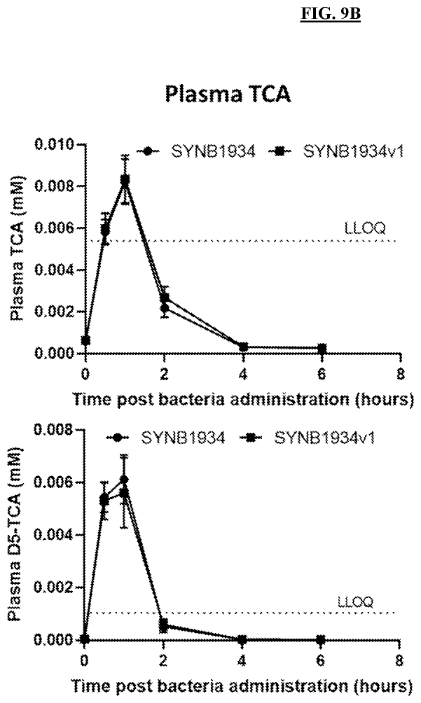

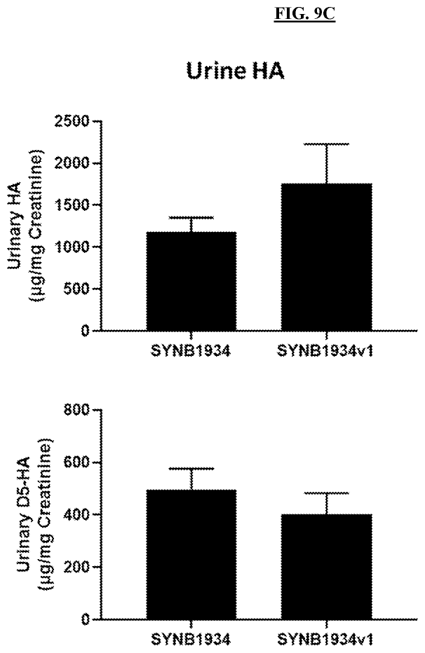

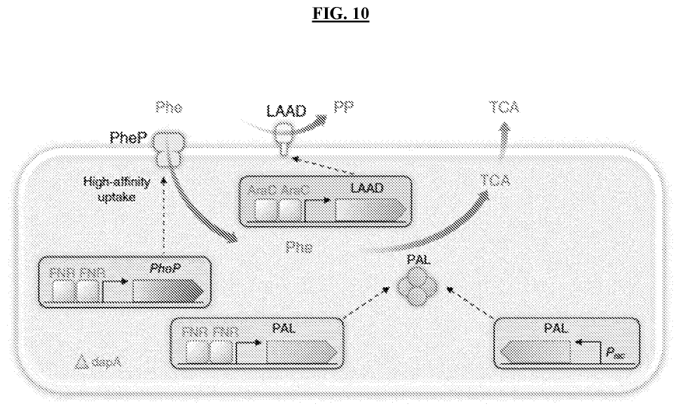

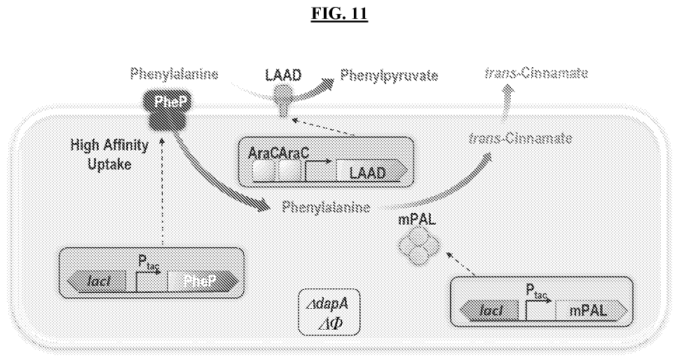

In brief, N-myristoyl-D-Asparagine was quantitated in bacterial supernatant by LC-MS/MS using a Thermo Vanquish UHPLC-Altis TSQ MS system. Standard was prepared at concentrations from 0.032 to 20 μg/mL in 80% acetonitrile. Samples were extracted with acetonitrile to 80%. Two microliters were injected onto a Thermo Hypersil Gold 5 μM C18, 2.1×100 mm column using 0.1% formic acid (A) and acetonitrile with 0.1% formic acid (B) at 0.4 mL/min and 40° C. Analytes were separated using a gradient from 30 to 98% B over 2 minutes followed by wash and equilibration steps. Compounds were detected by tandem mass spectroscopy with selected reaction monitoring in electrospray negative ion mode using the N-myristoyl-D-asparagine specific collision induced mass transitions: 343/211, 343/326, 343/133. Peak areas of the 343/211 ion pair were used to calculate unknown concentrations against the standard calibration curve. Results are shown in FIG. 8 and demonstrate that colibactin precursor production was eliminated in SYNB1618Δclb and. SYNB1934Δclb strains.

### Example 5

### Activity of a Δpks Strain Following Administration to Non-Human Primates

The objective of this study was to evaluate the pharmacokinetics following oral administration in non-human primates. The experimental design is shown in Table 4.

TABLE 4
Experimental Design

| | | | Dose | |
|---|---|---|---|---|
| | No. | | Volume | Dose |
| Group | Animals | Treatment | (mL) | Regimen |
| 1 | 6 | Peptone | 6.1 | PO |
| | | Sodium bicarbonate | 5 | |
| | | D5-Phe | 7.7 | |
| | | SYN8256 | 3 | |
| 2 | 6 | Peptone | 6.1 | |
| | | Sodium bicarbonate | 5 | |
| | | D5-Phe | 7.7 | |
| | | SYNB1934 | 3 | |

### Administration of Treatment

Test Articles were administered to the appropriate animals by oral gavage on Day 1. Dose formulations were administered by oral gavage using a disposable catheter attached to a plastic syringe. Following dosing, the gavage tube was rinsed with 5 mL of the animal drinking water, into the animal's stomach. Each group was dosed with a clean gavage tube. The first day of dosing was designated as Day 1. Animals were fasted overnight the day before dosing (Day −1). Peptone was provided at 3.05 grams and was chosen because it simulates a low-protein diet in humans and allows detection of Phe in the blood after feeding and metabolites of Phe in the urine. NHPs were dosed with 10e11 bacteria. D5-Phe (20 mg/mL) was dispensed for all animals on Day 1. The in-life procedures, observations, and measurements were performed for all animals, including alternates as applicable. Food was returned following the final blood collection.

### Sample Collection and Processing

Blood was collected by venipuncture from an appropriate peripheral vein on Day 1 at 0 hr pre-dose, and at 30 min, 1 hr, 2 hr, 4 hr, and 6 hr post-dose for bioanalysis. After collection, samples were transferred to the appropriate laboratory for processing and stored at −80° C. Samples were collected according to the following table. On Day 1, prior to dosing, a clean collection pan was inserted to assist in urine collection at room temperature. At conclusion of 6 hours post-dose, the total amount of urine was measured and recorded. Urine samples were collected and immediately frozen on dry ice. Target volume was 1 mL and the anticoagulant was heparin (sodium). Samples were placed on crushed wet ice until centrifugation. The samples were centrifuged as per standard procedures. The resultant plasma was separated, transferred in two aliquots and immediately frozen on dry ice and stored at −80° C.

Phe, d5-Phe, TCA, d5-TCA, HA, d5-HA, Creatinine Quantitation in NHP Plasma or Urine by LC-MS/MS

Phenylalanine, d5-phenylalanine, trans-cinnamic acid and d5-trans-cinnamic acid in NHP plasma as well as hippuric acid, d5-hippuric acid and creatinine in NHP urine were quantitated in nonhuman primate urine by LC-MS/MS using a Thermo Ultimate 3000 UHPLC-TSQ Quantum MS system. Standard solutions from 0.16 to 250 μg/mL were made in water. Neat plasma, 40 fold diluted urine, and standards in water were derivatized with 50 mM each of 2-hydrazinoquinoline, dipyridyl disulfide, and triphenylphosphine in acetonitrile containing 1 ug/mL 13C915N-phenylalanine and d5-creatinine as internal standards and incubated at 60° C. for one hour. Following derivatization, 20 μL of standards and samples were diluted with 180 μL 0.1% formic acid/acetonitrile (140:40). Ten microliters were injected onto a Phenomenex Luna 5 um C18(2) 100 A, 100×2 mm column using 0.1% formic acid (A) and acetonitrile with 0.1% formic acid at 0.5 mL/min after an initial 10% B hold for 0.5 minutes using a gradient from 10 to 97% B over 1.5 minutes followed by wash and equilibration steps. Compounds were detected by tandem mass spectroscopy with selected reaction monitoring in electrospray positive ion mode using the following ion pairs: Phenylalanine 307/186, d5-phenylalanine 312/186, trans-cinnamic acid 290/131, d5-trans-cinnamic acid 295/136, hippuric acid, 321/160, d5-hippuric acid 326/160, 13C9 15N-phenylalanine 317/187, creatinine 114/44, d5-creatinine 119/49. Chromatograms are integrated and analyte/internal standard peak area ratios were used to calculate unknown concentrations.

### Example 6

### In Vitro Activity Profile of Δpks Strains Comprising a Gene Expression System for the Degradation of Oxalate

The Δpks EcN strain described in Example 1 (deleted clbA-clbR gene and promoter sequences, intact clbS gene sequence with the operably linked promoter deleted) is engineered to further comprise an expression systems for the degradation of oxalate, as described in PCT/US2016/049781 filed Aug. 31, 2016, the contents of which is herein incorporated by reference in its entirety, to assess the effect of Δpks on the ability of the strains to consume oxalate.

Strains are grown in shake flasks and subsequently activated in an anaerobic chamber followed by concentration and freezing at ≤−65° C. in glycerol-based formulation buffer (PBS+25% Glycerol). In assay media containing 10 mM oxalate, activated cells are resuspended to OD600=5 and incubated statically at 37° C. Supernatant samples are removed at 30 and 60 min to determine the concentrations of oxalate. Concentrations are determined by liquid chromatography-tandem mass spectrometry (LC-MS/MS).

Oxalate is quantitated in bacterial supernatant by LC-MS/MS using a Thermo Vanquish UHPLC-Altis TSQ MS system. Standards are prepared at 0.8 to 1000 μg/mL in water. Samples and standards are diluted ten-fold with 10 mM ammonium acetate that includes 1 μg/mL 13C2-oxalate as an internal standard. Ten microliters are injected onto a Waters Acquity HSS T3 1.8 um 100 A 2.1×100 mm column using 10 mM ammonium acetate (A) and methanol (B) at 0.4 mL/min and 50° C. Analytes are separated after an initial 100% A hold for 0.5 minutes using a gradient from 0 to 95% B over 1.5 minutes followed by wash and equilibration steps. Compounds are detected by tandem mass spectroscopy with selected reaction monitoring in electrospray negative ion mode using the following ion pairs: Oxalate 89/61, 13C2-oxalate 91/62. Chromatograms are integrated and oxalate/13C2-oxalate (analyte/internal standard) peak area ratios are used to calculate unknown concentrations.

### Example 7

### Pharmacodynamics of a Δpks Strain Capable of Catabolizing Oxalate Following Administration to Non-Human Primates

The in vivo activity of a Δpks EcN strain, e.g., as described in Example 1, engineered to further comprise an expression system for the degradation of oxalate, as described in Example 6, was compared to a strain comprising the expression system for the degradation of oxalate but not the Δpks deletion.

In vivo activity single dose cross-over studies were performed to assess the ability of the strains to metabolize gastrointestinal and diet-derived oxalate and 13 C2-oxalate in a nonhuman primate model of acute hyperoxaluria. Urinary recovery of oxalate and 13 C2-oxalate was significantly decreased regardless of the presence of the pks deletion, as compared to vehicle control, indicating that both strains are capable of consuming oxalate in nonhuman primates with acute hyperoxaluria. Results are shown in Table 5 and Table 6.

TABLE 5
Percent change in Urine Oxalate as compared to vehicle control

| | | % change as compared to |
|---|---|---|
| Strain | Timepoint | vehicle |
| SYNB8802 | 6 h | 73% |
| SYNB8802v1 (SYNB8802, | 8 h | 65% |
| further having Δpks) | | |

TABLE 6
Percent change in Urine 13 C2-oxalate
as compared to vehicle control

| | | % change as compared to |
|---|---|---|
| Strain | Timepoint | vehicle |
| SYNB8802 | 6 h | 75% |
| SYNB8802v1 (SYNB8802 | 8 h | 82% |
| further having Δpks) | | |

Exemplary Sequences

>Brevibacterium_aurantiacum_MGL-SEQ ID NO: 101
ATGACGAGCCTTCACCCCGAAACTTTAATGGTGCACGGGGGTATGAAGGGATTGACAGAGG
CGGGTGTTCACGTCCCGGCGATCGACCTGTCGACAACGAATCCTGTTAATGACGTAGCGACA
GGGGGTGATTCTTACGAATGGCTTGCAACGGGTCACACGTTGAAGGATGGTGATTCTGCTGT
TTACCAGCGTTTATGGCAGCCCGGTGTAGCTCGTTTCGAGACGGCTCTGGCTGGGCTGGAGC
ACGCGGAGGAGGCTGTAGCTTTCGCCACGGGTATGGCCGCCATGACCGCGGCCTTATTGGCA
GCAGTATCAGCTGGCACGCCTCATATCGTCGCAGTCCGCCCATTATACGGCGGTTCAGACCA
TCTTTTAGAGACTGGTTTACTGGGGACAACTGTCACGTGGGCGAAAGAAGCTGACATCGCCA
GTGCAATCCAAGACGATACAGGGTTAGTGATCGTGGAGACGCCAGCCAATCCTAGTCTTGAT
CTGGTAGACCTGGATTCTGTCGTGTCAGCGGCAGGGAACGTCCCTGTCCTTGTAGATAATAC
GTTTTGTACTCCTGTCCTTCAGCAACCTATCTCGCACGGCGCCGCACTTGTATTACATTCGGC
AACAAAGTATTTAGGGGGGCATGGGGACGCCATGGGCGGGATCATCGCGACTAATGCTGAT
TGGGCTATGCGCCTGCGTCAGGTCCGTGCGATCACTGGTGCATTACTGCATCCGATGGGCGC
GTACTTGTTACACCGCGGTTTGCGTACTCTGGCTGTTCGCATGCGTGCCGCTCAGACAACTGC
TGGTGAGCTTGCTGAGCGCTTAGACGCTCATCCCGCTATTTCAGTTGTGCACTACCCAGGATT
GAAGGGGCAAGATCCTCGTGGATTGTTAGGCCGTCAGATGAGTGGAGGTGGGGCAATGATT
GCTATGGAGCTGGCAGGGGGATTCGATGCGGCACGCTCCTTTGTTGAGCATTGTAACTTGGT
CGTGCATGCCGTATCACTTGGGGGAGCAGACACATTAATTCAACATCCGGCAAGTTTGACAC
ACCGCCCGGTCGCAGCAACCGCCAAGCCTGGTGATGGTTTGATCCGCTTGAGCGTGGGGCTT
GAGCACGTTGACGACTTGGCCGATGACTTGATCGCTGCTTTGGACGCCTCTCGCGCTGCTGC
ATGA
>Citrobacter_freundii_MGL-SEQ ID NO: 102
ATGTCTGATTGTCGTGCCTACGGCTTTAACACACAGATCGTCCACGCCGGACAACAACCGGA
CCCAAGCACTGGTGCATTGTCGACGCCAATCTTTCAGACTTCTACTTTCGTTTTCGATTCGGC
AGAACAGGGGGCCGCACGTTTCGCGTTGGAGGAACCGGGATACATCTATACGCGCTTGGGC
AATCCAACTACTGATGCGCTTGAAAAGAAACTTGCAGTGCTGGAACGTGGGGACGCGGCGT
TAGCCACAGCGAGCGGCATCTCCGCTATTACCACAAGTCTTCTTACTCTTTGCCAGCAAGGG
GACCATATCGTATCAGCTAGTGCAATCTACGGTTGCACTCACGCATTCTTAAGTCATTCGCTT
CCTAAATTTGGCATTAACGTTTCATTCGTCGACGCAGCGAAGCCCGAAGAAATCCGCGCCGC
CATGCGTCCAGAGACAAAAGTGGTATATATCGAGACGCCCGCGAACCCTACTTTATCCTTAG
TGGATATTGAGACTGTTGCTGGAATCGCGCACCAGCAAGGAGCCTTGTTAGTCGTGGACAAC
ACTTTTATGTCTCCGTATTGTCAACAACCATTACTGCTGGGTGCTGACATCGTGGTCCATAGT
GTTACCAAGTACATTAACGGGCATGGGGATGTTATTGGCGGTGTCATCGTAGGTAAACAGGA
GTTCATCGACCAAGCACGCTTTGTGGGATTAAAGGACATTACGGGCGGATGCATGAGCCCCT
TTAATGCATGGCTGACACTTCGCGGGGTAAAGACGTTAGGGATCCGTATGGAGCGTCATTGC
GAAAACGCATTGAAAATCGCGCGTTTTCTTGAGGGCCATCCTGCTATTACACGTGTCTATTAT
CCTGGATTGCCGTCACACCCCCAATACGAACTTGGGAAGCGCCAAATGTCGCTTCCTGGGGG
AATCATTTCGTTTGAAATTGCTGGTGGCCTTGAAGCAGGTCGCCGTATGATTAACAGCGTGG
AGTTATGTTTGCTGGCAGTTAGCTTAGGTGATACGGAGACGTTGATTCAGCATCCGGCGAGC
ATGACACATTCCCCTGTTGCTCCCGAGGAGCGTCTGAAGGCTGGCATCACTGATGGACTGAT
CCGTTTGTCCGTCGGCCTTGAAGATCCGGAAGACATCATTAACGACCTGGAGCACGCCATTC
GCAAGGCAACGTTTtaa
>Porphyromonas_gingivalis_MGL-SEQ ID NO: 103
ATGAAGAAAGAGGACTTAATGCGTTCGGGGTTCGCAACTCGTGCTATTCATGGTGGAGCCAT
CGAAAATGCTTTTGGGTGCCTGGCCACCCCCATCTACCAAACTTCAACGTTTGTTTTCGATAC
GGCTGAACAGGGTGGACGTCGCTTTGCGGGTGAAGAAGATGGGTACATTTACACTCGCTTAG
GAAACCCGAACTGTACCCAGGTTGAGGAGAAGCTGGCGATGTTAGAAGGAGGGGAAGCAG
CGGCCTCCGCAAGTAGTGGTATCGGAGCGATCTCTTCAGCTATCTGGGTGTGCGTCAAGGCG
GGAGATCATATTGTAGCGGGAAAAACTTTGTACGGTTGCACGTTTGCGTTCTTGACCCACGG
ACTTTCTCGCTATGGAGTTGAGGTCACCTTGGTGGACACGCGCCATCCAGAAGAGGTTGAGG
CCGCTATTCGTCCAAACACTAAGTTGGTCTATTTGGAAACTCCCGCAAACCCTAACATGTAC
CTTACTGATATCAAAGCTGTGTGTGATATCGCGCACAAGCATGAGGGTGTTCGCGTCATGGT
GGATAATACCTACTGTACACCTTACATCTGTCGCCCACTGGAGTTAGGTGCTGATATTGTTGT
CCATTCCGCCACAAAATATCTGAACGGACATGGAGATGTTATCGCTGGGTTTGTAGTAGGGA
AAGAGGATTACATCAAGGAGGTCAAATTGGTTGGTGTTAAGGACTTGACAGGAGCCAATAT
GTCACCCTTCGACGCATACCTTATTAGTCGTGGTATGAAGACGTTACAAATCCGTATGGAAC
AACATTGCCGTAACGCACAAACAGTTGCTGAGTTTCTGGAGAAGCATCCTGCGGTTGAGGCG
GTGTACTTCCCGGGCTTGCCTTCATTCCCGCAATATGAGTTGGCCAAGAAGCAGATGGCACT
TCCGGGAGCGATGATTGCGTTTGAAGTTAAGGGAGGCTGCGAAGCCGGTAAGAAACTTATG
AACAATCTTCATCTGTGTTCCTTAGCCGTCTCCTTAGGCGATACCGAGACTTTAATCCAACAC
CCGGCCTCAATGACGCACTCGCCCTATACGCCCGAGGAACGCGCCGCAAGCGACATCTCGG
AGGGGCTTGTACGCCTTTCTGTTGGATTGGAAAATGTTGAGGATATTATCGCGGACCTGAAG
CATGGCCTGGATAGTCTTATCtaa
>Streptomyces_sp590_MDC-SEQ ID NO: 104
ATGTCCCCGACGGCGTTTCCAGCGGCCGAAACAGCTACTGCCCCTGCAACTGCCGTCGAT
CCTGGGCCAGAACTGGACGGCGGAGATTTCGCCCTTCCAGAGGGCGGGCTGGATGACGATC
GTCGCTTACGTGCATTGGACGCAGTTGACGAGTATTTGACCCGCAAGCGCAAGCATTTGGTT
GGGTACCAAGCTACCCAGGATATGCAGGGAACGGCCTTGGATTTAGCCCGTTTCATGCCCAA
CAACATCAACAACCTGGGAGATCCTTTCCAGTCGGGTGGGTATAAACCAAATACGAAAGTC
GTTGAGCGTGCCGTACTGGACTACTATGCAAAATTGTGGCACGCAGAACGTCCACACGACCC
AGCTGACCCAGAAAGCTACTGGGGTTACATGTTATCGATGGGCTCAACTGAGGGCAACATGT
ACGCCCTGTGGAATGCACGTGACTACCTGTCGGGTAAGGCTTTGATTCAGCCTCCCACGGCA
CCATTTGACGCTGTTCGCTACGTGAAGGCTGACCCCGATCGCCGCAATCCTAACGCACACCA
CCCAGTCGCATTCTACTCGGAGGATACCCACTATTCTTTTGCTAAAGCCGTTGCGGTGCTGGG
TGTCGAAACTTTCCACGCTGTGGGTCTGGAGAAATACGCTGACGAGTGCCCCTTGGTGGATC
CAGTAACCGGCCTTCGTACCTGGCCGACCGAAGTTCCATCGCGCCCGGGGCCGTCGGGTTTA
AGCTGGGACGGCCCTGGTGAGATTGATGTTGATGCGCTTGCAGTACTGGTCGAGTTCTTCGC
AGCGAAGGGTCACCCCGTCTTCGTCAACCTTAACTTGGGGTCTACATTTAAAGGAGCACATG
ATGACGTACGTGCGGTATGTGAACGCTTATTACCAATCTTCGAGCGCCATGGCTTAGTACAA
CGTGAAGTTGTATATGGGAGCTGTCCCCAAACCGGCCGCCCTTTAGTGGATGTACGTCGCGG
ATTTTGGATCCACGTAGATGGGGCACTTGGGGCGGGGTATGCCCCTTTTCTGCGTCTTGCCGC
CGAAGACCCGGAAGGTTATGGTTGGACCCCTGAGGCAGAATTACCTGAGTTCGACTTCGGCT
TACGTTTGCCGACGGCGGGGCATGGAGAAGTTGATATGGTTAGCAGCATCGCCATGAGTGG
ACATAAGTGGGCAGGCGCGCCGTGGCCATGCGGCATCTATATGACGAAAGTGAAATATCAG
ATTAGTCCACCGTCACAGCCCGATTATATTGGTGCTCCTGACACAACATTTGCCGGTTCCCGT
AACGGCTTTTCGCCGTTAATTTTGTGGGATCATTTATCGCGCTACTCGTACCGCGACCAGGTA
GAGCGCATCCGCGAAGCACAGGAGCTTGCAGCATATTTGGAACGCCGCCTTACCGCTATGG
AGCGCGAGCTGGGAGTGGAACTTTGGCCAGCCCGCACACCGGGTGCTGTAACCGTACGTTTT
CGCAAACCCTCTGCTGAGCTGGTTGCGAAGTGGTCCTTGTCGTCGCAGGATGTTTTAATGGT
GCCGGGTGATGAAACTACGCGTCGTAGTTACGTTCATGTGTTCGTGATGCCTTCTGTTGATCG
TGCAAAGTTAGATGCGTTGCTGGCAGAATTGGCCGAAGATCCCGTCATCTTGGGTGCGCCTta
a
ABC transporter from EcN-MetNIQ
>MetN-SEQ ID NO: 105
Atgataaaactttcgaatatcaccaaagtgttccaccagggcacccgcaccatccaggcgttgaacaacgtcagcctgcatgtgccagctggacaaattt
atggcgttatcggtgcctcaggcgcgggtaagagtacgcttatacgttgtgtaaacctgctggagcgcccaaccgagggtagcgtgctggtcgatggcc
aggaactgaccacgctgtcagaatccgagttgaccaaagctcgtcgccagattggtatgattttccagcattttaacctgctctcttcgcgtactgtttttggc
aacgtggctctgccgctggagctggacaacacaccgaaagacgagatcaaacgtcgcgtgacggaattgctgtcattggttggtcttggcgataagcat
gatagctacccgtcgaatctttccggtgggcagaaacaacgtgtggcgattgcccgtgcattagccagcaatcccaaagtattgctgtgtgatgaagccac
cagcgcgctggacccggcaacgacacgttctattctcgaactgctgaaagacatcaaccgccgtctgggtttgacgattctgttgatcactcacgaaatgg
acgttgtgaagcgcatttgtgattgcgtggcggtcatcagcaatggcgaactgatcgagcaggacacggtaagtgaagtgttctcgcatccgaaaacgcc
gctggcgcagaagtttattcagtcgaccctgcatctggatatcccggaagattaccaggaacgtctgcaagcggagccatttactgactgcgtgccgatg
ctgcgtctggagtttaccggtcaatcggtcgatgccccactgctttctgaaaccgcgcgtcgtttcaacgtcaacaacaacattattagcgcgcagatggat
tacgccggtggcgttaagttcggcatcatgctgactgaaatgcacggcacacaacaagatacgcaagccgccattgcctggctgcaggaacaccatgta
aaagtagaggtactgggttatgtctga
>MetI-SEQ ID NO: 106
Atgtctgagccgatgatgtggctgctggttcgtggcgtatgggaaacgctggcaatgaccttcgtatccggtttttttggctttgtgattggtctgccgg
ttggcgttctgctttatgtcacgcgtccggggcaaattattgctaacgcgaagttgtatcgtaccatttctgcgattgtgaacattttccgttccatcccgttcatt
atcttgctggtatggatgattccgtttacccgcgttattgtcggtacatcgattggattgcaggcagcgattgttccgttaaccgttggtgcagcaccgtttattg
cccgtatggtcgagaacgctctgctggagatcccaaccgggttaattgaagcttcccgcgcaatgggggccacgccaatgcagatcgtccgtaaagtgc
tgttaccggaagcgttgccgggtctggtgaatgcggcaactatcaccctgattaccctggttggttattccgcgatgggtggtgcagtcggtgccggtggtt
taggtcagattggctatcagtatggctacatcggctacaacgcgacggtgatgaatacggtactggtattgctggtcattctggtttatttaattcagttcgcag
gcgaccgcatcgtccgggctgtcactcgcaagtaa
>MetQ-SEQ ID NO: 107
Atggcgttcaaattcaaaacctttgcggcagtgggagccctgattggatcactggcactggtaggctgcggtcaggatgaaaaagatccaaaccacatta
aagtcggcgtgattgttggtgccgaacagcaggttgcagaagtcgcgcagaaagttgcgaaagacaaatatggcctggacgttgagctggtaaccttca
acgactatgttctgccaaacgaagcattgagcaaaggcgatatcgacgccaacgccttccagcataaaccgtaccttgatcagcaactgaaagatcgtgg
ctacaaactggtcgcagtaggcaacacatttgtttatccgattgctggttactccaagaaaatcaaatcactggatgaactgcaggatggttcgcaggttgc
cgtgccaaacgacccaactaaccttggtcgttcactgctgctgctgcaaaaagtgggcttgatcaaactgaaagatggcgttggcctgctgccgaccgttc
ttgatgttgttgagaacccaaaaaatctgaaaattgttgaactggaagcaccgcagctaccgcgctctctggacgacgcgcaaatcgctctggcagttatc
aataccacctatgccagccagattggcctgactccagcgaaagacggtatctttgtcgaagataaagagtccccgtacgtaaacctgatcgtaacgcgtg
aagacaacaaagacgccgaaaacgtgaagaaattcgttcaggcttatcagtctgacgaagtttacgaagcagcaaacaaagtgtttaacggcggcgctg
ttaaaggctggtaa
>Brevibacterium_aurantiacum_MGL-SEQ ID NO: 108
MTSLHPETLMVHGGMKGLTEAGVHVPAIDLSTTNPVNDVATGGDSYEWLATGHTLKDGDSAV
YQRLWQPGVARFETALAGLEHAEEAVAFATGMAAMTAALLAAVSAGTPHIVAVRPLYGGSDH
LLETGLLGTTVTWAKEADIASAIQDDTGLVIVETPANPSLDLVDLDSVVSAAGNVPVLVDNTFCT
PVLQQPISHGAALVLHSATKYLGGHGDAMGGIIATNADWAMRLRQVRAITGALLHPMGAYLLH
RGLRTLAVRMRAAQTTAGELAERLDAHPAISVVHYPGLKGQDPRGLLGRQMSGGGAMIAMEL
AGGFDAARSFVEHCNLVVHAVSLGGADTLIQHPASLTHRPVAATAKPGDGLIRLSVGLEHVDDL
ADDLIAALDASRAAA*
>Citrobacter_freundii_MGL-SEQ ID NO: 109
MSDCRAYGFNTQIVHAGQQPDPSTGALSTPIFQTSTFVFDSAEQGAARFALEEPGYIYTRLGN
PTTDALEKKLAVLERGDAALATASGISAITTSLLTLCQQGDHIVSASAIYGCTHAFLSHSLPKFGIN
VSFVDAAKPEEIRAAMRPETKVVYIETPANPTLSLVDIETVAGIAHQQGALLVVDNTFMSPYCQQ
PLLLGADIVVHSVTKYINGHGDVIGGVIVGKQEFIDQARFVGLKDITGGCMSPFNAWLTLRGVKT
LGIRMERHCENALKIARFLEGHPAITRVYYPGLPSHPQYELGKRQMSLPGGIISFEIAGGLEAGRR
MINSVELCLLAVSLGDTETLIQHPASMTHSPVAPEERLKAGITDGLIRLSVGLEDPEDIINDLEHAI
RKATF*
>Porphyromonas_gingivalis_MGL-SEQ ID NO: 110
MKKEDLMRSGFATRAIHGGAIENAFGCLATPIYQTSTFVFDTAEQGGRRFAGEEDGYIYTRLGNP
NCTQVEEKLAMLEGGEAAASASSGIGAISSAIWVCVKAGDHIVAGKTLYGCTFAFLTHGLSRYG
VEVTLVDTRHPEEVEAAIRPNTKLVYLETPANPNMYLTDIKAVCDIAHKHEGVRVMVDNTYCTP
YICRPLELGADIVVHSATKYLNGHGDVIAGFVVGKEDYIKEVKLVGVKDLTGANMSPFDAYLIS
RGMKTLQIRMEQHCRNAQTVAEFLEKHPAVEAVYFPGLPSFPQYELAKKQMALPGAMIAFEVK
GGCEAGKKLMNNLHLCSLAVSLGDTETLIQHPASMTHSPYTPEERAASDISEGLVRLSVGLENVE
DIIADLKHGLDSLI*
>Streptomyces_sp590_MDC-SEQ ID NO: 111
MSPTAFPAAETATAPATAVDPGPELDGGDFALPEGGLDDDRRLRALDAVDEYLTRKRKHLVGY
QATQDMQGTALDLARFMPNNINNLGDPFQSGGYKPNTKVVERAVLDYYAKLWHAERPHDPAD
PESYWGYMLSMGSTEGNMYALWNARDYLSGKALIQPPTAPFDAVRYVKADPDRRNPNAHHPV
AFYSEDTHYSFAKAVAVLGVETFHAVGLEKYADECPLVDPVTGLRTWPTEVPSRPGPSGLSWDG
PGEIDVDALAVLVEFFAAKGHPVFVNLNLGSTFKGAHDDVRAVCERLLPIFERHGLVQREVVYG
SCPQTGRPLVDVRRGFWIHVDGALGAGYAPFLRLAAEDPEGYGWTPEAELPEFDFGLRLPTAGH
GEVDMVSSIAMSGHKWAGAPWPCGIYMTKVKYQISPPSQPDYIGAPDTTFAGSRNGFSPLILWD
HLSRYSYRDQVERIREAQELAAYLERRLTAMERELGVELWPARTPGAVTVRFRKPSAELVAKW
SLSSQDVLMVPGDETTRRSYVHVFVMPSVDRAKLDALLAELAEDPVILGAP*
ABC transporter from EcN-MetNIQ
>MetN-SEQ ID NO: 112
MIKLSNITKVFHQGTRTIQALNNVSLHVPAGQIYGVIGASGAGKSTLIRCVNLLERPTEGSVLV
DGQELTTLSESELTKARRQIGMIFQHFNLLSSRTVFGNVALPLELDNTPKDEIKRRVTELLSLVGL
GDKHDSYPSNLSGGQKQRVAIARALASNPKVLLCDEATSALDPATTRSILELLKDINRRLGLTILL
ITHEMDVVKRICDCVAVISNGELIEQDTVSEVFSHPKTPLAQKFIQSTLHLDIPEDYQERLQAEPFT
DCVPMLRLEFTGQSVDAPLLSETARRFNVNNNIISAQMDYAGGVKFGIMLTEMHGTQQDTQAAI
AWLQEHHVKVEVLGYV*
>MetI-SEQ ID NO: 113
MSEPMMWLLVRGVWETLAMTFVSGFFGFVIGLPVGVLLYVTRPGQIIANAKLYRTISAIVNIF
RSIPFIILLVWMIPFTRVIVGTSIGLQAAIVPLTVGAAPFIARMVENALLEIPTGLIEASRAMGATPM
QIVRKVLLPEALPGLVNAATITLITLVGYSAMGGAVGAGGLGQIGYQYGYIGYNATVMNTVLVL
LVILVYLIQFAGDRIVRAVTRK*
>MetQ-SEQ ID NO: 114
MAFKFKTFAAVGALIGSLALVGCGQDEKDPNHIKVGVIVGAEQQVAEVAQKVAKDKYGLD
VELVTFNDYVLPNEALSKGDIDANAFQHKPYLDQQLKDRGYKLVAVGNTFVYPIAGYSKKIKSL
DELQDGSQVAVPNDPTNLGRSLLLLQKVGLIKLKDGVGLLPTVLDVVENPKNLKIVELEAPQLPR
SLDDAQIALAVINTTYASQIGLTPAKDGIFVEDKESPYVNLIVTREDNKDAENVKKFVQAYQSDE
VYEAANKVFNGGAVKGW*
>yjeH-SEQ ID NO: 115
atgagtggactcaaacaagaactggggctggcccagggcatcggcctactatcgacgtcattattaggcactggcgtgtttgccgttcctgcgttag
ctgcgctagtagcaggcaataacagcctgtgggcgtggcccgttttgattatcttagtgttcccgattgcgattgtgtttgcgattctgggtegccactatccc
agcgcaggcggcgtcgcacacttcgtcggtatggcgtttggttcgcggcttgagcgagtcaccggctggttgtttttatcggtcattcccgtgggtttgcct
gccgcgctacaaattgctgccggattcggccaggcaatgtttggctggcatagcgggcaactgttgttggcagaactcggtacgctggcgctggtgtggt
atatcggtactcgaggtgccagttccagtgctaatctacaaacagttattgccgggcttatcgtcgcactgattgtcgctatctggtggggggcgatatcaa
acctgcgaatatccccttccctgcgccaggaaatatcgaacttaccgggttattcgctgcgttatcagtgatgttctggtgttttgtcggtctggaagcatttgc
ccatcttgcctcggaatttaaaaatccagagcgtgattttcctcgtgctttgatgattggcctgctgctggcaggattagtctattggggctgtacggtagtcgt
cttacacttcgacgcctatggtgaacaaatggcggcggcagcatcgcttcccaaaattgtagtgcagttattcggtgtaggagcgttatggattgcctgcgt
aattggctatctggcctgctttgccagtctcaacatttatatacagagcttcgcccgcctggtctggtcgcaggcgcaacataatcctgaccattacctggca
cgcctctcttctcgccatattccgaataatgccctcaatgcggtgctcggctgctgcgtggtgagcacgttggtgattcatgctttagagatcaatctggacg
ctcttattatttatgccaatggcatctttattatgatttatctgttatgcatgctggcaggctgtaaattattgcaaggacgttatcgactactggcagtggttggcg
ggctattatgcgttctgttactggcaatggtcggctggaaaagtctctacgcgctgatcatgctggcggggttatggctgtttctgccaaaacgaaaaacgc
cggaaaatggcataaccacataa
>Citrobacter_freundii_WT_MGL-SEQ ID NO: 116
atgtctgactgtcgtgcttacggattcaatacccagatcgttcatgcgggccaacaacccgacccttccactggegcgctcagtacgcctattttccagacg
tcaaccttcgtttttgacagtgccgaacagggcgccgcccgctttgcgcttgaagaacccggctacatctatacgcgccttggaaaccccaccaccgacg
cgctggagaaaaagctggctgtgctggaaagaggcgatgccgcgctggcaactgcatccggtatttcagccatcaccacctcgttgctgaccctttgcca
gcagggcgaccatatcgtttccgccagtgccatttacggctgcactcacgcttttctgtcacacagcctgccgaagttcggcattaacgtcagcttcgtcga
cgccgccaagccggaagaaatccgcgcggccatgcgcccggaaaccaaagtggtgtatatcgaaacaccagcaaacccaacgctctcgctggttgat
attgaaacggtcgccgggatcgcccatcagcaaggcgcattgctggtcgtggataacacctttatgtcaccctactgccagcaacctctgctgttaggtgc
cgacatcgtggtgcacagcgtgaccaagtacatcaacggccacggggacgtgattggcggtgtgattgttggcaagcaggaatttatcgaccaggcac
gattcgtcgggctgaaagatatcaccggcggctgcatgagtccgttcaacgcctggctgacgctgcgcggcgtgaaaacgctgggcatccgaatggag
cgccattgtgaaaacgcgttaaaaattgcccgcttcctggaagggcatccggccattacccgcgtgtattaccctggcttgccttcacatccgcagtatgag
ctgggtaaacgtcagatgagcctgccgggaggaattatcagcttcgaaatcgccggcggcctcgaggctggcagacggatgatcaattctgtagaattgt
gcctcctcgcggtcagtctcggtgacaccgaaaccctcattcagcacccagcgtctatgacacattcgcccgttgcgccagaggaacggcttaaagcag
gtattaccgacggacttatccgtctttctgttggtcttgaagatccagaagatattattaacgaccttgaacacgccatcagaaaggcaacattctga
>Porphyromonas_gingivalis_WT_MGL-SEQ ID NO: 117
Atgaaaaaagaagaccttatgcgtagtggctttgccacacgtgccatccatggaggcgctatcgagaacgccttcggctgcttagccactcccatctacc
aaacatcgactttcgtttttgacactgccgaacagggaggccgccgctttgccggagaggaagacggatacatctatacccgtctgggcaaccccaactg
cacccaagtggaagagaaactggccatgctcgagggcggagaagccgccgcatcggcctcatccggtatcggagccatcagctctgccatctgggtat
gcgtgaaggccggcgaccatatcgtagccggcaagacgctctacggctgcaccttcgccttcctcacccacggactgagccgctacggtgtggaagtc
accctcgtggatacccgccatccggaagaggtggaggctgccattcgcccgaatacgaagctcgtctatctggagactccggccaaccccaatatgtac
ctgaccgacatcaaggcagtctgcgacatcgctcataagcacgaaggcgtacgcgtcatggtggacaatacctactgcacgccctatatctgccgtccg
ctggagctgggtgccgacatcgtggtacacagcgcgaccaagtacctgaacggacatggcgacgtcatcgccggattcgtcgttggtaaagaggacta
catcaaggaggtgaagctcgtcggcgtcaaggacctcacgggggccaatatgagtccgttcgatgcttatctgatcagccgcggcatgaagacgctgca
gatacgtatggagcagcactgccgcaatgctcagaccgtagccgaattcctcgaaaagcatccggccgtagaagcagtttatttccccggacttccgagc
ttcccccaatacgaattggccaagaagcagatggcactgcccggagccatgatcgccttcgaagtgaagggcggttgcgaagccggtaagaagctgat
gaacaacctgcacctctgctccctcgccgtgagcttggggatacggaaaccctcatccagcatccggccagcatgacccactegccctacacacccg
aagagcgtgctgccagcgacatatccgaaggactggtacgcctgtccgtgggtctggagaacgtggaggacatcatcgccgacctcaaacacggtctg
gacagcctgatctaa
>Brevibacterium_aurantiacum_WT_MGL-SEQ ID NO: 118
atgacctcactgcacccagaaacgctcatggtccacggcggaatgaaaggcctcaccgaggcaggagtccacgtaccggccategacctctcgacca
ccaacccagtcaacgatgtcgccaccggcggtgactcctacgaatggctcgccaccggccatacgctcaaggacggcgactcggccgtctaccagcg
cctctggcagcccggtgtcgcacgcttcgagaccgcgctggccgggctcgaacacgctgaggaagcagtcgccttcgccacgggcatggccgcaat
gactgccgcacttctcgcggccgtcagcgcaggaacaccccacatcgtcgcagtgcgtcccctctatggeggaagcgaccacctcctcgaaaccgga
ctgctggggacaacagtcacatgggcaaaggaagccgacatcgcctcggcgatccaagatgacaccggactcgtcattgtcgagaccccggcaaacc
ccagcctggaccttgttgatctcgacagtgtcgtctcagccgccggcaacgtgcctgtgctggtggacaacacattctgcacacctgttctccagcagccc
atctcccacggagcggccctcgtactgcacagcgcgacaaaatacctcggcggtcatggcgatgccatgggggcatcatcgccaccaacgccgact
gggcgatgcgcctgcgacaggtccgagccatcacaggagccctgctccaccccatgggcgcgtatctccttcatcggggcttgcgcactctggccgtg
cgcatgcgcgcggctcagaccaccgccggtgagctcgctgagcgcctggacgcgcaccctgccatctccgtcgtccactacccgggactgaaaggcc
aggacccacgcggactgctcggacgccaaatgtccggtggtggtgcgatgatcgcgatggagctcgccggtggattcgacgccgcccgcagcttcgt
cgaacactgcaacctcgtcgtccacgcggtgtccctgggcggcgctgacactctcatccagcatccggcgtcactgactcacaggccagttgcggcca
cggcgaagcccggcgatggtctcatccgactctctgtgggactcgaacacgtcgatgacctggcagacgatctcatcgctgccctcgacgcgagtcgg
gccgctgcctga
>Streptomyces_sp590_WT_MetDC-SEQ ID NO: 119
atgagcccgaccgccttccccgccgccgagaccgcgaccgcgccegcgaccgccgtcgatcccggtccggagctggacggcggtgacttcgccctc
cccgagggcggcctggacgacgaccggcggctgcgcgcgctcgacgccgtggacgagtacctgacccgcaagcgcaagcacctggteggctacc
aggccacccaggacatgcagggcaccgcactggacctcgcccggttcatgccgaacaacatcaacaaccteggcgacccgttccagagcggcggat
acaagcccaacaccaaggtcgtcgagcgggccgtgctcgactactacgcgaagctctggcacgccgagcgcccgcacgacccggccgacccggag
tcgtactggggctacatgctgtccatgggctcgaccgagggcaacatgtacgccctctggaacgccagggactacctgagcggcaaggcgctgatcca
gccgccgaccgcccccttcgacgcggtgcgctacgtcaaggccgaccccgaccgacggaacccgaacgcccaccacccggtggccttctactccga
ggacacccactactccttcgccaaggccgtggccgtcctcggcgtggagaccttccacgccgtcggcctggagaagtacgccgacgagtgcccgctg
gtcgacccggtgaccgggctgcgcacctggcccaccgaggtgccctcccgcccgggtccgtccggcctgtcctgggacggccccggcgagatagac
gtcgacgccctcgccgtactcgtcgagttcttcgccgccaagggtcacccggtcttcgtcaacctcaaccteggcagcaccttcaagggcgcccacgac
gacgtccgcgccgtctgcgagcgcttgctgccgatcttcgagcggcacgggctcgtccagcgcgaggtggtctacggcagctgcccgcagaccggcc
ggccgctggtggacgtgcgccgcggcttctggatccacgtggacggcgcgctcggcgccggctacgcgccgttcctgcggctggccgccgaggacc
cggaagggtacggctggacgcccgaggcggagctgcccgagttcgacttcggcctgcggctgcccaccgccgggcacggcgaggtggacatggtc
tcctcgatcgcgatgagcggccacaagtgggccggcgcgccgtggccgtgcggcatctacatgaccaaggtgaagtaccagatctegccgccgtccc
agccggactacatcggcgccccggacaccaccttcgccggctcccgcaacggcttctccccgctgatcctctgggaccacctgtcceggtactectacc
gggaccaggtggagcggatccgcgaggcccaggagctggccgcctacctggagcggcggctgaccgccatggagcgcgaactcggcgtcgagct
ctggccggcccgtaccccgggcgccgtcaccgtacggttccgcaagccgagcgccgagctggtggccaagtggtcgctgtcctcccaggacgtgctg
atggtcccgggcgacgagaccacccggcgcagctacgtgcacgtcttcgtgatgccctcggtcgaccgggccaagctggacgcgctgctcgccgaac
tcgccgaggacccggtgatcctgggcgcaccgtag
OxIT coding region (oxalate:formate antiporter from O. formigenes)-SEQ ID NO: 120
ATGAATAATCCACAAACAGGACAATCAACAGGCCTCTTGGGCAATCGTTGGTTCTACTTGGT
ATTAGCAGTTTTGCTGATGTGTATGATCTCGGGTGTCCAATATTCCTGGACACTGTACGCTAA
CCCGGTTAAAGACAACCTTGGCGTTTCTTTGGCTGCGGTTCAGACGGCTTTCACACTCTCTCA
GGTCATTCAAGCTGGTTCTCAGCCTGGTGGTGGTTACTTCGTTGATAAATTCGGTCCAAGAAT
TCCATTGATGTTCGGTGGTGCGATGGTTCTCGCTGGCTGGACCTTCATGGGTATGGTTGACAG
TGTTCCTGCTCTGTATGCTCTTTATACTCTGGCCGGTGCAGGTGTTGGTATCGTTTACGGTAT
CGCGATGAACACGGCTAACAGATGGTTCCCGGACAAACGCGGTCTGGCTTCCGGTTTCACCG
CTGCCGGTTACGGTCTGGGTGTTCTGCCGTTCCTGCCACTGATCAGCTCCGTTCTGAAAGTTG
AAGGTGTTGGCGCAGCATTCATGTACACCGGTTTGATCATGGGTATCCTGATTATCCTGATC
GCTTTCGTTATCCGTTTCCCTGGCCAGCAAGGCGCCAAAAAACAAATCGTTGTTACCGACAA
GGATTTCAATTCTGGCGAAATGCTGAGAACACCACAATTCTGGGTTCTGTGGACCGCATTCT
TTTCCGTTAACTTTGGTGGTTTGCTGCTGGTTGCCAACAGCGTCCCTTACGGTCGCAGCCTCG
GTCTTGCCGCAGGTGTGCTGACGATCGGTGTTTCGATCCAGAACCTGTTCAATGGTGGTTGC
CGTCCTTTCTGGGGTTTCGTTTCCGATAAAATCGGCCGTTACAAAACCATGTCCGTCGTTTTC
GGTATCAATGCTGTTGTTCTCGCACTTTTCCCGACGATTGCTGCCTTGGGCGATGTAGCCTTT
ATCGCCATGTTGGCAATCGCATTCTTCACATGGGGTGGTAGCTACGCTCTGTTCCCATCGACC
AACAGCGATATTTTCGGTACGGCATACTCTGCCAGAAACTATGGTTTCTTCTGGGCTGCAAA
AGCAACTGCCTCGATCTTCGGTGGTGGTCTGGGTGCTGCAATTGCAACCAACTTCGGATGGA
ATACCGCTTTCCTGATTACTGCGATTACTTCTTTCATCGCATTTGCTCTGGCTACCTTCGTTAT
TCCAAGAATGGGCCGTCCAGTCAAGAAAATGGTCAAATTGTCTCCAGAAGAAAAAGCTGTA
CATTAA
OxIT (oxalate:formate antiporter from O. formigenes)-SEQ ID NO: 121
MNNPQTGQSTGLLGNRWFYLVLAVLLMCMISGVQYSWTLYANPVKDNLGVSLAAVQTAFTLS
QVIQAGSQPGGGYFVDKFGPRIPLMFGGAMVLAGWTFMGMVDSVPALYALYTLAGAGVGIVY
GIAMNTANRWFPDKRGLASGFTAAGYGLGVLPFLPLISSVLKVEGVGAAFMYTGLIMGILIILIAF
VIRFPGQQGAKKQIVVTDKDFNSGEMLRTPQFWVLWTAFFSVNFGGLLLVANSVPYGRSLGLAA
GVLTIGVSIQNLFNGGCRPFWGFVSDKIGRYKTMSVVFGINAVVLALFPTIAALGDVAFIAMLAIA
FFTWGGSYALFPSTNSDIFGTAYSARNYGFFWAAKATASIFGGGLGAAIATNFGWNTAFLITAITS
FIAFALATFVIPRMGRPVKKMVKLSPEEKAVH*
frc (formyl-CoA transferase from O. formigenes)-SEQ ID NO: 122
ATGACTAAACCATTAGATGGAATTAATGTGCTTGACTTTACCCACGTCCAGGCAGGTCCTGC
CTGTACACAGATGATGGGTTTCTTGGGCGCAAACGTCATCAAGATTGAAAGACGTGGTTCCG
GAGATATGACTCGTGGATGGCTGCAGGACAAACCAAATGTTGATTCCCTGTATTTCACGATG
TTCAACTGTAACAAACGTTCGATTGAACTGGACATGAAAACCCCGGAAGGCAAAGAGCTTC
TGGAACAGATGATCAAGAAAGCCGACGTCATGGTCGAAAACTTCGGACCAGGCGCACTGGA
CCGTATGGGCTTTACTTGGGAATACATTCAGGAACTGAATCCACGCGTCATTCTGGCTTCCGT
TAAAGGCTATGCAGAAGGCCACGCCAACGAACACCTGAAAGTTTATGAAAACGTTGCACAG
TGTTCCGGCGGTGCTGCAGCTACCACCGGTTTCTGGGATGGTCCTCCAACCGTTTCCGGCGCT
GCTCTGGGTGACTCCAACTCCGGTATGCACCTGATGATCGGTATTCTGGCCGCTCTGGAAAT
GCGTCACAAAACCGGCCGTGGTCAGAAAGTTGCCGTCGCTATGCAGGACGCTGTTCTGAATC
TGGTTCGTATCAAACTGCGTGACCAGCAACGTCTGGAAAGAACCGGCATTCTGGCTGAATAC
CCACAGGCTCAGCCTAACTTTGCCTTCGACAGAGACGGTAACCCACTGTCCTTCGACAACAT
CACTTCCGTTCCACGTGGTGGTAACGCAGGTGGCGGCGGCCAGCCAGGCTGGATGCTGAAAT
GTAAAGGTTGGGAAACCGATGCGGACTCCTACGTTTACTTCACCATCGCTGCAAACATGTGG
CCACAGATCTGCGACATGATCGACAAGCCAGAATGGAAAGACGACCCAGCCTACAACACAT
TCGAAGGTCGTGTTGACAAGCTGATGGACATCTTCTCCTTCATCGAAACCAAGTTCGCTGAC
AAGGACAAATTCGAAGTTACCGAATGGGCTGCCCAGTACGGCATTCCTTGCGGTCCGGTCAT
GTCCATGAAAGAACTGGCTCACGATCCTTCCCTGCAGAAAGTTGGTACCGTCGTTGAAGTTG
TCGACGAAATTCGTGGTAACCACCTGACCGTTGGCGCACCGTTCAAATTCTCCGGATTCCAG
CCGGAAATTACCCGTGCTCCGCTGTTGGGCGAACATACCGACGAAGTTCTGAAAGAACTGG
GTCTTGACGATGCCAAGATCAAGGAACTGCATGCAAAACAGGTAGTTTGA
Frc (Formyl-CoA transferase from O. formigenes)-SEQ ID NO: 123
MTKPLDGINVLDFTHVQAGPACTQMMGFLGANVIKIERRGSGDMTRGWLQDKPNVDSLYFTMF
NCNKRSIELDMKTPEGKELLEQMIKKADVMVENFGPGALDRMGFTWEYIQELNPRVILASVKGY
AEGHANEHLKVYENVAQCSGGAAATTGFWDGPPTVSGAALGDSNSGMHLMIGILAALEMRHK
TGRGQKVAVAMQDAVLNLVRIKLRDQQRLERTGILAEYPQAQPNFAFDRDGNPLSFDNITSVPR
GGNAGGGGQPGWMLKCKGWETDADSYVYFTIAANMWPQICDMIDKPEWKDDPAYNTFEGRV
DKLMDIFSFIETKFADKDKFEVTEWAAQYGIPCGPVMSMKELAHDPSLQKVGTVVEVVDEIRGN
HLTVGAPFKFSGFQPEITRAPLLGEHTDEVLKELGLDDAKIKELHAKQVV*
oxc (oxalylCoA decarboxylase from O. formigenes) also referred
to as oxdc (oxalate decarboxylase)-SEQ ID NO: 124
ATGAGTAACGACGACAATGTAGAGTTGACTGATGGCTTTCATGTTTTGATCGATGCCCTGAA
AATGAATGACATCGATACCATGTATGGTGTTGTCGGCATTCCTATCACGAACCTGGCTCGTA
TGTGGCAAGATGACGGTCAGCGTTTTTACAGCTTCCGTCACGAACAACACGCAGGTTATGCA
GCTTCTATCGCCGGTTACATCGAAGGAAAACCTGGCGTTTGCTTGACCGTTTCCGCCCCTGGC
TTCCTGAACGGCGTGACTTCCCTGGCTCATGCAACCACCAACTGCTTCCCAATGATCCTGTTG
AGCGGTTCCAGTGAACGTGAAATCGTCGATTTGCAACAGGGCGATTACGAAGAAATGGATC
AGATGAATGTTGCACGTCCACACTGCAAAGCTTCTTTCCGTATCAACAGCATCAAAGACATT
CCAATCGGTATCGCTCGTGCAGTTCGCACCGCTGTATCCGGACGTCCAGGTGGTGTTTACGTT
GACTTGCCAGCAAAACTGTTCGGTCAGACCATTTCTGTAGAAGAAGCTAACAAACTGCTCTT
CAAACCAATCGATCCAGCTCCGGCACAGATTCCTGCTGAAGACGCTATCGCTCGCGCTGCTG
ACCTGATCAAGAACGCCAAACGTCCAGTTATCATGCTGGGTAAAGGCGCTGCATACGCACA
ATGCGACGACGAAATCCGCGCACTGGTTGAAGAAACCGGCATCCCATTCCTGCCAATGGGT
ATGGCTAAAGGCCTGCTGCCTGACAACCATCCACAATCCGCTGCTGCAACCCGTGCTTTCGC
ACTGGCACAGTGTGACGTTTGCGTACTGATCGGCGCTCGTCTGAACTGGCTGATGCAGCACG
GTAAAGGCAAAACCTGGGGCGACGAACTGAAGAAATACGTTCAGATCGACATCCAGGCTAA
CGAAATGGACAGCAACCAGCCTATCGCTGCACCAGTTGTTGGTGACATCAAGTCCGCCGTTT
CCCTGCTCCGCAAAGCACTGAAAGGCGCTCCAAAAGCTGACGCTGAATGGACCGGCGCTCT
GAAAGCCAAAGTTGACGGCAACAAAGCCAAACTGGCTGGCAAGATGACTGCCGAAACCCCA
TCCGGAATGATGAACTACTCCAATTCCCTGGGCGTTGTTCGTGACTTCATGCTGGCAAATCC
GGATATTTCCCTGGTTAACGAAGGCGCTAATGCACTCGACAACACTCGTATGATTGTTGACA
TGCTGAAACCACGCAAACGTCTTGACTCCGGTACCTGGGGTGTTATGGGTATTGGTATGGGC
TACTGCGTTGCTGCAGCTGCTGTTACCGGCAAACCGGTTATCGCTGTTGAAGGCGATAGCGC
ATTCGGTTTCTCCGGTATGGAACTGGAAACCATCTGCCGTTACAACCTGCCAGTTACCGTTAT
CATCATGAACAATGGTGGTATCTATAAAGGTAACGAAGCAGATCCACAACCAGGCGTTATCT
CCTGTACCCGTCTGACCCGTGGTCGTTACGACATGATGATGGAAGCATTTGGCGGTAAAGGT
TATGTTGCCAATACTCCAGCAGAACTGAAAGCTGCTCTGGAAGAAGCTGTTGCTTCCGGCAA
ACCATGCCTGATCAACGCGATGATCGATCCAGACGCTGGTGTCGAATCTGGCCGTATCAAGA
GCCTGAACGTTGTAAGTAAAGTTGGCAAGAAATAA
Oxc (oxalylCoA decarboxylase from O. formigenes) SEQ ID NO: 125
MSNDDNVELTDGFHVLIDALKMNDIDTMYGVVGIPITNLARMWQDDGQRFYSFRHEQHAGYA
ASIAGYIEGKPGVCLTVSAPGFLNGVTSLAHATTNCFPMILLSGSSEREIVDLQQGDYEEMDQMN
VARPHCKASFRINSIKDIPIGIARAVRTAVSGRPGGVYVDLPAKLFGQTISVEEANKLLFKPIDPAP
AQIPAEDAIARAADLIKNAKRPVIMLGKGAAYAQCDDEIRALVEETGIPFLPMGMAKGLLPDNHP
QSAAATRAFALAQCDVCVLIGARLNWLMQHGKGKTWGDELKKYVQIDIQANEMDSNQPIAAP
VVGDIKSAVSLLRKALKGAPKADAEWTGALKAKVDGNKAKLAGKMTAETPSGMMNYSNSLG
VVRDFMLANPDISLVNEGANALDNTRMIVDMLKPRKRLDSGTWGVMGIGMGYCVAAAAVTG
KPVIA VEGDSAFGFSGMELETICRYNLPVTVIIMNNGGIYKGNEADPQPGVISCTRLTRGRYDMM
MEAFGGKGYVANTPAELKAALEEAVASGKPCLINAMIDPDAGVESGRIKSLNVVSKVGKK*
ScAAE3 (oxalate-CoA ligase from S. cerevisiae)-SEQ ID NO: 126
ATGACCAGTGCAGCTACGGTGACCGCGAGCTTTAATGACACTTTTTCTGTGAGCGATAATGT
CGCGGTAATCGTACCGGAAACCGATACGCAGGTCACCTACCGTGATCTTTCCCACATGGTAG
GACACTTTCAAACAATGTTCACGAACCCGAATAGTCCTCTGTACGGGGCGGTCTTTCGTCAA
GACACGGTAGCGATTAGCATGCGTAACGGCCTTGAATTTATTGTGGCTTTCCTTGGAGCCAC
GATGGATGCGAAAATTGGTGCGCCACTGAATCCCAATTATAAAGAGAAGGAGTTTAATTTTT
ACCTGAATGACTTAAAGTCCAAAGCCATCTGCGTGCCGAAAGGCACCACCAAACTGCAAAG
TTCAGAAATTCTTAAGAGTGCGTCCACGTTCGGGTGCTTTATTGTGGAACTGGCGTTTGACGC
CACCCGTTTTCGTGTTGAATATGACATTTACTCCCCGGAGGACAATTATAAACGTGTGATCTA
CCGCAGCCTTAACAATGCTAAGTTTGTCAACACAAACCCTGTCAAGTTCCCGGGTTTCGCCC
GCAGCTCGGATGTTGCACTTATTTTGCATACCTCAGGCACCACTAGTACCCCAAAGACCGTA
CCCCTCTTGCATCTGAATATTGTCCGTTCAACCCTGAATATCGCCAACACTTACAAACTTACC
CCGCTGGATCGCTCCTATGTTGTAATGCCGCTGTTTCATGTACATGGATTAATCGGCGTCTTA
CTGAGTACGTTCCGCACCCAGGGCAGTGTAGTCGTCCCGGACGGCTTTCATCCGAAGCTCTT
CTGGGATCAGTTTGTTAAATATAACTGCAATTGGTTTAGTTGCGTCCCAACGATCTCTATGAT
TATGTTGAATATGCCCAAACCGAATCCGTTTCCGCACATTCGCTTTATCCGCTCATGTAGCAG
CGCGCTGGCGCCAGCAACGTTTCACAAGCTGGAAAAAGAATTTAATGCCCCAGTTCTGGAA
GCGTACGCGATGACAGAAGCATCTCATCAGATGACCAGTAACAATCTGCCTCCCGGTAAAC
GTAAACCGGGGACCGTGGGCCAACCTCAAGGTGTAACCGTAGTAATCCTGGATGACAACGA
TAACGTTCTGCCGCCCGGCAAAGTTGGCGAGGTGTCGATCCGTGGGGAGAACGTCACCCTGG
GCTACGCTAATAACCCGAAAGCTAACAAAGAAAACTTCACTAAACGTGAAAACTATTTCCGT
ACCGGGGATCAGGGCTACTTCGACCCGGAGGGCTTTCTCGTGCTGACCGGCCGCATTAAAGA
ATTGATCAATCGCGGTGGTGAAAAAATTAGTCCTATTGAACTGGACGGAATCATGCTCTCGC
ATCCTAAAATCGACGAGGCGGTGGCGTTCGGCGTTCCAGATGATATGTATGGCCAAGTCGTT
CAGGCGGCAATCGTGTTGAAAAAGGGGGAAAAGATGACCTATGAAGAATTAGTGAATTTCC
TGAAAAAGCATTTAGCAAGCTTTAAAATCCCAACCAAAGTCTACTTTGTGGATAAGCTGCCT
AAAACGGCCACCGGGAAGATTCAACGTCGCGTAATCGCCGAAACCTTCGCGAAATCTAGTC
GCAACAAAAGCAAACTTtaa
ScAAE3 (Oxalate-CoA ligase from S. cerevisiae)-SEQ ID NO: 127
MTSAATVTASFNDTFSVSDNVAVIVPETDTQVTYRDLSHMVGHFQTMFTNPNSPLYGAVFRQDT
VAISMRNGLEFIVAFLGATMDAKIGAPLNPNYKEKEFNFYLNDLKSKAICVPKGTTKLQSSEILKS
ASTFGCFIVELAFDATRFRVEYDIYSPEDNYKRVIYRSLNNAKFVNTNPVKFPGFARSSDVALILH
TSGTTSTPKTVPLLHLNIVRSTLNIANTYKLTPLDRSYVVMPLFHVHGLIGVLLSTFRTQGSVVVP
DGFHPKLFWDQFVKYNCNWFSCVPTISMIMLNMPKPNPFPHIRFIRSCSSALAPATFHKLEKEFN
APVLEAYAMTEASHQMTSNNLPPGKRKPGTVGQPQGVTVVILDDNDNVLPPGKVGEVSIRGEN
VTLGYANNPKANKENFTKRENYFRTGDQGYFDPEGFLVLTGRIKELINRGGEKISPIELDGIMLSH
PKIDEAVAFGVPDDMYGQVVQAAIVLKKGEKMTYEELVNFLKKHLASFKIPTKVYFVDKLPKT
ATGKIQRRVIAETFAKSSRNKSKL*
yfdE (Acetyl-CoA:oxalate CoA-transferase from E. coli)-SEQ ID NO: 128
atgACAAATAATGAAAGCAAAGGGCCGTTTGAAGGCTTATTAGTTATCGATATGACACATGTC
CTTAATGGACCTTTCGGAACTCAACTTCTTTGTAATATGGGCGCAAGGGTAATTAAAGTTGA
GCCGCCGGGTCATGGTGATGATACCCGCACATTTGGTCCCTATGTGGATGGACAGTCACTCT
ATTACAGTTTTATTAATCATGGCAAAGAGAGTGTGGTTCTTGATTTAAAGAATGATCACGAT
AAAAGTATATTTATAAATATGCTCAAACAAGCTGATGTATTAGCTGAGAATTTTCGCCCAGG
TACAATGGAAAAACTGGGGTTTTCATGGGAAACGCTTCAAGAAATCAACCCGCGCCTCATAT
ATGCTTCATCGTCAGGTTTCGGACATACCGGTCCGCTAAAAGATGCTCCTGCCTACGATACC
ATCATTCAGGCAATGAGCGGGATAATGATGGAAACAGGATATCCTGATGCTCCGCCAGTGC
GCGTTGGTACATCTCTTGCGGATCTATGCGGCGGTGTCTATTTATTCAGCGGAATAGTGAGT
GCACTTTATGGCCGCGAAAAGAGCCAGAGAGGGGCGCATGTCGATATAGCGATGTTTGATG
CCACGCTGAGTTTTCTGGAGCATGGTCTGATGGCATATATCGCAACTGGGAAGTCACCACAA
CGTCTGGGAAATCGCCATCCCTACATGGCACCTTTTGATGTTTTCAATACTCAGGATAAGCC
GATTACGATTTGTTGTGGTAATGACAAGCTTTTTTCTGCGTTATGCCAGGCACTGGAGCTTAC
GGAACTGGTTAATGATCCCCGATTTAGCAGCAATATTTTACGCGTACAAAACCAGGCTATTC
TTAAACAATATATTGAGCGGACGTTAAAAACGCAGGCAGCTGAAGTTTGGTTAGCCAGAAT
ACATGAAGTTGGTGTACCCGTCGCGCCGTTATTAAGTGTGGCTGAGGCCATTAAATTGCCAC
AAACTCAGGCGAGAAATATGTTGATTGAAGCCGGGGGAATAATGATGCCGGGTAATCCGAT
AAAAATCAGCGGCTGCGCGGACCCGCATGTTATGCCGGGAGCGGCAACGCTCGACCAGCAT
GGGGAACAAATTCGCCAGGAGTTCTCATCAtaa
yfdE (Acetyl-CoA:oxalate CoA-transferase from E. coli)-SEQ ID NO: 129
MTNNESKGPFEGLLVIDMTHVLNGPFGTQLLCNMGARVIKVEPPGHGDDTRTFGPYVDGQSLY
YSFINHGKESVVLDLKNDHDKSIFINMLKQADVLAENFRPGTMEKLGFSWETLQEINPRLIYASSS
GFGHTGPLKDAPAYDTIIQAMSGIMMETGYPDAPPVRVGTSLADLCGGVYLFSGIVSALYGREKS
QRGAHVDIAMFDATLSFLEHGLMAYIATGKSPQRLGNRHPYMAPFDVFNTQDKPITICCGNDKL
FSALCQALELTELVNDPRESSNILRVQNQAILKQYIERTLKTQAAEVWLARIHEVGVPVAPLLSV
AEAIKLPQTQARNMLIEAGGIMMPGNPIKISGCADPHVMPGAATLDQHGEQIRQEFSS*
yfdW (formyl CoA transferase from E. coli)-SEQ ID NO: 130
SYYHHHHHHLESTSLYKKAGLMSTPLQGIKVLDFTGVQSGPSCTQMLAWFGADVIKIERPGVGD
VTRHQLRDIPDIDALYFTMLNSNKRSIELNTKTAEGKEVMEKLIREADILVENFHPGAIDHMGFT
WEHIQEINPRLIFGSIKGFDECSPYVNVKAYENVAQAAGGAASTTGFWDGPPLVSAAALGDSNT
GMHLLIGLLAALLHREKTGRGQRVTMSMQDAVLNLCRVKLRDQQRLDKLGYLEEYPQYPNGT
FGDAVPRGGNAGGGGQPGWILKCKGWETDPNAYIYFTIQEQNWENTCKAIGKPEWITDPAYST
AHARQPHIFDIFAEIEKYTVTIDKHEAVAYLTQFDIPCAPVLSMKEISLDPSLRQSGSVVEVEQPLR
GKYLTVGCPMKFSAFTPDIKAAPLLGEHTAAVLQELGYSDDEIAAMKQNHAI
yfdU (oxalyl-CoA decarboxylase E. coli)-SEQ ID NO: 131
MSDQLQMTDGMHIIVEALKQNNIDTIYGVVGIPVTDMARHAQAEGIRYIGFRHEQSAGYAAAAS
GFLTQKPGICLTVSAPGFLNGLTALANATVNGFPMIMISGSSDRAIVDLQQGDYEELDQMNAAKP
YAKAAFRVNQPQDLGIALARAIRVSVSGRPGGVYLDLPANVLAATMEKDEALTTIVKVENPSPA
LLPCPKSVTSAISLLAKAERPLIILGKGAAYSQADEQLREFIESAQIPFLPMSMAKGILEDTHPLSA
AAARSFALANADVVMLVGARLNWLLAHGKKGWAADTQFIQLDIEPQEIDSNRPIAVPVVGDIAS
SMQGMLAELKQNTFTTPLVWRDILNIHKQQNAQKMHEKLSTDTQPLNYFNALSAVRDVLRENQ
DIYLVNEGANTLDNARNIIDMYKPRRRLDCGTWGVMGIGMGYAIGASVTSGSPVVAIEGDSAFG
FSGMEIETICRYNLPVTIVIFNNGGIYRGDGVDLSGAGAPSPTDLLHHARYDKLMDAFRGVGYNV
TTTDELRHALTTGIQSRKPTIINVVIDPAAGTESGHITKLNPKQVAGN
Methionine import system permease protein MetP (Bacillus subtilis)-
SEQ ID NO: 132
atgtttgagaagtattttccaaatgttgacttgaccgagttatggaatgccacatatgaaactctgtat
atgacattgatttccttactgtttgccttcgtaatcggcgtcatcctgggattgctgttattcttaaca
tctaaggggtctctttggcaaaataaagcagtaaattccgttatcgcagccgttgtcaacatctttcgt
tcaattcccttccttattttaatcatcctgcttcttggtttcactaaattcttagtgggaacaattttg
ggaccaaatgcggctcttcccgcgttagtcatcggtagtgctcccttttatgctcgtctggtcgaaatc
gcacttcgtgaagtggacaaaggagtgattgaggcggcgaaatcgatgggggctaagacgagcactatt
atttttaaggttcttatccccgagtccatgcccgcgctgatttccggaattacagtgactgcgattgca
ttgatcgggtcaaccgccatcgcaggagctattggttctggtggattgggaaacttagcatacgttgaa
ggctatcaatcgaataatgcggatgtgaccttcgtggccacagttttcatcctgattattgttttcatc
attcagatcattggtgaccttattaccaacatcatcgataaacgc

1) An engineered microorganism comprising one or more gene(s) or gene cassette(s) encoding a therapeutic molecule and a modified pks island as compared to a corresponding microorganism comprising an unmodified pks island.

2) An engineered microorganism comprising a modified pks island, wherein the modified pks island comprises a modified clb sequence selected from a clbA, clbB, clbC, clbD, clbE, clbF, clbG, clbH, clbI, clbJ, clbK, clbL, clbM, clbN, clbO, clbP, clbQ, clbR, or clbS gene or operably linked promoter sequence, as compared to a corresponding microorganism comprising an unmodified pks island.

3) The engineered microorganism of claim 2, wherein the modified pks island comprises a modified clb sequence selected from a clbA, clbB, clbC, clbD, clbE, clbF, clbG, clbH, clbI, clbJ, clbK, clbL, clbM, clbN, clbO, clbP, clbQ, clbR, or clbS gene sequence, as compared to a corresponding microorganism comprising an unmodified pks island.

4) The engineered microorganism of claim 2 or 3, wherein the modified pks island comprises a modified clb sequence selected from a clbA, clbB, clbC, clbD, clbE, clbF, clbG, clbH, clbI, clbJ, clbK, clbL, clbM, clbN, clbO, clbP, clbQ, clbR, or clbS promoter sequence, as compared to a corresponding microorganism comprising an unmodified pks island.

5) The engineered microorganism of any one of claims 2-4, wherein the modified pks island comprises a modified clb sequence selected from a clbA, clbB, clbC, clbD, clbE, clbF, clbG, clbH, clbI, clbJ, clbK, clbL, clbM, clbN, clbO, clbP, clbQ, or clbR gene sequence, as compared to a corresponding microorganism comprising an unmodified pks island.

6) The engineered microorganism of any one of claims 2-4, wherein the modified pks island comprises a modified clb sequence selected from a clbA, clbB, clbC, clbD, clbE, clbF, clbG, clbH, clbI, clbJ, clbK, clbL, clbM, clbN, clbO, clbP, clbQ, or clbR promoter sequence, as compared to a corresponding microorganism comprising an unmodified pks island.

7) The engineered microorganism of any one of claims 1-6, wherein the modified pks island comprises a deletion in a clb sequence as compared to a corresponding microorganism comprising an unmodified pks island.

8) The engineered microorganism of claim 7, wherein the deletion in the clb sequence is a partial deletion as compared to a corresponding microorganism comprising an unmodified pks island.

9) The engineered microorganism of claim 7, wherein the deletion in the clb sequence is a full deletion as compared to a corresponding microorganism comprising an unmodified pks island.

10) The engineered microorganism of any one of claims 1-9, wherein the modified pks island comprises a deletion in a clb gene sequence as compared to a corresponding microorganism comprising an unmodified pks island.

11) The engineered microorganism of any one of claims 1-10, wherein the modified pks island comprises a deletion in a clb promoter sequence as compared to a corresponding microorganism comprising an unmodified pks island.

12) The engineered microorganism of any one of claims 1-11, wherein the modified pks island comprises a full or partial deletion of the clbA, clbB, clbC, clbD, clbE, clbF, clbG, clbH, clbI, clbJ, clbK, clbL, clbM, clbN, clbO, clbP, clbQ, and clbR gene sequences, as compared to a corresponding microorganism comprising an unmodified pks island.

13) The engineered microorganism of any one of claims 1-12, wherein the modified pks island comprises an intact or unmodified clbS gene sequence, as compared to a corresponding microorganism comprising an unmodified pks island.

14) The engineered microorganism of any one of claims 1-13, wherein the modified pks island comprises a full or partial deletion of the promoter operably linked to clbS, as compared to a corresponding microorganism comprising an unmodified pks island.

15) The engineered microorganism of any one of claims 2-14, further comprising a gene or gene cassette for producing a therapeutic molecule.

16) The engineered microorganism of any one of claims 1-15, wherein the therapeutic molecule is a phenylalanine metabolizing enzyme.

17) The engineered microorganism of any one of claims 1-15, wherein the therapeutic molecule is an oxalate metabolic enzyme.

18) The engineered microorganism of any one of claims 1-17, wherein the therapeutic molecule is capable of reducing a deleterious molecule.

19) The engineered microorganism of any one of claims 1-18, wherein the engineered microorganism is an engineered bacterium.

20) The engineered microorganism of any one of claims 1-19, wherein the engineered microorganism is an engineered Escherichia.

21) The engineered microorganism of any one of claims 1-20, wherein the engineered microorganism is an engineered Escherichia coli.

22) The engineered microorganism of any one of claims 1-21, wherein the engineered microorganism is an engineered Escherichia coli Nissle.

23) The engineered microorganism of any one of claims 1-22, wherein the engineered microorganism produces less colibactin compared to a corresponding microorganism comprising an unmodified pks island.

24) The engineered microorganism of any one of claims 1-23, wherein the engineered microorganism produces less colibactin precursor or metabolite compared to a corresponding microorganism comprising an unmodified pks island.

25) The engineered microorganism of any one of claims 1-24, wherein the engineered microorganism produces less N-myristoyl-D-asparagine compared to a corresponding microorganism comprising an unmodified pks island.

26) A pharmaceutical composition comprising the engineered microorganism of any one of claims 1-25.

27) A method of treatment comprising administering the pharmaceutical composition of claim 26 to a subject in need thereof.

28) A method of reducing a deleterious molecule comprising administering the pharmaceutical composition of claim 26 to a subject in need thereof.

29) A method of reducing oxalate comprising administering the pharmaceutical composition of claim 26 to a subject in need thereof, wherein the therapeutic molecule is a oxalate metabolic enzyme.

30) A method of treating HOX comprising administering the pharmaceutical composition of claim 26 to a subject in need thereof, wherein the therapeutic molecule is a oxalate metabolic enzyme.

31) A method of reducing phenylalanine comprising administering the pharmaceutical composition of claim 26 to a subject in need thereof, wherein the therapeutic molecule is a phenylalanine metabolizing enzyme.

32) A method of treating hyperphenylalaninemia comprising administering the pharmaceutical composition of claim 26 to a subject in need thereof, wherein the therapeutic molecule is a phenylalanine metabolizing enzyme.

33) A method of treating PKU comprising administering the pharmaceutical composition of claim 26 to a subject in need thereof, wherein the therapeutic molecule is a phenylalanine metabolizing enzyme.

34) A method of reducing methionine levels comprising administering the pharmaceutical composition of claim 26 to a subject in need thereof, wherein the therapeutic molecule is a methionine metabolizing enzyme.

35) A method of treating homocysteinuria comprising administering the pharmaceutical composition of claim 26 to a subject in need thereof, wherein the therapeutic molecule is a methionine metabolizing enzyme.

36) A method of assessing pks island activity comprising detecting N-myristoyl-D-asparagine using LC-MS/MS.


SEQUENCE LISTING (171 sequences)

SEQ ID NO: 1 - DNA, 1287 bp, Oxalobacter formigenes
atgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 2 - DNA, 1707 bp, Oxalobacter formigenes
atgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataa

SEQ ID NO: 3 - DNA, 1632 bp, Saccharomyces cerevisiae
atgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaa

SEQ ID NO: 4 - DNA, 1146 bp, Escherichia coli
atgacaaataatgaaagcaaagggccgtttgaaggcttattagttatcgatatgacacatgtccttaatggacctttcggaactcaacttctttgtaatatgggcgcaagggtaattaaagttgagccgccgggtcatggtgatgatacccgcacatttggtccctatgtggatggacagtcactctattacagttttattaatcatggcaaagagagtgtggttcttgatttaaagaatgatcacgataaaagtatatttataaatatgctcaaacaagctgatgtattagctgagaattttcgcccaggtacaatggaaaaactggggttttcatgggaaacgcttcaagaaatcaacccgcgcctcatatatgcttcatcgtcaggtttcggacataccggtccgctaaaagatgctcctgcctacgataccatcattcaggcaatgagcgggataatgatggaaacaggatatcctgatgctccgccagtgcgcgttggtacatctcttgcggatctatgcggcggtgtctatttattcagcggaatagtgagtgcactttatggccgcgaaaagagccagagaggggcgcatgtcgatatagcgatgtttgatgccacgctgagttttctggagcatggtctgatggcatatatcgcaactgggaagtcaccacaacgtctgggaaatcgccatccctacatggcaccttttgatgttttcaatactcaggataagccgattacgatttgttgtggtaatgacaagcttttttctgcgttatgccaggcactggagcttacggaactggttaatgatccccgatttagcagcaatattttacgcgtacaaaaccaggctattcttaaacaatatattgagcggacgttaaaaacgcaggcagctgaagtttggttagccagaatacatgaagttggtgtacccgtcgcgccgttattaagtgtggctgaggccattaaattgccacaaactcaggcgagaaatatgttgattgaagccgggggaataatgatgccgggtaatccgataaaaatcagcggctgcgcggacccgcatgttatgccgggagcggcaacgctcgaccagcatggggaacaaattcgccaggagttctcatcataa

SEQ ID NO: 5 - protein, 428 aa, Oxalobacter formigenes
MTKPLDGINVLDFTHVQAGPACTQMMGFLGANVIKIERRGSGDMTRGWLQDKPNVDSLYFTMFNCNKRSIELDMKTPEGKELLEQMIKKADVMVENFGPGALDRMGFTWEYIQELNPRVILASVKGYAEGHANEHLKVYENVAQCSGGAAATTGFWDGPPTVSGAALGDSNSGMHLMIGILAALEMRHKTGRGQKVAVAMQDAVLNLVRIKLRDQQRLERTGILAEYPQAQPNFAFDRDGNPLSFDNITSVPRGGNAGGGGQPGWMLKCKGWETDADSYVYFTIAANMWPQICDMIDKPEWKDDPAYNTFEGRVDKLMDIFSFIETKFADKDKFEVTEWAAQYGIPCGPVMSMKELAHDPSLQKVGTVVEVVDEIRGNHLTVGAPFKFSGFQPEITRAPLLGEHTDEVLKELGLDDAKIKELHAKQVV

SEQ ID NO: 6 - protein, 568 aa, Oxalobacter formigenes
MSNDDNVELTDGFHVLIDALKMNDIDTMYGVVGIPITNLARMWQDDGQRFYSFRHEQHAGYAASIAGYIEGKPGVCLTVSAPGFLNGVTSLAHATTNCFPMILLSGSSEREIVDLQQGDYEEMDQMNVARPHCKASFRINSIKDIPIGIARAVRTAVSGRPGGVYVDLPAKLFGQTISVEEANKLLFKPIDPAPAQIPAEDAIARAADLIKNAKRPVIMLGKGAAYAQCDDEIRALVEETGIPFLPMGMAKGLLPDNHPQSAAATRAFALAQCDVCVLIGARLNWLMQHGKGKTWGDELKKYVQIDIQANEMDSNQPIAAPVVGDIKSAVSLLRKALKGAPKADAEWTGALKAKVDGNKAKLAGKMTAETPSGMMNYSNSLGVVRDFMLANPDISLVNEGANALDNTRMIVDMLKPRKRLDSGTWGVMGIGMGYCVAAAAVTGKPVIAVEGDSAFGFSGMELETICRYNLPVTVIIMNNGGIYKGNEADPQPGVISCTRLTRGRYDMMMEAFGGKGYVANTPAELKAALEEAVASGKPCLINAMIDPDAGVESGRIKSLNVVSKVGKK

SEQ ID NO: 7 - protein, 543 aa, Saccharomyces cerevisiae
MTSAATVTASFNDTFSVSDNVAVIVPETDTQVTYRDLSHMVGHFQTMFTNPNSPLYGAVFRQDTVAISMRNGLEFIVAFLGATMDAKIGAPLNPNYKEKEFNFYLNDLKSKAICVPKGTTKLQSSEILKSASTFGCFIVELAFDATRFRVEYDIYSPEDNYKRVIYRSLNNAKFVNTNPVKFPGFARSSDVALILHTSGTTSTPKTVPLLHLNIVRSTLNIANTYKLTPLDRSYVVMPLFHVHGLIGVLLSTFRTQGSVVVPDGFHPKLFWDQFVKYNCNWFSCVPTISMIMLNMPKPNPFPHIRFIRSCSSALAPATFHKLEKEFNAPVLEAYAMTEASHQMTSNNLPPGKRKPGTVGQPQGVTVVILDDNDNVLPPGKVGEVSIRGENVTLGYANNPKANKENFTKRENYFRTGDQGYFDPEGFLVLTGRIKELINRGGEKISPIELDGIMLSHPKIDEAVAFGVPDDMYGQVVQAAIVLKKGEKMTYEELVNFLKKHLASFKIPTKVYFVDKLPKTATGKIQRRVIAETFAKSSRNKSKL

SEQ ID NO: 8 - protein, 381 aa, Escherichia coli
MTNNESKGPFEGLLVIDMTHVLNGPFGTQLLCNMGARVIKVEPPGHGDDTRTFGPYVDGQSLYYSFINHGKESVVLDLKNDHDKSIFINMLKQADVLAENFRPGTMEKLGFSWETLQEINPRLIYASSSGFGHTGPLKDAPAYDTIIQAMSGIMMETGYPDAPPVRVGTSLADLCGGVYLFSGIVSALYGREKSQRGAHVDIAMFDATLSFLEHGLMAYIATGKSPQRLGNRHPYMAPFDVFNTQDKPITICCGNDKLFSALCQALELTELVNDPRFSSNILRVQNQAILKQYIERTLKTQAAEVWLARIHEVGVPVAPLLSVAEAIKLPQTQARNMLIEAGGIMMPGNPIKISGCADPHVMPGAATLDQHGEQIRQEFSS

SEQ ID NO: 9 - protein, 437 aa, Escherichia coli
SYYHHHHHHLESTSLYKKAGLMSTPLQGIKVLDFTGVQSGPSCTQMLAWFGADVIKIERPGVGDVTRHQLRDIPDIDALYFTMLNSNKRSIELNTKTAEGKEVMEKLIREADILVENFHPGAIDHMGFTWEHIQEINPRLIFGSIKGFDECSPYVNVKAYENVAQAAGGAASTTGFWDGPPLVSAAALGDSNTGMHLLIGLLAALLHREKTGRGQRVTMSMQDAVLNLCRVKLRDQQRLDKLGYLEEYPQYPNGTFGDAVPRGGNAGGGGQPGWILKCKGWETDPNAYIYFTIQEQNWENTCKAIGKPEWITDPAYSTAHARQPHIFDIFAEIEKYTVTIDKHEAVAYLTQFDIPCAPVLSMKEISLDPSLRQSGSVVEVEQPLRGKYLTVGCPMKFSAFTPDIKAAPLLGEHTAAVLQELGYSDDEIAAMKQNHAI

SEQ ID NO: 10 - protein, 564 aa, Escherichia coli
MSDQLQMTDGMHIIVEALKQNNIDTIYGVVGIPVTDMARHAQAEGIRYIGFRHEQSAGYAAAASGFLTQKPGICLTVSAPGFLNGLTALANATVNGFPMIMISGSSDRAIVDLQQGDYEELDQMNAAKPYAKAAFRVNQPQDLGIALARAIRVSVSGRPGGVYLDLPANVLAATMEKDEALTTIVKVENPSPALLPCPKSVTSAISLLAKAERPLIILGKGAAYSQADEQLREFIESAQIPFLPMSMAKGILEDTHPLSAAAARSFALANADVVMLVGARLNWLLAHGKKGWAADTQFIQLDIEPQEIDSNRPIAVPVVGDIASSMQGMLAELKQNTFTTPLVWRDILNIHKQQNAQKMHEKLSTDTQPLNYFNALSAVRDVLRENQDIYLVNEGANTLDNARNIIDMYKPRRRLDCGTWGVMGIGMGYAIGASVTSGSPVVAIEGDSAFGFSGMEIETICRYNLPVTIVIFNNGGIYRGDGVDLSGAGAPSPTDLLHHARYDKLMDAFRGVGYNVTTTDELRHALTTGIQSRKPTIINVVIDPAAGTESGHITKLNPKQVAGN

SEQ ID NO: 11-100 - skipped (90 entries)

SEQ ID NO: 101 - DNA, 1182 bp, Brevibacterium aurantiacum
atgacgagccttcaccccgaaactttaatggtgcacgggggtatgaagggattgacagaggcgggtgttcacgtcccggcgatcgacctgtcgacaacgaatcctgttaatgacgtagcgacagggggtgattcttacgaatggcttgcaacgggtcacacgttgaaggatggtgattctgctgtttaccagcgtttatggcagcccggtgtagctcgtttcgagacggctctggctgggctggagcacgcggaggaggctgtagctttcgccacgggtatggccgccatgaccgcggccttattggcagcagtatcagctggcacgcctcatatcgtcgcagtccgcccattatacggcggttcagaccatcttttagagactggtttactggggacaactgtcacgtgggcgaaagaagctgacatcgccagtgcaatccaagacgatacagggttagtgatcgtggagacgccagccaatcctagtcttgatctggtagacctggattctgtcgtgtcagcggcagggaacgtccctgtccttgtagataatacgttttgtactcctgtccttcagcaacctatctcgcacggcgccgcacttgtattacattcggcaacaaagtatttaggggggcatggggacgccatgggcgggatcatcgcgactaatgctgattgggctatgcgcctgcgtcaggtccgtgcgatcactggtgcattactgcatccgatgggcgcgtacttgttacaccgcggtttgcgtactctggctgttcgcatgcgtgccgctcagacaactgctggtgagcttgctgagcgcttagacgctcatcccgctatttcagttgtgcactacccaggattgaaggggcaagatcctcgtggattgttaggccgtcagatgagtggaggtggggcaatgattgctatggagctggcagggggattcgatgcggcacgctcctttgttgagcattgtaacttggtcgtgcatgccgtatcacttgggggagcagacacattaattcaacatccggcaagtttgacacaccgcccggtcgcagcaaccgccaagcctggtgatggtttgatccgcttgagcgtggggcttgagcacgttgacgacttggccgatgacttgatcgctgctttggacgcctctcgcgctgctgcatga

SEQ ID NO: 102 - DNA, 1197 bp, Citrobacter freundii
atgtctgattgtcgtgcctacggctttaacacacagatcgtccacgccggacaacaaccggacccaagcactggtgcattgtcgacgccaatctttcagacttctactttcgttttcgattcggcagaacagggggccgcacgtttcgcgttggaggaaccgggatacatctatacgcgcttgggcaatccaactactgatgcgcttgaaaagaaacttgcagtgctggaacgtggggacgcggcgttagccacagcgagcggcatctccgctattaccacaagtcttcttactctttgccagcaaggggaccatatcgtatcagctagtgcaatctacggttgcactcacgcattcttaagtcattcgcttcctaaatttggcattaacgtttcattcgtcgacgcagcgaagcccgaagaaatccgcgccgccatgcgtccagagacaaaagtggtatatatcgagacgcccgcgaaccctactttatccttagtggatattgagactgttgctggaatcgcgcaccagcaaggagccttgttagtcgtggacaacacttttatgtctccgtattgtcaacaaccattactgctgggtgctgacatcgtggtccatagtgttaccaagtacattaacgggcatggggatgttattggcggtgtcatcgtaggtaaacaggagttcatcgaccaagcacgctttgtgggattaaaggacattacgggcggatgcatgagcccctttaatgcatggctgacacttcgcggggtaaagacgttagggatccgtatggagcgtcattgcgaaaacgcattgaaaatcgcgcgttttcttgagggccatcctgctattacacgtgtctattatcctggattgccgtcacacccccaatacgaacttgggaagcgccaaatgtcgcttcctgggggaatcatttcgtttgaaattgctggtggccttgaagcaggtcgccgtatgattaacagcgtggagttatgtttgctggcagttagcttaggtgatacggagacgttgattcagcatccggcgagcatgacacattcccctgttgctcccgaggagcgtctgaaggctggcatcactgatggactgatccgtttgtccgtcggccttgaagatccggaagacatcattaacgacctggagcacgccattcgcaaggcaacgttttaa

SEQ ID NO: 103 - DNA, 1200 bp, Porphyromonas gingivalis
atgaagaaagaggacttaatgcgttcggggttcgcaactcgtgctattcatggtggagccatcgaaaatgcttttgggtgcctggccacccccatctaccaaacttcaacgtttgttttcgatacggctgaacagggtggacgtcgctttgcgggtgaagaagatgggtacatttacactcgcttaggaaacccgaactgtacccaggttgaggagaagctggcgatgttagaaggaggggaagcagcggcctccgcaagtagtggtatcggagcgatctcttcagctatctgggtgtgcgtcaaggcgggagatcatattgtagcgggaaaaactttgtacggttgcacgtttgcgttcttgacccacggactttctcgctatggagttgaggtcaccttggtggacacgcgccatccagaagaggttgaggccgctattcgtccaaacactaagttggtctatttggaaactcccgcaaaccctaacatgtaccttactgatatcaaagctgtgtgtgatatcgcgcacaagcatgagggtgttcgcgtcatggtggataatacctactgtacaccttacatctgtcgcccactggagttaggtgctgatattgttgtccattccgccacaaaatatctgaacggacatggagatgttatcgctgggtttgtagtagggaaagaggattacatcaaggaggtcaaattggttggtgttaaggacttgacaggagccaatatgtcacccttcgacgcataccttattagtcgtggtatgaagacgttacaaatccgtatggaacaacattgccgtaacgcacaaacagttgctgagtttctggagaagcatcctgcggttgaggcggtgtacttcccgggcttgccttcattcccgcaatatgagttggccaagaagcagatggcacttccgggagcgatgattgcgtttgaagttaagggaggctgcgaagccggtaagaaacttatgaacaatcttcatctgtgttccttagccgtctccttaggcgataccgagactttaatccaacacccggcctcaatgacgcactcgccctatacgcccgaggaacgcgccgcaagcgacatctcggaggggcttgtacgcctttctgttggattggaaaatgttgaggatattatcgcggacctgaagcatggcctggatagtcttatctaa

SEQ ID NO: 104 - DNA, 1674 bp, Streptomyces sp.
atgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatgcagggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 105 - DNA, 1032 bp, Escherichia coli
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaggaacaccatgtaaaagtagaggtactgggttatgtctga

SEQ ID NO: 106 - DNA, 654 bp, Escherichia coli
atgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaa

SEQ ID NO: 107 - DNA, 816 bp, Escherichia coli
atggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 108 - protein, 393 aa, Brevibacterium aurantiacum
MTSLHPETLMVHGGMKGLTEAGVHVPAIDLSTTNPVNDVATGGDSYEWLATGHTLKDGDSAVYQRLWQPGVARFETALAGLEHAEEAVAFATGMAAMTAALLAAVSAGTPHIVAVRPLYGGSDHLLETGLLGTTVTWAKEADIASAIQDDTGLVIVETPANPSLDLVDLDSVVSAAGNVPVLVDNTFCTPVLQQPISHGAALVLHSATKYLGGHGDAMGGIIATNADWAMRLRQVRAITGALLHPMGAYLLHRGLRTLAVRMRAAQTTAGELAERLDAHPAISVVHYPGLKGQDPRGLLGRQMSGGGAMIAMELAGGFDAARSFVEHCNLVVHAVSLGGADTLIQHPASLTHRPVAATAKPGDGLIRLSVGLEHVDDLADDLIAALDASRAAA

SEQ ID NO: 109 - protein, 398 aa, Citrobacter freundii
MSDCRAYGFNTQIVHAGQQPDPSTGALSTPIFQTSTFVFDSAEQGAARFALEEPGYIYTRLGNPTTDALEKKLAVLERGDAALATASGISAITTSLLTLCQQGDHIVSASAIYGCTHAFLSHSLPKFGINVSFVDAAKPEEIRAAMRPETKVVYIETPANPTLSLVDIETVAGIAHQQGALLVVDNTFMSPYCQQPLLLGADIVVHSVTKYINGHGDVIGGVIVGKQEFIDQARFVGLKDITGGCMSPFNAWLTLRGVKTLGIRMERHCENALKIARFLEGHPAITRVYYPGLPSHPQYELGKRQMSLPGGIISFEIAGGLEAGRRMINSVELCLLAVSLGDTETLIQHPASMTHSPVAPEERLKAGITDGLIRLSVGLEDPEDIINDLEHAIRKATF

SEQ ID NO: 110 - protein, 399 aa, Porphyromonas gingivalis
MKKEDLMRSGFATRAIHGGAIENAFGCLATPIYQTSTFVFDTAEQGGRRFAGEEDGYIYTRLGNPNCTQVEEKLAMLEGGEAAASASSGIGAISSAIWVCVKAGDHIVAGKTLYGCTFAFLTHGLSRYGVEVTLVDTRHPEEVEAAIRPNTKLVYLETPANPNMYLTDIKAVCDIAHKHEGVRVMVDNTYCTPYICRPLELGADIVVHSATKYLNGHGDVIAGFVVGKEDYIKEVKLVGVKDLTGANMSPFDAYLISRGMKTLQIRMEQHCRNAQTVAEFLEKHPAVEAVYFPGLPSFPQYELAKKQMALPGAMIAFEVKGGCEAGKKLMNNLHLCSLAVSLGDTETLIQHPASMTHSPYTPEERAASDISEGLVRLSVGLENVEDIIADLKHGLDSLI

SEQ ID NO: 111 - protein, 557 aa, Streptomyces sp.
MSPTAFPAAETATAPATAVDPGPELDGGDFALPEGGLDDDRRLRALDAVDEYLTRKRKHLVGYQATQDMQGTALDLARFMPNNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGAVTVRFRKPSAELVAKWSLSSQDVLMVPGDETTRRSYVHVFVMPSVDRAKLDALLAELAEDPVILGAP

SEQ ID NO: 112 - protein, 343 aa, Escherichia coli
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEIKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDAPLLSETARRFNVNNNIISAQMDYAGGVKFGIMLTEMHGTQQDTQAAIAWLQEHHVKVEVLGYV

SEQ ID NO: 113 - protein, 217 aa, Escherichia coli
MSEPMMWLLVRGVWETLAMTFVSGFFGFVIGLPVGVLLYVTRPGQIIANAKLYRTISAIVNIFRSIPFIILLVWMIPFTRVIVGTSIGLQAAIVPLTVGAAPFIARMVENALLEIPTGLIEASRAMGATPMQIVRKVLLPEALPGLVNAATITLITLVGYSAMGGAVGAGGLGQIGYQYGYIGYNATVMNTVLVLLVILVYLIQFAGDRIVRAVTRK

SEQ ID NO: 114 - protein, 271 aa, Escherichia coli
MAFKFKTFAAVGALIGSLALVGCGQDEKDPNHIKVGVIVGAEQQVAEVAQKVAKDKYGLDVELVTFNDYVLPNEALSKGDIDANAFQHKPYLDQQLKDRGYKLVAVGNTFVYPIAGYSKKIKSLDELQDGSQVAVPNDPTNLGRSLLLLQKVGLIKLKDGVGLLPTVLDVVENPKNLKIVELEAPQLPRSLDDAQIALAVINTTYASQIGLTPAKDGIFVEDKESPYVNLIVTREDNKDAENVKKFVQAYQSDEVYEAANKVFNGGAVKGW

SEQ ID NO: 115 - DNA, 1257 bp, Unknown
  source: /note=“Description of Unknown: yjeH sequence”
atgagtggactcaaacaagaactggggctggcccagggcatcggcctactatcgacgtcattattaggcactggcgtgtttgccgttcctgcgttagctgcgctagtagcaggcaataacagcctgtgggcgtggcccgttttgattatcttagtgttcccgattgcgattgtgtttgcgattctgggtcgccactatcccagcgcaggcggcgtcgcacacttcgtcggtatggcgtttggttcgcggcttgagcgagtcaccggctggttgtttttatcggtcattcccgtgggtttgcctgccgcgctacaaattgctgccggattcggccaggcaatgtttggctggcatagcgggcaactgttgttggcagaactcggtacgctggcgctggtgtggtatatcggtactcgaggtgccagttccagtgctaatctacaaacagttattgccgggcttatcgtcgcactgattgtcgctatctggtgggcgggcgatatcaaacctgcgaatatccccttccctgcgccaggaaatatcgaacttaccgggttattcgctgcgttatcagtgatgttctggtgttttgtcggtctggaagcatttgcccatcttgcctcggaatttaaaaatccagagcgtgattttcctcgtgctttgatgattggcctgctgctggcaggattagtctattggggctgtacggtagtcgtcttacacttcgacgcctatggtgaacaaatggcggcggcagcatcgcttcccaaaattgtagtgcagttattcggtgtaggagcgttatggattgcctgcgtaattggctatctggcctgctttgccagtctcaacatttatatacagagcttcgcccgcctggtctggtcgcaggcgcaacataatcctgaccattacctggcacgcctctcttctcgccatattccgaataatgccctcaatgcggtgctcggctgctgcgtggtgagcacgttggtgattcatgctttagagatcaatctggacgctcttattatttatgccaatggcatctttattatgatttatctgttatgcatgctggcaggctgtaaattattgcaaggacgttatcgactactggcagtggttggcgggctattatgcgttctgttactggcaatggtcggctggaaaagtctctacgcgctgatcatgctggcggggttatggctgtttctgccaaaacgaaaaacgccggaaaatggcataaccacataa

SEQ ID NO: 116 - DNA, 1197 bp, Citrobacter freundii
atgtctgactgtcgtgcttacggattcaatacccagatcgttcatgcgggccaacaacccgacccttccactggcgcgctcagtacgcctattttccagacgtcaaccttcgtttttgacagtgccgaacagggcgccgcccgctttgcgcttgaagaacccggctacatctatacgcgccttggaaaccccaccaccgacgcgctggagaaaaagctggctgtgctggaaagaggcgatgccgcgctggcaactgcatccggtatttcagccatcaccacctcgttgctgaccctttgccagcagggcgaccatatcgtttccgccagtgccatttacggctgcactcacgcttttctgtcacacagcctgccgaagttcggcattaacgtcagcttcgtcgacgccgccaagccggaagaaatccgcgcggccatgcgcccggaaaccaaagtggtgtatatcgaaacaccagcaaacccaacgctctcgctggttgatattgaaacggtcgccgggatcgcccatcagcaaggcgcattgctggtcgtggataacacctttatgtcaccctactgccagcaacctctgctgttaggtgccgacatcgtggtgcacagcgtgaccaagtacatcaacggccacggggacgtgattggcggtgtgattgttggcaagcaggaatttatcgaccaggcacgattcgtcgggctgaaagatatcaccggcggctgcatgagtccgttcaacgcctggctgacgctgcgcggcgtgaaaacgctgggcatccgaatggagcgccattgtgaaaacgcgttaaaaattgcccgcttcctggaagggcatccggccattacccgcgtgtattaccctggcttgccttcacatccgcagtatgagctgggtaaacgtcagatgagcctgccgggaggaattatcagcttcgaaatcgccggcggcctcgaggctggcagacggatgatcaattctgtagaattgtgcctcctcgcggtcagtctcggtgacaccgaaaccctcattcagcacccagcgtctatgacacattcgcccgttgcgccagaggaacggcttaaagcaggtattaccgacggacttatccgtctttctgttggtcttgaagatccagaagatattattaacgaccttgaacacgccatcagaaaggcaacattctga

SEQ ID NO: 117 - DNA, 1200 bp, Porphyromonas gingivalis
atgaaaaaagaagaccttatgcgtagtggctttgccacacgtgccatccatggaggcgctatcgagaacgccttcggctgcttagccactcccatctaccaaacatcgactttcgtttttgacactgccgaacagggaggccgccgctttgccggagaggaagacggatacatctatacccgtctgggcaaccccaactgcacccaagtggaagagaaactggccatgctcgagggcggagaagccgccgcatcggcctcatccggtatcggagccatcagctctgccatctgggtatgcgtgaaggccggcgaccatatcgtagccggcaagacgctctacggctgcaccttcgccttcctcacccacggactgagccgctacggtgtggaagtcaccctcgtggatacccgccatccggaagaggtggaggctgccattcgcccgaatacgaagctcgtctatctggagactccggccaaccccaatatgtacctgaccgacatcaaggcagtctgcgacatcgctcataagcacgaaggcgtacgcgtcatggtggacaatacctactgcacgccctatatctgccgtccgctggagctgggtgccgacatcgtggtacacagcgcgaccaagtacctgaacggacatggcgacgtcatcgccggattcgtcgttggtaaagaggactacatcaaggaggtgaagctcgtcggcgtcaaggacctcacgggggccaatatgagtccgttcgatgcttatctgatcagccgcggcatgaagacgctgcagatacgtatggagcagcactgccgcaatgctcagaccgtagccgaattcctcgaaaagcatccggccgtagaagcagtttatttccccggacttccgagcttcccccaatacgaattggccaagaagcagatggcactgcccggagccatgatcgccttcgaagtgaagggcggttgcgaagccggtaagaagctgatgaacaacctgcacctctgctccctcgccgtgagcttgggcgatacggaaaccctcatccagcatccggccagcatgacccactcgccctacacacccgaagagcgtgctgccagcgacatatccgaaggactggtacgcctgtccgtgggtctggagaacgtggaggacatcatcgccgacctcaaacacggtctggacagcctgatctaa

SEQ ID NO: 118 - DNA, 1182 bp, Brevibacterium aurantiacum
atgacctcactgcacccagaaacgctcatggtccacggcggaatgaaaggcctcaccgaggcaggagtccacgtaccggccatcgacctctcgaccaccaacccagtcaacgatgtcgccaccggcggtgactcctacgaatggctcgccaccggccatacgctcaaggacggcgactcggccgtctaccagcgcctctggcagcccggtgtcgcacgcttcgagaccgcgctggccgggctcgaacacgctgaggaagcagtcgccttcgccacgggcatggccgcaatgactgccgcacttctcgcggccgtcagcgcaggaacaccccacatcgtcgcagtgcgtcccctctatggcggaagcgaccacctcctcgaaaccggactgctggggacaacagtcacatgggcaaaggaagccgacatcgcctcggcgatccaagatgacaccggactcgtcattgtcgagaccccggcaaaccccagcctggaccttgttgatctcgacagtgtcgtctcagccgccggcaacgtgcctgtgctggtggacaacacattctgcacacctgttctccagcagcccatctcccacggagcggccctcgtactgcacagcgcgacaaaatacctcggcggtcatggcgatgccatgggcggcatcatcgccaccaacgccgactgggcgatgcgcctgcgacaggtccgagccatcacaggagccctgctccaccccatgggcgcgtatctccttcatcggggcttgcgcactctggccgtgcgcatgcgcgcggctcagaccaccgccggtgagctcgctgagcgcctggacgcgcaccctgccatctccgtcgtccactacccgggactgaaaggccaggacccacgcggactgctcggacgccaaatgtccggtggtggtgcgatgatcgcgatggagctcgccggtggattcgacgccgcccgcagcttcgtcgaacactgcaacctcgtcgtccacgcggtgtccctgggcggcgctgacactctcatccagcatccggcgtcactgactcacaggccagttgcggccacggcgaagcccggcgatggtctcatccgactctctgtgggactcgaacacgtcgatgacctggcagacgatctcatcgctgccctcgacgcgagtcgggccgctgcctga

SEQ ID NO: 119 - DNA, 1674 bp, Streptomyces sp.
atgagcccgaccgccttccccgccgccgagaccgcgaccgcgcccgcgaccgccgtcgatcccggtccggagctggacggcggtgacttcgccctccccgagggcggcctggacgacgaccggcggctgcgcgcgctcgacgccgtggacgagtacctgacccgcaagcgcaagcacctggtcggctaccaggccacccaggacatgcagggcaccgcactggacctcgcccggttcatgccgaacaacatcaacaacctcggcgacccgttccagagcggcggatacaagcccaacaccaaggtcgtcgagcgggccgtgctcgactactacgcgaagctctggcacgccgagcgcccgcacgacccggccgacccggagtcgtactggggctacatgctgtccatgggctcgaccgagggcaacatgtacgccctctggaacgccagggactacctgagcggcaaggcgctgatccagccgccgaccgcccccttcgacgcggtgcgctacgtcaaggccgaccccgaccgacggaacccgaacgcccaccacccggtggccttctactccgaggacacccactactccttcgccaaggccgtggccgtcctcggcgtggagaccttccacgccgtcggcctggagaagtacgccgacgagtgcccgctggtcgacccggtgaccgggctgcgcacctggcccaccgaggtgccctcccgcccgggtccgtccggcctgtcctgggacggccccggcgagatagacgtcgacgccctcgccgtactcgtcgagttcttcgccgccaagggtcacccggtcttcgtcaacctcaacctcggcagcaccttcaagggcgcccacgacgacgtccgcgccgtctgcgagcgcttgctgccgatcttcgagcggcacgggctcgtccagcgcgaggtggtctacggcagctgcccgcagaccggccggccgctggtggacgtgcgccgcggcttctggatccacgtggacggcgcgctcggcgccggctacgcgccgttcctgcggctggccgccgaggacccggaagggtacggctggacgcccgaggcggagctgcccgagttcgacttcggcctgcggctgcccaccgccgggcacggcgaggtggacatggtctcctcgatcgcgatgagcggccacaagtgggccggcgcgccgtggccgtgcggcatctacatgaccaaggtgaagtaccagatctcgccgccgtcccagccggactacatcggcgccccggacaccaccttcgccggctcccgcaacggcttctccccgctgatcctctgggaccacctgtcccggtactcctaccgggaccaggtggagcggatccgcgaggcccaggagctggccgcctacctggagcggcggctgaccgccatggagcgcgaactcggcgtcgagctctggccggcccgtaccccgggcgccgtcaccgtacggttccgcaagccgagcgccgagctggtggccaagtggtcgctgtcctcccaggacgtgctgatggtcccgggcgacgagaccacccggcgcagctacgtgcacgtcttcgtgatgccctcggtcgaccgggccaagctggacgcgctgctcgccgaactcgccgaggacccggtgatcctgggcgcaccgtag

SEQ ID NO: 120 - DNA, 1257 bp, Oxalobacter formigenes
atgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 121 - protein, 418 aa, Oxalobacter formigenes
MNNPQTGQSTGLLGNRWFYLVLAVLLMCMISGVQYSWTLYANPVKDNLGVSLAAVQTAFTLSQVIQAGSQPGGGYFVDKFGPRIPLMFGGAMVLAGWTFMGMVDSVPALYALYTLAGAGVGIVYGIAMNTANRWFPDKRGLASGFTAAGYGLGVLPFLPLISSVLKVEGVGAAFMYTGLIMGILIILIAFVIRFPGQQGAKKQIVVTDKDFNSGEMLRTPQFWVLWTAFFSVNFGGLLLVANSVPYGRSLGLAAGVLTIGVSIQNLFNGGCRPFWGFVSDKIGRYKTMSVVFGINAVVLALFPTIAALGDVAFIAMLAIAFFTWGGSYALFPSTNSDIFGTAYSARNYGFFWAAKATASIFGGGLGAAIATNFGWNTAFLITAITSFIAFALATFVIPRMGRPVKKMVKLSPEEKAVH

SEQ ID NO: 122 - DNA, 1287 bp, Oxalobacter formigenes; identical to SEQ ID NO: 1

SEQ ID NO: 123 - protein, 428 aa, Oxalobacter formigenes; identical to SEQ ID NO: 5

SEQ ID NO: 124 - DNA, 1707 bp, Oxalobacter formigenes; identical to SEQ ID NO: 2

SEQ ID NO: 125 - protein, 568 aa, Oxalobacter formigenes; identical to SEQ ID NO: 6

SEQ ID NO: 126 - DNA, 1632 bp, Saccharomyces cerevisiae; identical to SEQ ID NO: 3

SEQ ID NO: 127 - protein, 543 aa, Saccharomyces cerevisiae; identical to SEQ ID NO: 7

SEQ ID NO: 128 - DNA, 1146 bp, Escherichia coli; identical to SEQ ID NO: 4

SEQ ID NO: 129 - protein, 381 aa, Escherichia coli; identical to SEQ ID NO: 8

SEQ ID NO: 130 - protein, 437 aa, Escherichia coli; identical to SEQ ID NO: 9

SEQ ID NO: 131 - protein, 564 aa, Escherichia coli; identical to SEQ ID NO: 10

SEQ ID NO: 132 - DNA, 666 bp, Bacillus subtilis
atgtttgagaagtattttccaaatgttgacttgaccgagttatggaatgccacatatgaaactctgtatatgacattgatttccttactgtttgccttcgtaatcggcgtcatcctgggattgctgttattcttaacatctaaggggtctctttggcaaaataaagcagtaaattccgttatcgcagccgttgtcaacatctttcgttcaattcccttccttattttaatcatcctgcttcttggtttcactaaattcttagtgggaacaattttgggaccaaatgcggctcttcccgcgttagtcatcggtagtgctcccttttatgctcgtctggtcgaaatcgcacttcgtgaagtggacaaaggagtgattgaggcggcgaaatcgatgggggctaagacgagcactattatttttaaggttcttatccccgagtccatgcccgcgctgatttccggaattacagtgactgcgattgcattgatcgggtcaaccgccatcgcaggagctattggttctggtggattgggaaacttagcatacgttgaaggctatcaatcgaataatgcggatgtgaccttcgtggccacagttttcatcctgattattgttttcatcattcagatcattggtgaccttattaccaacatcatcgataaacgc

SEQ ID NO: 133 - DNA, 723 bp, Artificial Sequence
  source: /note=“Description of Artificial Sequence: Synthetic polynucleotide”
atgagtaatatcagtttgtattgtttgccatattcaggtggttctgccgccatgtattataaatggcgtagcgtgctgtcggacaatattactttgcggcctttagaacctgcggggaggggaactagaatacgccagccgctgtgtcttacgatggtggatgccgtcgctgacctttatcaacaatttgtgaaacactacacaggtggagactacgccatttttgggcatagtctcggagggatcatggccttcgaactggtgcattatattctcgatcatggacatgacatgccatgcgcgctgtttttttccggctgtcgcccacccgatcgggcctctcatgaagtaatactgcataccttgcccgatcaggcgtttatggaagagatcgtcaagctgggcggaactccggttgatgtctttcgtaataaagagttaatgacaattttcacccccatcattaaaaacgattatcggctctatgagcagtatgtatttcaggccaaggcgcgcacattaacctgtccgatcgtgctatttcatggcgatgctgacaatctggtaatgcaggatgaattacttgcatgggaaaaattcaccacacgaaagacgcggactattatatttcctgctgccgatcatttttttgtcgataagcattttgaacaggtggtaggttatgtgaaccagacgattgaatcactcgaaatagtagggtag

SEQ ID NO: 134 - DNA, 1515 bp, Artificial Sequence
  source: /note=“Description of Artificial Sequence: Synthetic polynucleotide”
atgacaataatggaacacgttagcattaaaacattatatcatctcctgtgctgtatgctgctctttatttccgctatgtgcgctttggcgcaagaacatgagcctatcggggcgcaagatgagcgcctgtcgacattaattcaccaacggatgcaggaggccaaggtcccagccctttccgtaagtgtgaccattaagggggtacgtcagcgatttgtctacggtgttgccgatgtggctagtcagaaagcgaatactctagacacagtttacgagctgggatcgatgagtaaggcgtttaccggacttgtggtgcaaatactgattcaggaaggcagactccggcaaggggatgatatcattacctatctgccggaaatgcgcttgaattatcagggaaaacctgcttccctgaccgtggctgatttcctttatcatacatcaggattgcctttttcaacactggctcggctggaaaaccctatgcctgggagcgctgtggcacagcaactgcgcaacgagaatctgctgtttgcgccgggtgcgaagtttagctatgcctccgccaattatgatgtgttgggcgcggtgattgaaaatgtgacgggaaaaacctttacagaggtcattgcggaacgactcacgcagccgctgggcatgtcggcgactgtggcagttaagggggatgagattattgtcaacaaggcaagcggctataaactgggattcggcaaacccgttctgtttcatgcgcctctggcccggaaccatgttcctgccgcctatatccatagcactctgcctgatatggaaatatggatagacgcctggttgcacagaaaggctttgccggcaacgctgcgtgaggcgatgagtaacagttggcgtggtaatagtgatgttccgcttgccgcagacaatcgtatcctctatgccagcggttggtttatcgaccagaatcaaggcccttacatcagtcacggtgggcagaatccaaacttttcttcttgcattgcgttgcgaccggatcagcagattggcattgttgcgctggcaaatatgaattcgaatctgatactacagctttgcgcggatatcgataattatctgcgcattggcaaatatgctgacggcgctggtgatgcaattacagccaccgatacccttttcgtctacctcacgttgttgctgtgtttttggggggcggtggttgtagtgcgcggtgctttccgtgtttatcgcgcaacggcgcatggccctggaaaacagcagaggttacgtttacgcgtacgtgactatatcatcgccttggcggttcctgggctcgtggccgccatgctctatgtcgcaccgggtatactatctccaggacttgactggcgttttatcttggtatggggtccatcgagcgtgttggcgataccgttcggaattatcctgttagctttcgttctgacattaaatcatcaaattaaacgaattctattacacaacaaggagtgggacgatgagtaa

SEQ ID NO: 135 - DNA, 2460 bp, Artificial Sequence
  source: /note=“Description of Artificial Sequence: Synthetic polynucleotide”
atggcaaaggatgattttacctgtggctcactggatattgccattattggcatgagcgggcgtttttccggtgcggaatcggtgcccgagtggtgggataagttgctggcgggtgaggagttcactcaaccgacatgcacagaagacgataatgggaacccttggattaggctgcgcaatataatcaccgctccctatgacttcgatgctgcatttttcaatattccgcctggcgaagccctgttgatggatccgcaacaacgaatatttcttgaatgctgctacaacgctcttgagcatgcgggctatattcccacgcaacttaagcgagtcggcgtctatggggcgacctacgccaataactattttatcgatcgcgtctatccatacctgaagatgagcggcgatcaccattatcttcaggcgcagattggtaacgaaaaggattacttgtgtgctcaggttgcctacaagttggggtttaccggacccgctgtgagtgtacagaccgcctgttccagttcgttggtggcagcatatttagcatgcgaggggttattgacttttcaagccgatgttgcgttggcaggtggggtcaccttgggttttttacaggcgcacggctacagtccgcaaggtgataagctggtatcgcaagacggacactgcgccccgtttagcgctgaggcgaccggcacggtatacagcagcggtgcgggagttgtggtgctaaaacgtcttgaggatgcactacgcgaccaagacagagtgtatgcggtgattaaaggaggtgcggtaaataacgacggcgggcgtcggctggggtttgtcgcccccagcgtagaaggacaggtagaagcgatcaatacggcgcttgccgccgcagaagtcgtaccgactgatatcgcactgatcgaaacgcacggtaccggtaccccgttgggtgatgagattgagcttgaagctctgcaccgggttttcgcaccggcatgtgcgccgcacagcattcaattgggtgcggtaaaagccaacttgggtcacctgggagtcgcttccggtatcgtcagcttaatgaaaaccgcactgacgctatatactggtttagttcccccgcagatcaacctagtaaataaacataaaaaactgttgcagccagcctcgccattttatctcagtgatgtagtgacttctgtcccacaaacgaaaaggatccatgccaccgttagttcgtttgggctgggagggaccaatgctcatttggttctgcaaaactggtgcgagacgcctgcgcaagcggtgcaagaaaatgagcgccgcctgtttttcttcagcgccaaaacgccattagcccttagacaacagttggatgctcattatcacgctctggcgacctatgcggaagcagataaagatcgtatcgcctatactctcgcgcaacgccgggcacatttcccgtatcgctgtgcgctggcggcagacagtgtggtagcgttgcgtgcaagtttggcgaaactgcgggatgctgacatgtcttttacccctatcaatatggaaactaccttggtgttcctatatccggaccgggacgataagctggagagtgccctgacgcatttgctggcttgtcaacctaacttgcgccagcggcaccagcgcctgtctcaggatgttgcacaaatctgcgaaccagccgattggacgccagcattgcgtcagtttatccagcaggtgagtcttagtgaatggctgatcgaacaaagtatctcgcctgtgcagcacatcggttacctgacgggggctgcagcagcgcagtatgttgcgcgcatcatctcactggaaaacgctgttcagcaggtgattgtggctgaaacgacaccggagcagacgctggccggcaacagtgaactgagcgaaatattggctaatctggcggtgacggagggaacgctgatgctggaaatcggtcgcgcggggacgttttccatattgtatcaccagcatgcgcagtgggttgggcaaacagtattttcccccatgctgaatacggatacgcctgaggacatcctgccgctgttgggcactctttggcaacgaggagtgactatttgtttaccggaaatgccagccgtgcagactataggcttacccggttattcgtttgatcgcgtccggtatgaaattcagtccagtgatgcgagagagaacgctatgttaccagtgagttatttgtcggtcagcgactttgtggagaaaacctggcgttcattgttatgcatcgatcattatgacgaacacgcggttattttcgaatacggagcgacctcgatgcacgttatctctttcgtcgacagctgtaaccacatttataaaatcggactgactgccgccgatatttatgccagacccgctatccgtgagcacagtgaatttatctccgaatgtgttgatggtattttatga

SEQ ID NO: 136 - DNA, 4368 bp, Artificial Sequence
  source: /note=“Description of Artificial Sequence: Synthetic polynucleotide”
atgatgtcgggcaatccgttgtcgtggccacaggaacagtgccacatcattgatcaactgtatccttacagtgccgtaaacatcattggtggtgtggtcaccattgagggaattgtcgatctgccccgactacacgcggccatccagtccgccattcggcaatttgatgcgttgcgtatgtggtttgtgatgggagaggagagtgaagtggtcagccaagtgcagccttatcactggcgcgatatccgccatttgactttttccccagattatgacaaggagaacctacgcccagcggcgatcgaaacatttgttgatgagtggttccggcagccctttaccctgctggcgcacgatttgttcgagtttgtcacctttacgtgcggtgaacaatacagcggttatttatttaaagcccatcacggcattgctgatggttggtcaatggcgttgttgtcaaaccatgtcaagcgagcatacgaacagcaggacgtaccagatgatgcgtcaccagcctatagcgcatttctggcgcagcagcaatcttatcaggcgtcaacgcgctttgccgttgatcgtggctggtggcgtgactatatcgacgaataccgcgactgctttccagacagttcccccatcgttactactgagggcatcagttgttccacttggcttgagccagccatgattaaccgtctctacaggttgtgtaatcgttatggctgtacgcttaatacgctgtttattgcactcttcgctctctatcgcgccagagtatggggggaggaaaaaggcgtaataggcgtgccgttagccaatcggcatactcgcgaggcgcggcgttgctttggcatgtttactaatcaattgccgctggcctatcgactggtgcgcacggaacgattctgtgaacgggtggcctttttccagcgcgaactgaaacgcggttttaaacacagcaaatatcccattaccttattcaaccaggatttggcggaacaggggggagggaagttgcgggcgttcgattattgcgttaattattataactttacctatgaacgccacattgcaggtgccgcgcaacgcgtggagagttactacagcggcgagcagtcctataagctacagattgttttgcaaactgttaataatcataaagaaagccttaggttgagtctggaggcgctgcgtagcgctttcactccgcaccaactgacagcgatgaaaaatgggctgttggatttagtcactgcattggatcggcaacctgacgctcgtctaggcgatctcgaagtctatcctgcgccgcacgtcgcattggcatgtggctcactcaaaccctcatttacttcccgatttgcagcgcaagttgtggagcatggtgatcgcacagcactaattgataatgaacagtctttgacttatcggcagttggatgacgctgttgagcgggttgcgcgttatctgcgccaacaggggatcggacgcggtcaagtggttgggattatcgctgagcatagcgctcaaacggtgatggttatttatggtattttgcgctgcggcgcggcatttctgccgctaaatcccgcgttgccaacgacccgcctttacgccatgtgtcgtaaagcgcaggtagcgcatatcctttacgatccggcgatgcatgaattgacgcaggcgctggcgtttcccgcgtccagcttgctccaggccttagccacttcggcgcttgctagagaaccttggccagctattgaaccgcaggatctggcatatgtgctgtttacctccggatcgaccggcgagcctaagggcgtgcaagtctctcatggcaatctggctaactatctccatttcgccgccgaacgttactttacagcgcaggaccgggctgcgctctactcttcgctgtccttcgatttgaccatcactacgctgttcgcccccttatgtgtgggcgctagcatcagcgtttgtcggcacgccgagagcgaaacattgctgcgcatggccgtggttgatcagcccaacacagtgatcaaactgacgcctgcacacctgcggttgttgtgtgcggctggcataagcagtgagcagattcgtaccttggtggttggcggggaggatttcaagcgggatctggcgcgtaaggcggctgctctctttcctcaggccgtgatctataacgaatatgggccgacagaggccaccgttggttgcatgatctatcgctacaccgggcaagaaacgctgccttcattgcccatcggtatggcgatcgacggttgccaggtagcaatttgctccccttggggctgtccggtaccggaaggcgaaactggtgaattggtgatttatggcgcgtcagtgacgcagggctatatcgatgcgccgcaacaaacggccgccgcctaccttaaagataccaatggggtgatgattggctatcgcagcggtgatattggctacgccatcgctcctaatacgctggtttatcagggacgtaaagacgatcaggtcaaaattaatggctatcgtattgagctgtgcgaaatcgaacaagcgttgttgagcgcacctcaggtagaaagtgcggcggtggcggtgattgatgatgtgcaggggcagcacagcgggttgctagcctgcgtgacaccgtcatctgttgatgtagctaccgtaatgcaacatctgcgtcagcaattacccacctacatgcaacccaagcagtgctgtgctatcgcccaactgccgctatcgcacaatggcaaggtggacgtgcgtcagatggtcgcaacggtccgaaacaccgcgcctgcatcgggtagcgagcggctgggagatgcggcgataaggcattcagttcgggtatgtgtggaaggggcgctggagcaaactgagtttgatgataacgaaaatctttatgtcttggggttggactccatcaagagtatccagattgcagcgcagctcaggcaccacggctggacgatgtcagcggtgcaggtgatggagtgtggaacagtgaacgccatctgtgagtttctcgctagtcatacaacggtgtcacagctcgcgcagtatgctcataacacccgtatcgatctacccgcattgcgttggtttacgcagttggcgttgccagtgcccaatgtttataaccatgtcattgtgctgaaagtgttgccgggctgcccgctcgagcagttacacaacaggctgcatacattaatccagcagcagccagctttgcacagtgcgctggacgctgaggggcggttgctagtatgcgaccctaatgtgtgttatcccaacgaggtgctcacggaatattcgacggcgcagtggacgttggcagaggtgatcgctcagtgcaatagcatgcttgacgtgacaaatggtcgagtctttacggcagctctgttacacgccccgcagcctgcgtctagcacgctggtactctgtgcccatcacttgtgcgtcgatatgcactcttggtatttaatcctcagcacgctggacgccgtcagcactgtcaatggcaccagcaacagcggactgcatcgctggaatgactatctggcgagcaaaacggtagattccgccacacacgaaagctggcgcacagtatgtcaaacgctaccgctgcatttcccacccgtgtcattgccagatgactcattacctcgtacgcgcgcttggcgtgaagactttcgccatccttgcgtcaggaggttgtttgaatccagtggtaataccgcgtacagcgccgaaacctatgtgctaacggcattggcgctggtgctgcgttactacagcgaagagccttggtgtcgcatcgagatggaaggcatgggacgcggctgctggcccgatgagcctgacgttgcggatacggtcgggtggtttactctattttatccttgggcgatccctctgcatggcgatatggcgacactgttatctgcgatcgcttccgatttggcaaaacgcacgcatggtgggggagattacggtctgctgcaaatgcggcatgcaccggaagactctcttgcgcaagggatccgaatgaattacatcggtgtgcaggcgcaaccttcactccgctattttcatattgatcactttaatagtgatatctataccgcgcctgaaaacgcgctcggctgcgtgctggagtttaatattgcgcgctcggcagctgatggtctctccttccactgccgtttcgatcccacgcgcattgcgctgaatgatgtgcaactcctgctggcgcgctacaagaacagcctcactgatctggatgcctggctgtgccaacacagcgccacgctgacgggcgcgccgactttgtggacactataa

SEQ ID NO: 137 - DNA, 1440 bp, Artificial Sequence
  source: /note=“Description of Artificial Sequence: Synthetic polynucleotide”
atgacggtgaataagcttgagccagacaacggcacgccgaacgatgagctgttcacggtggcgggtatgtttgacggctcgctgtataaattactcctgcgcatggcgttgccgatgtttgtcggtatgttaacgcaggtgacctatgccatcgccgatattttttggctgagccacattgatgtcaccaacagcggcatcattgccggtgtggggctggttttccccgttggaatggggttatttgccatcgccaacggcattcaaatcggtatgggatctttgctgtcccgcgccatcggcatgcagcggttggatcgtgcacagcggattttgtcggtcggtatcatcatcgcgctattctttgcgatcgtgatcaccgtacttggctatgtttatgcccagccgctattgcgctcactgggggccacgaagagcatcatcggctacgcaacggagttctattattattcgctgctgaccgtgttcagcatcatgttgattggcgtcatgatggggctgtttcaaggcgcgggtaagatcatggtcatcatgaaggcctcattgttgggggcgttggtaaatattatgctcgatccaatcatgatctttgtattcgattttggtgtgaaaggggtggcattggcgtcgtttctggcgcaactgagtatggtcgcctatttcatctacacattgatgggattacatatcgggttgagtatacgcatcgcgttacgcccattctcctggaaaatctaccgagagtttctctccgtcggcatggcgcagatgctgatgcaactgatcattgcggtcggcatagtgatttataatttttttatcgtcaggctggacgtaaacgcgatggcagcgtttacgctcactggtcgcatcgactatttcatcatcacgccgatgctggctatcgcgaccgcgttgctgactgtggtggggcaaaactggggacacggaaatgtcacccgcacgctgaatgcctattgggccgccgtcgcgctggccttctctattgtgctggtgttagccgttatgcatattgtactggcaccctggatgtatcccctgtttactcgtgttgttgccgtgagtgactatgcggtactgcaaacccgtatcatggcactggcactaccctttgtcgctatcagcctgttagcaagtgaatattatcaggccattggtaagccttggtattcggtgttgttgacgctgatgcgtcacgtgtttatatctgtccctgtcgtatacctgctggcgattgtgttagagatgcgtattaccggcgtctattttggggcgatgagcggcacttttgtggctgcgttgttggcgtggcgtctgctgcgtctttcacctcgtctgctgcgatggaatcaggaggcggtgcgctcccaacacttggatatggaggtggcaccatga

SEQ ID NO: 138 - DNA, 1464 bp, Artificial Sequence
  source: /note=“Description of Artificial Sequence: Synthetic polynucleotide”
atgagtgagcagagctatcgtagcgcagggacactgttggcacagttggcgtccggagaaaccacctcagtggcgttggtgaatcactatttttcacgtatggcgcagtttaacaagccgttgaatgcggtggtgcagcagcactatgcgttggcgttggaggccgccgcgcgcgccgatcgtgagcgactggaggggcgcgcaaggggcgtgttgcacggattaccttgtactgttaaggagtcgtttgacgtgcaagggtggttaaccacgtccggtgcccattatctgaaagataaccgggcgacgcaggatgctccttccatcgcacgcttacgcgctgccggggcgatcctgatgggaaaaaccaatgtgccgatgatgactgccgattggcaaacctataacgatctgtatggcaccacccataacctgtgggataggcaacgttcacccggcggatcgtccggaggagcggcggtggcggtggcggcggatttcacccccgtggaatttggcagcgatttgtttggctcactgcgcattcccgcacactacacaggtgtctatgcccatcgttgtagcttggggttgatgtcggtcagaggacatgtgcccggtggtggtccgcaagcgactgatgagccggatctctcgacggcgggccccatggcgcgcagcgcggcggatctgcgtctgatgatgcgcgcattgagcacattctgggtggaaccgccgcgtattcccgattttagccgttatcaggccaaagcaaactaccgcgtgtgcacgtggtttagtgcgccccaccatgagatagatcaacagatcgcacagcgttttcaatcgtttatcgacaagttacgcgcacagcctggcgtggaggttgatgatgctatgcccgccgatatcgatcccgacgcactgtttgatatagcagtcaaactcagtggtcggctggtaagtacggcgctcaatggacgccagcgtcttacagccgggctggctgcgctgggcttcaggcttgtgggcaagctggcggatgtgcctgaggggataacgagctattaccagggcatgctgaaagacagtggcgaacagcggaatacagacaaactccgccacgaatacagccgggtgatagaaacccttttcgcgcgttatgacgtgttgttgacacctgtcagcccggtgctggcgtttgcgcacatgcagcagccggtgcgtaagcgcaagcttatcgtcaatggcgaaccgcaagattacaacgaacatttgttctggaacatgttagcaacggtttttggtttgcctgccaccgtttacccattggcgaaaacgatggatgagcttccgtgcggcatacagatcatttccgggcattttcacgatgatgtgacgattaactttgctgagttctgcgaaagcatcagtggcggattcacggtaccggaagggtactag

SEQ ID NO: 139 - DNA, 6465 bp, Artificial Sequence
  source: /note=“Description of Artificial Sequence: Synthetic polynucleotide”
atgacttacagtgaaagcgatattgccattgttggcatgaactgccggtaccccggcgtgcacagcgttgcggcattcgaaacggtgcttcgcaccggatgcaacattttggaccccaaagtgaccccgagcaacggccacaatcacattacgctgaacaacgtctatgagcacatggcggagttcgatgccaacttttttggctacagccgtgcagaagcagaaatcatggatccacagcagcgcgtatttctgacctgtgcttgggagatgtttgaacaaagcggttacaacccgaagcagcacgatgcgcgcgtcggcctatatgcgggcgtgagtaccagcttctatttgcttactcacctgatgaacaacccagacaagcttgcacaactcggtggtctccaaatcatggtcggcaatgacaaagatcatttgacgtcccaactggcttaccgcctgaatattaccggcccttgcgtgacggttcaggccagttgtgccacctcgctggtggcagtgcatctggcctgcgaagggctgctgagcggtcagtgcgatatggcgctagcgggtggcgtcacgtttcgcatggaggagcagcgcagctatgaatcgcacggtgatgggctgcaagcagaggatggcctgattcatacttttgatgcgcaggcgagcggtacggtctacagcagtggtttgggcatggtgctgctcaaacgggctacagacgcgcaggtgcaaggggataacatccttgccgtcatcaagggaagcgccatcaacaatgatggcggtgcgcgcagtggttataccgtgccgggtgttgatggtcaggaagctgtgatgattgaggcgcatagcttggcggaggtgacgccgcagcaaattcagtatctagaactgcatggcagcggcacgccgttgggtgacgctatcgaattcgcggccatcaaacgtgtgtttgggacgccagcgccgaatgcgacaccgtggcgtctgggggctgtgaaacctaacgtgggtcacgtagaaatggcatctggcatcaccagtctcataaaaacggtgctgagtcttactaaccgggtgttttaccctacgctgaattttcaacgggctaatccgcaactggggttggaggacagcccgtttgaagtggtgtcccgtttaacgccttggccggagggcaccacgccacgcaccgctggtgtgagtgcgttcggattgggcggcaccaacgcacatctggtggtacaagcgccgttatcgacgccgcaggcgagggcgcagcagatggggccttgtgtcgttgtactctcggcaaaaaaccacaatgccctggaacagatgcaaaacgccctgctggcgaaactggccgcacatccagagatacgcttacaggacgtggcttacaccctgcgtcacgggcgtttctccgccccggtacgtaagtgtgtcatcgcagagaattgcacccagctagcccgacaactccgcgacgcaccgatggtggaggcaaccacgggttgcacgatctactggagattggggcaccgtttcgttgttgcactcgagacgcttagtgactggctggcgtgctctgaggtgttgtcacaggcggtgggacaactgctcgagcattttccgctcgaaccagcctgtctgcaagatctctcccctgcgcaacggacgtttatcagccagtatgcgctgatcgcgttgattgacgaacgggaaacgttgaatgtggtgctatgcggcgacggtgatggtggctatgctgccgccgtactacgaggagactgcacactagagcaagcatggcacaggttgaatgcgggccaaccgtttgatgatgtgccgacgaatcccctcttgcagcccgatgtctgctctttgatgctggacgacgcggcgagtgatgccaatcgcaccgcgctagaggcgttagggcagttatggttggctggcgtcagcctagattggcgctgggtggatgcggcggagcgtatgttgggcagtcaacgcatcgccctgccggggaccgtttttacaccgcagcgctattgggtcgaggccgtacggcctgccacgttctcccacgaatcgtcgaataatctcttatcccgcgcaacaaaatccgatattattgcagtggtgacggagatatgggaacgcacgcttggtgtcagcattgatgatcaccacgccagcttcttcgaactgggtgggcattcgctgttagcgtcaaccattttatatgatattcagcaacgttacggcatcacctgtacgctaagcgcattctttgccgatcccaccatcgagggattaagttgctatctgctcgaacaaggcggcagtgagacagcggtttcagcattgcctgatacggtctttgcccctgaccagcagcacctaccttttccgctgactgacgtgcaacaggcctattgggttgggcggcgaaaatccttgggattgggcaatatttccacccatatctatgtggaatatgaactacaggggctagatgaaacagcgttcaaccgcgcgctcaatgcggtgattgcgcggcacagcatgttgcgtgcaattgtcaacgatgacggtatgcagcagatattgccaaacgtgccggaataccacgttgccttctataccacccagtgcgaggatgcgtttcaacagcgttgccgtgagctgcgtgacaccctctcacatcagatgattgattgtagtcgctggcctctgtttcagatggaggtcgtggttgatccgcagcaaaaagcccggctacatgtatctatcgacctgctgatagccgatgcatggagtctggagttgttcattcgggaactagcttatcactaccgccatccgcaggcggcattgcctacattgacgtacagtttccgcgactatgtgctgaccttaaaatcctacgagaaaacgccgcagtttgaacgggcacgtgactactggcgcgcccgcatcgaaactctgccgccggggccacgattgccgctgcgtaccgaccccacgaagctggaaaaccccacattcgtgcgccgtagctattgtctatctcgtgctatctggcagcgcttgaaaacgcaggcgggtcagatgagtatcacgcctactacactgctgctcacggggtttgctcaggtgttggcgcgctttagttcctcgccgcactttagccttaacctgacgctgtttaatcgtctgccgttgcacgcagatatcaatcacttaattggcgattttactgcactgacgttgctggagattgatatgtcgcagggagaaaccttgcaggcgcgggcaaacgtgatccactctcaactgtggcgcgatttggataaccgcttgtttggcggtatccaggtctctcgactgctggtacaaactcaccgcgatccggcgaaatcggtgattcctatcgtgttcaccagcttgctcaatcaatatgaggcgagctgggaaacggatgatacgctgttcaaccaaccgcaggatgatctgtacagtatttcacagacaccgcaggtgtggctcgatcatcaggtaatggagcgtaacggggaattgcactttaattgggacgtggtggaacaattgtttgaaccggcgctaatggatcagatgttccagtgttattgccaactcctgcacgccctagcccagcgaccacagctctggcatgagactcaagatgtgctggcgctgcccaccgttagtgcaccggtcacgcaggctcctgcacctacggccttgttgcaccatgggttactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacactggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaaatcgagcgctgcattgcgcgacatcccgatgtggagcagtcagtggtggtggcagtgggtaattctcaacatcgtcggctggtcgcttttgccaaactgcacgatcgccaccaggcgcaggcattgcaagctaaggaagcggaggcggcggcactggcgcagggtattattgtgaatccggcacagcgtctagcgttcaaactcaaggagccacatattcgcgcgctggatggtctgggcattgcactgacggcaccggcggatagcacacgttacatcaaacgccgcagctatcgtcatttcagcgcgcaaaaaaccacgctggcacagttggggcaattgctgtcgggcttggggcagatgcgtctacccggtctaccttttgccaagtatgcctatgcgtccgccggggggctatacccggtgcaaacctacgtgtacctgcatccagacaagatcgaagagggagtatccggtatttactacttcgacccgcgacagagctgtcttatgccggtagcaccagaagtcgagctgaacagtggttttcatgccggacctaatcagtctattgccgatcgggcggcattcacgctgtttatggtggctgatatggcggtgatctcgccattctatgggcaagaggcagcttggcacttctcggtgatggaagcaggtactctctgccatttactggaagaagatgcgccgcgctacggattggggctgtgccaacttgggatggcagacttttccgctgtggcatcgcattttcaattgtcgccacatcatcgctatgtccattgcaccgtggggggcgcgatagggcaagaggcggcaagtgctgcagcattgctgcgcgatttctccacctatgagaaaccgaaggaaaccgctgcgccgctggacatgcagagctacaaagatgccatgctgcgcggcctgcgtcagcaactgcctgactatatggtgccgagtgatctgatgttagcgaccgattttccgttaaccgctaacggcaagctagatcggcaaaaattacagctgcagggcgaacaaattgcccaccagcgtgacggcgtgggtccaatccaggtggacagtgcgttacaacagcggctggtggcgctctggcaggaggtactcggcgtgagccacgtgtcggccgaagacgatttcttctcgctggggggcagttctatagaattggtgcgtattcagcaggcacttgaggcgattatcgggcaggagattcccattgtcgatctgttccgtctgccaaccatcgcggatgtggcgcgttaccttgatgagcaactgcacaatctacccgccgcccacgatatcgtattagcgcaggctgaggtttcgcaggtcagcgctgcgcgcgagaatctggcgttgcggcgtaaacgtgcccaacagggagaaaagggcgatgagtga

SEQ ID NO: 140 - DNA, 6501 bp, Artificial Sequence
  source: /note=“Description of Artificial Sequence: Synthetic polynucleotide”
atgacgatacatcatgccgcattggcgcgaatgttaccggcggaaaaaaaggaaaagctattacgacagttggcgcaatcaggcgtgtcgcccagccgcattcccatcatcaaagcggatccggcgcaggcaatcccgctgtcgtttaatcaggagcggttgtggtttttgcaaaaatacgacagtaccgccactaactacaacctttatgtggtgtatcgtttgcatggcgtggtggatatgccgatgctcacggaggcattgcgccatgtgcaagcacgccacgccatcttgcgtacccgtatcatagtacgtaacgatcggccttgccaggtgatagatgatgcgtcttcgttagttctcgatacggtcacgctggcagcgcaggccccaacttcagcgctagatgcggtaattcagcaggtgataaacacccggttcgatctggcacgcggtcctctgtggggggtaactcagattattcaacccgatcaaggttgtcatttggtgttttgcgcgcaccatatcatcatcgacggtatctcgttacggctattgttcgatgagttacagcagcagtatgctcgattgcatgctggtaatgaaacgtcgctgcctccgccgccgctgcaatatgccgattatgccttctggcagcgcgaatggttccaggataccttgctggcgaacgaacttgcctactggcgtgcgcgtttgcaagacgcgcctctgctgtcgacatttccatcgctgcacccgcgcccggcacaaccctctacacatggttcccggttcagcatcaccctggatgaaacattaagcctcgcgttgaaacacgtagcccgcacgcaggaaacaacgccgttcgtgctgatgctgactgccttccaactggtgctgatgcgttacgctcagcaacagcgattggtgataggcatgccagtatcggggcgcattcgcccggaattgcagagtagcattggttattatgccagcactgcggttatctacaccgattttaatggggttgaggtagggcgcgaggcgctccagcgtgtgaaggccagcgtgaaagagacgcaggggcgccagcaactgccgtttgaaaacttggtgaatatgctggatctccctcgcagcctttcccattcaccgttgttccagatcctctatatctaccataaccacgtgaccccacgcgcttttaccttggctggtgcgtattgggagcaggtgacgtatcacaatcagaccgtcaaatacgacatgacagtcgaggtgttccaaaacgacgccacgttcgacgtctcctttgagtacgatttggggttatacgatgctgatgtggtgaagcagattgccgaggcgctgcgtcagcactgcttatcgttgacatcatcactggagaccccgataggggcgatccctctgcatgcaccggagaccgcaacgccgcggcgtgatccgctcaacgccactaacgtcccgtggctcgggccgcaggatgtgctgcgtatcattgaacagcgctgtgtgcagcacccaaagcaactggcaatacagcagcatgacggcacactgacctacgctgagctctgggcgcgtgtgcagttcatcgcgatgcgttttcgagcgcatggcatacaaccgggcgatcgtattggcgtgctgttgccacgtcacagggatgtgattgcaaccatgttggcgacgtggtttgtgggggcgtgctacgtgccgttcgatattcatcagcctgccgcgcgtttgcaacgccttatgcaacgcgcgcgtttggtctgtctggtggtccgtcagccgggagagtggggtgaaattgtacaactgtcgttgccggaattgatgcaggacatgtcgaatgcgatccggtattctacaccttgcgcgctgttgccggatatgcaggcctacctgttgtttacctccggcagtaccggtgaacctaaaggcgtgtgcgttgttcaccgcgggttgctgaacttgttgttggatatgcagcgtacctttgcggttggctcgcaagaccggctgctctcggtgacgacgccaacatttgatatctcattcttggagtatctgttgccgctgatctccggtgcgagtctctatctgacagaggcggaacgcgccgcagacagcttccgtatgattccgctgattgccgactatcgaccaacgctgatgcaggcgacgccctcgttctggcacgggctgttgatggcgggttggcgtggcgacccggaactatgtgtgctggcaggcggtgaagcgctgccaacgaaagtggcggaagaactgttgcgctgttgtggttcattgtggaacctctacggtcccaccgaaacaaccatttggtcgctgaaatcgcagataacccaagcggaaaacatcaccctcggcgctcccattgccaatacccgtatatacattctggacaatgagggccatccagtgccgcaaggcgttgacggcgagctttacatcgccggggatggtgtggcgcaagggtatgatgggcagcctgagttgaacgcacagttcttcttgtcagaaccaggggtccccggtggcaggatgttccgcactggcgatctggtcaggagtgatgcgcaggggcagttgttttttgttgggcgcaaagatagccagatcaaactgcgcggttatcgtattgagttaggcgaaattgaacggacgttggcacggcatccgcatgtggacgccgcggtggtggcctgtattgaaagagcaccgttacacaaagcactggcagcattcatcatcaccagtgagcctccctcgctattcgagcaactgaaaaacgagcttcggcagcaactgccagactatatggtgccaacgctgtggcagagggtggccgacttcccgaacactgacaacggtaaaatcgatcgtaagcggttagcggaaaatttcgttgccgatagctcccttgtgtcgccgcagacgcaagcgctgagcgacacggaacagatgttgctggcgctgtggatgcgctatttgccgataaaaaacgtcgatcctgagtgcgatttctttcgcttggggggacattcgctgttggcggtaacgttggtagcagagatcaaccgcacttttcactgcgccttgaccctgaaggacattttccactactccacactacgggcactgagtgcgcgtattgcacagcaatctatcacggacgctgccgcgtctcaggatgactgggtgatagtgcacgaccctgagcatcgtcatcaaccgttcccattgacggatgttcagcgcgcctactggcttggacgacagacgggtgctacctcgatcgcgacccacatctaccatgaatttgacgtagaacactttaatgttacgcgttttacccatgcggtgaatgcgctgatcgctcgccatgaaatgctacgtgcgcgggtactccccgacggtactcagcagattctggcgcaagtgccggcgtatcagttagagcagcgcgatctgagtgctttgtcccctaacgcacgaaacgatgccttgatggcgatccgcgatcggctgtcgcatcatgtgcatcccgcagatcgttggccgctgtttgatttcagttattcggcttgcacggcgcaacatggccgcttgcatttcagtctcgatctgctgattgccgatgctctgagtatgcgcacgctacagcaggagttgatgatgctgtaccgtgagccccatgtgtcactgccgttgctaccgttctcttttcgtgactacgtgcaggcgctgttggtagagcaggcgagtgaagcctatgcacgcgatcaggcctattggcaacgggcgctgccgcagctgtatggcccaccaacgctgcccgtacagggcgatttggcgcaactgtctgcgatcaggttcgtacgtcgccgtcatcggctgtcagcccacaactggggagtgctgagcgcgctggcccaacgcacacgtatcaccaagacggcattgttgttgacagtctttagccaagtgctggcacgttggagccttagcccgacgtttacgctcaatctgacgttgttcaaccgcccgcagggttaccccaacgcagaggcagttattggtgattttaccgctgtcagcttgctgaatgtttgttacgacagccagcactcttatgcccacaacgctcagcgtattcaggtgcaactgtgggaagatctcgaacatcgtcgtttcagtgggatccgcgccagcgaggcgctgatccatagcggtcgtttccatgcgccgatgccggtggtattcactagtatgttggatatcgacggggagacgactgcgcaagaccctcgggacacaacccgttttactctgtgtccggacgccaatattacccaaacaccgcaggtgtggctcgatcaccaggtgatcgagttggctggggagttgcatttcaactgggacgcggtcgagcaactgtttgataccacgctgctggatcagatgtttggtgcttattgtcatgcgctgcaggcgctggttgccatgccgcaaagttggtggggggtaaatagttctctggcgctgcccaccgttagtgcaccggtcacgcaggctcctgcacctacggccttgttgcaccatggattactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacgctggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaagtcaaaagccaccttgaacagctcgacagcgtagggagtgccgccgtggtgtgccaccagggacagctgtatgcctttatcactgccgcagaaaacctgcatcctgacgatactgacgccctgttagcgcgtgttcgtgctcagttagccgtgcagttgccgtattacctgctgccccaacatttctttcttctcaaggtgctgccgatgacaggcaacggcaaaattgatcaggcggcaatggttcaagaggttatccaacgtatgtcgcaatctacatcacagaagtcgagggccctcgcgcacgcctcgccctatgaacagcaggtggccgctctctggtgcgaggtactacaacgagaacagatcggactgaatgacaacttttttgaagcggggggcggctcaatccagatcgtgctgttgcatcgccgtattgaggagatttttaaggttacggtacctattgctgagctatttcgcttaaccaccgtgaaaagaattgccggttatttgcaggctatgcaggacaatgcacgggcggtgaatcaaacacagcagcgtgatgcttcccgatcccgcgcccagcaacgacttgtacgtcgtcaccagcgtcagcgttaa

SEQ ID NO: 141 - DNA, 3033 bp, Artificial Sequence
  source: /note=“Description of Artificial Sequence: Synthetic polynucleotide”
atggcagagaatgattttggtatagctatcattgggatggcggggcgtttccctcaagccgatacggtacaggcgttttgggaaaatcttctggcaagccgagagtgcatttccttttacagcgatgaggaactgctggcgatgggaatcagtcctgaatttgttcagcacccagattacgtcaaggccaaaggggaggtagctgacatcgataaatttgatgctgctttcttcggtatcgcaccccgagaggcggagctgatggatcctcagcaccgtgtcctgttggaaaccgcgtgggcagccttcgaagatgcgggctatgtggctgccgattatccgggggatgtgggtatttttgctggcaaaagcatggattcctatttgatgctgaacttgatgccgcactttaaacgcgttttctcttctggcagtttgcaggcggccattggtaacgacaaagacagtatcactactaccatcgcctatcacctcaacctgcgcggcccggcgatcacggtgcagacttcctcatcgacatccttggtggcggtgtgcgtggcatgccaaagtctgttgacttggcagtgtgacatggcgattgctggcggagtaacgctggggccaccagcaaaaaccggctacttgtcgcaggaaggtgggatcaccgccgccgatggccactgtcgggcgttttccgataacagcagcggatttgtgcccggcaccggcgcggggctggtggtgttaaagcgtgttgatgaagcgctgcgtgatggcgataacatctatgcggtcatcaaaggatttgccgtcaacaacgacggttcggagaagatcagttataccgcgccgagcgtggatgcccaagcgcgcgccattgctcaggcacagcggttggcaggtttgacaccgcaggatatcacttatgtggaagcccacggtaccggtacgcgtttgggcgatccggttgagttttctgcgctaagccaagcctttgcgggcgcgtcacaaaagcagtactgtgcgctggggtcggtaaaaaccaatatcggccatttggatacggcagcgggcgtcgcagggttgataaaaaccgcgctggcagtgcagcagggaataattcccgcaacattgcactttgaacggcccaatgcacagatcgatctgaccaatagtccgttttacatcaacactacctgtcaaccgtggcaaccggaaagcggcatccgtcgcgcgggcgtcacctcgcttggcatgggcggcaccaatgcccatgttgtgctggaacaagcgccagccgttgacttgcaagcgcgagcgcctgtacctgcctacagtattttgccgttttctgccaagaccgacagtgccttatcctcggggctggcgcgttttgccgattttctgcaacatgaatcgctgccggataggcgggatctggcgtggacactctcacagggacgtaaagccttcgcacatcgcgccgcgctcgtaaccagagatctacatgctgccgggacgctgttgcagcaggccgcgacagcgccgtttgctcgtggtgtagcgcagacacagctcgggctggggctgttgttttccgggcagggtagccaatatcagcgcatgggccatcagctctatcaggtttggccggcctacgccgatgccttcgaccgttgcgcgacgttactcgagcgtgaataccaactggatatccgccatgagttgttcagagcagaagtctcgttagcccaaggcgaacgccttgcgcaaacctgcctcacacaaccgctgctgttcagcgtcgagtatgccttagcccaattgtggctcagttggggaatcacgccaacggtaatgattggtcattcgctgggcgaatgggtggcagcgacgttggcaggcgtgttctctttagaggatgcgttgcgcttggtggcgcgtcgagcagagctgatgcatcaggcaccaagcggtgccatgttgatggtggcgctgcccgaagcacagattcgcgcactgattaccgccccgctggcgattgccgctgttaacgcccctgactattcggttatcgccgggcctacgtcggagatcctcgccgtcagccagcgtctgacggagcagaacatcatcaacaaacgattgcatacctctcatgccttccactctagcatgatgcaagatgcggcgcaggcgctacgtcaggcatttgagaatgtacgactgaacccgccgacgctcaccatcatctctaccgtaaccggcgcgcacgtcagtgccgacactctcaccacaccggactactggattgaacagatgctgatgcctgtgcagttctccgctgcactgcaagaggcgcaagctacatttgatgtcgattttctggaaatcgggccaggtgccaccctgacccagctgaccaacggtcatgcattaggtgatcgtctcgcgttcagctcgctgccagcaggtgcccgcagcagcgatgagcacaaacatatcttagataccgtggcagcgctttgggtgcgagggcataacatcgatttgtctgcgtttgcaggtgagcaaccgcgccgtgtctctctaccgacctacgcctttgacaaaatccgttactgggtcgacagtccagaagaacagaggagcgccgtaacgccggtggcggacgcgggaagtgtcatccctagcgaaccgtcggtgcgccgtcagccgcgtccggcgttttcggtgccttacgcggctccagagagcaaaacgcagcgcggcttagtggcgatttgtgaagcattgctgggaattgacggcttgggcattgacgacaacttcttcgaagctggcggacattcattgatgctgggcatgttgctggcgcaggtacaggaacggttcgccgtcactctttcctttttcgacgtgatggaggatgccagcgtgcgcgcgttagcgcagttggtcgagcaggagcagcaggatgatggagggtccgcacttgccgtgctagttaacgacatgattaatgagtga

SEQ ID NO: 142 - DNA, 4797 bp, Artificial Sequence
  source: /note=“Description of Artificial Sequence: Synthetic polynucleotide”
atggaacagcaagggattatgagacagttgcctaccgacgaccaaacgatagtcgactatttgtatcgtatcgccggagaatatggggaaaaagccgctgtattgatgggggacgcggcgctgagctatcacgatcttaatgcacgctctaaccaactggcgcattatctgcgtgggctggggatcggcgaggatcgtgtggtagctatccgcctgccgcgcggcatggcaatgctgattgccattttcgctattgtaaaagctggtggtgcctatttaccgctggcgtacaatgcaccgcgcagccgcattgaaaatatactgagcaacagcggcgctgtttgtctgatcggtactgacgatggtgatcgctggccgattcctcgcgtcgaaatcgacagcgcggcggtcagtgccatgccaacgacggatctgcgttaccgaccacatgcgcggcaactggcgtatattatttacacctccggttccaccggtgtccctaaaggggtggcgacggaacatgcagcgctgctaaaccgaattgtctggatgcagaacgcttatcctatcagttctcaggacgtgctgtttcagaagacggtgtacacctttgacgtctctgtctgggagatgttctggtgggcgatgtacggcgcatctgtagtgctgttaccgtccggactggagagcgatccgcgaaccttggctcgactgattcagcgtcaccgcgtgtcggttgtgcactttgtcccttcgatgctgaacctgtttgtcgagtatctggagatgaaacaggatcctcgtttgaccgcctcattgcgattagtgttttccagcggtgagaaactcacggtccacagtgtggctcgcttttatcaatcggtggcgcagggtgatcttataaatctctatggcccgaccgaagcagcaatcgatgtcagtcatcaccgctgcctgcgcgggtacgactacgacgatatccctatcggtcaagcgattgacggttgccgactctatgtgctggatgaccatggtaatccggtagcagacggcgaagagggcgaactgtatctcgctggtatcgggctggcacgtggctatctcaacaacgtggcgttaactgatcgctgttttactatacatccaactttgcgccatttagggaaaccggagcggctgtataaaactggagatctggtgtggcgcgacggggaaagccaacaaattcattacattggccgtaatgattttcagattaaaattagagggttgcgcgttgaattgggagaaatcgaagcccatgcgatgcgtttcccgggggtacagcaggcagtcgtggtggcggatcaggatgatcccgacaatcagttgatttacgcttttgtcgtcagtagcgtgccgctcaatttggcggccttaatggacgcactgtccaaaaacttgcctgcctacatgctgccgaaccgtttgttggcaatgtcagagttaccactctccgacaatggcaagtgctgtcgtaaaacgttgctcgacttggcgcgggcgtactcagccagtcgcgtcgatttacgtgaaactcccgccgtgcgctacctaccgttgtcatcggctcaatcgtcgatgtggtttatgcaacaattggcgccgcatactgcactatacaataaccccaccgccttgctgcttgaaggagaactggatcgcacgcggatggacggcgcgattcgtcaattgatgagtcggcatactctgttacgtgctatggcggaaacccacaatggacaaccagtattggcggtgcctcagtgcgtatcgtcgcaggcgttgctgaccatagtgccactcccctcggtgagtgatgataacgcgctacaggcgatgatcaaccagcgtgcggcacaccccatgcccttaacgtcaggcacaccgctgtgccggtttgaactgttgacgctcgatgacgatcgtagcgtattgttgattcatctgcaccacatcatcagtgatggctggtcgaaaggcgttttgttacgcgaattgcaggccgcatataacggtgaatcgctgacgccagaaccgttgttggagtatgccgactacatggagtaccaagaagagtggcgtcagagtgacgcctatcaggacgcgatgcgttactggcaaaataccctggcggggacgttgccgattcttgatatccccaccgatcaaccgcgtcagaaggtggcacgttaccaaggcgcgtttgttgccttcgcattgtctgccaacacatgcgagcgagtgttggcggcggcgcgtgcgcagcgcgtgtcgttgtataactatctgctgaccgccttcgtcctcctgctgcatcgtaatgcgcgtcaacaggaatacatcgtcggtatgccgattgctgcgcggctgacgaaagaacaggagcatatgatcgcgccgctggtcaacgtactaccgctgcgcttacctcttgacgaagcggcatcgttttccgagttagtacagacgatcagaggtattctgtttgccgctttcaggcaccagcgtctcgaatttaccgacatagtgcgcgcagtgaatgtggatcgcagcgccggacatttcccaatatatcagtgcatgttccaactcgacaatatgcctctggcgagcccgacactaaacggtgtcaacgttacaccattattgttggataccagtgcatctcaggtggatatttcgctaagtatgcagcatatcgatgggcgcatcaccggcactttcgaatacgacgctgggttatacagtgcggatcgtattcagcacctggtggcgcagtggagagaactgctagatgaggccagcagccaacccacgcagttagtccgcgatttgattcgtttcacccctcgagagcacgcttggctggcgcggcacaacgccactgaagttgctctgccgcccgtagataacctgctggcgctagtgttaccgcactgccagcagcgccctacacaggtggctttgcgccacgctgacgacgccatgacctatggcgaattgcaacaggccacgatgcagatgtgtacttggctgcgtgcgcaaggcgtaaaacgcggagaatctgtcgcactgcaactgcctttttgtttcgaattgattatcgcacaattggcgatcctctctttgggggcgagctatgtgccgctggatggtaacgcgccagcggcccgcaatgccctgatcttggcgcaggcaacgccgtgcatgctgctggtggcgcaaccgctggaatcccctcatgggctgacgataccttgggtgctagtgcctgactggcgtagcctgctgacagaaataccgaacctgccagttagtgttgcgccagatgctttagattgcgatgcggtggtgatctttacctccggtactaccgggcagcccaaaggcgtccgactcagccaacgtaatctggtcaacttaaccgcatcctttatatccagctatcaagtgacgcatcaggatgttttgctacccattacctcagtcgcgtctgctagctttgtcggggaggtactgccgctgctcgccgctggcggtaccttagtattggcgcaaaaagcgcagagtttggatagtgatgcgctcattgcgctgctggcatctcagcgggtgactatcctgagcaccaccccatcgctttctgccagcctctctgtgctggctcagtcgatgggatcgctgcgcttgtttctgtgcggtggtgaagcgttagagtatgagcagatagcgccgcttctgccgcacatggcagtggttaacggttatggcctgaccgaaagcggtatctgctcgacctactttcctgtcgcaaagcgtagggagcaagaaacgggagcattgcccattggccgcccgattcagaacacccaagcttatgtggtggatgcatataatcgtttggtaccgccaggtgcctgtggagaactctgtttctctggtctgggcatttcgcccggctaccttgatgcacgacaggatcccgagcgctttgtcgagttgccggaataccctggcgttcgggtgctgaaaactggcgatcgtgctcgttgggcaaccgacgggatgttgttctatctcggcagacaagatcgccaagtgcagatccgtggataccgagttgaactgggcgacatcgaaagcctactgaaacagcatccggatattgctgatgcgtgggtcgatgtgcgacgcaatgcggcggcaacgccgctactagtggccttctattgcagcgtcaacggcgttgcgctggatgctcagcaattgcgcgtatggctcagcctgcggcttccgttgcatatgctgccgttgctctacgtgccgctgagcgccatgccgctaggtgtaaacggcaaaatcgatccccagtgcctgccgctggtcgatctgcggcagttggaagggccgggcgagtatgtccctccggcaaccgaactggaacagcgtctggcggagatctggcagcagttgcttggcctggagcgtgtcggcaccaccacaaatttcttcgatctcggtggacactcacttctgctagtacagatgcagcaatacatcgggcagcagtgcggtcagcacgtggcgttggttgacctgctgcgcttcactaccatcaaacgcttggcggaatttctgctggccccggacgcagcgcaagggaccacaggagatcagacacaactgcgtgcggcgaagcagcgtttggcctttggtcacacgcgctgggcagccacaaccgacagtcatcactga

SEQ ID NO: 143 - DNA, 1269 bp, Artificial Sequence
  source: /note=“Description of Artificial Sequence: Synthetic polynucleotide”
atgacgaaggatgtcgcactgatgttccctggctccggttcgcaatatgtaggcatggcacggtggctgtatgagcgttatccgcaggtgcgtactctgtttgatgaagctagccagatcacggaacgggacatggcagcgctgtgcctgtcgggtactttggtacaacttgctgagccgacggcaatggcgctggctatctataccaccagcgtggcccactttgtcgcgtggcagcagtttttggcgcaaaccggtgtccatgtcaacctacgttatatgttgggtcatagtttgggcgaatatgctgcgctgacctgtagtggtgcgctgagcttttcccaagcactggcgctggtggcgatgcgtagccgtttagccagtgagatcgcccgcgaaatggacgcgagtaccactatcatcaagcaagggaatcaggcactggtggcagcggcgtgcgaggtggcggagcgtgaaacccgacagcaggtcggtattgcctgcttcaattcgccgcaacagtttatgttatccggacaaaactcggccattatcgccgccgagcaatacctgctggatcatgatcgccaagtcgaagtagtgccgctgattggtggtgttccttaccacagcccactattgaaaccttgcggacagcaattgcgcaaggcgctggatcgctgcgaatggcgacgtccgtgctgcccagtgatctccaatgtcaacgctcaaccctatcccgatacggtcacgccagtccagtggctagaacaacagctctctcagccagtgcagtggcagcgctctctcacctacctgacaggacacttgtcaccgatcgcgatagagatcggtccgcaaagtgtgctgaaaaacctgctgctggagaatcgctatccagcaccagtttacgcatttgacaatcgtcatgatcgtgcgcaactagcattggtgctgggggataacatggcggtgaagaccgatccggaagccgtgcgccgccaacgcatcacgctgctcactaacgccctgacagccacacgccatcaccgcgccgccgatgtggctgccagtgcgcaattgaaagaattgctcagtcgtttttttgagcgcatccagcagattgagcagcgcggcacatccagcgaagaggacattgcttttttgcatgagcttcttgaacagggatttcaactcaaaggcagtagtcaggcagaaatcgacgcctgccacgcgcggttggcttccgacaacggcggacaggcgtaa

SEQ ID NO: 144 - DNA, 1131 bp, Artificial Sequence
  source: /note=“Description of Artificial Sequence: Synthetic polynucleotide”
atgtgcactgaaaattatgagctggctcagcaagaggcagtcctgtttgccaaacaacatctcgccctggctgcacagaatattgaacgtcagcagtttattgtgccagacattatttcttgcgtggcgcaggccgggtacttaggtgcgtcaatcccccaaaaatatggcggacgaggttacgattcttatcaactgtgcgctttgcatgaagtgatggctggggttcacggttcgttagaaaatctcataacagtgactggcatggtcagcacgctgctgcaacgcgtgggcagcgcagcgcaaaaagcccattatttgccaaagctggcaacgggggaattgatcggcgcgattgccctcacagagccgaatatcggcagtgacttagtgaacgtggaaacagaattacagcaggatggcgacggttggcggctgaacgggaaaaagaaatggatcacgctagggcagattgctgatttttttattgttttaatccactgtggtaatcaattagcgacagtgcttatcgatcgcaataccgacggctttactatcactccactcaacgacatgttggggttacgcggcaatatgttggcagagctgcattttaatgactgccgcctgaaggaggatgcattgctcggtccgctcacccctggggtaccgctggcggtgaattttgcgctcaatgaagggcggttcaccaccgcttgtggcagcctaggattatgtcgggccgctgtggatgtggcagcgcgctacatccgccagcgtaaacagttcaagcggcggttattttctcatggcattgttcagcacttgtttgccacaatgctgacccaaacgcgcagtgcgcaactaatgtgcttcagcgcggcggaataccgcgaaacgctgcacccggccatgataaaccaaatcctgatggcgaagtatgtcgcttccaaagctgcggtggacgtggccgggaaggcggtgcaactgctcggtgcgaatggttgccatgccgattatgccgtcgaacgttactaccgcgacgccaaaatcatggaaatcatcgaagggacatcgcaaattcatgaaatccaaattgcgatgaattacatgatggggagtgaagcatga

SEQ ID NO: 145 - DNA, 249 bp, Artificial Sequence
  source: /note=“Description of Artificial Sequence: Synthetic polynucleotide”
atgaaaaagcaagatatgaaagccgccattcgggaatttctttcacgctcattacgtgggcatacgttgaacgatgatgacgatattttttctctcgggcttgtccattcgttatttactgtgcaaatcatactgtttatagaaaaaaattttcaggttgagctggaagtgagtgagttgaaaacagaacagattgctaccgtcaataaaatagtggagctcattcagcgacaaacaggcctggagtaa

SEQ ID NO: 146 - DNA, 870 bp, Artificial Sequence
  source: /note=“Description of Artificial Sequence: Synthetic polynucleotide”
atgatgaacgtggcggtaataggtgcaggagtaatgggaactggcgtcgctcataacatggcgcaatacggcatatcaactaacgttgtggatatttctcaatctcagttggataaatgccgacagatgatcgaagcgaacttacgcttatataattttcacccgcagcataaaaaaaagacgcatagcacagcggagataatggaaaatatccgtttcacaactgagttggacgacattgtagaatgtgatttggtgattgagaatattactgaggatatagagaaaaaaaatgcactatacacacggatgaatacgatctgcggcgcgtcgacagtgtttggtgttaatacgtccgccatatctatcactgcgttgtcaaaattaatgcgacatccggagaatgtagtcggcgtccattttatgaacccggtaccgctgatgcacaccgtcgaattaatccgtggtgtacatactgcagagcgtacactgaacatatttcaccacttatttgctcagttgaataaaaccggcatcgtggtcaatgattctccgggctttgtcactaatcgtgccatgatgatttttgttaacgaagccatatttatggtgcaggagcagatcgcccgtgctgaggatatcgatacgttgtttaaaacctgcttcgggcacaagatggggccactacaaactgccgatttgattggcttggatacgattttgcaatcgttgcaggtgctatatgaaagttttaatgatgataagtatcgccccagctttttattaaagaaaatggtcgatgcagggtatttgggcgtgaaatcagggcaggggttttatcgctatcaacagacgtacgccgagcagtga

SEQ ID NO: 147 - DNA, 2601 bp, Artificial Sequence
  source: /note=“Description of Artificial Sequence: Synthetic polynucleotide”
atggaatacgcaagcgaaatgaacggcatggaaatcgccattattggtatggcggtccgtttcccgcagtcccggacgttacacgaattctggcataacatcgttcagggtaaagagtgcgtcaccttctttagcgaggaggagttgctggccgaaggcgtcgaacagagtactctggacaacccggcctatgtacgggccaagccctatatcgagggcatatgcgactttgatgctgcatttttcggttacagtcacaaagaggcgcagactctggatccgaaatcccgcgtattacatgaagttgcctaccatgcgctggaagatgcaggctatgcccaacgtaccagcgatctgatcaccggggtattcgtgggggcgtcagaagatgtggattggctacggcgttcgctgtcacagattggcggcgatgcgctgaatcgttttgagtctggcatctatggtcataaggatctgctcgcacatctcattgcctacagtctcaatctcaacggtccggtgtatagtctctacaccagttgttcgacttctctgagtgcaacgcatatcgcttgccgcagcttgttgtttggcgaatgtgatctggcgttggcgggtggaattactatcgatttaccgcagaagtcaggctacttctgtcaacagggcatgatccactccaccgatggccactgccgtccttttgacagtcaggcttctggcaccctgtttggcgatggcgcgggtgtggttgtgcttcggcgtttggaagatgctctggcagcgggcgatcgcatctatgcggtgatccggggtagcgcggtcaacaatgacggtaaacaaaaaatcggttttgtcgcgcctggtcatgagggacagaaggcggtcatttgtgcggcctgtcatctggcagaagtaagcccagaaagcatcggctacgttgaaacccacggtaccggcacccgtattggcgatcctatcgagttcgctgcgttgacggaggcgttcgatacttcacaccgccaatactgtgcactaggggctgtaaaggcgaatattggccacacccacgcggcggcgggcgtggctgggttaatcaagacggcgctggtacttcatcaccggaccattccgccgctcgccaactatcaaatgcctaactcgaagctggatctggcgcattcaccgttttatatcccgatacagccgcaggagtggcccgcatcgcggatgccgccccgcgctggcgtcagttcttttggcattggcggtactaatgtgcatatgattttggaagggctgaatcctgcggtgcgcgatgaccatgaccaagtgcgagcaccggtgtttatccctctctccgcgccgtctttcgagcagttggatgagctgacgcaacagcttaccccgttgctggctacccttgatgcgtcaacgctagcctacacacaacaagtggcgcgccccgtgtttgattgccgccgagtgatacaagtggaaaacgatggtacgcaagcgatgctggcatcgctggataacctaatgcccgacgctccttggggcctacactgtccagatctacgtactacgaacgattgtacttacgcacagtggctggcgcattcggcacattatcaacgcgaagcgactgctctgacggcattactcgacggcatgaatattccacccgcttattgccacgctgaaacgtgggcggcacaagcgaacagcagcctgctaatcagaggctgccagactatcgccgcgctaaaaacctggatgaacctgctaccgacattaacactgctgtcgggtgctggaacaggccttttgcctgccgctgcagccagtggcatgattgcgacgcaagacgtgttgcatttgctgtgggaaatggagcaaaaggcgcttcatctctggcttcctgagcgccatgaacctatccccggctacgtgctggcttggcaaggaaatcccatcaccgatgcacagcgtaacgacagagggttttggagtgaagcgctgttggcagatacccgagaactgggggagggcgttcacagtatcaactgggttaggctgccgccggaaataagagaagacgttgatgtattgcgctacgtggcgcaactgtggtgtgcaggtatcaatgtggattgggccgtgtggtacggcactccgctgccgcaacgcgggagcgcatcagcatatcccttcgcacacaaccactacccattgcctggacgggtaatgggtagtgtggagacccaacccgaagcaggacccgaaacgcaccacccttatcaggcacgtcccgtgttaagcgtacctttcgtagccgcacactcgcggggtatgcagtatatcacaggtctgatggaactgttgctggaaatatcgccggtcggggtggacgacgacttttttgaattaggcgggcattcgctgctggtgacgcaattgacctcccgtttagaacgcgatttcaacgtacatatcgatcttttgaccctgatggaaaaccccaatccgcgcaatatctacgcgcatatcgcggcgcaactggggggcgaagacaacctcgaaatagcctgtcagtaa

SEQ ID NO: 148 - DNA, 9621 bp, Artificial Sequence
  source: /note=“Description of Artificial Sequence: Synthetic polynucleotide”
atggataatacctctggagattttccatgtaataagatggacacgcgtaagcagttaccgctaacaccaagtcaacaggggtttttattccattccttaaaggataagaaaaggagtaactaccatgagcattttacatgcattttttctcagcatgtagatagcgcccacttcaagtgggcgctggaaacgttatttcgaaagcatgagtgttttcgcactgattataactgggagattgatgagcgcccttgtcaggtggtgaagaccgatgtgttgccggatatatatgtgttagactgtgagcaagaggaaatacgttttctactagctaatgatgacattatcattcctgtcccgcaggatgacggtattgatgctataattcctcaactgctacaggctgatttaaaatacccattttccttgaaaacgatcccagtccgggcctaccttattcagtcaacgaaagaaagtgcttttatactatcataccatcatattgtgatggatggctggagcttatcccttttcattaaacagttgctccaactctatggagcggctgtggtcagtggtgtgagggatgatagcgccattatcccctcatctctgaaaccccttgtagacacactgtcggcccgacgtcacacctttcagcacgactattgggctgcatatcttcgggagggaacaccaacttgtatcgtgccgctgtcacaatatcacacagatactgaagccgagaacaattcttacgttaatcaaacaaatcatgtggagatcaatttgtctccggatgtgtgtcagaaaatacagacgctatgcagcgattatcgtatcacccccgcagtaatcttctatgtggcctggggcatcctgctacaacgttggtgctatgctgacgatgtgttattcggcgcgacaatatcagggcgaaatataccaattgatggtatagaagaaacactagggctatttattaacacgttgccgctgcgtctgcgtgatgatggggcgacactgttgcaacacttacaacgtatgcaccaaacactaatagctcactacagcaatgaacatgatgccttagccagcatacaacggttggtacataaagaaggtcatgctggggatctttttaataccttagtggtgttggagaattatcccgttgatatgacattattgtcatgcgcgtcgcccgtggcaattcgccatctcagtgtacatgaacaaacgcactatccgctgaccttgaccatcactcaacagaagggattccgtttcagtattgcatatgcccttaactacctgaccaacaacatggcgcaagcgttgctgatgcacctgagttatctgcttgaacaactggtggacaatccgcagcgccccattgctgcgttggtaaacttgtccccatgccagcaggcgcaggtacttcaaccctatctggaacgcatggcatgccgggattgggatagtcaatccaacgtcatcgaacaatttcatcaagttgctgcgacttcgccagcacaggtcgcagtggttgatgaattgtgcgcgttgacctattcggagttggcagcacaagctgagcagctcgcggcttatttggtacagcagggtgttatggttggcgatactgtaggcataattagcgaacgccgggtaaacaccgtggttgccatcatcgccatcatgttgatcggggctgcttatgtgcccatcagccccgactacccagtgggtaggatgcaggaaattattgatgacagcggcttggcgctgttgctggtacatggcaaaccgctagatgcattaaacgttgcgcagagtgacctctgtgcatttcccgtcgcgccctcggtggtatttccggttatcacaccagattctcgcgcttatgtgatttattcatcggggtcgacgggtaagccaaaaggtatcgcggtggcgcaccgcggtttattgcgcctgatacaaggcgacagcccgctgaaggtggagagcggtgagacaacgctgctgacctgcccatttgagtttgatgtttcggtgtttgagatgtggtctaccttgctcaaccacggcaaactagtattactcagcaaacaggcattacttgatatcaatcacattcgccgcacgatcgctgatgaacaggtggcgcgcgcctggtttacctcatccttgtttaactcctatgtggcagaaggtgccgatttcttcggtatgttacaacacattacggtgggcggcgaagcagtatctgcgtggcatgtcaacgacgtgatgcaaaaatacccgcatctggtggtgacaaacggttatgggccgacggaaaacactatttttaccaccgcatatcgtttcaacgggttgcaacccgcccgagtcccgataggatacgcggtaccgggcacctcgctctacattaccgatctccatgggcatttgttgcctatcggtgccaccggtgaactagtggcgggtggagtgggggtcgccatcggctatcagaacaacccggcgctaagtgcgacggtttttgtccctgatccttttattcccggcggtatgatgtacaaaactggcgattatgcccggttgctggatgatggctgtgttgactgttttgggcgtaaagacggtcaaattaagatcaatggacaacgaattgaaaccggagagatcgagcagcgcctgctggagtgctccggcattatcgaggcggtagtggttccttaccgcgtacgtgaaacgctgcatattgcggcagtggtctgcgtcaatgatagctatgatgaggtggaagttcgagggcaattggctgacagattgccgccattcgctattccggaatccttggtggtggtgacggagattgcaaaaagccatagtggcaaggccgacttggcgcagttgcggtatctcctgcccgcaactcagtgcaacgccgtgtccaccacgatatcagaggtgcatagtgacatggaacatgcgctgcatgctatctggcaacgcgtacttgatagacaagacattgatagcaatgcctcctttttcgccctcggtggcacctcattggataccatcagggttaaaggggatattaagcggcaacttggcttggagattgatattaccgatctctttaagtacccaacgctcacggcgttagcacattttctcgatactgccgtatcgccggaggatgcaattccaacgcgtgctgttgtctacagcgacatgccggtggcgattgtcggtatggcgggacgtttccccggtgcggcgaatattgcagcgttgtggacgctggttgtaggaggggaatcgggattaacactgttcagtgatgaagagttgcgcgcgcatggtgtgacacctgacacgcttaaacaagcgaattatataaaaaccaaagggattgttgatgatcacgaatggtttgatgcggatttctttggttatactcccaacgaggcggaatgtatggatccgcaaattcgcttattgcatcagtgctgctggcaaactctggaacacgctgggtgcgatcctgccacctttactggtgcgattggcatttatgccggactgctgacatccccccactggcttaatgcggtaatgcaagacactaccgactctaccgccctgtacaaggccagtatcctgaatatccattccgtcacagcattgattgcccatgcgcttaacctcaccggccctgccgtgacgcttgacaccgcctgctccacttcggcagtggctatccaccaggcctgcatcgcactacgtaaccgggattgcgatgcggcattggcgggcggcgtttctatcgagatgcctgcgtaccggggctatgaatatcatgaaggcatgatcaatgcgcgagatggtgtctgtcgtcctttcgatagccaagcctctggcacagtcaccggtgatggcttaggaatgctgctgctaaaacgtctggacgacgcgctggcggatcgggactgcatctatggtgtgatcaaaggttcggcggtcaataatgatggcaataacaaaatcggctataccgcgccgagtgtgatcgggcaatcaacggtaatccgcaccagtttgcgccgtgccggttttgatagcgacagtatcggattggtggaagcgcacggcaccggtacggtgctgggtgatcctattgagttacgtgcgcttaatgaggtgtttggcccaacacctgttccgttttgtgtggtttcagcgctgaaaagtaatattggccacctcaactccgctgcgggagtggcgggcgtcatcaaaacaacgctcgccctgcatcatcaagtgttgccaccaacagcgcactttcgccaactcaatcctgctatagatctatcgcgttctgcattgtacgttaaccagcaagtccaaccgtggccgtcgacgcgtcctcgtcgtgcactggtgagctccttcggcatcggcggcaccaatgccagcatcgcactagaagcacatcaacatgaggacgacccttcagcgacgggggtacgcgacagctatctgttgttgttctccgctaaaacacccgctgcgttagagttgcgcgtggcctccacactggaatatgtcaagcatggagtaggggtgcgcctgccggatgtggcttatacattgcaaactggacgcacagcctttgaccatcggcgggcttatttggtgagtcgtgggtcgaaaatcgatctctcctgtgccacgatattgcaagcggaaatcttcaatggtcagcgcacgacagcggagatctgcttcatgtttcctggtcaaggtagccaatatcacggcatggccagcgcgctctatgctcatcaacccatgtttcgccagcacatggatcgctgctttgctgcattccaacgctattcgacggtcgatctcaaggcgttgttgtttgacgatgaggatacgcgggatattgatcaaacgcagttcacacaaccggcgttgttctgtgtcgaatacagcctagcgcgcaccttgattgatctggggattacgccggacagtatgatcgggcacagtctgggcgagtatgttgcggcctgtattgctggcgtatttactcttgaggatgcgctgcacgtcattgaggcgcgcggacgtttgatgcagtccatgcgtcccggtagcatgatggcggtctaccttagtcgcgaacagttgaccccatggctagctgcagaacggggtattgaactggcagctaataatagcgcgcatttttgcgtagtcgcgggcgagcaggcggccatttcccgtttgagcacacgcttagtcgagggcgggatacagcacaggcgcctgaaaacctctcacgcattccactcggccatgatgacgccgatgctgcacgattttgcacagttgctggggcaaatcccgatgcacgcgccgcacaagcgctttatatctaatgtaagcggtacatggattactgaggagcaagctacctcgccggattactgggtgcagcaagtgcgcaacgcggtgctgtttagcgaaggtgcggcgcaactgttggtacaacccacgctgtttatcgaatgtgggccgggtaatacgctctctacctttattcaaggacataaccaatacagcgatcagccgaccctgttgacgctacgcaaagccaacgcggcgatcgatgatgagcacatgttgcatcgtacgctggcggcgctgtgggtcaggggggagaatattgattggagacgctttaaccagacggcactcggcaagcacattccattgccggattaccccttcgaacagacttattactatcgctatggtgctgcactttccggttatcgccagtatccaaatcctctgcgccgtccgcaagatgagtggctccagcgtgtgctgtggcggatgcacgacacatccttgcgggaggcgttctatgcgccgggcgaattgatcatcattattagtgctgacggcgacaagttacagcagacgctgatgagtagtggtgtcgacagcatcacaatgccgctgccgatatcgtcagaggacgacgtgtgggataatgaccgtattctgacgcatttccacgacatctgcgcacttttagcacacaaaacctaccgacagttacattgcttgtatgctcccggtgcggaggcaggatcatcgttgacacagtcgctctcgggactttatcgtgttgctcgctggtgtatgcacagcaccacgccgctggcgtccttaacagtattgacccatggtgcgtttcgcgtacaggaagaggataatcccgaaccgacgctggcagcgttgtcgggcgcagtaaacgtgtttgcccaagagctgcacccgaccgaagtacgattgatcgatatcgatgcgcagagcagtgatgagaatctgaacttgctaacccagcgtctggccccgaaacaagaaacggtaatggcgctcaggcaggggatgctttacctgcgacgctttatcccgacacggctgttggcgcacttaccccctcaaacagggtgtataccgggcaacgtactgtggattatcggcggtgaaaaggggattggccgcatgatcggcgaagcgcttgctcagcgtgagggagtccgtgtggtactgagcagccgcacaggttatcaccatgaagcggtgcagcaggacgcattagacgttatccactgcgacgtgacgcaggcggaggcggttagagcttgtctggcaactctgctcgaacgctatggacggttggatggcgtgatttttgctgccgacgctaccaccacattgacactgcatcaattgagcgaatctgcgctacgcgacacgctaacggtgaaagaacggggtactgctaatgtgctgcatgcgttagcgcaacggaatctgctggatgagcgtctgctactgctgttctgtaactcgttggctgccgtgaatgcggagattggccagacaggctatgctaccgccagcgcctatttggatgcactggcacagcaactgcgtacccgctacaaggtgaatgcgctcagtatcgggttggatgcattgcgtgagcagggcatgttgttggatgctataaacggcagtgaatacgatgtcttgcgtggactgcgcccattgatgacgggaacgttgctacaggcttataaacaacaaggggctgacaccagctactacgcgcgattatcccccgagtccgattggctgctggatgagcaccggatatccggtatcgccacgctgccgggaaccggctatctggcgctggcgtatgaagctctgcgccattactttgtgcaagaccaaatctgcattgatgaattggtctttttggcaccgttgactgtgatggacaactgcagtgttgacgtttttgttgacatttcacctaacgggcaaggagttagtgtcgaggtgaaatcaatgacggagcgctttagtggcacgttaacaacccatgccagaggcagggcgacgcgtctgatggtagacgataatgttgtgtgcgatctcacggggctgatgcgcgagatgcacactatcactcctccaacaaaggaattatcgagcacgcacttccactatgggccgcgctggcacagcgtacaacaactgtatggcaataccgcccagactcaggttttcgcaacgctggccctgcccaccgttgccgctaatgatacgatcgcactgcaccctgcgctgttggacatcgccagcagtgttgtcgaacaactgcctggttttcatactgattcggtacctttcctttatcaggatttacgcctgtaccgcccgttgccgaacaccttacatgtggcgctgactgtcaatcggcacgatgaggagggtgacagctacgctttcacgctctacgacatggcaggcgagatggttgcccgctgtgcggcaatggtgaagcgcaaggtacagctccacatacaagatgtcgatgacgacacgcgactgcgcgtgcccagtgccgataactaccaactgcggctggccgctgagggggagggggcaggaaagctagcgttgtgccctacgccgcgcttagcgctgggggattcacaagtagagattgaggtactggccaccggactgaactttaaggatgtgctgttcaccacgggattgctccggcagcagccgggtgaggctccgctgcaattgggactggagtgcgccggacgcattactcgcgtgggtaaaaatgtcactgaatttgccccgggagaggatgttatggcggtgctaaacggtggttttgtccagtacgcacgggtagaaagcgattgcgtagtgagaaaaccagcccattgtcgcatcgaacaggcggctgcgctgcctatcgcatacctcaccgcctattacgcactggtggtgcgcgctaatttgcaacccggagaacgagtattgatccacagtgcggcggggggcgttggcttggctgcgctacatattgccaaacgctgcggagcacagattttcgccacagcaggtagcgagcagaaacgcgattacttgctttcgctaggcgtacatgccgtagctgattcacacgacgaacagttcgctgccactctgctgaccgcatcggacggacaggggatggatgtgatccttaactccctcacaggccgtctgcttgacgctagcctcgcgctgctggcaccgctgggccgttttcttgagctgggcagcaaggacattgtggaagacaaagcgctaccgatgcgtttcttcgcccaaggcggcacctttattccgattaattttcacgcggcgcatggtgcgtttagtcgctacctgcaacagattgtcgcttggatagatgataacacgctaccgctccttccatgcaaatccgtaccattgcccgaggttgcacgcgccttcgccaccctgaccacgccgcagcatattggcaaggtggtggtaacgcatcgcactgcggcaggcatggaccggctgaacgccatgatagcagaaaggcgcctcggcggctatgcgctcagcatgagcaatgccgaggtgatgcgccaattgtggccgatactgaacactcgcagtccgtgggcgcaactgctgctctcacctcgggcgatcgatagattagcgcggggcaaccgggtggatcgcggtgtaccgtctgccgctaacgatacgattactcagcagacagtgaaaaagcggccccgtccggaaattggcgtgccttacagccccgcgacacgtgaagtggaacgcgtgctctgccaaatcctggaagagtatctgggtctggatagagtaggcattgacgacaactatgccgaattaggggcaacctcactcgatatggtgcaactgagtgggcaaatggcgcgtcactatccgcaagtgagcgtggtgtcgctgtacaaccacgccaccgtgcgccagctggcgacgttttgccagcccccggagggcgagtcaaatgcgccatcaccacagcctgcagtacagacgaatacgcgcgcgaatcagatagcaaaacgtgctttgcaaattgcgaaaaatactgcgcgcagtcacacgtctttgcattaa

SEQ ID NO: 149 - DNA, 213 bp, Artificial Sequence
  source: /note=“Description of Artificial Sequence: Synthetic polynucleotide”
atggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaa

SEQ ID NO: 150 - DNA, 735 bp, Artificial Sequence
  source: /note=“Description of Artificial Sequence: Synthetic polynucleotide”
atgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattga

SEQ ID NO: 151 - DNA, 513 bp, Artificial Sequence
  source: /note=“Description of Artificial Sequence: Synthetic polynucleotide”
atggctgttccatcatcaaaagaagagttaattaaagctattaatagtaatttttctttattaaataagaagctagaatctattacgccccaactcgcctttgaacctctattggaagggcacgcgaaggggactacgattagcgtagcgaatctggtttcctatctgattggctggggagagctggtgttacactggcatgaccaagaggcaaaaggaaaaactattatttttcctgaggaaggatttaaatggaatgaattggggcgtttagcacagaaattctaccgtgactatgaggatattacagagtacgaagttttattggcacggttaaaggaaaataagcagcaactcgtggctttgattgaacgattcagtaacgacgagctttacggtaaaccttggtataataaatggacccgaggtcgtatgattcaatttaataccgcctcgccttataaaaatgcttcggggaggttaaataaactgcagaaatgtcttgcagaatag

SEQ ID NO: 152 - protein, 240 aa, Artificial Sequence
  source: /note=“Description of Artificial Sequence: Synthetic polypeptide”
MSNISLYCLPYSGGSAAMYYKWRSVLSDNITLRPLEPAGRGTRIRQPLCLTMVDAVADLYQQFVKHYTGGDYAIFGHSLGGIMAFELVHYILDHGHDMPCALFFSGCRPPDRASHEVILHTLPDQAFMEEIVKLGGTPVDVFRNKELMTIFTPIIKNDYRLYEQYVFQAKARTLTCPIVLFHGDADNLVMQDELLAWEKFTTRKTRTIIFPAADHFFVDKHFEQVVGYVNQTIESLEIVG

SEQ ID NO: 153 - protein, 504 aa, Artificial Sequence
  source: /note=“Description of Artificial Sequence: Synthetic polypeptide”
MTIMEHVSIKTLYHLLCCMLLFISAMCALAQEHEPIGAQDERLSTLIHQRMQEAKVPALSVSVTIKGVRQRFVYGVADVASQKANTLDTVYELGSMSKAFTGLVVQILIQEGRLRQGDDIITYLPEMRLNYQGKPASLTVADFLYHTSGLPFSTLARLENPMPGSAVAQQLRNENLLFAPGAKFSYASANYDVLGAVIENVTGKTFTEVIAERLTQPLGMSATVAVKGDEIIVNKASGYKLGFGKPVLFHAPLARNHVPAAYIHSTLPDMEIWIDAWLHRKALPATLREAMSNSWRGNSDVPLAADNRILYASGWFIDQNQGPYISHGGQNPNFSSCIALRPDQQIGIVALANMNSNLILQLCADIDNYLRIGKYADGAGDAITATDTLFVYLTLLLCFWGAVVVVRGAFRVYRATAHGPGKQQRLRLRVRDYIIALAVPGLVAAMLYVAPGILSPGLDWRFILVWGPSSVLAIPFGIILLAFVLTLNHQIKRILLHNKEWDDE

SEQ ID NO: 154 - protein, 819 aa, Artificial Sequence
  source: /note=“Description of Artificial Sequence: Synthetic polypeptide”
MAKDDFTCGSLDIAIIGMSGRFSGAESVPEWWDKLLAGEEFTQPTCTEDDNGNPWIRLRNIITAPYDFDAAFFNIPPGEALLMDPQQRIFLECCYNALEHAGYIPTQLKRVGVYGATYANNYFIDRVYPYLKMSGDHHYLQAQIGNEKDYLCAQVAYKLGFTGPAVSVQTACSSSLVAAYLACEGLLTFQADVALAGGVTLGFLQAHGYSPQGDKLVSQDGHCAPFSAEATGTVYSSGAGVVVLKRLEDALRDQDRVYAVIKGGAVNNDGGRRLGFVAPSVEGQVEAINTALAAAEVVPTDIALIETHGTGTPLGDEIELEALHRVFAPACAPHSIQLGAVKANLGHLGVASGIVSLMKTALTLYTGLVPPQINLVNKHKKLLQPASPFYLSDVVTSVPQTKRIHATVSSFGLGGTNAHLVLQNWCETPAQAVQENERRLFFFSAKTPLALRQQLDAHYHALATYAEADKDRIAYTLAQRRAHFPYRCALAADSVVALRASLAKLRDADMSFTPINMETTLVFLYPDRDDKLESALTHLLACQPNLRQRHQRLSQDVAQICEPADWTPALRQFIQQVSLSEWLIEQSISPVQHIGYLTGAAAAQYVARIISLENAVQQVIVAETTPEQTLAGNSELSEILANLAVTEGTLMLEIGRAGTFSILYHQHAQWVGQTVFSPMLNTDTPEDILPLLGTLWQRGVTICLPEMPAVQTIGLPGYSFDRVRYEIQSSDARENAMLPVSYLSVSDFVEKTWRSLLCIDHYDEHAVIFEYGATSMHVISFVDSCNHIYKIGLTAADIYARPAIREHSEFISECVDGIL

SEQ ID NO: 155 - protein, 1455 aa, Artificial Sequence
  source: /note=“Description of Artificial Sequence: Synthetic polypeptide”
MMSGNPLSWPQEQCHIIDQLYPYSAVNIIGGVVTIEGIVDLPRLHAAIQSAIRQFDALRMWFVMGEESEVVSQVQPYHWRDIRHLTFSPDYDKENLRPAAIETFVDEWFRQPFTLLAHDLFEFVTFTCGEQYSGYLFKAHHGIADGWSMALLSNHVKRAYEQQDVPDDASPAYSAFLAQQQSYQASTRFAVDRGWWRDYIDEYRDCFPDSSPIVTTEGISCSTWLEPAMINRLYRLCNRYGCTLNTLFIALFALYRARVWGEEKGVIGVPLANRHTREARRCFGMFTNQLPLAYRLVRTERFCERVAFFQRELKRGFKHSKYPITLFNQDLAEQGGGKLRAFDYCVNYYNFTYERHIAGAAQRVESYYSGEQSYKLQIVLQTVNNHKESLRLSLEALRSAFTPHQLTAMKNGLLDLVTALDRQPDARLGDLEVYPAPHVALACGSLKPSFTSRFAAQVVEHGDRTALIDNEQSLTYRQLDDAVERVARYLRQQGIGRGQVVGIIAEHSAQTVMVIYGILRCGAAFLPLNPALPTTRLYAMCRKAQVAHILYDPAMHELTQALAFPASSLLQALATSALAREPWPAIEPQDLAYVLFTSGSTGEPKGVQVSHGNLANYLHFAAERYFTAQDRAALYSSLSFDLTITTLFAPLCVGASISVCRHAESETLLRMAVVDQPNTVIKLTPAHLRLLCAAGISSEQIRTLVVGGEDFKRDLARKAAALFPQAVIYNEYGPTEATVGCMIYRYTGQETLPSLPIGMAIDGCQVAICSPWGCPVPEGETGELVIYGASVTQGYIDAPQQTAAAYLKDTNGVMIGYRSGDIGYAIAPNTLVYQGRKDDQVKINGYRIELCEIEQALLSAPQVESAAVAVIDDVQGQHSGLLACVTPSSVDVATVMQHLRQQLPTYMQPKQCCAIAQLPLSHNGKVDVRQMVATVRNTAPASGSERLGDAAIRHSVRVCVEGALEQTEFDDNENLYVLGLDSIKSIQIAAQLRHHGWTMSAVQVMECGTVNAICEFLASHTTVSQLAQYAHNTRIDLPALRWFTQLALPVPNVYNHVIVLKVLPGCPLEQLHNRLHTLIQQQPALHSALDAEGRLLVCDPNVCYPNEVLTEYSTAQWTLAEVIAQCNSMLDVTNGRVFTAALLHAPQPASSTLVLCAHHLCVDMHSWYLILSTLDAVSTVNGTSNSGLHRWNDYLASKTVDSATHESWRTVCQTLPLHFPPVSLPDDSLPRTRAWREDFRHPCVRRLFESSGNTAYSAETYVLTALALVLRYYSEEPWCRIEMEGMGRGCWPDEPDVADTVGWFTLFYPWAIPLHGDMATLLSAIASDLAKRTHGGGDYGLLQMRHAPEDSLAQGIRMNYIGVQAQPSLRYFHIDHFNSDIYTAPENALGCVLEFNIARSAADGLSFHCRFDPTRIALNDVQLLLARYKNSLTDLDAWLCQHSATLTGAPTLWTL

SEQ ID NO: 156 - protein, 479 aa, Artificial Sequence
  source: /note=“Description of Artificial Sequence: Synthetic polypeptide”
MTVNKLEPDNGTPNDELFTVAGMFDGSLYKLLLRMALPMFVGMLTQVTYAIADIFWLSHIDVTNSGIIAGVGLVFPVGMGLFAIANGIQIGMGSLLSRAIGMQRLDRAQRILSVGIIIALFFAIVITVLGYVYAQPLLRSLGATKSIIGYATEFYYYSLLTVFSIMLIGVMMGLFQGAGKIMVIMKASLLGALVNIMLDPIMIFVFDFGVKGVALASFLAQLSMVAYFIYTLMGLHIGLSIRIALRPFSWKIYREFLSVGMAQMLMQLIIAVGIVIYNFFIVRLDVNAMAAFTLTGRIDYFIITPMLAIATALLTVVGQNWGHGNVTRTLNAYWAAVALAFSIVLVLAVMHIVLAPWMYPLFTRVVAVSDYAVLQTRIMALALPFVAISLLASEYYQAIGKPWYSVLLTLMRHVFISVPVVYLLAIVLEMRITGVYFGAMSGTFVAALLAWRLLRLSPRLLRWNQEAVRSQHLDMEVAP

SEQ ID NO: 157 - protein, 487 aa, Artificial Sequence
  source: /note=“Description of Artificial Sequence: Synthetic polypeptide”
MSEQSYRSAGTLLAQLASGETTSVALVNHYFSRMAQFNKPLNAVVQQHYALALEAAARADRERLEGRARGVLHGLPCTVKESFDVQGWLTTSGAHYLKDNRATQDAPSIARLRAAGAILMGKTNVPMMTADWQTYNDLYGTTHNLWDRQRSPGGSSGGAAVAVAADFTPVEFGSDLFGSLRIPAHYTGVYAHRCSLGLMSVRGHVPGGGPQATDEPDLSTAGPMARSAADLRLMMRALSTFWVEPPRIPDFSRYQAKANYRVCTWFSAPHHEIDQQIAQRFQSFIDKLRAQPGVEVDDAMPADIDPDALFDIAVKLSGRLVSTALNGRQRLTAGLAALGFRLVGKLADVPEGITSYYQGMLKDSGEQRNTDKLRHEYSRVIETLFARYDVLLTPVSPVLAFAHMQQPVRKRKLIVNGEPQDYNEHLFWNMLATVFGLPATVYPLAKTMDELPCGIQIISGHFHDDVTINFAEFCESISGGFTVPEGY

SEQ ID NO: 158 - protein, 2154 aa, Artificial Sequence
  source: /note=“Description of Artificial Sequence: Synthetic polypeptide”
MTYSESDIAIVGMNCRYPGVHSVAAFETVLRTGCNILDPKVTPSNGHNHITLNNVYEHMAEFDANFFGYSRAEAEIMDPQQRVFLTCAWEMFEQSGYNPKQHDARVGLYAGVSTSFYLLTHLMNNPDKLAQLGGLQIMVGNDKDHLTSQLAYRLNITGPCVTVQASCATSLVAVHLACEGLLSGQCDMALAGGVTFRMEEQRSYESHGDGLQAEDGLIHTFDAQASGTVYSSGLGMVLLKRATDAQVQGDNILAVIKGSAINNDGGARSGYTVPGVDGQEAVMIEAHSLAEVTPQQIQYLELHGSGTPLGDAIEFAAIKRVFGTPAPNATPWRLGAVKPNVGHVEMASGITSLIKTVLSLTNRVFYPTLNFQRANPQLGLEDSPFEVVSRLTPWPEGTTPRTAGVSAFGLGGTNAHLVVQAPLSTPQARAQQMGPCVVVLSAKNHNALEQMQNALLAKLAAHPEIRLQDVAYTLRHGRFSAPVRKCVIAENCTQLARQLRDAPMVEATTGCTIYWRLGHRFVVALETLSDWLACSEVLSQAVGQLLEHFPLEPACLQDLSPAQRTFISQYALIALIDERETLNVVLCGDGDGGYAAAVLRGDCTLEQAWHRLNAGQPFDDVPTNPLLQPDVCSLMLDDAASDANRTALEALGQLWLAGVSLDWRWVDAAERMLGSQRIALPGTVFTPQRYWVEAVRPATFSHESSNNLLSRATKSDIIAVVTEIWERTLGVSIDDHHASFFELGGHSLLASTILYDIQQRYGITCTLSAFFADPTIEGLSCYLLEQGGSETAVSALPDTVFAPDQQHLPFPLTDVQQAYWVGRRKSLGLGNISTHIYVEYELQGLDETAFNRALNAVIARHSMLRAIVNDDGMQQILPNVPEYHVAFYTTQCEDAFQQRCRELRDTLSHQMIDCSRWPLFQMEVVVDPQQKARLHVSIDLLIADAWSLELFIRELAYHYRHPQAALPTLTYSFRDYVLTLKSYEKTPQFERARDYWRARIETLPPGPRLPLRTDPTKLENPTFVRRSYCLSRAIWQRLKTQAGQMSITPTTLLLTGFAQVLARFSSSPHFSLNLTLFNRLPLHADINHLIGDFTALTLLEIDMSQGETLQARANVIHSQLWRDLDNRLFGGIQVSRLLVQTHRDPAKSVIPIVFTSLLNQYEASWETDDTLFNQPQDDLYSISQTPQVWLDHQVMERNGELHFNWDVVEQLFEPALMDQMFQCYCQLLHALAQRPQLWHETQDVLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEIERCIARHPDVEQSVVVAVGNSQHRRLVAFAKLHDRHQAQALQAKEAEAAALAQGIIVNPAQRLAFKLKEPHIRALDGLGIALTAPADSTRYIKRRSYRHFSAQKTTLAQLGQLLSGLGQMRLPGLPFAKYAYASAGGLYPVQTYVYLHPDKIEEGVSGIYYFDPRQSCLMPVAPEVELNSGFHAGPNQSIADRAAFTLFMVADMAVISPFYGQEAAWHFSVMEAGTLCHLLEEDAPRYGLGLCQLGMADFSAVASHFQLSPHHRYVHCTVGGAIGQEAASAAALLRDFSTYEKPKETAAPLDMQSYKDAMLRGLRQQLPDYMVPSDLMLATDFPLTANGKLDRQKLQLQGEQIAHQRDGVGPIQVDSALQQRLVALWQEVLGVSHVSAEDDFFSLGGSSIELVRIQQALEAIIGQEIPIVDLFRLPTIADVARYLDEQLHNLPAAHDIVLAQAEVSQVSAARENLALRRKRAQQGEKGDE

SEQ ID NO: 159 - protein, 2166 aa, Artificial Sequence
  source: /note=“Description of Artificial Sequence: Synthetic polypeptide”
MTIHHAALARMLPAEKKEKLLRQLAQSGVSPSRIPIIKADPAQAIPLSFNQERLWFLQKYDSTATNYNLYVVYRLHGVVDMPMLTEALRHVQARHAILRTRIIVRNDRPCQVIDDASSLVLDTVTLAAQAPTSALDAVIQQVINTRFDLARGPLWGVTQIIQPDQGCHLVFCAHHIIIDGISLRLLFDELQQQYARLHAGNETSLPPPPLQYADYAFWQREWFQDTLLANELAYWRARLQDAPLLSTFPSLHPRPAQPSTHGSRFSITLDETLSLALKHVARTQETTPFVLMLTAFQLVLMRYAQQQRLVIGMPVSGRIRPELQSSIGYYASTAVIYTDFNGVEVGREALQRVKASVKETQGRQQLPFENLVNMLDLPRSLSHSPLFQILYIYHNHVTPRAFTLAGAYWEQVTYHNQTVKYDMTVEVFQNDATFDVSFEYDLGLYDADVVKQIAEALRQHCLSLTSSLETPIGAIPLHAPETATPRRDPLNATNVPWLGPQDVLRIIEQRCVQHPKQLAIQQHDGTLTYAELWARVQFIAMRFRAHGIQPGDRIGVLLPRHRDVIATMLATWFVGACYVPFDIHQPAARLQRLMQRARLVCLVVRQPGEWGEIVQLSLPELMQDMSNAIRYSTPCALLPDMQAYLLFTSGSTGEPKGVCVVHRGLLNLLLDMQRTFAVGSQDRLLSVTTPTFDISFLEYLLPLISGASLYLTEAERAADSFRMIPLIADYRPTLMQATPSFWHGLLMAGWRGDPELCVLAGGEALPTKVAEELLRCCGSLWNLYGPTETTIWSLKSQITQAENITLGAPIANTRIYILDNEGHPVPQGVDGELYIAGDGVAQGYDGQPELNAQFFLSEPGVPGGRMFRTGDLVRSDAQGQLFFVGRKDSQIKLRGYRIELGEIERTLARHPHVDAAVVACIERAPLHKALAAFIITSEPPSLFEQLKNELRQQLPDYMVPTLWQRVADFPNTDNGKIDRKRLAENFVADSSLVSPQTQALSDTEQMLLALWMRYLPIKNVDPECDFFRLGGHSLLAVTLVAEINRTFHCALTLKDIFHYSTLRALSARIAQQSITDAAASQDDWVIVHDPEHRHQPFPLTDVQRAYWLGRQTGATSIATHIYHEFDVEHFNVTRFTHAVNALIARHEMLRARVLPDGTQQILAQVPAYQLEQRDLSALSPNARNDALMAIRDRLSHHVHPADRWPLFDFSYSACTAQHGRLHFSLDLLIADALSMRTLQQELMMLYREPHVSLPLLPFSFRDYVQALLVEQASEAYARDQAYWQRALPQLYGPPTLPVQGDLAQLSAIRFVRRRHRLSAHNWGVLSALAQRTRITKTALLLTVFSQVLARWSLSPTFTLNLTLFNRPQGYPNAEAVIGDFTAVSLLNVCYDSQHSYAHNAQRIQVQLWEDLEHRRFSGIRASEALIHSGRFHAPMPVVFTSMLDIDGETTAQDPRDTTRFTLCPDANITQTPQVWLDHQVIELAGELHFNWDAVEQLFDTTLLDQMFGAYCHALQALVAMPQSWWGVNSSLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEVKSHLEQLDSVGSAAVVCHQGQLYAFITAAENLHPDDTDALLARVRAQLAVQLPYYLLPQHFFLLKVLPMTGNGKIDQAAMVQEVIQRMSQSTSQKSRALAHASPYEQQVAALWCEVLQREQIGLNDNFFEAGGGSIQIVLLHRRIEEIFKVTVPIAELFRLTTVKRIAGYLQAMQDNARAVNQTQQRDASRSRAQQRLVRRHQRQR

SEQ ID NO: 160 - protein, 1010 aa, Artificial Sequence
  source: /note=“Description of Artificial Sequence: Synthetic polypeptide”
MAENDFGIAIIGMAGRFPQADTVQAFWENLLASRECISFYSDEELLAMGISPEFVQHPDYVKAKGEVADIDKFDAAFFGIAPREAELMDPQHRVLLETAWAAFEDAGYVAADYPGDVGIFAGKSMDSYLMLNLMPHFKRVFSSGSLQAAIGNDKDSITTTIAYHLNLRGPAITVQTSSSTSLVAVCVACQSLLTWQCDMAIAGGVTLGPPAKTGYLSQEGGITAADGHCRAFSDNSSGFVPGTGAGLVVLKRVDEALRDGDNIYAVIKGFAVNNDGSEKISYTAPSVDAQARAIAQAQRLAGLTPQDITYVEAHGTGTRLGDPVEFSALSQAFAGASQKQYCALGSVKTNIGHLDTAAGVAGLIKTALAVQQGIIPATLHFERPNAQIDLTNSPFYINTTCQPWQPESGIRRAGVTSLGMGGTNAHVVLEQAPAVDLQARAPVPAYSILPFSAKTDSALSSGLARFADFLQHESLPDRRDLAWTLSQGRKAFAHRAALVTRDLHAAGTLLQQAATAPFARGVAQTQLGLGLLFSGQGSQYQRMGHQLYQVWPAYADAFDRCATLLEREYQLDIRHELFRAEVSLAQGERLAQTCLTQPLLFSVEYALAQLWLSWGITPTVMIGHSLGEWVAATLAGVFSLEDALRLVARRAELMHQAPSGAMLMVALPEAQIRALITAPLAIAAVNAPDYSVIAGPTSEILAVSQRLTEQNIINKRLHTSHAFHSSMMQDAAQALRQAFENVRLNPPTLTIISTVTGAHVSADTLTTPDYWIEQMLMPVQFSAALQEAQATFDVDFLEIGPGATLTQLTNGHALGDRLAFSSLPAGARSSDEHKHILDTVAALWVRGHNIDLSAFAGEQPRRVSLPTYAFDKIRYWVDSPEEQRSAVTPVADAGSVIPSEPSVRRQPRPAFSVPYAAPESKTQRGLVAICEALLGIDGLGIDDNFFEAGGHSLMLGMLLAQVQERFAVTLSFFDVMEDASVRALAQLVEQEQQDDGGSALAVLVNDMINE

SEQ ID NO: 161 - protein, 1598 aa, Artificial Sequence
  source: /note=“Description of Artificial Sequence: Synthetic polypeptide”
MEQQGIMRQLPTDDQTIVDYLYRIAGEYGEKAAVLMGDAALSYHDLNARSNQLAHYLRGLGIGEDRVVAIRLPRGMAMLIAIFAIVKAGGAYLPLAYNAPRSRIENILSNSGAVCLIGTDDGDRWPIPRVEIDSAAVSAMPTTDLRYRPHARQLAYIIYTSGSTGVPKGVATEHAALLNRIVWMQNAYPISSQDVLFQKTVYTFDVSVWEMFWWAMYGASVVLLPSGLESDPRTLARLIQRHRVSVVHFVPSMLNLFVEYLEMKQDPRLTASLRLVFSSGEKLTVHSVARFYQSVAQGDLINLYGPTEAAIDVSHHRCLRGYDYDDIPIGQAIDGCRLYVLDDHGNPVADGEEGELYLAGIGLARGYLNNVALTDRCFTIHPTLRHLGKPERLYKTGDLVWRDGESQQIHYIGRNDFQIKIRGLRVELGEIEAHAMRFPGVQQAVVVADQDDPDNQLIYAFVVSSVPLNLAALMDALSKNLPAYMLPNRLLAMSELPLSDNGKCCRKTLLDLARAYSASRVDLRETPAVRYLPLSSAQSSMWFMQQLAPHTALYNNPTALLLEGELDRTRMDGAIRQLMSRHTLLRAMAETHNGQPVLAVPQCVSSQALLTIVPLPSVSDDNALQAMINQRAAHPMPLTSGTPLCRFELLTLDDDRSVLLIHLHHIISDGWSKGVLLRELQAAYNGESLTPEPLLEYADYMEYQEEWRQSDAYQDAMRYWQNTLAGTLPILDIPTDQPRQKVARYQGAFVAFALSANTCERVLAAARAQRVSLYNYLLTAFVLLLHRNARQQEYIVGMPIAARLTKEQEHMIAPLVNVLPLRLPLDEAASFSELVQTIRGILFAAFRHQRLEFTDIVRAVNVDRSAGHFPIYQCMFQLDNMPLASPTLNGVNVTPLLLDTSASQVDISLSMQHIDGRITGTFEYDAGLYSADRIQHLVAQWRELLDEASSQPTQLVRDLIRFTPREHAWLARHNATEVALPPVDNLLALVLPHCQQRPTQVALRHADDAMTYGELQQATMQMCTWLRAQGVKRGESVALQLPFCFELIIAQLAILSLGASYVPLDGNAPAARNALILAQATPCMLLVAQPLESPHGLTIPWVLVPDWRSLLTEIPNLPVSVAPDALDCDAVVIFTSGTTGQPKGVRLSQRNLVNLTASFISSYQVTHQDVLLPITSVASASFVGEVLPLLAAGGTLVLAQKAQSLDSDALIALLASQRVTILSTTPSLSASLSVLAQSMGSLRLFLCGGEALEYEQIAPLLPHMAVVNGYGLTESGICSTYFPVAKRREQETGALPIGRPIQNTQAYVVDAYNRLVPPGACGELCFSGLGISPGYLDARQDPERFVELPEYPGVRVLKTGDRARWATDGMLFYLGRQDRQVQIRGYRVELGDIESLLKQHPDIADAWVDVRRNAAATPLLVAFYCSVNGVALDAQQLRVWLSLRLPLHMLPLLYVPLSAMPLGVNGKIDPQCLPLVDLRQLEGPGEYVPPATELEQRLAEIWQQLLGLERVGTTTNFFDLGGHSLLLVQMQQYIGQQCGQHVALVDLLRFTTIKRLAEFLLAPDAAQGTTGDQTQLRAAKQRLAFGHTRWAATTDSHH

SEQ ID NO: 162 - protein, 422 aa, Artificial Sequence
  source: /note=“Description of Artificial Sequence: Synthetic polypeptide”
MTKDVALMFPGSGSQYVGMARWLYERYPQVRTLFDEASQITERDMAALCLSGTLVQLAEPTAMALAIYTTSVAHFVAWQQFLAQTGVHVNLRYMLGHSLGEYAALTCSGALSFSQALALVAMRSRLASEIAREMDASTTIIKQGNQALVAAACEVAERETRQQVGIACFNSPQQFMLSGQNSAIIAAEQYLLDHDRQVEVVPLIGGVPYHSPLLKPCGQQLRKALDRCEWRRPCCPVISNVNAQPYPDTVTPVQWLEQQLSQPVQWQRSLTYLTGHLSPIAIEIGPQSVLKNLLLENRYPAPVYAFDNRHDRAQLALVLGDNMAVKTDPEAVRRQRITLLTNALTATRHHRAADVAASAQLKELLSRFFERIQQIEQRGTSSEEDIAFLHELLEQGFQLKGSSQAEIDACHARLASDNGGQA

SEQ ID NO: 163 - protein, 376 aa, Artificial Sequence
  source: /note=“Description of Artificial Sequence: Synthetic polypeptide”
MCTENYELAQQEAVLFAKQHLALAAQNIERQQFIVPDIISCVAQAGYLGASIPQKYGGRGYDSYQLCALHEVMAGVHGSLENLITVTGMVSTLLQRVGSAAQKAHYLPKLATGELIGAIALTEPNIGSDLVNVETELQQDGDGWRLNGKKKWITLGQIADFFIVLIHCGNQLATVLIDRNTDGFTITPLNDMLGLRGNMLAELHFNDCRLKEDALLGPLTPGVPLAVNFALNEGRFTTACGSLGLCRAAVDVAARYIRQRKQFKRRLFSHGIVQHLFATMLTQTRSAQLMCFSAAEYRETLHPAMINQILMAKYVASKAAVDVAGKAVQLLGANGCHADYAVERYYRDAKIMEIIEGTSQIHEIQIAMNYMMGSEA

SEQ ID NO: 164 - protein, 82 aa, Artificial Sequence
  source: /note=“Description of Artificial Sequence: Synthetic polypeptide”
MKKQDMKAAIREFLSRSLRGHTLNDDDDIFSLGLVHSLFTVQIILFIEKNFQVELEVSELKTEQIATVNKIVELIQRQTGLE

SEQ ID NO: 165 - protein, 289 aa, Artificial Sequence
  source: /note=“Description of Artificial Sequence: Synthetic polypeptide”
MMNVAVIGAGVMGTGVAHNMAQYGISTNVVDISQSQLDKCRQMIEANLRLYNFHPQHKKKTHSTAEIMENIRFTTELDDIVECDLVIENITEDIEKKNALYTRMNTICGASTVFGVNTSAISITALSKLMRHPENVVGVHFMNPVPLMHTVELIRGVHTAERTLNIFHHLFAQLNKTGIVVNDSPGFVTNRAMMIFVNEAIFMVQEQIARAEDIDTLFKTCFGHKMGPLQTADLIGLDTILQSLQVLYESFNDDKYRPSFLLKKMVDAGYLGVKSGQGFYRYQQTYAEQ

SEQ ID NO: 166 - protein, 866 aa, Artificial Sequence
  source: /note=“Description of Artificial Sequence: Synthetic polypeptide”
MEYASEMNGMEIAIIGMAVRFPQSRTLHEFWHNIVQGKECVTFFSEEELLAEGVEQSTLDNPAYVRAKPYIEGICDFDAAFFGYSHKEAQTLDPKSRVLHEVAYHALEDAGYAQRTSDLITGVFVGASEDVDWLRRSLSQIGGDALNRFESGIYGHKDLLAHLIAYSLNLNGPVYSLYTSCSTSLSATHIACRSLLFGECDLALAGGITIDLPQKSGYFCQQGMIHSTDGHCRPFDSQASGTLFGDGAGVVVLRRLEDALAAGDRIYAVIRGSAVNNDGKQKIGFVAPGHEGQKAVICAACHLAEVSPESIGYVETHGTGTRIGDPIEFAALTEAFDTSHRQYCALGAVKANIGHTHAAAGVAGLIKTALVLHHRTIPPLANYQMPNSKLDLAHSPFYIPIQPQEWPASRMPPRAGVSSFGIGGTNVHMILEGLNPAVRDDHDQVRAPVFIPLSAPSFEQLDELTQQLTPLLATLDASTLAYTQQVARPVFDCRRVIQVENDGTQAMLASLDNLMPDAPWGLHCPDLRTTNDCTYAQWLAHSAHYQREATALTALLDGMNIPPAYCHAETWAAQANSSLLIRGCQTIAALKTWMNLLPTLTLLSGAGTGLLPAAAASGMIATQDVLHLLWEMEQKALHLWLPERHEPIPGYVLAWQGNPITDAQRNDRGFWSEALLADTRELGEGVHSINWVRLPPEIREDVDVLRYVAQLWCAGINVDWAVWYGTPLPQRGSASAYPFAHNHYPLPGRVMGSVETQPEAGPETHHPYQARPVLSVPFVAAHSRGMQYITGLMELLLEISPVGVDDDFFELGGHSLLVTQLTSRLERDFNVHIDLLTLMENPNPRNIYAHIAAQLGGEDNLEIACQ

SEQ ID NO: 167 - protein, 3206 aa, Artificial Sequence
  source: /note=“Description of Artificial Sequence: Synthetic polypeptide”
MDNTSGDFPCNKMDTRKQLPLTPSQQGFLFHSLKDKKRSNYHEHFTCIFSQHVDSAHFKWALETLFRKHECFRTDYNWEIDERPCQVVKTDVLPDIYVLDCEQEEIRFLLANDDIIIPVPQDDGIDAIIPQLLQADLKYPFSLKTIPVRAYLIQSTKESAFILSYHHIVMDGWSLSLFIKQLLQLYGAAVVSGVRDDSAIIPSSLKPLVDTLSARRHTFQHDYWAAYLREGTPTCIVPLSQYHTDTEAENNSYVNQTNHVEINLSPDVCQKIQTLCSDYRITPAVIFYVAWGILLQRWCYADDVLFGATISGRNIPIDGIEETLGLFINTLPLRLRDDGATLLQHLQRMHQTLIAHYSNEHDALASIQRLVHKEGHAGDLFNTLVVLENYPVDMTLLSCASPVAIRHLSVHEQTHYPLTLTITQQKGFRFSIAYALNYLTNNMAQALLMHLSYLLEQLVDNPQRPIAALVNLSPCQQAQVLQPYLERMACRDWDSQSNVIEQFHQVAATSPAQVAVVDELCALTYSELAAQAEQLAAYLVQQGVMVGDTVGIISERRVNTVVAIIAIMLIGAAYVPISPDYPVGRMQEIIDDSGLALLLVHGKPLDALNVAQSDLCAFPVAPSVVFPVITPDSRAYVIYSSGSTGKPKGIAVAHRGLLRLIQGDSPLKVESGETTLLTCPFEFDVSVFEMWSTLLNHGKLVLLSKQALLDINHIRRTIADEQVARAWFTSSLFNSYVAEGADFFGMLQHITVGGEAVSAWHVNDVMQKYPHLVVTNGYGPTENTIFTTAYRFNGLQPARVPIGYAVPGTSLYITDLHGHLLPIGATGELVAGGVGVAIGYQNNPALSATVFVPDPFIPGGMMYKTGDYARLLDDGCVDCFGRKDGQIKINGQRIETGEIEQRLLECSGIIEAVVVPYRVRETLHIAAVVCVNDSYDEVEVRGQLADRLPPFAIPESLVVVTEIAKSHSGKADLAQLRYLLPATQCNAVSTTISEVHSDMEHALHAIWQRVLDRQDIDSNASFFALGGTSLDTIRVKGDIKRQLGLEIDITDLFKYPTLTALAHFLDTAVSPEDAIPTRAVVYSDMPVAIVGMAGRFPGAANIAALWTLVVGGESGLTLFSDEELRAHGVTPDTLKQANYIKTKGIVDDHEWFDADFFGYTPNEAECMDPQIRLLHQCCWQTLEHAGCDPATFTGAIGIYAGLLTSPHWLNAVMQDTTDSTALYKASILNIHSVTALIAHALNLTGPAVTLDTACSTSAVAIHQACIALRNRDCDAALAGGVSIEMPAYRGYEYHEGMINARDGVCRPFDSQASGTVTGDGLGMLLLKRLDDALADRDCIYGVIKGSAVNNDGNNKIGYTAPSVIGQSTVIRTSLRRAGFDSDSIGLVEAHGTGTVLGDPIELRALNEVFGPTPVPFCVVSALKSNIGHLNSAAGVAGVIKTTLALHHQVLPPTAHFRQLNPAIDLSRSALYVNQQVQPWPSTRPRRALVSSFGIGGTNASIALEAHQHEDDPSATGVRDSYLLLFSAKTPAALELRVASTLEYVKHGVGVRLPDVAYTLQTGRTAFDHRRAYLVSRGSKIDLSCATILQAEIFNGQRTTAEICFMFPGQGSQYHGMASALYAHQPMFRQHMDRCFAAFQRYSTVDLKALLFDDEDTRDIDQTQFTQPALFCVEYSLARTLIDLGITPDSMIGHSLGEYVAACIAGVFTLEDALHVIEARGRLMQSMRPGSMMAVYLSREQLTPWLAAERGIELAANNSAHFCVVAGEQAAISRLSTRLVEGGIQHRRLKTSHAFHSAMMTPMLHDFAQLLGQIPMHAPHKRFISNVSGTWITEEQATSPDYWVQQVRNAVLFSEGAAQLLVQPTLFIECGPGNTLSTFIQGHNQYSDQPTLLTLRKANAAIDDEHMLHRTLAALWVRGENIDWRRFNQTALGKHIPLPDYPFEQTYYYRYGAALSGYRQYPNPLRRPQDEWLQRVLWRMHDTSLREAFYAPGELIIIISADGDKLQQTLMSSGVDSITMPLPISSEDDVWDNDRILTHFHDICALLAHKTYRQLHCLYAPGAEAGSSLTQSLSGLYRVARWCMHSTTPLASLTVLTHGAFRVQEEDNPEPTLAALSGAVNVFAQELHPTEVRLIDIDAQSSDENLNLLTQRLAPKQETVMALRQGMLYLRRFIPTRLLAHLPPQTGCIPGNVLWIIGGEKGIGRMIGEALAQREGVRVVLSSRTGYHHEAVQQDALDVIHCDVTQAEAVRACLATLLERYGRLDGVIFAADATTTLTLHQLSESALRDTLTVKERGTANVLHALAQRNLLDERLLLLFCNSLAAVNAEIGQTGYATASAYLDALAQQLRTRYKVNALSIGLDALREQGMLLDAINGSEYDVLRGLRPLMTGTLLQAYKQQGADTSYYARLSPESDWLLDEHRISGIATLPGTGYLALAYEALRHYFVQDQICIDELVFLAPLTVMDNCSVDVFVDISPNGQGVSVEVKSMTERFSGTLTTHARGRATRLMVDDNVVCDLTGLMREMHTITPPTKELSSTHFHYGPRWHSVQQLYGNTAQTQVFATLALPTVAANDTIALHPALLDIASSVVEQLPGFHTDSVPFLYQDLRLYRPLPNTLHVALTVNRHDEEGDSYAFTLYDMAGEMVARCAAMVKRKVQLHIQDVDDDTRLRVPSADNYQLRLAAEGEGAGKLALCPTPRLALGDSQVEIEVLATGLNFKDVLFTTGLLRQQPGEAPLQLGLECAGRITRVGKNVTEFAPGEDVMAVLNGGFVQYARVESDCVVRKPAHCRIEQAAALPIAYLTAYYALVVRANLQPGERVLIHSAAGGVGLAALHIAKRCGAQIFATAGSEQKRDYLLSLGVHAVADSHDEQFAATLLTASDGQGMDVILNSLTGRLLDASLALLAPLGRFLELGSKDIVEDKALPMRFFAQGGTFIPINFHAAHGAFSRYLQQIVAWIDDNTLPLLPCKSVPLPEVARAFATLTTPQHIGKVVVTHRTAAGMDRLNAMIAERRLGGYALSMSNAEVMRQLWPILNTRSPWAQLLLSPRAIDRLARGNRVDRGVPSAANDTITQQTVKKRPRPEIGVPYSPATREVERVLCQILEEYLGLDRVGIDDNYAELGATSLDMVQLSGQMARHYPQVSVVSLYNHATVRQLATFCQPPEGESNAPSPQPAVQTNTRANQIAKRALQIAKNTARSHTSLH

SEQ ID NO: 168 - protein, 70 aa, Artificial Sequence
  source: /note=“Description of Artificial Sequence: Synthetic polypeptide”
MDKFKEKNPLSLRERQVLRMLAQGDEYSQISHNLNISINTVKFHVKNIKHKIQARNTNHAIHIANRNEII

SEQ ID NO: 169 - protein, 244 aa, Artificial Sequence
  source: /note=“Description of Artificial Sequence: Synthetic polypeptide”
MRIDILIGHTSFFHQTSRDNFLHYLNEEEIKRYDQFHFVSDKELYILSRILLKTALKRYQPDVSLQSWQFSTCKYGKPFIVFPQLAKKIFFNLSHTIDTVAVAISSHCELGVDIEQIRDLDNSYLNISQHFFTPQEATNIVSLPRYEGQLLFWKMWTLKEAYIKYRGKGLSLGLDCIEFHLTNKKLTSKYRGSPVYFSQWKICNSFLALASPLITPKITIELFPMQSQLYHHDYQLIHSSNGQN

SEQ ID NO: 170 - protein, 170 aa, Artificial Sequence
  source: /note=“Description of Artificial Sequence: Synthetic polypeptide”
MAVPSSKEELIKAINSNFSLLNKKLESITPQLAFEPLLEGHAKGTTISVANLVSYLIGWGELVLHWHDQEAKGKTIIFPEEGFKWNELGRLAQKFYRDYEDITEYEVLLARLKENKQQLVALIERFSNDELYGKPWYNKWTRGRMIQFNTASPYKNASGRLNKLQKCLAE

SEQ ID NO: 171 - protein, 532 aa, Photorhabdus luminescens
MKAKDVQPTIIINKNGLISLEDIYDIAIKQKKVEISTEITELLTHGREKLEEKLNSGEVIYGINTGFGGNANLVVPFEKIAEHQQNLLTFLGAGTGDYMSKPCIKASQFTMLLSVCKGWSATRPIVAQAIVDMINHDIVPLVPRYGSVGASGDLIPLSYIARALCGKGKVYYMGAEIDAAEAIKRAGLTPLSLKAKEGLALINGTRVMSGISAITVIKLEKLFKASISAIALAVEALLASHEHYDARIQQVKNHPGQNAVASALRNLLAGSTQVNLLSGVKEQANKACRHQEITQLNDTLQEVYSIRCAPQVLGIVPESLATARKILEREVISANDNPLIDPENGDVLHGGNFMGQYVARTMDALKLDIALIANHLHAIVALMMDNRFSRGLPNSLSPTPGMYQGFKGVQLSQTALVAAIRHDCAASGIHTIATEQYNQDIVSLGLHAAQDVLEMEQKLRNIVSMTILVACQAIHLRGNISEIAPETAKFYHAVREISSPLITDRALDEDIIRIADAIINDQLPLPEIMLEE